(19) United States
(12) Patent Application Publication
(10) Pub. No.: US 2003/0064411 A1
(43) Pub. Date: Apr. 3, 2003

(54) Nucleic acid molecules, polypeptides and uses therefor, including diagnosis and treatment of Alzheimer's disease

(76) Inventors: Herath Mudiyanselage Athula Chandrasiri Herath, Abingdon (GB); Rajesh Bhikhu Parekh, Near Wendlebury (GB); Christian Rohlff, Oxford (DE)

Correspondence Address: PENNIE AND EDMONDS, 1155 AVENUE OF THE AMERICAS, NEW YORK, NY, 100362711

(21) Appl. No.: 10/014,340

(22) Filed: Dec. 10, 2001

Related U.S. Application Data
(60) Provisional application No. 60/254,431, filed on Dec. 8, 2000.

(51) Int. Cl. G01N 33/53; G01N 33/567; G06F 19/00; G01N 33/48; G01N 33/50

(52) U.S. Cl. 435/7.2; 702/19

(57) ABSTRACT

The present invention provides methods and compositions for screening, diagnosis and prognosis of Alzheimer's disease, for monitoring the effectiveness of Alzheimer's disease treatment and for drug development. Alzheimer's disease-Associated Features (ADFs) detectable by two-dimensional electrophoresis of brain tissue are described. The invention further provides Alzheimer's disease-Associated Protein Isoforms (ADPIs) detectable in brain tissue, preparations comprising isolated ADPIs, antibodies specific for ADPIs and kits comprising the aforesaid.

### 1. INTRODUCTION

The present invention relates to the identification of proteins and protein isoforms that are associated with Alzheimer's disease and its onset and development, and of genes and nucleic acid molecules, encoding the same, and to their use for e.g., clinical screening, diagnosis, treatment, as well as for drug screening and drug development.

### 2. BACKGROUND OF THE INVENTION

Alzheimer's Disease (AD) is an increasingly prevalent form of neurodegeneration that accounts for approximately 50%-60% of the overall cases of dementia among people over 65 years of age. It currently affects an estimated 15 million people worldwide and owing to the relative increase of elderly people in the population its prevalence is likely to increase over the next 2 to 3 decades. Alzheimer's disease is a progressive disorder with a mean duration of around 8.5 years between onset of clinical symptoms and death. Death of pyramidal neurons and loss of neuronal synapses in brains regions associated with higher mental functions results in the typical symptomology, characterized by gross and progressive impairment of cognitive function (Francis et al., 1999, J. Neurol. Neurosurg. Psychiatry 66:137-47). Currently, a diagnosis of Alzheimer's disease requires a careful medical history and physical examination; a detailed neurological and psychiatric examination; laboratory blood studies to exclude underlying metabolic and medical illnesses that masquerade as AD; a mental status assessment and formal cognitive tests; and a computed tomographic scan or magnetic resonance image of the brain (Growdon, J H., 1995, Advances in the diagnosis of Alzheimer's disease. In: Iqbal, K., Mortimer, J A., Winblad, B., Wisniewski, H M eds Research Advances in Alzheimer's Disease and Related Disorders. New York, N.Y.: John Wiley & Sons Inc. 1995:139-153). Due to the time consuming nature of these tests, their expense, and their inconvenience to patients, it would be highly desirable to measure a substance or substances in body samples, such as samples of tissue, cerebrospinal fluid (CSF), blood or urine, that would lead to a positive diagnosis of Alzheimer's disease or that would help to exclude AD from the differential diagnosis. Since the CSF bathes the brain, changes in its protein composition may most accurately reveal alterations in brain protein expression patterns that are causatively or diagnostically linked to the disease.

Current candidate biomarkers for Alzheimer's disease include: (1) mutations in presenilin 1 (PS1), presenilin 2 (PS2) and amyloid precursor protein (APP) genes; (2) the detection of alleles of apoplipoprotein E (ApoE); and (3) altered concentrations of amyloid -peptides (A), tau protein, and neuronal thread protein (NTP) in the CSF. See, e.g., Neurobiology of Aging 19:109-116 (1998) for a review. Mutations in PS1, PS2 and APP genes are indicative of early-onset familial Alzheimer's disease. However, early-onset familial Alzheimer's disease is relatively rare; only 120 families worldwide are currently known to carry deterministic mutations (Neurobiology of Aging 19:109-116 (1998)). The detection of the e4 allele of ApoE has been shown to correlate with late-onset and sporadic forms of Alzheimer's disease. However, e4 alone cannot be used as a biomarker for Alzheimer's disease, since e4 has been detected in many individuals not suffering from Alzheimer's disease and the absence of e4 does not exclude Alzheimer's disease from the diagnosis (Neurobiology of Aging 19:109-116 (1998)).

A decrease in the A peptide A 42 and an increase in tau protein in the CSF of Alzheimer's disease have been shown to correlate with the presence of Alzheimer's disease (Neurobiology of Aging 19:109-116 (1998)). However, the specificity and sensitivity of A 42 and tau protein as biomarkers of Alzheimer's disease are limited. For example, it has been difficult to determine a cutoff level of CSF tau protein that is diagnostically informative. Also, elevated levels of NTP in the CSF of postmortem subjects have been shown to correlate with the presence of Alzheimer's disease (Neurobiology of Aging 19:109-116 (1998)). Therefore, a need exists to identify sensitive and specific biomarkers for the diagnosis of Alzheimer's disease in living subjects.

Therefore, a need exists to identify sensitive and specific biomarkers for the diagnosis of Alzheimer's disease in living subjects. Additionally, there is a clear need for new therapeutic agents for Alzheimer's disease that work quickly, potently, specifically and with fewer side effects.

### 3. SUMMARY OF THE INVENTION

The present invention provides methods and compositions for screening, diagnosis and treatment of Alzheimer's disease and for screening and development of drugs for treatment of Alzheimer's disease.

A first aspect of the invention provides methods for identification of Alzheimer's disease that comprise analyzing a sample of brain tissue by two-dimensional electrophoresis to detect the presence or level of at least one Alzheimer's Disease-Associated Feature (ADF), e.g., one or more of the ADFs disclosed herein, or any combination thereof. These methods are also suitable for clinical screening, prognosis, monitoring the results of therapy, for identifying patients most likely to respond to a particular therapeutic treatment, drug screening and development, and identification of new targets for drug treatment.

A second aspect of the invention provides methods for diagnosis of Alzheimer's disease that comprise detecting in a sample of brain tissue the presence or level of at least one Alzheimer's Disease-Associated Protein Isoform (ADPI), e.g., one or more of the ADPIs disclosed herein or any combination thereof.

A third aspect of the invention provides antibodies, e.g., monoclonal and polyclonal and chimeric (bispecific) antibodies capable of immunospecific binding to a ADPI, e.g., a ADPI disclosed herein.

A fourth aspect of the invention provides a preparation comprising an isolated ADPI, i.e., a ADPI substantially free from proteins or Protein Isoforms having a significantly different isoelectric point or a significantly different apparent molecular weight from the ADPI.

A fifth aspect of the invention provides kits that may be used in the above recited methods and that may comprise single or multiple preparations, or antibodies, together with other reagents, labels, substrates, if needed, and directions for use. The kits may be used for diagnosis of disease, or may be assays for the identification of new diagnostic and/or therapeutic agents.

A sixth aspect of the invention provides methods of treating Alzheimer's disease, comprising administering to a subject a therapeutically effective amount of an agent that modulates (e.g., upregulates or downregulates) the expression or activity (e.g. enzymatic or binding activity), or both, of a ADF or a ADPI in subjects having Alzheimer's disease.

A seventh aspect of the invention provides methods of screening for agents that modulate (e.g., upregulate or downregulate) a characteristic of, e.g., the expression or the enzymatic or binding activity, of a ADF, a ADPI, a ADPI analog, or a ADPI-related polypeptide.

Each of the above and below described aspects can also directed to the treatment, diagnosis, prognosis of a neuropsychiatric or neurological diseases or conditions, and compositions therefor. Such diseases or conditions include disturbances in structure or function of the central nervous system resulting from developmental abnormality, injury or toxin, or a mental illness arising from the same. Such disorders include, without limitation, dementing illnesses such as Alzheimer's disease, vascular dementia and Lewy body dementia, as well as schizophrenia, Parkinson's disease, multiple sclerosis, and depression.

Other objects and advantages will become apparent from a review of the ensuing detailed description taken in conjunction with the following illustrative drawing.

### 4. BRIEF DESCRIPTION OF THE FIGURES

FIG. 1 is an image obtained from 2-dimensional electrophoresis of normal tissue, which has been annotated to identify ten landmark features, designated BR1 to BR10;

FIG. 2 shows nucleic acid sequence of ADPI-41 (FIG. 2a) and the corresponding amino acid sequence (FIG. 2b)where the tryptic peptides identified by mass spectrometry are underlined, and the conserved motifs are in italics;

FIG. 3 shows the nucleic acid sequence (FIG. 3a) and the corresponding amino acid sequence (FIG. 3b) of the splice variant identified for ADPI-41. The protein sequence (FIG. 3b) shows in bold the amino acids unique to this clone, the tryptic digest peptides identified by mass spectroscopy are underlined and the conserved motifs are in italics; and

FIG. 4 is a flow chart depicting the characterization of a Feature and relationship of a Feature and Protein Isoform. A Feature may be further characterized as or by a Protein Isoform having a particular peptide sequence associated with its pI and MW. As depicted herein, a Feature may comprise one or more Protein Isoform(s), which have indistinguishable pI and MWs using the Preferred Technology, but which have distinct peptide sequences. The peptide sequence of the Protein Isoform can be utilized to search database(s) for previously identified proteins comprising such peptide sequence. In some instances, it can be ascertained whether a commercially available antibody exists which may recognize the previously-identified protein and/or a variant thereof. It should be noted that the ADPI may either correspond to the previously-identified protein, or be a variant of the previously-identified protein.

### 5. DETAILED DESCRIPTION OF THE INVENTION

The present invention described in detail below provides methods, compositions and kits useful, e.g., for screening, diagnosis and treatment of Alzheimer's disease in a mammalian subject, and for drug screening and drug development. The invention also encompasses the administration of therapeutic compositions to a mammalian subject to treat or prevent Alzheimer's disease. The mammalian subject may be a non-human mammal, but is preferably human, more preferably a human adult, i.e. a human subject at least 21 (more particularly at least 35, at least 50, at least 60, at least 70, or at least 80) years old. For clarity of disclosure, and not by way of limitation, the invention will be described with respect to the analysis of brain tissue samples, which may be taken from a subject at risk of having or developing Alzheimer's disease. However, as one skilled in the art will appreciate, based on the present description the assays and techniques described below can be applied to other types of samples, including blood, serum, plasma, or saliva. The methods and compositions of the present invention are useful, such as for example, screening, diagnosis and treatment of a living subject, but may also be used for postmortem diagnosis in a subject, for example, to identify family members of the subject who are at risk of developing the same disease.

Each of the above and below described aspects can also be directed to the treatment, diagnosis, prognosis of a neuropsychiatric or neurological diseases or conditions, and compositions therefor. Such diseases or conditions include disturbances in structure or function of the central nervous system resulting from developmental abnormality, injury or toxin, or a mental illness arising from the same. Such disorders include, without limitation, dementing illnesses such as Alzheimer's disease, vascular dementia and Lewy body dementia, as well as schizophrenia, Parkinson's disease, multiple sclerosis, and depression.

The following definitions are provided to assist in the review of the instant disclosure.

5.1. Definitions

“Feature” refers to a spot detected in a 2D gel, and the term “Alzheimer's Disease-Associated Feature” (ADF) refers to a feature that is differentially present in a sample from a subject having Alzheimer's disease compared with a sample from a subject free from Alzheimer's disease. A feature or spot detected in a 2D gel is characterized by its isoelectric point (pI) and molecular weight (MW), preferably as determined by 2D gel electrophoresis, particularly utilizing the Preferred Technology described herein. As used herein, a feature is “differentially present” in a first sample with respect to a second sample when a method for detecting the said feature (e.g., 2D electrophoresis) gives a different signal when applied to the first and second samples. An ADF (or a Protein Isoform, i.e. ADPI, as defined infra) is “increased” in the first sample with respect to the second if the method of detection indicates that the ADF, or ADPI, is more abundant in the first sample than in the second sample, or if the ADF, or ADPI, is detectable in the first sample and substantially undetectable in the second sample. Conversely, a ADF, or ADPI, is “decreased” in the first sample with respect to the second if the method of detection indicates that the ADF, or ADPI, is less abundant in the first sample than in the second sample or if the ADF, or ADPI, is undetectable in the first sample and detectable in the second sample.

Particularly, the relative abundance of a feature in two samples may be determined in reference to its normalized signal, in two steps. First, the signal obtained upon detecting the feature in a sample is normalized by reference to a suitable background parameter, e.g., (a) to the total protein in the sample being analyzed (e.g., total protein loaded onto a gel); (b) to an Expression Reference Feature (ERF) i.e., a feature whose abundance is substantially invariant, within the limits of variability of the Preferred Technology, in the population of subjects being examined, e.g. the ERFs disclosed below, or (c) more preferably to the total signal detected as the sum of each of all proteins in the sample. Secondly, the normalized signal for the feature in one sample or sample set is compared with the normalized signal for the same feature in another sample or sample set in order to identify features that are “differentially present” in the first sample (or sample set) with respect to the second.

“Fold change” includes “fold increase” and “fold decrease” and refers to the relative increase or decrease in abundance of a ADF or the relative increase or decrease in expression or activity of a polypeptide (e.g. a ADPI, as defined infra.) in a first sample or sample set compared to a second sample (or sample set). An ADF or polypeptide fold change may be measured by any technique known to those of skill in the art, albeit the observed increase or decrease will vary depending upon the technique used. Preferably, fold change is determined herein as described in the Examples infra.

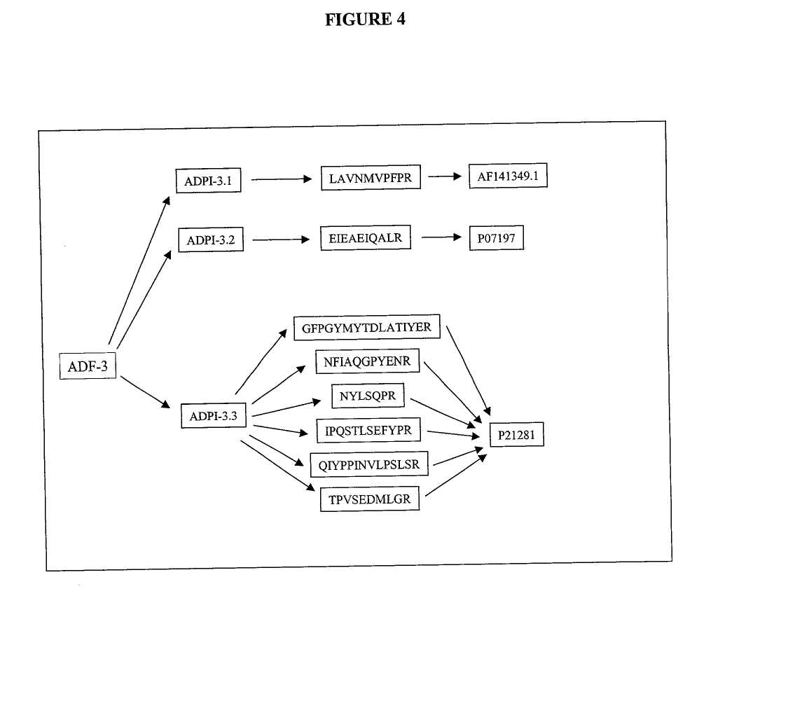

“Alzheimer's Disease-Associated Protein Isoform” (ADPI) refers to a protein that is differentially present in a sample from a subject having Alzheimer's Disease compared with a sample from a subject free from Alzheimer's Disease, or that is differentially present in a sample from a subject having one or more particular Alzheimer's Disease compared with a sample from a subject free from such one or more particular Alzheimer's Disease or having a distinct Alzheimer's Disease. As used herein, an ADPI is “differentially present” in a first sample with respect to a second sample when a method for detecting the said feature, (e.g., 2D electrophoresis or immunoassay) gives a different signal when applied to the first and second samples (refer to ADF definition). An ADPI is characterised by one or more peptide sequences of which it is comprised, and further by a pI and MW, preferably determined by 2D electrophoresis, particularly utilising the Preferred Technology as described herein. Typically, ADPIs are identified or characterised by the amino acid sequencing of ADFs (FIG. 4).

FIG. 4 is a flow chart depicting the characterization of an ADF and relationship of a ADF and ADPI(s). An ADF may be further characterized as, or by, an ADPI having a particular peptide sequence associated with its pI and MW. As depicted herein, a ADF may comprise one or more ADPI(s), which have indistinguishable pIs and MWs using the Preferred Technology, but which have distinct peptide sequences. The peptide sequence(s) of the ADPI can be utilized to search database(s) for previously-identified proteins comprising such peptide sequence(s). In some instances, it can be ascertained whether a commercially available antibody exists which may recognize the previously-identified protein and/or a variant thereof. It should be noted that the ADPI may either correspond to the previously-identified protein, or be a variant of the previously-identified protein.

“Variant” as used herein refers to a polypeptide which is a member of a family of polypeptides that are encoded by a single gene or from a gene sequence within a family of related genes and which differ in their pI or MW, or both. Such variants can differ in their amino acid composition (e.g. as a result of alternative mRNA or premRNA processing, e.g. alternative splicing or limited proteolysis) and in addition, or in the alternative, may arise from differential post-translational modification (e.g., glycosylation, acylation, phosphorylation).

“Modulate” in reference to expression or activity of a ADF, ADPI or a ADPI-related polypeptide refers to any change, e.g., upregulation or downregulation, increase or decrease, of the expression or activity of the ADF, ADPI or a ADPI-related polypeptide. Those skilled in the art, based on the present disclosure, will understand that such modulation can be determined by assays known to those of skill in the art.

“ADPI analog” refers to a polypeptide that possesses similar or identical function(s) as a ADPI but need not necessarily comprise an amino acid sequence that is similar or identical to the amino acid sequence of the ADPI, or possess a structure that is similar or identical to that of the ADPI. As used herein, an amino acid sequence of a polypeptide is “similar” to that of a ADPI if it satisfies at least one of the following criteria: (a) the polypeptide has an amino acid sequence that is at least 30% (more preferably, at least 35%, at least 40%, at least 45%, at least 50%, at least 55%, at least 60%, at least 65%, at least 70%, at least 75%, at least 80%, at least 85%, at least 90%, at least 95% or at least 99%) identical to the amino acid sequence of the ADPI; (b) the polypeptide is encoded by a nucleotide sequence that hybridizes under stringent conditions to a nucleotide sequence encoding at least 5 amino acid residues (more preferably, at least 10 amino acid residues, at least 15 amino acid residues, at least 20 amino acid residues, at least 25 amino acid residues, at least 40 amino acid residues, at least 50 amino acid residues, at least 60 amino residues, at least 70 amino acid residues, at least 80 amino acid residues, at least 90 amino acid residues, at least 100 amino acid residues, at least 125 amino acid residues, or at least 150 amino acid residues) of the ADPI; or (c) the polypeptide is encoded by a nucleotide sequence that is at least 30% (more preferably, at least 35%, at least 40%, at least 45%, at least 50%, at least 55%, at least 60%, at least 65%, at least 70%, at least 75%, at least 80%, at least 85%, at least 90%, at least 95% or at least 99%) identical to the nucleotide sequence encoding the ADPI. As used herein, a polypeptide with “similar structure” to that of a ADPI refers to a polypeptide that has a similar secondary, tertiary or quarternary structure as that of the ADPI. The structure of a polypeptide can determined by methods known to those skilled in the art, including but not limited to, X-ray crystallography, nuclear magnetic resonance, and crystallographic electron microscopy.

“ADPI fusion protein” refers to a polypeptide that comprises (i) an amino acid sequence of a ADPI, a ADPI fragment, a ADPI-related polypeptide or a fragment of a ADPI-related polypeptide and (ii) an amino acid sequence of a heterologous polypeptide (i.e., a non-ADPI, non-ADPI fragment or non-ADPI-related polypeptide).

“ADPI homolog” refers to a polypeptide that comprises an amino acid sequence similar to that of a ADPI but does not necessarily possess a similar or identical function as the ADPI.

“ADPI ortholog” refers to a non-human polypeptide that (i) comprises an amino acid sequence similar to that of a ADPI and (ii) possesses a similar or identical function to that of the ADPI.

“ADPI-related polypeptide” refers to a ADPI homolog, a ADPI analog, a variant of ADPI, a ADPI ortholog, or any combination thereof.

“Chimeric Antibody” refers to a molecule in which different portions are derived from different animal species, such as those having a human immunoglobulin constant region and a variable region derived from a murine mAb. (See, e.g., Cabilly et al., U.S. Pat. No. 4,816,567; and Boss et al., U.S. Pat. No. 4,816,397, which are incorporated herein by reference in their entirety.)

“Derivative” refers to a polypeptide that comprises an amino acid sequence of a second polypeptide that has been altered by the introduction of amino acid residue substitutions, deletions or additions. The derivative polypeptide possesses a similar or identical function as the second polypeptide.

“Fragment” refers to a peptide or polypeptide comprising an amino acid sequence of at least 5 amino acid residues (preferably, at least 10 amino acid residues, at least 15 amino acid residues, at least 20 amino acid residues, at least 25 amino acid residues, at least 40 amino acid residues, at least 50 amino acid residues, at least 60 amino residues, at least 70 amino acid residues, at least 80 amino acid residues, at least 90 amino acid residues, at least 100 amino acid residues, at least 125 amino acid residues, at least 150 amino acid residues, at least 175 amino acid residues, at least 200 amino acid residues, or at least 250 amino acid residues) of the amino acid sequence of a second polypeptide. The fragment of a ADPI may or may not possess a functional activity of the second polypeptide.

The “percent identity” of two amino acid sequences or of two nucleic acid sequences can be or is generally determined by aligning the sequences for optimal comparison purposes (e.g., gaps can be introduced in either sequence for best alignment with the other sequence) and comparing the amino acid residues or nucleotides at corresponding positions. The “best alignment” is an alignment of two sequences that results in the highest percent identity. The percent identity is determined by the number of identical amino acid residues or nucleotides in the sequences being compared (i.e., % identity=# of identical positions/total# of positions×100).

The determination of percent identity between two sequences can be accomplished using a mathematical algorithm known to those of skill in the art. An example of a mathematical algorithm for comparing two sequences is the algorithm of Karlin and Altschul (1990) Proc. Natl. Acad. Sci. USA 87:2264-2268, modified as in Karlin and Altschul (1993) Proc. Natl. Acad. Sci. USA 90:5873-5877. The NBLAST and XBLAST programs of Altschul, et al. (1990) J. Mol. Biol. 215:403-410 have incorporated such an algorithm. BLAST nucleotide searches can be performed with the NBLAST program, score=100, wordlength=12 to obtain nucleotide sequences homologous to a nucleic acid molecules of the invention. BLAST protein searches can be performed with the XBLAST program, score=50, wordlength=3 to obtain amino acid sequences homologous to a protein molecules of the invention. To obtain gapped alignments for comparison purposes, Gapped BLAST can be utilized as described in Altschul et al. (1997) Nucleic Acids Res. 25:3389-3402. Alternatively, PSI-Blast can be used to perform an iterated search which detects distant relationships between molecules (Id.). When utilizing BLAST, Gapped BLAST, and PSI-Blast programs, the default parameters of the respective programs (e.g., XBLAST and NBLAST) can be used. See http://www.ncbi.nlm.nih.gov.

Another example of a mathematical algorithm utilized for the comparison of sequences is the algorithm of Myers and Miller, CABIOS (1989). The ALIGN program (version 2.0) which is part of the GCG sequence alignment software package has incorporated such an algorithm. Other algorithms for sequence analysis known in the art include ADVANCE and ADAM as described in Torellis and Robotti (1994) Comput. Appl. Biosci., 10 :3-5; and FASTA described in Pearson and Lipman (1988) Proc. Natl. Acad. Sci. 85:2444-8. Within FASTA, ktup is a control option that sets the sensitivity and speed of the search.

“Diagnosis” refers to diagnosis, prognosis, monitoring, characterizing, selecting patients, including participants in clinical trials, and identifying patients at risk for or having a particular disorder or those most likely to respond to a particular therapeutic treatment, or for assessing or monitoring a patient's response to a particular therapeutic treatment.

“Treatment” refers to therapy, prevention and prophylaxis and particularly refers to the administration of medicine or the performance of medical procedures with respect to a patient, for either prophylaxis (prevention) or to cure the infirmity or malady in the instance where the patient is afflicted.

“Agent” refers to all materials that may be used to prepare pharmaceutical and diagnostic compositions, or that may be compounds, nucleic acids, polypeptides, fragments, isoforms, variants, or other materials that may be used independently for such purposes, all in accordance with the present invention.

“Brain tissue” refers to homogenate brain samples of anatomically-defined areas of the brain.

5.2 Alzheimer's Disease-Associated Features (ADFs)

In one aspect of the invention, two-dimensional electrophoresis is used to analyze brain tissue from a subject, preferably a living subject, in order to detect or quantify the expression of one or more Alzheimer's Disease-Associated Features (ADFs) for screening, treatment or diagnosis of Alzheimer's disease.

By way of example and not of limitation, using the Preferred Technology, a number of samples from subjects having Alzheimer's disease and samples from subjects free from Alzheimer's disease are separated by two-dimensional electrophoresis, and the fluorescent digital images of the resulting gels are matched to a chosen representative primary master gel image. This process allows any gel feature, characterised by its pI and MW, to be identified and examined on any gel of the study. In particular, the amount of protein present in a given feature can be measured in each gel; this feature abundance can be averaged amongst gels from similar samples (e.g. gels from samples from subjects having Alzheimer's disease). Finally, statistical analyses can be conducted on the thus created sample sets, in order to compare 2 or more sample sets to each other.

As used herein, “two-dimensional electrophoresis” (2D-electrophoresis) means a technique comprising isoelectric focusing, followed by denaturing electrophoresis; this generates a two-dimensional gel (2D-gel) containing a plurality of separated proteins. Preferably, the step of denaturing electrophoresis uses polyacrylamide electrophoresis in the presence of sodium dodecyl sulfate (SDS-PAGE). Especially preferred are the highly accurate and automatable methods and apparatus (“the Preferred Technology”) described in PCT Publication No WO 98/23950 and in U.S. Pat. No 6,064,754, both filed Dec. 1, 1997, each of which is incorporated herein by reference in its entirety with particular reference to the experimental protocol. Briefly, the Preferred Technology provides efficient, computer-assisted methods and apparatus for identifying, selecting and characterizing biomolecules (e.g. proteins, including glycoproteins) in a biological sample. A two-dimensional array is generated by separating biomolecules on a two-dimensional gel according to their electrophoretic mobility and isoelectric point. A computer-generated digital profile of the array is generated, representing the identity, apparent molecular weight, isoelectric point, and relative abundance of a plurality of biomolecules detected in the two-dimensional array, thereby permitting computer-mediated comparison of profiles from multiple biological samples, as well as computer aided excision of separated proteins of interest.

A particular scanner for detecting fluorescently labeled proteins is described in WO 96/36882 and in the Ph.D. thesis of David A. Basiji, entitled “Development of a High-throughput Fluorescence Scanner Employing Internal Reflection Optics and Phase-sensitive Detection (Total Internal Reflection, Electrophoresis)”, University of Washington (1997), Volume 58/12-B of Dissertation Abstracts International, page 6686, the contents of each of which are incorporated herein by reference. These documents describe an image scanner designed specifically for automated, integrated operation at high speeds. The scanner can image gels that have been stained with fluorescent dyes or silver stains, as well as storage phosphor screens. The Basiji thesis provides a phase-sensitive detection system for discriminating modulated fluorescence from baseline noise due to laser scatter or homogeneous fluorescence, but the scanner can also be operated in a non-phase-sensitive mode. This phase-sensitive detection capability would increase the sensitivity of the instrument by an order of magnitude or more compared to conventional fluorescence imaging systems. The increased sensitivity would reduce the sample-preparation load on the upstream instruments while the enhanced image quality simplifies image analysis downstream in the process.

A more highly preferred scanner is the Apollo 2 scanner (Oxford Glycosciences, Oxford, UK), which is a modified version of the above described scanner. In the Apollo 2 scanner, the gel is transported through the scanner on a precision lead-screw drive system. This is preferable to laying the glass plate on the belt-driven system that is described in the Basiji thesis, as it provides a reproducible means of accurately transporting the gel past the imaging optics.

In the Apollo 2 scanner, the gel is secured against three alignment stops that rigidly hold the glass plate in a known position. By doing this in conjunction with the above precision transport system, the absolute position of the gel can be predicted and recorded. This ensures that co-ordinates of each feature on the gel can be determined more accurately and communicated, if desired, to a cutting robot for excision of the feature. In the Apollo 2 scanner, the carrier that holds the gel has four integral fluorescent markers for use to correct the image geometry. These markers are a quality control feature that confirms that the scanning has been performed correctly.

In comparison to the scanner described in the Basiji thesis, the optical components of the Apollo 2 scanner have been inverted. In the Apollo 2 scanner, the laser, mirror, waveguide and other optical components are above the glass plate being scanned. The scanner described in the Basiji thesis has these components underneath. In the Apollo 2 scanner, the glass plate is mounted onto the scanner gel side down, so that the optical path remains through the glass plate. By doing this, any particles of gel that may break away from the glass plate will fall onto the base of the instrument rather than into the optics. This does not affect the functionality of the system, but increases its reliability.

Still more preferred is the Apollo 3 scanner, in which the signal output is digitized to the full 16-bit data without any peak saturation or without square root encoding of the signal. A compensation algorithm has also been applied to correct for any variation in detection sensitivity along the path of the scanning beam. This variation is due to anomalies in the optics and differences in collection efficiency across the waveguide. A calibration is performed using a perspex plate with an even fluorescence throughout. The data received from a scan of this plate are used to determine the multiplication factors needed to increase the signal from each pixel level to a target level. These factors are then used in subsequent scans of gels to remove any internal optical variations.

As defined above, a “feature” refers to a spot detected in a 2D gel, and the term “Alzheimer's Disease-Associated Feature” (ADF) refers to a feature that is differentially present in a sample (e.g. a sample of brain tissue) from a subject having Alzheimer's disease compared with a sample (e.g. a sample of brain tissue) from a subject free from Alzheimer's disease.

The ADFs disclosed herein have been identified by comparing tissue samples from subjects having Alzheimer's disease against tissue samples from subjects free from Alzheimer's disease. Subjects free from Alzheimer's disease include subjects with no known disease or condition (normal subjects) and subjects with diseases (including neurological and neurodegenerative diseases) other than Alzheimer's disease. Thus, the present invention, in as much as features are provided and characterized in subjects free from Alzheimer's disease and subjects having Alzheimer's disease, provides for the identification and characterization of features (ADFs), and by virtue of the amino acid sequence characterization of features provides protein isoforms (ADPIs), which are diagnostic or indicative of Alzheimer's disease.

The tissue of subjects having Alzheimer's disease was compared with tissue of subjects free from Alzheimer's Disease for each of the following five anatomically defined regions of the brain: hippocampus, entorhinal cortex, amygdala, frontal cortex and neocortex. In addition, a comparison was made between areas which are affected early and severely in Alzheimer's disease (the entorhinal cortex and hippocampus) and those which are only affected later in the disease progression. This comparison will provide information on early markers of Alzheimer's disease. In addition, all Alzheimer's disease samples were compared with all control samples to reveal proteins which are altered across the Alzheimer's disease brain.

The ADFs identified through the methods and apparatus of the Preferred Technology are listed in Table I. Table I shows ADFs that were found to be altered in one or more of the five tissue types examined in subjects having Alzheimer's disease as compared with the tissue of subjects free from Alzheimer's disease. ADFs were classified into one of 3 groups for each disease condition. The first group consists of ADFs that are present in at least 50% of disease or control samples and have a fold change (increase or decrease) of at least 1.5, (a+: fold increase of at least 1.5 relative to controls, a−: fold decrease of at least 1.5 relative to control). The second group consists of ADFs that are present in at least 50% of disease or controls samples with a fold change of at least 1.5 where the p-value for the fold change is less than 0.05 (b+: fold increase of at least 1.5 with p<0.05, b−: fold decrease of at least 1.5 with p<0.05). The third group consists of ADFs that are present in at least 50% of either the disease or the control samples but is absent from all samples in the other group, (c+: present in at least 50% of patient samples and absent from all control samples, c−: present in at least 50% of control samples and absent from all disease samples). These ADFs can be described by apparent molecular weight (MW) and isoelectric point (pI) as provided in Table I.

Blank cells in Table I reflect an ADF which did not meet the criteria for inclusion in any of the above groups for a given condition.

Table I. ADFs Altered in Brain Tissue of Subjects having Alzheimer's Disease*

TABLE I

| | | MW | All | selected | | | | | | EC/HC vs |
|---|---|---|---|---|---|---|---|---|---|---|
| ADF# | pI | (Da) | HC | HC | FC | NC | EC | Am | ALL | FC/NC/Am |
| ADF-1 | 5.07 | 41958 | b+ | a+ | | a− | a− | | a+ | b+ |
| ADF-3 | 5.53 | 51762 | b− | | | | | a− | | |
| ADF-5 | 5.88 | 50355 | a+ | | | | | | | |
| ADF-6 | 5.39 | 48281 | b+ | b+ | b+ | a+ | a+ | a+ | b+ | b+ |
| ADF-8 | 4.96 | 40177 | b+ | b+ | | a− | a− | a+ | b+ | b+ |
| ADF-9 | 4.94 | 55118 | | a− | | a+ | | a+ | a+ | a− |
| ADF-10 | 5.16 | 46069 | b+ | b+ | | | a+ | a+ | b+ | b+ |
| ADF-11 | 4.81 | 39554 | | | b− | | a− | | | |
| ADF-12 | 4.88 | 38224 | | a+ | | | | | | b+ |
| ADF-13 | 5.41 | 67038 | | | | | | a− | | |
| ADF-15 | 4.64 | 50029 | | | | a− | a− | a− | | |
| ADF-16 | 5.57 | 48424 | a− | | | a+ | | a− | a− | |
| ADF-22 | 5.19 | 102608 | | | | a+ | | a− | | |
| ADF-23 | 5.22 | 38803 | b− | b− | | | a+ | a− | | b− |
| ADF-24 | 5.47 | 77449 | | | a+ | | | a+ | | |
| ADF-25 | 5.06 | 52537 | | | b+ | a− | | | | a− |
| ADF-26 | 5.13 | 48692 | b+ | b+ | a+ | a+ | a+ | a+ | b+ | b+ |
| ADF-27 | 5.02 | 44949 | b+ | b+ | | | a+ | a+ | a+ | b+ |
| ADF-29 | 4.9 | 39388 | a+ | a+ | | a+ | a− | | | b+ |
| ADF-31 | 5.36 | 46573 | b+ | a+ | a− | | | a− | a+ | b+ |
| ADF-33 | 9.07 | 42881 | a− | | | a− | | | | |
| ADF-35 | 7.84 | 42531 | | | | | | a+ | | |
| ADF-37 | 7.57 | 30563 | b− | b− | | | | | | |
| ADF-39 | 7.26 | 42323 | | a+ | | | | | | |
| ADF-41 | 9.84 | 32806 | | | | | a− | | | |
| ADF-42 | 7.92 | 54422 | | b− | | a− | | | | |
| ADF-54 | 7.53 | 90365 | | b− | | | | | | |
| ADF-56 | 7.45 | 91453 | b− | b− | | a− | | | | |
| ADF-61 | 8.02 | 65431 | | | | a− | a− | | | |
| ADF-62 | 5.57 | 92331 | | a− | | a+ | | a+ | | |
| ADF-66 | 6.6 | 115748 | | b− | | | a+ | a− | | |
| ADF-67 | 7.57 | 49062 | a− | a− | a+ | a− | | a− | a− | b+ |
| ADF-68 | 5.63 | 91712 | | | | | a− | | | |
| ADF-70 | 6.78 | 116449 | | | | | a+ | | | |
| ADF-72 | 5.8 | 91999 | | | | | | a− | | |
| ADF-77 | 5.28 | 46016 | b+ | b+ | | a− | | | a+ | b+ |
| ADF-78 | 4.88 | 55952 | | | | | a− | c+ | | |
| ADF-79 | 6.04 | 51058 | | | | | a− | a+ | | b− |
| ADF-81 | 4.83 | 42229 | a− | | | a+ | a− | | | |
| ADF-82 | 8.93 | 40442 | | | | c− | | a− | a− | |
| ADF-85 | 5.71 | 41191 | | | | a− | | a− | | b+ |
| ADF-87 | 5.48 | 34110 | | | | | | a− | | |
| ADF-88 | 5.22 | 24404 | | | | a− | | | | |
| ADF-90 | 5.12 | 40883 | b+ | b+ | | a+ | | | b+ | b+ |
| ADF-91 | 6.95 | 39784 | | | | a− | | a− | | b+ |
| ADF-92 | 8.98 | 34521 | | | | a+ | a+ | a− | | |
| ADF-94 | 5.84 | 43662 | | | | | | a+ | | |
| ADF-95 | 6.65 | 40709 | | | | a+ | a− | a+ | | |
| ADF-97 | 5.12 | 19201 | b+ | b+ | | | | a+ | | |
| ADF-98 | 6.35 | 14707 | b+ | a+ | a+ | a− | | a+ | b+ | |
| ADF-101 | 8.99 | 15085 | | | | a− | | | | |
| ADF-102 | 5.13 | 38984 | | b− | | | a+ | a− | | b− |
| ADF-103 | 7.14 | 20196 | a+ | a+ | a+ | a− | a− | | | b+ |
| ADF-105 | 4.81 | 38327 | | | | a+ | a− | a− | | |
| ADF-107 | 7.05 | 18005 | | | | | a− | | | |
| ADF-108 | 5.74 | 34823 | | | | | | a+ | | |
| ADF-109 | 8.03 | 47014 | | | | a− | | | | |
| ADF-111 | 4.87 | 43065 | | | | a+ | c− | a+ | | |
| ADF-112 | 6.94 | 76640 | | | | a− | a− | | | |
| ADF-113 | 8.39 | 50091 | | | | a− | | | | |
| ADF-115 | 7.4 | 78857 | | | | | a− | | | |
| ADF-117 | 4.95 | 47483 | | | | | a+ | a+ | | |
| ADF-119 | 4.95 | 41245 | b+ | a+ | | c− | | a+ | b+ | b+ |
| ADF-120 | 5.06 | 46276 | b+ | b+ | b+ | c+ | a+ | | b+ | b+ |
| ADF-121 | 6.34 | 60952 | | | | | | | | b− |
| ADF-124 | 6.8 | 70243 | | b− | b− | a− | | | b− | a+ |
| ADF-125 | 4.84 | 43088 | | | | | c+ | a− | | |
| ADF-126 | 5.15 | 42785 | | | | | c− | a+ | | |
| ADF-127 | 5.21 | 40033 | | | a− | a+ | | a− | | |
| ADF-128 | 5.8 | 28231 | | | | | a− | a− | | |
| ADF-129 | 5.58 | 15554 | b− | b− | | | a− | | | |
| ADF-130 | 4.9 | 41328 | | | | | a− | a+ | a− | |
| ADF-131 | 5.13 | 33239 | b− | | | | a− | a− | | |
| ADF-132 | 5.06 | 40834 | a+ | a+ | | a− | a− | | a+ | b+ |
| ADF-133 | 6.81 | 42650 | | | a− | a− | a− | | | |
| ADF-138 | 9.72 | 26137 | | | | a− | | a+ | | |
| ADF-139 | 9.81 | 44026 | | b− | | | c− | a+ | | |
| ADF-140 | 4.58 | 15950 | | | | | c− | | | |
| ADF-141 | 4.66 | 41227 | | | | a− | | c− | | |
| ADF-142 | 6.53 | 39774 | a− | | | a− | | | | a+ |
| ADF-143 | 8.99 | 32577 | | | | a− | | | | |
| ADF-144 | 4.96 | 43397 | b+ | b+ | | c+ | c− | a+ | | b+ |
| ADF-146 | 5.25 | 11857 | | | | | | a− | | |
| ADF-148 | 5.47 | 48199 | b+ | b+ | b+ | a+ | a+ | | b+ | a+ |
| ADF-149 | 5.13 | 43744 | | | | | | a+ | | a+ |
| ADF-150 | 5.12 | 46104 | b+ | b+ | a+ | | a+ | a+ | a+ | b+ |
| ADF-151 | 4.84 | 38334 | a+ | | | c+ | a− | a− | | a+ |
| ADF-152 | 5.32 | 48338 | b+ | b+ | b+ | a+ | a+ | | b+ | b+ |
| ADF-153 | 4.99 | 53853 | | | | | a+ | | | |
| ADF-154 | 4.85 | 39486 | b+ | | | a+ | a− | | | b+ |
| ADF-155 | 5.56 | 66533 | | | | a+ | | | | b− |
| ADF-156 | 5.16 | 47248 | | | b+ | a+ | | a+ | | |
| ADF-157 | 5.45 | 42821 | | | | | a+ | | | b− |
| ADF-159 | 5.19 | 48581 | b+ | b+ | b+ | a+ | a+ | a+ | b+ | b+ |
| ADF-160 | 4.86 | 47558 | | | | | a+ | | | b− |
| ADF-162 | 5.26 | 48579 | b+ | b+ | a+ | a+ | a+ | a+ | b+ | b+ |
| ADF-163 | 5.03 | 41674 | b+ | a+ | b+ | | | a+ | b+ | b+ |
| ADF-165 | 6.42 | 24608 | b+ | b+ | | | | | | b+ |
| ADF-172 | 9.32 | 10855 | | b− | | a+ | a+ | | | |
| ADF-173 | 6.76 | 64255 | | b− | | | | | | b− |
| ADF-175 | 9.55 | 36150 | | | | a+ | a− | c− | | a+ |
| ADF-176 | 6.94 | 63236 | b− | b− | | | | | | b− |
| ADF-182 | 9.36 | 32746 | | | | a− | | | | |
| ADF-183 | 5.42 | 23634 | b− | b− | | | | | | |
| ADF-188 | 4.96 | 93776 | | | | a+ | a− | a+ | | |
| ADF-189 | 5.83 | 65453 | | | | a+ | | | | |
| ADF-191 | 6.9 | 50019 | | b− | | | | | | |
| ADF-193 | 5.91 | 64954 | a− | b− | | | | a+ | | b− |
| ADF-194 | 7.16 | 49777 | | b− | | | | | | |
| ADF-196 | 7.07 | 51831 | | | b+ | | a+ | a+ | | |
| ADF-202 | 7.14 | 63134 | b− | b− | | | | | | b− |
| ADF-204 | 6.29 | 11406 | b+ | a+ | b+ | | | | b+ | b+ |
| ADF-208 | 5.26 | 19241 | | | | | | a+ | | |
| ADF-209 | 5.03 | 11738 | | b+ | | | | a+ | | |
| ADF-216 | 7.76 | 21455 | | | | | a+ | | | |
| ADF-217 | 7.53 | 10660 | | | | a+ | a+ | | | |
| ADF-220 | 7.17 | 26848 | | | a+ | a+ | a+ | | | |
| ADF-223 | 7.08 | 42854 | b− | b− | | | | a− | | |
| ADF-228 | 6.41 | 74736 | | | | | c+ | | | |
| ADF-229 | 7.77 | 46953 | b− | b− | | | | | | |
| ADF-230 | 4.87 | 53678 | | | | | | c− | | |
| ADF-232 | 5 | 37305 | | | b− | | | | | |
| ADF-236 | 4.64 | 18906 | | | | c+ | a+ | | | |
| ADF-237 | 4.67 | 25143 | b− | a− | | a+ | a− | a− | b− | |
| ADF-240 | 9.56 | 32166 | a− | | | a+ | | | | |
| ADF-243 | 5.35 | 39493 | | | | | a+ | a− | | |
| ADF-245 | 5.82 | 26143 | a− | | | | | c− | | |
| ADF-250 | 5.07 | 167558 | | | | | a+ | | | b+ |
| ADF-251 | 5.52 | 171859 | | | | | a+ | a− | | a+ |
| ADF-252 | 5.37 | 126296 | | | | | a+ | | | |
| ADF-253 | 6.56 | 138727 | | b− | | | | | | |
| ADF-254 | 6.63 | 138817 | | a− | | a+ | | | | |
| ADF-255 | 6.77 | 138133 | a− | b− | | | | | | |
| ADF-256 | 6.91 | 136744 | a− | b− | | | | | | |
| ADF-257 | 5.42 | 52510 | b+ | a+ | | a− | a+ | | | |
| ADF-258 | 8.85 | 42977 | b+ | b+ | | c− | a+ | a+ | | |
| ADF-259 | 7.16 | 42747 | | | a− | | | | | |
| ADF-260 | 9.64 | 42986 | | b− | | a+ | | a− | | a− |
| ADF-261 | 5.26 | 41426 | | | | | a+ | a− | | |
| ADF-262 | 5.02 | 40870 | b+ | a+ | b+ | c− | a− | | | b+ |
| ADF-263 | 5.58 | 40684 | | b− | | | | | | |
| ADF-264 | 5.53 | 40345 | | | | a+ | | | | |
| ADF-265 | 6.77 | 41249 | | | | | | a+ | | |
| ADF-266 | 7.41 | 40715 | | | | | a+ | | | b+ |
| ADF-267 | 5.28 | 39907 | | | | | a+ | | | |
| ADF-268 | 5.32 | 39751 | b− | a− | a− | a− | a− | | b− | |
| ADF-269 | 9.72 | 40657 | | a− | | a− | a+ | | | |
| ADF-270 | 4.91 | 40409 | b+ | b+ | a+ | | a− | | b+ | a+ |
| ADF-271 | 5.01 | 39858 | a+ | | | | | | a+ | a+ |
| ADF-272 | 8.13 | 40619 | | | | | a+ | a− | | a+ |
| ADF-273 | 5.7 | 39437 | b− | b− | | | | | | |
| ADF-274 | 5.05 | 39078 | | | | | a+ | | | |
| ADF-275 | 4.94 | 39423 | b+ | a+ | a+ | | a− | a+ | b+ | b+ |
| ADF-276 | 7.51 | 39969 | b− | b− | | | | | | |
| ADF-277 | 4.98 | 39146 | | | b− | a+ | a+ | a− | | |
| ADF-278 | 7.25 | 39463 | | a− | a− | | | c+ | | |
| ADF-279 | 8.09 | 39011 | | | | | | a− | | |
| ADF-280 | 5.83 | 37149 | | | | | | | | b− |
| ADF-281 | 5.4 | 37687 | | | a− | | | | | |
| ADF-282 | 5.69 | 37186 | | b− | | | | | | a− |
| ADF-283 | 5.58 | 36661 | | | | | | a− | | b+ |
| ADF-284 | 6.28 | 36705 | b− | b− | | a+ | a− | a− | b− | |
| ADF-285 | 5.7 | 36544 | | | | a+ | | | | |
| ADF-286 | 7.79 | 36912 | | | | | | | | b+ |
| ADF-287 | 6.86 | 35923 | | | b− | | | | | |
| ADF-288 | 6.17 | 35958 | | a− | | | a− | a+ | | b− |
| ADF-289 | 9.18 | 36921 | | | | c+ | a+ | c− | | |
| ADF-290 | 6.63 | 36052 | | a+ | | | | | | |
| ADF-291 | 7.79 | 36208 | | | a− | a− | | a+ | | |
| ADF-292 | 9.67 | 36144 | | | | a− | c+ | | | |
| ADF-293 | 7.04 | 34992 | | b− | | | | | | |
| ADF-294 | 5.16 | 34573 | | | | a− | a− | a− | b− | |
| ADF-295 | 5.39 | 34366 | | | | | | | | b− |
| ADF-296 | 4.76 | 34122 | | | | a− | | a− | | |
| ADF-297 | 5.03 | 33778 | a− | a− | | a+ | a− | | b− | |
| ADF-298 | 5.37 | 33180 | | | | a− | | c− | | |
| ADF-299 | 9.26 | 34579 | | | | | a− | | | |
| ADF-300 | 9.93 | 33122 | a− | a− | a+ | a− | a+ | | | a− |
| ADF-301 | 9.79 | 33445 | | | | | c+ | | | |
| ADF-302 | 7.09 | 32791 | | | | | a+ | | | a+ |
| ADF-303 | 4.91 | 32291 | b+ | | | | | a+ | | |
| ADF-304 | 5.36 | 31776 | | | | | | | | b− |
| ADF-305 | 4.6 | 32146 | a− | b− | | | | a− | | |
| ADF-306 | 4.67 | 31899 | | | | a+ | | | | |
| ADF-307 | 4.61 | 31610 | | | | | a− | | | |
| ADF-308 | 9.64 | 32303 | b− | b− | | a− | | a+ | | |
| ADF-309 | 4.69 | 30667 | | | | | a− | | | |
| ADF-311 | 5.33 | 28896 | | b+ | | | | a+ | | |
| ADF-312 | 7.39 | 28434 | | | | | a+ | a− | | |
| ADF-313 | 5.04 | 28096 | | | | a− | | a+ | | |
| ADF-314 | 5.63 | 27914 | | | | | a− | a+ | | |
| ADF-315 | 6.13 | 25520 | | | a+ | a− | | | | |
| ADF-316 | 6.05 | 25158 | | | | a+ | a+ | | | |
| ADF-317 | 4.61 | 25514 | | | | | a− | | | |
| ADF-318 | 7.15 | 24544 | b− | b− | | a+ | a− | a− | b− | |
| ADF-319 | 9.97 | 24740 | b− | b− | | | a+ | | | b− |
| ADF-320 | 6.44 | 23908 | | | | | | a− | | |
| ADF-321 | 6.28 | 23240 | | b+ | b+ | | | | | b+ |
| ADF-322 | 5.89 | 22204 | b− | | | a− | | | | |
| ADF-323 | 5.23 | 22730 | | a− | | | | | | |
| ADF-324 | 7.43 | 22516 | | b− | | | | | | |
| ADF-325 | 7.24 | 22428 | b− | | | | | | | |
| ADF-326 | 9.69 | 23002 | | a− | | | | c− | | |
| ADF-327 | 5.48 | 21128 | | | | | a− | | | |
| ADF-328 | 4.94 | 21882 | b− | b− | b− | | a− | | b− | |
| ADF-329 | 5.02 | 21444 | | | a− | | | | | |
| ADF-330 | 9.53 | 21378 | | | | a+ | | | | |
| ADF-331 | 9.32 | 21202 | | | | | a+ | | | b+ |
| ADF-332 | 10.02 | 21013 | a− | b− | | | | c− | | |
| ADF-333 | 7.8 | 20396 | | | | | c+ | | | |
| ADF-334 | 7.08 | 18540 | | a− | | | a− | a+ | | |
| ADF-335 | 5.13 | 17557 | b− | | | | | c− | | b+ |
| ADF-336 | 4.52 | 16749 | | a− | a− | a+ | | | | a− |
| ADF-337 | 6.61 | 16325 | | a+ | | a− | a− | a− | | b+ |
| ADF-338 | 5.71 | 15618 | a+ | | a+ | a+ | a+ | a+ | b+ | |
| ADF-339 | 4.68 | 16126 | | | | a− | | a− | | |
| ADF-340 | 4.31 | 15235 | | b− | | | | | | |
| ADF-341 | 4.68 | 15288 | | | a− | a− | a+ | a− | | |
| ADF-342 | 7.51 | 14929 | a− | b− | | a− | a− | | | |
| ADF-343 | 9.12 | 15079 | | | | a− | a+ | | | |
| ADF-344 | 4.77 | 14226 | b− | b− | | | a+ | | | |
| ADF-345 | 7.75 | 13620 | | | | a− | | | | |
| ADF-346 | 4.56 | 14197 | b− | b− | | | | | b− | |
| ADF-347 | 4.52 | 14033 | | | | | a− | | | |
| ADF-348 | 7.36 | 12068 | | | | a+ | | a+ | | |
| ADF-349 | 4.73 | 13198 | b+ | b+ | | a− | a+ | a− | | |
| ADF-350 | 5.03 | 12536 | | | | | a− | | | |
| ADF-351 | 5.91 | 10930 | b− | b− | | | | | | |
| ADF-352 | 6.12 | 10966 | b− | | | | | | | |
| ADF-353 | 7.35 | 10655 | | | a+ | a+ | a+ | | | |
| ADF-354 | 5.62 | 10558 | | | | | a+ | | | |
| ADF-355 | 9.64 | 10941 | a+ | | | | | a+ | | a+ |
| ADF-356 | 7.04 | 136798 | | a− | | | a+ | | | |
| ADF-357 | 5.17 | 115959 | | | | | a+ | | | |
| ADF-358 | 6.73 | 105628 | | a− | b+ | a+ | | | | |
| ADF-359 | 5.71 | 91189 | | | | | a− | | | |
| ADF-360 | 7.36 | 92269 | b− | b− | | a− | | | | |
| ADF-361 | 6.32 | 81004 | a+ | | | a+ | a+ | a− | | |
| ADF-362 | 6.81 | 76774 | b− | b− | | | | | | |
| ADF-363 | 6.36 | 75388 | | | | | a− | | | |
| ADF-364 | 6.09 | 73832 | | | | | | | | b− |
| ADF-365 | 6.47 | 74592 | | | | | | a− | | |
| ADF-366 | 6.34 | 73530 | b− | b− | a− | | | a− | | b− |
| ADF-367 | 5.35 | 66839 | | | | | | | | b− |
| ADF-368 | 5.49 | 65816 | | b− | | | | | | b− |
| ADF-369 | 6.7 | 69857 | | a+ | | | | | | |
| ADF-370 | 9.3 | 69099 | | b− | | a− | | a+ | | |
| ADF-371 | 5.98 | 64860 | | | | a+ | | | | |
| ADF-372 | 5.68 | 63210 | | | | a+ | | | | |
| ADF-373 | 6.61 | 65116 | | | b− | | | | | b− |
| ADF-374 | 7.01 | 65362 | | b− | | | | | | |
| ADF-375 | 6.12 | 63693 | | | | a− | | a− | | a− |
| ADF-376 | 6.52 | 65065 | | c− | b− | | a+ | a+ | | a− |
| ADF-377 | 9.59 | 44257 | | | | | c− | a+ | | |
| ADF-378 | 5.66 | 38000 | | | | | c− | | | |
| ADF-379 | 7.09 | 37275 | | | | | | a+ | | |
| ADF-380 | 8.69 | 32065 | | | | | a+ | a− | | |
| ADF-381 | 5.48 | 29061 | | a+ | | | a+ | c− | | a+ |
| ADF-382 | 6.36 | 22175 | a+ | a+ | b+ | a+ | a+ | a+ | b+ | |
| ADF-383 | 4.52 | 17290 | | | | c− | | a+ | | |
| ADF-384 | 9.43 | 16119 | a− | a− | | | a− | | b− | |
| ADF-386 | 5.94 | 12563 | b− | a− | | a+ | | a+ | | |
| ADF-387 | 5.33 | 11323 | b+ | b+ | | | | | | b+ |
| ADF-388 | 6.22 | 11486 | | a− | | | | | | |
| ADF-389 | 6.63 | 126012 | | b− | | | | | | |
| ADF-390 | 5.01 | 93773 | a+ | b− | | a− | a+ | c− | | a+ |
| ADF-391 | 7.62 | 88209 | | | | | | c− | | |
| ADF-392 | 5.47 | 75385 | | b− | | | a+ | | | |
| ADF-393 | 5.42 | 70689 | | | | a− | a− | | | |
| ADF-394 | 5.62 | 66442 | | | | | | a− | | |
| ADF-395 | 7.82 | 53530 | b− | b− | | | | a− | | |
| ADF-396 | 7.15 | 53305 | b− | | b− | | | | | |
| ADF-397 | 5.29 | 51553 | | | | | | a− | | |
| ADF-398 | 6.02 | 51914 | | b− | | | | | | |
| ADF-399 | 6.63 | 52447 | | | | | a− | | | b+ |
| ADF-400 | 9.14 | 52590 | b− | b− | | a+ | | a− | | a− |
| ADF-401 | 6.27 | 51636 | a− | | | | | a− | | |
| ADF-402 | 5.58 | 51111 | | | | | a+ | | | |
| ADF-403 | 7.48 | 51603 | | | | a+ | | | | a− |
| ADF-404 | 6.73 | 51384 | | | | | | | | b+ |
| ADF-405 | 4.83 | 50675 | b− | b− | | | | | | b− |
| ADF-406 | 6.53 | 51024 | | | b− | | | | | |
| ADF-407 | 4.9 | 50459 | b− | a− | | | a+ | | | |
| ADF-408 | 5.79 | 50212 | | b− | | | | | | |
| ADF-409 | 5.69 | 50034 | | | a+ | a− | | | | |
| ADF-410 | 5.11 | 49646 | | | b− | | a+ | a− | | a+ |
| ADF-411 | 5.33 | 49692 | b+ | b+ | | | a− | a− | a+ | |
| ADF-412 | 5.06 | 49556 | a+ | | | a− | a+ | | | a+ |
| ADF-413 | 7.59 | 50213 | a− | | | a− | a− | a+ | a− | |
| ADF-414 | 4.94 | 49167 | | | | | a+ | c+ | | a+ |
| ADF-415 | 5.61 | 48968 | | | | a− | | | | b+ |
| ADF-416 | 6.8 | 49576 | | | | | a+ | a− | | |
| ADF-417 | 5 | 48877 | | | | a− | | | | |
| ADF-418 | 4.82 | 47830 | | a+ | | | | | | |
| ADF-419 | 6.52 | 47975 | b+ | b+ | | | | a+ | b+ | b+ |
| ADF-420 | 4.85 | 16376 | | | a− | | | a− | | |
| ADF-421 | 7.61 | 15176 | | | | | c+ | | | |
| ADF-422 | 5.33 | 126111 | | a+ | | | | a− | | |
| ADF-423 | 6.21 | 61089 | | | | | | | | b− |
| ADF-424 | 4.83 | 52105 | | | | | | c+ | | |
| ADF-425 | 7.89 | 50602 | | b− | | | | a+ | | |
| ADF-426 | 7.68 | 50266 | | | | a+ | | | | |
| ADF-427 | 8.66 | 50180 | a− | b− | | | | | a− | |
| ADF-428 | 5.04 | 48509 | | | | | a+ | | | |
| ADF-429 | 7.56 | 47861 | a+ | a+ | | a− | | a+ | | b+ |
| ADF-430 | 5.67 | 46344 | | b− | | | | | | |
| ADF-431 | 7.65 | 46361 | | b− | | | | | | |
| ADF-432 | 9.42 | 46974 | | | | a− | | a− | | |
| ADF-433 | 9.55 | 46884 | | | | a+ | | c− | | |
| ADF-434 | 5.46 | 45846 | b− | b− | | | | a− | | b− |
| ADF-435 | 7.25 | 45448 | | | | a− | | | | |
| ADF-436 | 6.1 | 44528 | | b− | | | | a− | | |
| ADF-437 | 4.88 | 45571 | a+ | a+ | a− | | a− | a− | | b+ |
| ADF-438 | 6.15 | 44253 | | | | | | a+ | | |
| ADF-439 | 6.84 | 44245 | b− | b− | | | | | | |
| ADF-440 | 5.31 | 44519 | a+ | b+ | | | | | | |
| ADF-441 | 6.56 | 43942 | b− | b− | b− | | | | | |
| ADF-442 | 5.36 | 43816 | | | | | | a− | | a+ |
| ADF-443 | 4.91 | 43591 | b+ | a+ | | a+ | | | a+ | a+ |
| ADF-444 | 6.48 | 43383 | | | | | | a+ | | a− |
| ADF-445 | 5.55 | 42883 | | | | | | a− | | |
| ADF-446 | 6.64 | 43189 | | | | | | | | b− |
| ADF-447 | 5 | 43269 | b+ | b+ | | | a− | a+ | | |
| ADF-448 | 7.84 | 43648 | | b− | | a− | | | | |
| ADF-449 | 4.75 | 43300 | b+ | | | | a− | | | |
| ADF-450 | 5.1 | 42818 | b+ | b+ | a+ | a+ | a+ | | b+ | |
| ADF-451 | 6.59 | 42651 | | | | a− | a− | | | |
| ADF-452 | 8.65 | 42868 | | | b+ | a− | a− | a+ | a− | a− |
| ADF-453 | 6.54 | 41562 | b− | | | | | | | |
| ADF-454 | 6.17 | 40304 | | a+ | b+ | | | a+ | | |
| ADF-455 | 4.89 | 37155 | | | | | a+ | c− | | |
| ADF-456 | 5.05 | 35647 | b− | a− | | a+ | a− | a− | b− | a− |
| ADF-457 | 7.77 | 32435 | | a− | | a+ | | | | |
| ADF-458 | 5.13 | 32364 | | | | a− | a− | | b− | |
| ADF-459 | 7.67 | 31505 | b− | | | | | a− | | |
| ADF-460 | 6.61 | 15951 | | | | | | c− | | |
| ADF-461 | 5.16 | 103474 | | | b+ | a− | | | | |
| ADF-462 | 6.88 | 66241 | a− | | | c+ | a− | a− | | |
| ADF-463 | 6.58 | 63519 | | b− | | | | | | |
| ADF-464 | 6.01 | 58817 | | | | | | c− | | |
| ADF-465 | 6.94 | 59947 | | | | | | a− | | |
| ADF-466 | 7.05 | 60297 | | | | | | a+ | | |
| ADF-467 | 7.16 | 59914 | | b− | | | | a− | | |
| ADF-468 | 6.45 | 58723 | | b+ | | | | a− | | |
| ADF-469 | 5.48 | 55802 | | | | | | | | b− |
| ADF-470 | 6.5 | 56254 | a− | a− | | | | a− | | |
| ADF-471 | 6.1 | 55319 | | a+ | a+ | | | | | a− |
| ADF-472 | 5.24 | 54138 | | | a− | | | c− | | b+ |
| ADF-473 | 6.33 | 54465 | | b− | | | | a− | | |
| ADF-474 | 7.68 | 54716 | | b− | | | | | | |
| ADF-475 | 4.99 | 54607 | | | a− | | | a+ | | |
| ADF-476 | 6.45 | 54211 | b− | a− | b− | a+ | a− | | | |
| ADF-477 | 6.51 | 54406 | | b− | a− | | | c− | | |
| ADF-478 | 5.48 | 53235 | | | | | a− | | | b+ |
| ADF-479 | 6.09 | 53874 | | b− | | | | a− | | |
| ADF-480 | 4.94 | 53281 | a+ | c− | | a+ | a− | a− | | |
| ADF-481 | 6.18 | 53133 | | | | a− | | | | |
| ADF-482 | 6.15 | 51602 | | | | a− | | | | |
| ADF-483 | 7.81 | 50189 | a− | | | | | | | |
| ADF-484 | 8.91 | 50220 | | | | c− | | | | |
| ADF-485 | 6.27 | 48105 | | | | | a− | | | b+ |
| ADF-486 | 4.77 | 47947 | | | | | c− | a− | | |
| ADF-487 | 5.41 | 46720 | | a+ | | a− | | | | |
| ADF-488 | 7.09 | 46981 | | | a+ | | | a− | | |
| ADF-489 | 4.96 | 46544 | a+ | a+ | | | | c+ | | |
| ADF-490 | 6.25 | 46318 | | | | | a− | | | |
| ADF-491 | 5.23 | 45828 | b+ | a+ | a+ | a+ | | | b+ | b+ |
| ADF-492 | 7.5 | 44639 | b− | a− | | a+ | c− | | | |
| ADF-493 | 5.08 | 44646 | b+ | b+ | | | c+ | | | b+ |
| ADF-494 | 6.95 | 44465 | | | | a+ | | | | |
| ADF-495 | 7.46 | 43838 | b− | b− | | | a− | | | b− |
| ADF-496 | 7.63 | 43211 | | | | | a− | a+ | | |
| ADF-497 | 6.22 | 37984 | | | | | a− | | | |
| ADF-498 | 4.77 | 38566 | | | | | c+ | | | |
| ADF-499 | 5.34 | 23685 | | | | | a− | a− | | |
| ADF-500 | 6.06 | 60911 | | | | | a− | | | |
| ADF-501 | 5.33 | 58448 | | b+ | | | | | | |
| ADF-502 | 5.95 | 57646 | | b− | | | | | | |
| ADF-503 | 7.33 | 59558 | | b− | | | | | | |
| ADF-504 | 7.47 | 59218 | b− | | | | | | | b− |
| ADF-505 | 5.32 | 55892 | | | | | | | | b− |
| ADF-506 | 6.68 | 52348 | | | b+ | a+ | c+ | a+ | | |
| ADF-507 | 5.47 | 49332 | b+ | b+ | | | | | a+ | |
| ADF-508 | 4.94 | 46443 | a+ | | | | a+ | a+ | | |
| ADF-509 | 9.08 | 44787 | | | | | | a+ | | |
| ADF-510 | 5.06 | 39851 | b+ | | | | | c+ | b+ | a+ |
| ADF-511 | 7.71 | 35365 | | | | | | | | a+ |
| ADF-512 | 6.42 | 22602 | | | | a− | | | | |
| ADF-513 | 8.34 | 55698 | | | | a− | | | | |
| ADF-514 | 6.44 | 52514 | b+ | | | a+ | | | | b+ |
| ADF-515 | 7.52 | 49586 | a− | a+ | b− | a+ | | | | |
| ADF-516 | 7.77 | 44058 | | | | | a− | | | |
| ADF-517 | 4.86 | 40635 | | | | a+ | c− | a− | | |
| ADF-518 | 6.1 | 61221 | | | | | | | | b− |
| ADF-519 | 4.84 | 53906 | | | | | c− | | | |
| ADF-520 | 4.77 | 51522 | | | b− | | c− | c+ | | |
| ADF-521 | 5.24 | 47302 | | a− | | a+ | c+ | a− | | |
| ADF-522 | 5.7 | 46027 | | | | | c− | c+ | | |
| ADF-523 | 9.71 | 47395 | | | | | a− | c− | | |
| ADF-524 | 4.96 | 44893 | b+ | b+ | | | | | | b+ |
| ADF-525 | 4.88 | 43587 | | | | | a− | a− | | |
| ADF-526 | 4.61 | 38042 | | | | a+ | a− | a+ | | |
| ADF-527 | 8.1 | 13928 | | | | a− | | | | a+ |
| ADF-528 | 4.93 | 94807 | | | | | | a− | | |
| ADF-529 | 9.71 | 33463 | | | | a− | | | | |
| ADF-530 | 4.9 | 53903 | | | | | | c− | | |
| ADF-531 | 7.73 | 88666 | | | | | a− | a− | | |
| ADF-532 | 7.9 | 56000 | | | | | c− | | | |
| ADF-533 | 4.85 | 57995 | | | | | a− | | | |
| ADF-534 | 8.6 | 44086 | | | | | a− | a− | | |
| ADF-535 | 8.78 | 55742 | a+ | | | | a− | | | a+ |
| ADF-536 | 4.52 | 55713 | | | | | c+ | | | |
| ADF-537 | 4.78 | 44895 | | | | | a− | | | |
| ADF-538 | 4.73 | 74425 | | | | | c− | | | |
| ADF-539 | 5.52 | 49807 | | | | c+ | c− | | | |
| ADF-540 | 5.21 | 40933 | | | | c+ | | | | |
| ADF-541 | 8.65 | 41532 | | | | c− | a+ | | | |
| ADF-542 | 7.68 | 16766 | | | | a− | | | | |
| ADF-543 | 8.85 | 32258 | | | | | | a− | | |
| ADF-544 | 5.53 | 38484 | | | | | | a− | | |
| ADF-545 | 5.4 | 64958 | | | | c− | | | | |
| ADF-546 | 8.85 | 51770 | | | | c− | | | | |
| ADF-547 | 4.89 | 59987 | | | | | | c− | | |
| ADF-548 | 6.62 | 23128 | | | | | a− | | | |
| ADF-549 | 6.61 | 105070 | | | | c+ | a+ | | | |

*HC = hippocampus, Selected HC = hippocampal samples with a post-motrem time < 4h, EC = Entorhinal cortex, FC = Frontal Cortex, NC = Neocortex, AM = amygdala, Early = EC/HC vs. FC/NC/Am, ALL = all Alzheimer's disease samples vs all control samples.

For any given ADF, the signal obtained upon analysing brain tissue from subjects having Alzheimer's disease relative to the signal obtained upon analysing brain tissue from subjects free from Alzheimer's disease will depend upon the particular analytical protocol and detection technique that is used. Accordingly, those skilled in the art will understand that any laboratory, based on the present description, can establish a suitable reference range for any ADF in subjects free from Alzheimer's disease according to the analytical protocol and detection technique in use. In particular, at least one positive control brain tissue sample from a subject known to have Alzheimer's disease or at least one negative control brain tissue sample from a subject known to be free from Alzheimer's disease (and more preferably both positive and negative control samples) are included in each batch of test samples analysed. In one embodiment, the level of expression of a feature is determined relative to a background value, which is defined as the level of signal obtained from a proximal region of the image that (a) is equivalent in area to the particular feature in question; and (b) contains no substantial discernable protein feature.

In a preferred embodiment, the signal associated with an ADF in the brain tissue of a subject (e.g., a subject suspected of having or known to have Alzheimer's disease) is normalized with reference to one or more Expression Reference Features (ERFs) detected in the same 2D gel. As will be apparent to one of ordinary skill in the art, such ERFs may readily be determined by comparing different samples using techniques and protocols such as the Preferred Technology. Suitable ERFs include (but are not limited to) that described in the following table.

Table II. Expression Reference Features

TABLE II

| ERF# | pI | MW (Da) |
|---|---|---|
| ERF-1 | 6.56 | 31197 |
| ERF-2 | 4.64 | 27085 |
| ERF-3 | 7.53 | 25830 |
| ERF-4 | 7.15 | 25763 |
| ERF-5 | 6.94 | 23823 |
| ERF-6 | 7.21 | 23437 |
| ERF-7 | 6.18 | 21580 |
| ERF-8 | 5.57 | 21794 |
| ERF-9 | 5.51 | 19853 |
| ERF-10 | 5.17 | 20351 |
| ERF-11 | 5.72 | 15301 |
| ERF-12 | 4.85 | 12056 |
| ERF-13 | 4.98 | 77409 |
| ERF-14 | 5.4 | 78003 |
| ERF-15 | 5.86 | 16146 |

As those of skill in the art will readily appreciate, the measured MW and pI of a given feature or Protein Isoform will vary to some extent depending on the precise protocol used for each step of the 2D electrophoresis and for landmark matching. As used herein, the terms “MW” and “pI” are defined, respectively, to mean the apparent molecular weight in Daltons and the apparent isoelectric point of a feature or Protein Isoform as measured in careful accordance with the Reference Protocol identified in Section 6 below. When the Reference Protocol is followed and when samples are run in duplicate or a higher number of replicates, variation in the measured mean pI of a ADF or ADPI is typically less than 3% and variation in the measured mean MW of an ADF or ADPI is typically less than 5%. Where the skilled artisan wishes to diverge from the Reference Protocol, calibration experiments should be performed to compare the MW and pI for each ADF or Protein Isoform as detected (a) by the Reference Protocol and (b) by the divergent.

The ADFs of the invention can be used, for example, for detection, treatment, diagnosis, or for drug development. In one embodiment of the invention, brain tissue from a subject (e.g., a subject suspected of having Alzheimer's disease) is analyzed by 2D electrophoresis for quantitative detection of one or more of the following ADFs: ADF-1, ADF-3, ADF-5, ADF-6, ADF-8, ADF-9, ADF-10, ADF-11, ADF-12, ADF-13, ADF-15, ADF-16, ADF-22, ADF-23, ADF-24, ADF-25, ADF-26, ADF-27, ADF-29, ADF-31, ADF-33, ADF-35, ADF-37, ADF-39, ADF-42, ADF-54, ADF-56, ADF-61, ADF-62, ADF-66, ADF-67, ADF-68, ADF-70, ADF-72, ADF-77, ADF-78, ADF-79, ADF-81, ADF-82, ADF-85, ADF-87, ADF-88, ADF-90, ADF-91, ADF-92, ADF-94, ADF-95, ADF-97, ADF-98, ADF-101, ADF-102, ADF-103, ADF-105, ADF-107, ADF-108, ADF-109, ADF-111, ADF-112, ADF-113, ADF-115, ADF-117, ADF-119, ADF-120, ADF-121, ADF-124, ADF-125, ADF-126, ADF-127, ADF-128, ADF-129, ADF-130, ADF-131, ADF-132, ADF-133, ADF-138, ADF-139, ADF-140, ADF-141, ADF-142, ADF-143, ADF-144, ADF-146, ADF-148, ADF-149, ADF-150, ADF-151, ADF-152, ADF-153, ADF-154, ADF-155, ADF-156, ADF-157, ADF-159, ADF-160, ADF-162, ADF-163, ADF-165, ADF-172, ADF-173, ADF-175, ADF-176, ADF-182, ADF-183, ADF-188, ADF-189, ADF-191, ADF-193, ADF-194, ADF-196, ADF-202, ADF-204, ADF-208, ADF-209, ADF-216, ADF-217, ADF-220, ADF-223, ADF-228, ADF-229, ADF-230, ADF-232, ADF-236, ADF-237, ADF-240, ADF-243, ADF-245, ADF-250, ADF-251, ADF-252, ADF-253, ADF-254, ADF-255, ADF-256, ADF-257, ADF-258, ADF-259, ADF-260, ADF-261, ADF-262, ADF-263, ADF-264, ADF-265, ADF-266, ADF-267, ADF-268, ADF-269, ADF-270, ADF-271, ADF-272, ADF-273, ADF-274, ADF-275, ADF-276, ADF-277, ADF-278, ADF-279, ADF-280, ADF-281, ADF-282, ADF-283, ADF-284, ADF-285, ADF-286, ADF-287, ADF-288, ADF-289, ADF-290, ADF-291, ADF-292, ADF-293, ADF-294, ADF-295, ADF-296, ADF-297, ADF-298, ADF-299, ADF-300, ADF-301, ADF-302, ADF-303, ADF-304, ADF-305, ADF-306, ADF-307, ADF-308, ADF-309, ADF-311, ADF-312, ADF-313, ADF-314, ADF-315, ADF-316, ADF-317, ADF-318, ADF-319, ADF-320, ADF-321, ADF-322, ADF-323, ADF-324, ADF-325, ADF-326, ADF-327, ADF-328, ADF-329, ADF-330, ADF-331, ADF-332, ADF-333, ADF-334, ADF-335, ADF-336, ADF-337, ADF-338, ADF-339, ADF-340, ADF-341, ADF-342, ADF-343, ADF-344, ADF-345, ADF-346, ADF-347, ADF-348, ADF-349, ADF-350, ADF-351, ADF-352, ADF-353, ADF-354, ADF-355, ADF-356, ADF-357, ADF-358, ADF-359, ADF-360, ADF-361, ADF-362, ADF-363, ADF-364, ADF-365, ADF-366, ADF-367, ADF-368, ADF-369, ADF-370, ADF-371, ADF-372, ADF-373, ADF-374, ADF-375, ADF-376, ADF-377, ADF-378, ADF-379, ADF-380, ADF-381, ADF-382, ADF-383, ADF-384, ADF-386, ADF-387, ADF-388, ADF-389, ADF-390, ADF-391, ADF-392, ADF-393, ADF-394, ADF-395, ADF-396, ADF-397, ADF-398, ADF-399, ADF-400, ADF-401, ADF-402, ADF-403, ADF-404, ADF-405, ADF-406, ADF-407, ADF-408, ADF-409, ADF-410, ADF-411, ADF-412, ADF-413, ADF-414, ADF-415, ADF-416, ADF-417, ADF-418, ADF-419, ADF-420, ADF-421, ADF-422, ADF-423, ADF-424, ADF-425, ADF-426, ADF-427, ADF-428, ADF-429, ADF-430, ADF-431, ADF-432, ADF-433, ADF-434, ADF-435, ADF-436, ADF-437, ADF-438, ADF-439, ADF-440, ADF-441, ADF-442, ADF-443, ADF-444, ADF-445, ADF-446, ADF-447, ADF-448, ADF-449, ADF-450, ADF-451, ADF-452, ADF-453, ADF-454, ADF-455, ADF-456, ADF-457, ADF-458, ADF-459, ADF-460, ADF-461, ADF-462, ADF-463, ADF-464, ADF-465, ADF-466, ADF-467, ADF-468, ADF-469, ADF-470, ADF-471, ADF-472, ADF-473, ADF-474, ADF-475, ADF-476, ADF-477, ADF-478, ADF-479, ADF-480, ADF-481, ADF-482, ADF-483, ADF-484, ADF-485, ADF-486, ADF-487, ADF-488, ADF-489, ADF-490, ADF-491, ADF-492, ADF-493, ADF-494, ADF-495, ADF-496, ADF-497, ADF-498, ADF-499, ADF-500, ADF-501, ADF-502, ADF-503, ADF-504, ADF-505, ADF-506, ADF-507, ADF-508, ADF-509, ADF-510, ADF-511, ADF-512, ADF-513, ADF-514, ADF-515, ADF-516, ADF-517, ADF-518, ADF-519, ADF-520, ADF-521, ADF-522, ADF-523, ADF-524, ADF-525, ADF-526, ADF-527, ADF-528, ADF-529, ADF-530, ADF-531, ADF-532, ADF-533, ADF-534, ADF-535, ADF-536, ADF-537, ADF-538, ADF-539, ADF-540, ADF-541, ADF-542, ADF-543, ADF-544, ADF-545, ADF-546, ADF-547, ADF-548, ADF-549 in any suitable combination. An altered abundance of one or more in any suitable combination of such ADFs in the brain tissue from one region of the subject relative to brain tissue from a one region of a subject or subjects free from Alzheimer's disease (e.g., a control sample or a previously determined reference range) indicates the presence of Alzheimer's disease.

In yet another embodiment of the invention, brain tissue from a subject is analyzed by 2D electrophoresis for quantitative detection of one or more of the following ADFs: ADF-1, ADF-3, ADF-5, ADF-6, ADF-8, ADF-9, ADF-10, ADF-11, ADF-12, ADF-13, ADF-15, ADF-16, ADF-22, ADF-23, ADF-24, ADF-25, ADF-26, ADF-27, ADF-29, ADF-31, ADF-33, ADF-35, ADF-37, ADF-39, ADF-42, ADF-54, ADF-56, ADF-61, ADF-62, ADF-66, ADF-67, ADF-68, ADF-70, ADF-72, ADF-77, ADF-78, ADF-79, ADF-81, ADF-82, ADF-85, ADF-87, ADF-88, ADF-90, ADF-91, ADF-92, ADF-94, ADF-95, ADF-97, ADF-98, ADF-101, ADF-102, ADF-103, ADF-105, ADF-107, ADF-108, ADF-109, ADF-111, ADF-112, ADF-113, ADF-115, ADF-117, ADF-119, ADF-120, ADF-121, ADF-124, ADF-125, ADF-126, ADF-127, ADF-128, ADF-129, ADF-130, ADF-131, ADF-132, ADF-133, ADF-138, ADF-139, ADF-140, ADF-141, ADF-142, ADF-143, ADF-144, ADF-146, ADF-148, ADF-149, ADF-150, ADF-151, ADF-152, ADF-153, ADF-154, ADF-155, ADF-156, ADF-157, ADF-159, ADF-160, ADF-162, ADF-163, ADF-165, ADF-172, ADF-173, ADF-175, ADF-176, ADF-182, ADF-183, ADF-188, ADF-189, ADF-191, ADF-193, ADF-194, ADF-196, ADF-202, ADF-204, ADF-208, ADF-209, ADF-216, ADF-217, ADF-220, ADF-223, ADF-228, ADF-229, ADF-230, ADF-232, ADF-236, ADF-237, ADF-240, ADF-243, ADF-245, ADF-250, ADF-251, ADF-252, ADF-253, ADF-254, ADF-255, ADF-256, ADF-257, ADF-258, ADF-259, ADF-260, ADF-261, ADF-262, ADF-263, ADF-264, ADF-265, ADF-266, ADF-267, ADF-268, ADF-269, ADF-270, ADF-271, ADF-272, ADF-273, ADF-274, ADF-275, ADF-276, ADF-277, ADF-278, ADF-279, ADF-280, ADF-281, ADF-282, ADF-283, ADF-284, ADF-285, ADF-286, ADF-287, ADF-288, ADF-289, ADF-290, ADF-291, ADF-292, ADF-293, ADF-294, ADF-295, ADF-296, ADF-297, ADF-298, ADF-299, ADF-300, ADF-301, ADF-302, ADF-303, ADF-304, ADF-305, ADF-306, ADF-307, ADF-308, ADF-309, ADF-311, ADF-312, ADF-313, ADF-314, ADF-315, ADF-316, ADF-317, ADF-318, ADF-319, ADF-320, ADF-321, ADF-322, ADF-323, ADF-324, ADF-325, ADF-326, ADF-327, ADF-328, ADF-329, ADF-330, ADF-331, ADF-332, ADF-333, ADF-334, ADF-335, ADF-336, ADF-337, ADF-338, ADF-339, ADF-340, ADF-341, ADF-342, ADF-343, ADF-344, ADF-345, ADF-346, ADF-347, ADF-348, ADF-349, ADF-350, ADF-351, ADF-352, ADF-353, ADF-354, ADF-355, ADF-356, ADF-357, ADF-358, ADF-359, ADF-360, ADF-361, ADF-362, ADF-363, ADF-364, ADF-365, ADF-366, ADF-367, ADF-368, ADF-369, ADF-370, ADF-371, ADF-372, ADF-373, ADF-374, ADF-375, ADF-376, ADF-377, ADF-378, ADF-379, ADF-380, ADF-381, ADF-382, ADF-383, ADF-384, ADF-386, ADF-387, ADF-388, ADF-389, ADF-390, ADF-391, ADF-392, ADF-393, ADF-394, ADF-395, ADF-396, ADF-397, ADF-398, ADF-399, ADF-400, ADF-401, ADF-402, ADF-403, ADF-404, ADF-405, ADF-406, ADF-407, ADF-408, ADF-409, ADF-410, ADF-411, ADF-412, ADF-413, ADF-414, ADF-415, ADF-416, ADF-417, ADF-418, ADF-419, ADF-420, ADF-421, ADF-422, ADF-423, ADF-424, ADF-425, ADF-426, ADF-427, ADF-428, ADF-429, ADF-430, ADF-431, ADF-432, ADF-433, ADF-434, ADF-435, ADF-436, ADF-437, ADF-438, ADF-439, ADF-440, ADF-441, ADF-442, ADF-443, ADF-444, ADF-445, ADF-446, ADF-447, ADF-448, ADF-449, ADF-450, ADF-451, ADF-452, ADF-453, ADF-454, ADF-455, ADF-456, ADF-457, ADF-458, ADF-459, ADF-460, ADF-461, ADF-462, ADF-463, ADF-464, ADF-465, ADF-466, ADF-467, ADF-468, ADF-469, ADF-470, ADF-471, ADF-472, ADF-473, ADF-474, ADF-475, ADF-476, ADF-477, ADF-478, ADF-479, ADF-480, ADF-481, ADF-482, ADF-483, ADF-484, ADF-485, ADF-486, ADF-487, ADF-488, ADF-489, ADF-490, ADF-491, ADF-492, ADF-493, ADF-494, ADF-495, ADF-496, ADF-497, ADF-498, ADF-499, ADF-500, ADF-501, ADF-502, ADF-503, ADF-504, ADF-505, ADF-506, ADF-507, ADF-508, ADF-509, ADF-510, ADF-511, ADF-512, ADF-513, ADF-514, ADF-515, ADF-516, ADF-517, ADF-518, ADF-519, ADF-520, ADF-521, ADF-522, ADF-523, ADF-524, ADF-525, ADF-526, ADF-527, ADF-528, ADF-529, ADF-530, ADF-531, ADF-532, ADF-533, ADF-534, ADF-535, ADF-536, ADF-537, ADF-538, ADF-539, ADF-540, ADF-541, ADF-542, ADF-543, ADF-544, ADF-545, ADF-546, ADF-547, ADF-548, ADF-549, wherein the ratio of the one or more ADFs relative to an Expression Reference Feature (ERF) in one region of the brain indicates that Alzheimer's disease is present.

In a further embodiment of the invention, brain tissue from a subject is analyzed by 2D electrophoresis for quantitative detection of one or more ADFs, or any suitable combination of them, whose altered ADF/ERF ratio(s) in a test sample from one region relative to the ADF/ERF ratio(s) in a control sample from the same brain region indicates the presence of Alzheimer's disease, i.e., ADF-1, ADF-3, ADF-5, ADF-6, ADF-8, ADF-9, ADF-10, ADF-11, ADF-12, ADF-13, ADF-15, ADF-16, ADF-22, ADF-23, ADF-24, ADF-25, ADF-26, ADF-27, ADF-29, ADF-31, ADF-33, ADF-35, ADF-37, ADF-39, ADF-42, ADF-54, ADF-56, ADF-61, ADF-62, ADF-66, ADF-67, ADF-68, ADF-70, ADF-72, ADF-77, ADF-78, ADF-79, ADF-81, ADF-82, ADF-85, ADF-87, ADF-88, ADF-90, ADF-91, ADF-92, ADF-94, ADF-95, ADF-97, ADF-98, ADF-101, ADF-102, ADF-103, ADF-105, ADF-107, ADF-108, ADF-109, ADF-111, ADF-112, ADF-113, ADF-115, ADF-117, ADF-119, ADF-120, ADF-121, ADF-124, ADF-125, ADF-126, ADF-127, ADF-128, ADF-129, ADF-130, ADF-131, ADF-132, ADF-133, ADF-138, ADF-139, ADF-140, ADF-141, ADF-142, ADF-143, ADF-144, ADF-146, ADF-148, ADF-149, ADF-150, ADF-151, ADF-152, ADF-153, ADF-154, ADF-155, ADF-156, ADF-157, ADF-159, ADF-160, ADF-162, ADF-163, ADF-165, ADF-172, ADF-173, ADF-175, ADF-176, ADF-182, ADF-183, ADF-188, ADF-189, ADF-191, ADF-193, ADF-194, ADF-196, ADF-202, ADF-204, ADF-208, ADF-209, ADF-216, ADF-217, ADF-220, ADF-223, ADF-228, ADF-229, ADF-230, ADF-232, ADF-236, ADF-237, ADF-240, ADF-243, ADF-245, ADF-250, ADF-251, ADF-252, ADF-253, ADF-254, ADF-255, ADF-256, ADF-257, ADF-258, ADF-259, ADF-260, ADF-261, ADF-262, ADF-263, ADF-264, ADF-265, ADF-266, ADF-267, ADF-268, ADF-269, ADF-270, ADF-271, ADF-272, ADF-273, ADF-274, ADF-275, ADF-276, ADF-277, ADF-278, ADF-279, ADF-280, ADF-281, ADF-282, ADF-283, ADF-284, ADF-285, ADF-286, ADF-287, ADF-288, ADF-289, ADF-290, ADF-291, ADF-292, ADF-293, ADF-294, ADF-295, ADF-296, ADF-297, ADF-298, ADF-299, ADF-300, ADF-301, ADF-302, ADF-303, ADF-304, ADF-305, ADF-306, ADF-307, ADF-308, ADF-309, ADF-311, ADF-312, ADF-313, ADF-314, ADF-315, ADF-316, ADF-317, ADF-318, ADF-319, ADF-320, ADF-321, ADF-322, ADF-323, ADF-324, ADF-325, ADF-326, ADF-327, ADF-328, ADF-329, ADF-330, ADF-331, ADF-332, ADF-333, ADF-334, ADF-335, ADF-336, ADF-337, ADF-338, ADF-339, ADF-340, ADF-341, ADF-342, ADF-343, ADF-344, ADF-345, ADF-346, ADF-347, ADF-348, ADF-349, ADF-350, ADF-351, ADF-352, ADF-353, ADF-354, ADF-355, ADF-356, ADF-357, ADF-358, ADF-359, ADF-360, ADF-361, ADF-362, ADF-363, ADF-364, ADF-365, ADF-366, ADF-367, ADF-368, ADF-369, ADF-370, ADF-371, ADF-372, ADF-373, ADF-374, ADF-375, ADF-376, ADF-377, ADF-378, ADF-379, ADF-380, ADF-381, ADF-382, ADF-383, ADF-384, ADF-386, ADF-387, ADF-388, ADF-389, ADF-390, ADF-391, ADF-392, ADF-393, ADF-394, ADF-395, ADF-396, ADF-397, ADF-398, ADF-399, ADF-400, ADF-401, ADF-402, ADF-403, ADF-404, ADF-405, ADF-406, ADF-407, ADF-408, ADF-409, ADF-410, ADF-411, ADF-412, ADF-413, ADF-414, ADF-415, ADF-416, ADF-417, ADF-418, ADF-419, ADF-420, ADF-421, ADF-422, ADF-423, ADF-424, ADF-425, ADF-426, ADF-427, ADF-428, ADF-429, ADF-430, ADF-431, ADF-432, ADF-433, ADF-434, ADF-435, ADF-436, ADF-437, ADF-438, ADF-439, ADF-440, ADF-441, ADF-442, ADF-443, ADF-444, ADF-445, ADF-446, ADF-447, ADF-448, ADF-449, ADF-450, ADF-451, ADF-452, ADF-453, ADF-454, ADF-455, ADF-456, ADF-457, ADF-458, ADF-459, ADF-460, ADF-461, ADF-462, ADF-463, ADF-464, ADF-465, ADF-466, ADF-467, ADF-468, ADF-469, ADF-470, ADF-471, ADF-472, ADF-473, ADF-474, ADF-475, ADF-476, ADF-477, ADF-478, ADF-479, ADF-480, ADF-481, ADF-482, ADF-483, ADF-484, ADF-485, ADF-486, ADF-487, ADF-488, ADF-489, ADF-490, ADF-491, ADF-492, ADF-493, ADF-494, ADF-495, ADF-496, ADF-497, ADF-498, ADF-499, ADF-500, ADF-501, ADF-502, ADF-503, ADF-504, ADF-505, ADF-506, ADF-507, ADF-508, ADF-509, ADF-510, ADF-511, ADF-512, ADF-513, ADF-514, ADF-515, ADF-516, ADF-517, ADF-518, ADF-519, ADF-520, ADF-521, ADF-522, ADF-523, ADF-524, ADF-525, ADF-526, ADF-527, ADF-528, ADF-529, ADF-530, ADF-531, ADF-532, ADF-533, ADF-534, ADF-535, ADF-536, ADF-537, ADF-538, ADF-539, ADF-540, ADF-541, ADF-542, ADF-543, ADF-544, ADF-545, ADF-546, ADF-547, ADF-548, ADF-549.

In a preferred embodiment, brain tissue from a subject is analyzed for quantitative detection of a plurality of ADFs.

In a preferred embodiment of the invention, brain tissue from a subject (e.g., a subject suspected of having Alzheimer's disease) is analysed by 2D electrophoresis for quantitative detection of one or more of the following ADFs: ADF-1, ADF-6, ADF-8, ADF-9, ADF-10, ADF-16, ADF-26, ADF-27, ADF-31, ADF-67, ADF-77, ADF-82, ADF-90, ADF-98, ADF-119, ADF-120, ADF-124, ADF-130, ADF-132, ADF-148, ADF-150, ADF-152, ADF-159, ADF-162, ADF-163, ADF-204, ADF-237, ADF-268, ADF-270, ADF-271, ADF-275, ADF-284, ADF-294, ADF-297, ADF-318, ADF-328, ADF-338, ADF-346, ADF-382, ADF-384, ADF-411, ADF-413, ADF-419, ADF-427, ADF-443, ADF-450, ADF-452, ADF-456, ADF-458, ADF-491, ADF-507, ADF-510 in any suitable combination. An altered abundance of one or more in any suitable combination of such ADFs in the brain tissue from any region of the brain of the subject relative to brain tissue from any region of the brain of a subject or subjects free from Alzheimer's disease (e.g., a control sample or a previously determined reference range) indicates the presence of Alzheimer's disease.

In yet another embodiment of the invention, brain tissue from a subject is analyzed by 2D electrophoresis for quantitative detection of one or more of the following ADFs ADF-1, ADF-6, ADF-8, ADF-9, ADF-10, ADF-16, ADF-26, ADF-27, ADF-31, ADF-67, ADF-77, ADF-82, ADF-90, ADF-98, ADF-119, ADF-120, ADF-124, ADF-130, ADF-132, ADF-148, ADF-150, ADF-152, ADF-159, ADF-162, ADF-163, ADF-204, ADF-237, ADF-268, ADF-270, ADF-271, ADF-275, ADF-284, ADF-294, ADF-297, ADF-318, ADF-328, ADF-338, ADF-346, ADF-382, ADF-384, ADF-411, ADF-413, ADF-419, ADF-427, ADF-443, ADF-450, ADF-452, ADF-456, ADF-458, ADF-491, ADF-507, ADF-510 wherein the ratio of the one or more ADFs relative to an Expression Reference Feature (ERF) in any region of the brain indicates that Alzheimer's disease is present.

In a further embodiment of the invention, brain tissue from a subject is analyzed by 2D electrophoresis for quantitative detection of one or more ADFs, or any suitable combination of them, whose altered ADF/ERF ratio(s) in a test sample from any region of the brain relative to the ADF/ERF ratio(s) in a control sample from any region of the brain indicates the presence of Alzheimer's disease, i.e., ADF-1, ADF-6, ADF-8, ADF-9, ADF-10, ADF-16, ADF-26, ADF-27, ADF-31, ADF-67, ADF-77, ADF-82, ADF-90, ADF-98, ADF-119, ADF-120, ADF-124, ADF-130, ADF-132, ADF-148, ADF-150, ADF-152, ADF-159, ADF-162, ADF-163, ADF-204, ADF-237, ADF-268, ADF-270, ADF-271, ADF-275, ADF-284, ADF-294, ADF-297, ADF-318, ADF-328, ADF-338, ADF-346, ADF-382, ADF-384, ADF-411, ADF-413, ADF-419, ADF-427, ADF-443, ADF-450, ADF-452, ADF-456, ADF-458, ADF-491, ADF-507, ADF-510.

In a preferred embodiment, brain tissue from a subject is analyzed for quantitative detection of a plurality of ADFs.

The ADFs of the invention can be used, for example, for detection, treatment, diagnosis, or for drug development. In one embodiment of the invention, brain tissue from a subject (e.g., a subject suspected of having Alzheimer's disease) is analyzed by 2D electrophoresis for quantitative detection of one or more of the following ADFs: ADF-1, ADF-6, ADF-8, ADF-9, ADF-10, ADF-12, ADF-23, ADF-25, ADF-26, ADF-27, ADF-29, ADF-31, ADF-67, ADF-77, ADF-79, ADF-85, ADF-90, ADF-91, ADF-102, ADF-103, ADF-119, ADF-120, ADF-121, ADF-124, ADF-132, ADF-142, ADF-144, ADF-148, ADF-149, ADF-150, ADF-151, ADF-152, ADF-154, ADF-155, ADF-157, ADF-159, ADF-160, ADF-162, ADF-163, ADF-165, ADF-173, ADF-175, ADF-176, ADF-193, ADF-202, ADF-204, ADF-250, ADF-251, ADF-260, ADF-262, ADF-266, ADF-270, ADF-271, ADF-272, ADF-275, ADF-280, ADF-282, ADF-283, ADF-286, ADF-288, ADF-295, ADF-300, ADF-302, ADF-304, ADF-319, ADF-321, ADF-331, ADF-335, ADF-336, ADF-337, ADF-355, ADF-364, ADF-366, ADF-367, ADF-368, ADF-373, ADF-375, ADF-376, ADF-381, ADF-387, ADF-390, ADF-399, ADF-400, ADF-403, ADF-404, ADF-405, ADF-410, ADF-412, ADF-414, ADF-415, ADF-419, ADF-423, ADF-429, ADF-434, ADF-437, ADF-442, ADF-443, ADF-444, ADF-446, ADF-452, ADF-456, ADF-469, ADF-471, ADF-472, ADF-478, ADF-485, ADF-491, ADF-493, ADF-495, ADF-504, ADF-505, ADF-510, ADF-511, ADF-514, ADF-518, ADF-524, ADF-527, ADF-535 in any suitable combination. An altered abundance of one or more in any suitable combination of such ADFs in the brain tissue from the subject relative to brain tissue from a subject or subjects free from Alzheimer's disease (e.g., a control sample or a previously determined reference range) is an early indicator of the presence of Alzheimer's disease.

In yet another embodiment of the invention, brain tissue from a subject is analyzed by 2D electrophoresis for quantitative detection of one or more of the following ADFs ADF-1, ADF-6, ADF-8, ADF-9, ADF-10, ADF-12, ADF-23, ADF-25, ADF-26, ADF-27, ADF-29, ADF-31, ADF-67, ADF-77, ADF-79, ADF-85, ADF-90, ADF-91, ADF-102, ADF-103, ADF-119, ADF-120, ADF-121, ADF-124, ADF-132, ADF-142, ADF-144, ADF-148, ADF-149, ADF-150, ADF-151, ADF-152, ADF-154, ADF-155, ADF-157, ADF-159, ADF-160, ADF-162, ADF-163, ADF-165, ADF-173, ADF-175, ADF-176, ADF-193, ADF-202, ADF-204, ADF-250, ADF-251, ADF-260, ADF-262, ADF-266, ADF-270, ADF-271, ADF-272, ADF-275, ADF-280, ADF-282, ADF-283, ADF-286, ADF-288, ADF-295, ADF-300, ADF-302, ADF-304, ADF-319, ADF-321, ADF-331, ADF-335, ADF-336, ADF-337, ADF-355, ADF-364, ADF-366, ADF-367, ADF-368, ADF-373, ADF-375, ADF-376, ADF-381, ADF-387, ADF-390, ADF-399, ADF-400, ADF-403, ADF-404, ADF-405, ADF-410, ADF-412, ADF-414, ADF-415, ADF-419, ADF-423, ADF-429, ADF-434, ADF-437, ADF-442, ADF-443, ADF-444, ADF-446, ADF-452, ADF-456, ADF-469, ADF-471, ADF-472, ADF-478, ADF-485, ADF-491, ADF-493, ADF-495, ADF-504, ADF-505, ADF-510, ADF-511, ADF-514, ADF-518, ADF-524, ADF-527, ADF-535 wherein the ratio of the one or more ADFs relative to an Expression Reference Feature (ERF) is an early indicator of the presence of Alzheimer's disease.

In a further embodiment of the invention, brain tissue from a subject is analyzed by 2D electrophoresis for quantitative detection of one or more ADFs, or any suitable combination of them, whose altered ADF/ERF ratio(s) in a test sample relative to the ADF/ERF ratio(s) in a control sample is an early indicator of the presence of Alzheimer's disease, i.e., ADF-1, ADF-6, ADF-8, ADF-9, ADF-10, ADF-12, ADF-23, ADF-25, ADF-26, ADF-27, ADF-29, ADF-31, ADF-67, ADF-77, ADF-79, ADF-85, ADF-90, ADF-91, ADF-102, ADF-103, ADF-119, ADF-120, ADF-121, ADF-124, ADF-132, ADF-142, ADF-144, ADF-148, ADF-149, ADF-150, ADF-151, ADF-152, ADF-154, ADF-155, ADF-157, ADF-159, ADF-160, ADF-162, ADF-163, ADF-165, ADF-173, ADF-175, ADF-176, ADF-193, ADF-202, ADF-204, ADF-250, ADF-251, ADF-260, ADF-262, ADF-266, ADF-270, ADF-271, ADF-272, ADF-275, ADF-280, ADF-282, ADF-283, ADF-286, ADF-288, ADF-295, ADF-300, ADF-302, ADF-304, ADF-319, ADF-321, ADF-331, ADF-335, ADF-336, ADF-337, ADF-355, ADF-364, ADF-366, ADF-367, ADF-368, ADF-373, ADF-375, ADF-376, ADF-381, ADF-387, ADF-390, ADF-399, ADF-400, ADF-403, ADF-404, ADF-405, ADF-410, ADF-412, ADF-414, ADF-415, ADF-419, ADF-423, ADF-429, ADF-434, ADF-437, ADF-442, ADF-443, ADF-444, ADF-446, ADF-452, ADF-456, ADF-469, ADF-471, ADF-472, ADF-478, ADF-485, ADF-491, ADF-493, ADF-495, ADF-504, ADF-505, ADF-510, ADF-511, ADF-514, ADF-518, ADF-524, ADF-527, ADF-535.

In a preferred embodiment, brain tissue from a subject is analyzed for quantitative detection of a plurality of ADFs.

5.3 Alzheimer's Disease-Associated Protein Isoforms (ADPIs)

In another aspect of the invention, brain tissue from a subject is analyzed for quantitative detection of one or more Alzheimer's Disease-Associated Protein Isoforms (ADPIs), e.g. for screening, treatment or diagnosis of Alzheimer's disease or for development of pharmaceutical products. As is well known in the art, a given protein may be expressed as one or more variant forms that differ in amino acid composition (e.g. as a result of alternative mRNA or premRNA processing, e.g. alternative splicing or limited proteolysis) or as a result of differential post-translational modification (e.g., glycosylation, phosphorylation, acylation), or both, so that proteins of identical amino acid sequence can differ in their pI, MW, or both. “Alzheimer's Disease-Associated Protein Isoform” refers to a protein that is differentially present in brain tissue from a subject having Alzheimer's disease compared with brain tissue from a subject free from Alzheimer's disease.

ADPIs are described herein by the amino acid sequencing of ADFs, as depicted in FIG. 4 and described above. Three groups of ADPIs have been identified by partial amino acid sequencing of ADFs, using the methods and apparatus of the Preferred Technology. One skilled in the art can identify sequence information from proteins analysed by mass spectrometry and/or tandem mass spectrometry using various spectral interpretation methods and database searching tools. Examples of some of these methods and tools can be found at the Swiss Institute of Bioinformatics web site at http://www.expasy.com/, and the European Molecular Biology Laboratory web site at http://www.narrador.embl-heidelberg.de/GroupPages/PageLink/peptidesearchpage.html. Identification of ADPIs was performed primarily using the SEQUEST search program (Eng et al., 1994, J. Am. Soc. Mass Spectrom. 5:976-989) and the method described in PCT Application No. PCT/GB01/04034, which is incorporated herein by reference in its entirety.

ADPIs were classified into one of 3 groups for each statistical analysis. The first group consists of ADPIs that are present in at least 50% of disease or control samples and have a fold change (increase or decrease) of at least 1.5, (a+: fold increase of at least 1.5 relative to controls, a−: fold decrease of at least 1.5 relative to control). The second group consists of ADPIs that are present in at least 50% of disease or controls samples with a fold change of at least 1.5 where the p-value for the fold change is less than 0.05 (b+: fold increase of at least 1.5 with p<0.05, b−: fold decrease of at least 1.5 with p<0.05). The third group consists of ADPIs that are present in at least 50% of either the disease or the control samples but is absent from all samples in the other group, (c+: present in at least 50% of patient samples and absent from all control samples, c−: present in at least 50% of control samples and absent from all disease samples). Blank cells in Table III reflect an ADPI which did not meet the criteria for inclusion in any of the above groups for a given condition.

Table III ADPIs Altered in Brain Tissue of Subjects having Alzheimer's Disease

TABLE III

| | | All | selected | | | | | | EC/HC vs. |
|---|---|---|---|---|---|---|---|---|---|
| ADF# | ADPI# | HC | HC | FC | NC | EC | Am | ALL | FC/NC/Am |
| ADF-1 | ADPI-1 | b+ | a+ | | a− | a− | | a+ | b+ |
| ADF-3 | ADPI-3.1 | b− | | | | | a− | | |
| ADF-3 | ADPI-3.2 | b− | | | | | a− | | |
| ADF-3 | ADPI-3.3 | b− | | | | | a− | | |
| ADF-5 | ADPI-5.3 | a+ | | | | | | | |
| ADF-6 | ADPI-6 | b+ | b+ | b+ | a+ | a+ | a+ | b+ | b+ |
| ADF-8 | ADPI-8 | b+ | b+ | | a− | a− | a+ | b+ | b+ |
| ADF-9 | ADPI-9 | | a− | | a+ | | a+ | a+ | a− |
| ADF-10 | ADPI-10 | b+ | b+ | | | a+ | a+ | b+ | b+ |
| ADF-11 | ADPI-11 | | | b− | | a− | | | |
| ADF-12 | ADPI-12.1 | | a+ | | | | | | b+ |
| ADF-12 | ADPI-12.2 | | a+ | | | | | | b+ |
| ADF-13 | ADPI-13 | | | | | | a− | | |
| ADF-15 | ADPI-15 | | | | a− | a− | a− | | |
| ADF-16 | ADPI-16 | a− | | | a+ | | a− | a− | |
| ADF-22 | ADPI-22 | | | | a+ | | a− | | |
| ADF-23 | ADPI-23 | b− | b− | | | a+ | a− | | b− |
| ADF-24 | ADPI-24 | | | a+ | | | a+ | | |
| ADF-25 | ADPI-25.3 | | | b+ | a− | | | | a− |
| ADF-25 | ADPI-25.2 | | | b+ | a− | | | | a− |
| ADF-26 | ADPI-26 | b+ | b+ | a+ | a+ | a+ | a+ | b+ | b+ |
| ADF-27 | ADPI-27 | b+ | b+ | | | a+ | a+ | a+ | b+ |
| ADF-29 | ADPI-29 | a+ | a+ | | a+ | a− | | | b+ |
| ADF-31 | ADPI-31 | b+ | a+ | a− | | | a− | a+ | b+ |
| ADF-33 | ADPI-33.1 | a− | | | a− | | | | |
| ADF-35 | ADPI-35 | | | | | | a+ | | |
| ADF-37 | ADPI-37 | b− | b− | | | | | | |
| ADF-39 | ADPI-39 | | a+ | | | | | | |
| ADF-41 | ADPI-41 | | | | | a− | | | |
| ADF-42 | ADPI-42 | | b− | | a− | | | | |
| ADF-54 | ADPI-54 | | b− | | | | | | |
| ADF-56 | ADPI-56 | b− | b− | | a− | | | | |
| ADF-61 | ADPI-61 | | | | a− | a− | | | |
| ADF-62 | ADPI-62 | | a− | | a+ | | a+ | | |
| ADF-66 | ADPI-66 | | b− | | | a+ | a− | | |
| ADF-67 | ADPI-67 | a− | a− | a+ | a− | | a− | a− | b+ |
| ADF-68 | ADPI-68 | | | | | a− | | | |
| ADF-70 | ADPI-70 | | | | | a+ | | | |
| ADF-72 | ADPI-72 | | | | | | a− | | |
| ADF-77 | ADPI-77 | b+ | b+ | | a− | | | a+ | b+ |
| ADF-78 | ADPI-78.2 | b+ | b+ | | a− | | | a+ | b+ |
| ADF-78 | ADPI-78.3 | | | | | a− | c+ | | |
| ADF-79 | ADPI-79 | | | | | a− | a+ | | b− |
| ADF-81 | ADPI-81.2 | a− | | | a+ | a− | | | |
| ADF-82 | ADPI-82 | | | | c− | | a− | a− | |
| ADF-85 | ADPI-85 | | | | a− | | a− | | b+ |
| ADF-87 | ADPI-87.1 | | | | | | a− | | |
| ADF-88 | ADPI-88 | | | | a− | | | | |
| ADF-90 | ADPI-90 | b+ | b+ | | a+ | | | b+ | b+ |
| ADF-91 | ADPI-91.1 | | | | a− | | a− | | b+ |
| ADF-91 | ADPI-91.2 | | | | a− | | a− | | b+ |
| ADF-92 | ADPI-92 | | | | a+ | a+ | a− | | |
| ADF-94 | ADPI-94 | | | | | | a+ | | |
| ADF-95 | ADPI-95 | | | | a+ | a− | a+ | | |
| ADF-97 | ADPI-97 | b+ | b+ | | | | a+ | | |
| ADF-98 | ADPI-98 | b+ | a+ | a+ | a− | | a+ | b+ | |
| ADF-101 | ADPI-101 | | | | a− | | | | |
| ADF-102 | ADPI-102.1 | | b− | | | a+ | a− | | b− |
| ADF-103 | ADPI-103 | a+ | a+ | a+ | a− | a− | | | b+ |
| ADF-105 | ADPI-105.2 | | | | a+ | a− | a− | | |
| ADF-105 | ADPI-105.1 | | | | a+ | a− | a− | | |
| ADF-107 | ADPI-107 | | | | | a− | | | |
| ADF-108 | ADPI-108 | | | | | | a+ | | |
| ADF-109 | ADPI-109 | | | | a− | | | | |
| ADF-111 | ADPI-111.1 | | | | a+ | c− | a+ | | |
| ADF-112 | ADPI-112 | | | | a− | a− | | | |
| ADF-113 | ADPI-113 | | | | a− | | | | |
| ADF-115 | ADPI-115 | | | | | a− | | | |
| ADF-117 | ADPI-117.1 | | | | | a+ | a+ | | |
| ADF-119 | ADPI-119 | b+ | a+ | | c− | | a+ | b+ | b+ |
| ADF-120 | ADPI-120 | b+ | b+ | b+ | c+ | a+ | | b+ | b+ |
| ADF-121 | ADPI-121 | | | | | | | | b− |
| ADF-124 | ADPI-124 | | b− | b− | a− | | | b− | a+ |
| ADF-125 | ADPI-125 | | | | | c+ | a− | | |
| ADF-126 | ADPI-126 | | | | | c− | a+ | | |
| ADF-126 | ADPI-126 | | | | | c− | a+ | | |
| ADF-126 | ADPI-126 | | | | | c− | a+ | | |
| ADF-127 | ADPI-127 | | | a− | a+ | | a− | | |
| ADF-128 | ADPI-128 | | | | | a− | a− | | |
| ADF-129 | ADPI-129 | b− | b− | | | a− | | | |
| ADF-130 | ADPI-130 | | | | | a− | a+ | a− | |
| ADF-131 | ADPI-131 | b− | | | | a− | a− | | |
| ADF-132 | ADPI-132 | a+ | a+ | | a− | a− | | a+ | b+ |
| ADF-133 | ADPI-133 | | | a− | a− | a− | | | |
| ADF-138 | ADPI-138 | | | | a− | | a+ | | |
| ADF-139 | ADPI-139 | | b− | | | c− | a+ | | |
| ADF-140 | ADPI-140 | | | | | c− | | | |
| ADF-141 | ADPI-141 | | | | a− | | c− | | |
| ADF-142 | ADPI-142.2 | a− | | | a− | | | | a+ |
| ADF-142 | ADPI-142.1 | a− | | | a− | | | | a+ |
| ADF-143 | ADPI-143.1 | | | | a− | | | | |
| ADF-143 | ADPI-143.2 | | | | a− | | | | |
| ADF-144 | ADPI-144 | b+ | b+ | | c+ | c− | a+ | | b+ |
| ADF-146 | ADPI-146.2 | | | | | | a− | | |
| ADF-146 | ADPI-146.1 | | | | | | a− | | |
| ADF-148 | ADPI-148 | b+ | b+ | b+ | a+ | a+ | | b+ | a+ |
| ADF-149 | ADPI-149.1 | | | | | | a+ | | a+ |
| ADF-150 | ADPI-150 | b+ | b+ | a+ | | a+ | a+ | a+ | b+ |
| ADF-151 | ADPI-151.2 | a+ | | | c+ | a− | a− | | a+ |
| ADF-151 | ADPI-151.1 | a+ | | | c+ | a− | a− | | a+ |
| ADF-152 | ADPI-152 | b+ | b+ | b+ | a+ | a+ | | b+ | b+ |
| ADF-153 | ADPI-153.1 | | | | | a+ | | | |
| ADF-153 | ADPI-153.2 | | | | | a+ | | | |
| ADF-153 | ADPI-153.3 | | | | | a+ | | | |
| ADF-154 | ADPI-154 | b+ | | | a+ | a− | | b+ | |
| ADF-155 | ADPI-155 | | | | a+ | | | | |
| ADF-156 | ADPI-156.2 | | | b+ | a+ | | a+ | | |
| ADF-156 | ADPI-156.1 | | | b+ | a+ | | a+ | | |
| ADF-157 | ADPI-157 | | | | | a+ | | | b− |
| ADF-159 | ADPI-159.1 | b+ | b+ | b+ | a+ | a+ | a+ | b+ | b+ |
| ADF-159 | ADPI-159.2 | b+ | b+ | b+ | a+ | a+ | a+ | b+ | b+ |
| ADF-160 | ADPI-160 | | | | | a+ | | | b− |
| ADF-162 | ADPI-162.2 | b+ | b+ | a+ | a+ | a+ | a+ | b+ | b+ |
| ADF-162 | ADPI-162.3 | b+ | b+ | a+ | a+ | a+ | a+ | b+ | b+ |
| ADF-162 | ADPI-162.1 | b+ | b+ | a+ | a+ | a+ | a+ | b+ | b+ |
| ADF-163 | ADPI-163 | b+ | a+ | b+ | | | a+ | b+ | b+ |
| ADF-165 | ADPI-165 | b+ | b+ | | | | | | b+ |
| ADF-172 | ADPI-172 | | b− | | a+ | a+ | | | |
| ADF-173 | ADPI-173.1 | | b− | | | | | | b− |
| ADF-173 | ADPI-173.2 | | b− | | | | | | b− |
| ADF-175 | ADPI-175.2 | | | | a+ | a− | c− | | a+ |
| ADF-175 | ADPI-175.1 | | | | a+ | a− | c− | | a+ |
| ADF-176 | ADPI-176 | b− | b− | | | | | | b− |
| ADF-182 | ADPI-182 | | | | a− | | | | |
| ADF-183 | ADPI-183 | b− | b− | | | | | | |
| ADF-188 | ADPI-188.1 | | | | a+ | a− | a+ | | |
| ADF-189 | ADPI-189.2 | | | | a+ | | | | |
| ADF-191 | ADPI-191 | | b− | | | | | | |
| ADF-193 | ADPI-193 | a− | b− | | | | a+ | | b− |
| ADF-194 | ADPI-194.2 | | b− | | | | | | |
| ADF-196 | ADPI-196.2 | | | b+ | | a+ | a+ | | |
| ADF-196 | ADPI-196.3 | | | b+ | | a+ | a+ | | |
| ADF-196 | ADPI-196.1 | | | b+ | | a+ | a+ | | |
| ADF-196 | ADPI-196.4 | | | b+ | | a+ | a+ | | |
| ADF-202 | ADPI-202 | b− | b− | | | | | | b− |
| ADF-204 | ADPI-204 | b+ | a+ | b+ | | | | b+ | b+ |
| ADF-208 | ADPI-208 | | | | | | a+ | | |
| ADF-209 | ADPI-209 | | b+ | | | | a+ | | |
| ADF-216 | ADPI-216 | | | | | a+ | | | |
| ADF-217 | ADPI-217 | | | | a+ | a+ | | | |
| ADF-220 | ADPI-220 | | | a+ | a+ | a+ | | | |
| ADF-223 | ADPI-223 | b− | b− | | | | a− | | |
| ADF-228 | ADPI-228 | | | | | c+ | | | |
| ADF-229 | ADPI-229 | b− | b− | | | | | | |
| ADF-230 | ADPI-230.1 | | | | | | c− | | |
| ADF-230 | ADPI-230.2 | | | | | | c− | | |
| ADF-232 | ADPI-232.1 | | | b− | | | | | |
| ADF-232 | ADPI-232.2 | | | b− | | | | | |
| ADF-236 | ADPI-236.2 | | | | c+ | a+ | | | |
| ADF-236 | ADPI-236.3 | | | | c+ | a+ | | | |
| ADF-237 | ADPI-237.2 | b− | a− | | a+ | a− | a− | b− | |
| ADF-237 | ADPI-237.1 | b− | a− | | a+ | a− | a− | b− | |
| ADF-240 | ADPI-240 | a− | | | a+ | | | | |
| ADF-243 | ADPI-243 | | | | | a+ | a− | | |
| ADF-245 | ADPI-245 | a− | | | | | c− | | |
| ADF-250 | ADPI-250 | | | | | a+ | | | b+ |
| ADF-251 | ADPI-251 | | | | | a+ | a− | | a+ |
| ADF-252 | ADPI-252.1 | | | | | a+ | | | |
| ADF-252 | ADPI-252.2 | | | | | a+ | | | |
| ADF-253 | ADPI-253 | | b− | | | | | | |
| ADF-254 | ADPI-254 | | a− | | a+ | | | | |
| ADF-255 | ADPI-255 | a− | b− | | | | | | |
| ADF-256 | ADPI-256 | a− | b− | | | | | | |
| ADF-257 | ADPI-257.1 | b+ | a+ | | a− | a+ | | | |
| ADF-257 | ADPI-257.2 | b+ | a+ | | a− | a+ | | | |
| ADF-258 | ADPI-258 | b+ | b+ | | c− | a+ | a+ | | |
| ADF-259 | ADPI-259.1 | | | | a− | | | | |
| ADF-259 | ADPI-259.2 | | | a− | | | | | |
| ADF-260 | ADPI-260 | | b− | | a+ | | a− | | a− |
| ADF-261 | ADPI-261 | | | | | a+ | a− | | |
| ADF-262 | ADPI-262 | b+ | a+ | b+ | c− | a− | | | b+ |
| ADF-263 | ADPI-263 | | b− | | | | | | |
| ADF-264 | ADPI-264 | | | | a+ | | | | |
| ADF-265 | ADPI-265 | | | | | | a+ | | |
| ADF-266 | ADPI-266 | | | | | a+ | | | b+ |
| ADF-267 | ADPI-267 | | | | | a+ | | | |
| ADF-268 | ADPI-268.1 | b− | a− | a− | a− | | b− | | |
| ADF-268 | ADPI-268.2 | b− | a− | a− | a− | a− | | b− | |
| ADF-269 | ADPI-269 | | a− | | a− | | a+ | | |
| ADF-270 | ADPI-270 | b+ | b+ | a+ | | a− | | b+ | a+ |
| ADF-271 | ADPI-271 | a+ | | | | | | a+ | a+ |
| ADF-272 | ADPI-272 | | | | | a+ | a− | | a+ |
| ADF-273 | ADPI-273 | b− | b− | | | | | | |
| ADF-274 | ADPI-274 | | | | | a+ | | | |
| ADF-275 | ADPI-275 | b+ | a+ | a+ | | a− | a+ | b+ | b+ |
| ADF-276 | ADPI-276 | b− | b− | | | | | | |
| ADF-277 | ADPI-277 | | | b− | a+ | a+ | a− | | |
| ADF-278 | ADPI-278.1 | | a− | a− | | | c+ | | |
| ADF-278 | ADPI-278.2 | | a− | a− | | | c+ | | |
| ADF-279 | ADPI-279.1 | | | | | | a− | | |
| ADF-279 | ADPI-279.2 | | | | | | a− | | |
| ADF-280 | ADPI-280 | | | | | | | | b− |
| ADF-281 | ADPI-281 | | | a− | | | | | |
| ADF-282 | ADPI-282 | | b− | | | | | | a− |
| ADF-283 | ADPI-283 | | | | | | a− | | b+ |
| ADF-284 | ADPI-284 | b− | b− | | a+ | a− | a− | b− | |
| ADF-285 | ADPI-285 | | | | a+ | | | | |
| ADF-286 | ADPI-286 | | | | | | | | b+ |
| ADF-287 | ADPI-287 | | | b− | | | | | |
| ADF-288 | ADPI-288 | | a− | | | a− | a+ | | b− |
| ADF-289 | ADPI-289 | | | | c+ | a+ | c− | | |
| ADF-290 | ADPI-290 | | a+ | | | | | | |
| ADF-291 | ADPI-291 | | | a− | a− | | a+ | | |
| ADF-292 | ADPI-292 | | | | a− | c+ | | | |
| ADF-293 | ADPI-293 | | b− | | | | | | |
| ADF-294 | ADPI-294 | | | | a− | a− | a− | b− | |
| ADF-295 | ADPI-295 | | | | | | | | b− |
| ADF-296 | ADPI-296 | | | | a− | | a− | | |
| ADF-297 | ADPI-297 | a− | a− | | a+ | a− | | b− | |
| ADF-298 | ADPI-298 | | | | a− | | c− | | |
| ADF-299 | ADPI-299 | | | | | a− | | | |
| ADF-300 | ADPI-300 | a− | a− | a+ | a− | a+ | | | a− |
| ADF-301 | ADPI-301 | | | | | c+ | | | |
| ADF-302 | ADPI-302 | | | | | a+ | | | a+ |
| ADF-303 | ADPI-303 | b+ | | | | | a+ | | |
| ADF-304 | ADPI-304 | | | | | | | | b− |
| ADF-305 | ADPI-305 | a− | b− | | | | a− | | |
| ADF-306 | ADPI-306 | | | | a+ | | | | |
| ADF-307 | ADPI-307.1 | | | | | a− | | | |
| ADF-307 | ADPI-307.2 | | | | | a− | | | |
| ADF-308 | ADPI-308 | b− | b− | | a− | | a+ | | |
| ADF-309 | ADPI-309 | | | | | a− | | | |
| ADF-311 | ADPI-311 | | b+ | | | | a+ | | |
| ADF-312 | ADPI-312 | | | | | a+ | a− | | |
| ADF-313 | ADPI-313 | | | | a− | | a+ | | |
| ADF-314 | ADPI-314 | | | | | a− | a+ | | |
| ADF-315 | ADPI-315 | | | a+ | a− | | | | |
| ADF-316 | ADPI-316.1 | | | | a+ | a+ | | | |
| ADF-316 | ADPI-316.2 | | | | a+ | a+ | | | |
| ADF-317 | ADPI-317 | | | | | a− | | | |
| ADF-318 | ADPI-318 | b− | b− | | a+ | a− | a− | b− | |
| ADF-319 | ADPI-319 | b− | b− | | | a+ | | | b− |
| ADF-320 | ADPI-320.1 | | | | | | a− | | |
| ADF-320 | ADPI-320.2 | | | | | | a− | | |
| ADF-321 | ADPI-321 | | b+ | b+ | | | | | b+ |
| ADF-322 | ADPI-322 | b− | | | a− | | | | |
| ADF-323 | ADPI-323 | | a− | | | | | | |
| ADF-324 | ADPI-324 | | b− | | | | | | |
| ADF-325 | ADPI-325 | b− | | | | | | | |
| ADF-326 | ADPI-326 | | a− | | | | c− | | |
| ADF-327 | ADPI-327 | | | | | a− | | | |
| ADF-328 | ADPI-328 | b− | b− | b− | | a− | | b− | |
| ADF-329 | ADPI-329 | | | a− | | | | | |
| ADF-330 | ADPI-330 | | | | a+ | | | | |
| ADF-331 | ADPI-331 | | | | | a+ | | | b+ |
| ADF-332 | ADPI-332 | a− | b− | | | | c− | | |
| ADF-333 | ADPI-333.1 | | | | | c+ | | | |
| ADF-333 | ADPI-333.2 | | | | | c+ | | | |
| ADF-334 | ADPI-334 | | a− | | | a− | a+ | | |
| ADF-335 | ADPI-335 | b− | | | | | c− | | b+ |
| ADF-336 | ADPI-336 | | a− | a− | a+ | | | | a− |
| ADF-337 | ADPI-337 | | a+ | | a− | a− | a− | | b+ |
| ADF-338 | ADPI-338 | a+ | | a+ | a+ | a+ | a+ | b+ | |
| ADF-339 | ADPI-339 | | | | a− | | a− | | |
| ADF-340 | ADPI-340 | | b− | | | | | | |
| ADF-341 | ADPI-341 | | | a− | a− | a+ | a− | | |
| ADF-342 | ADPI-342 | a− | b− | | a− | a− | | | |
| ADF-343 | ADPI-343 | | | | a− | a+ | | | |
| ADF-344 | ADPI-344 | b− | b− | | | a+ | | | |
| ADF-345 | ADPI-345 | | | | a− | | | | |
| ADF-346 | ADPI-346 | b− | b− | | | | | b− | |
| ADF-347 | ADPI-347 | | | | | a− | | | |
| ADF-348 | ADPI-348 | | | | a+ | | a+ | | |
| ADF-349 | ADPI-349 | b+ | b+ | | a− | a+ | a− | | |
| ADF-350 | ADPI-350 | | | | | a− | | | |
| ADF-351 | ADPI-351 | b− | b− | | | | | | |
| ADF-352 | ADPI-352 | b− | | | | | | | |
| ADF-353 | ADPI-353 | | | a+ | a+ | a+ | | | |
| ADF-354 | ADPI-354 | | | | | a+ | | | |
| ADF-355 | ADPI-355 | a+ | | | | | a+ | | a+ |
| ADF-356 | ADPI-356 | | a− | | | a+ | | | |
| ADF-357 | ADPI-357 | | | | | a+ | | | |
| ADF-358 | ADPI-358 | | a− | b+ | a+ | | | | |
| ADF-359 | ADPI-359 | | | | | a− | | | |
| ADF-360 | ADPI-360 | b− | b− | | a− | | | | |
| ADF-361 | ADPI-361 | a+ | | | a+ | a+ | a− | | |
| ADF-362 | ADPI-362 | b− | b− | | | | | | |
| ADF-363 | ADPI-363 | | | | | a− | | | |
| ADF-364 | ADPI-364 | | | | | | | | b− |
| ADF-365 | ADPI-365.1 | | | | | | a− | | |
| ADF-365 | ADPI-365.2 | | | | | | a− | | |
| ADF-366 | ADPI-366 | b− | b− | a− | | | a− | | b− |
| ADF-367 | ADPI-367.1 | | | | | | | | b− |
| ADF-367 | ADPI-367.2 | | | | | | | | b− |
| ADF-368 | ADPI-368.1 | | b− | | | | | | b− |
| ADF-368 | ADPI-368.2 | | b− | | | | | | b− |
| ADF-369 | ADPI-369.1 | | a+ | | | | | | |
| ADF-369 | ADPI-369.2 | | a+ | | | | | | |
| ADF-370 | ADPI-370 | | b− | | a− | | a+ | | |
| ADF-371 | ADPI-371 | | | | a+ | | | | |
| ADF-372 | ADPI-372 | | | | a+ | | | | |
| ADF-373 | ADPI-373 | | | b− | | | | | b− |
| ADF-374 | ADPI-374 | | b− | | | | | | |
| ADF-375 | ADPI-375 | | | | a− | | a− | | a− |
| ADF-376 | ADPI-376 | | c− | b− | | a+ | a+ | | a− |
| ADF-377 | ADPI-377 | | | | | c− | a+ | | |
| ADF-378 | ADPI-378 | | | | | c− | | | |
| ADF-379 | ADPI-379 | | | | | | a+ | | |
| ADF-380 | ADPI-380 | | | | | a+ | a− | | |
| ADF-381 | ADPI-381 | | a+ | | | a+ | c− | | a+ |
| ADF-382 | ADPI-382 | a+ | a+ | b+ | a+ | a+ | a+ | b+ | |
| ADF-383 | ADPI-383 | | | | c− | | a+ | | |
| ADF-384 | ADPI-384 | a− | a− | | | a− | | b− | |
| ADF-386 | ADPI-386 | b− | a− | | a+ | | a+ | | |
| ADF-387 | ADPI-387 | b+ | b+ | | | | | | b+ |
| ADF-388 | ADPI-388 | | a− | | | | | | |
| ADF-389 | ADPI-389 | | b− | | | | | | |
| ADF-390 | ADPI-390 | a+ | b− | | a− | a+ | c− | | a+ |
| ADF-391 | ADPI-391 | | | | | | c− | | |
| ADF-392 | ADPI-392 | | b− | | | a+ | | | |
| ADF-393 | ADPI-393 | | | | a− | a− | | | |
| ADF-394 | ADPI-394 | | | | | | a− | | |
| ADF-395 | ADPI-395 | b− | b− | | | | a− | | |
| ADF-396 | ADPI-396 | b− | | b− | | | | | |
| ADF-397 | ADPI-397 | | | | | | a− | | |
| ADF-398 | ADPI-398 | | b− | | | | | | |
| ADF-399 | ADPI-399.1 | | | | | a− | | | b+ |
| ADF-399 | ADPI-399.2 | | | | | a− | | | b+ |
| ADF-400 | ADPI-400 | b− | b− | | a+ | | a− | | a− |
| ADF-401 | ADPI-401 | a− | | | | | a− | | |
| ADF-402 | ADPI-402 | | | | | a+ | | | |
| ADF-403 | ADPI-403 | | | | a+ | | | | a− |
| ADF-404 | ADPI-404.1 | | | | | | | | b+ |
| ADF-404 | ADPI-404.2 | | | | | | | | b+ |
| ADF-405 | ADPI-405 | b− | b− | | | | | | b− |
| ADF-406 | ADPI-406 | | | b− | | | | | |
| ADF-407 | ADPI-407 | b− | a− | | | a+ | | | |
| ADF-408 | ADPI-408 | | b− | | | | | | |
| ADF-409 | ADPI-409 | | | a+ | a− | | | | |
| ADF-410 | ADPI-410 | | | b− | | a+ | a− | | a+ |
| ADF-411 | ADPI-411 | b+ | b+ | | | a− | a− | a+ | |
| ADF-412 | ADPI-412.1 | a+ | | | a− | a+ | | | a+ |
| ADF-412 | ADPI-412.2 | a+ | | | a− | a+ | | | a+ |
| ADF-413 | ADPI-413 | a− | | | a− | a− | a+ | a− | |
| ADF-414 | ADPI-414 | | | | | a+ | c+ | | a+ |
| ADF-415 | ADPI-415 | | | | a− | | | | b+ |
| ADF-416 | ADPI-416 | | | | | a+ | a− | | |
| ADF-417 | ADPI-417 | | | | a− | | | | |
| ADF-418 | ADPI-418 | | a+ | | | | | | |
| ADF-419 | ADPI-419 | b+ | b+ | | | | a+ | b+ | b+ |
| ADF-420 | ADPI-420 | | | a− | | | a− | | |
| ADF-421 | ADPI-421 | | | | | c+ | | | |
| ADF-422 | ADPI-422 | | a+ | | | | a− | | |
| ADF-423 | ADPI-423 | | | | | | | | b− |
| ADF-424 | ADPI-424 | | | | | | c+ | | |
| ADF-425 | ADPI-425 | | b− | | | | a+ | | |
| ADF-426 | ADPI-426.1 | | | | a+ | | | | |
| ADF-426 | ADPI-426.2 | | | | a+ | | | | |
| ADF-427 | ADPI-427 | a− | b− | | | | | a− | |
| ADF-428 | ADPI-428 | | | | | a+ | | | |
| ADF-429 | ADPI-429 | a+ | a+ | | a− | | a+ | | b+ |
| ADF-430 | ADPI-430 | | b− | | | | | | |
| ADF-431 | ADPI-431 | | b− | | | | | | |
| ADF-432 | ADPI-432 | | | | a− | | a− | | |
| ADF-433 | ADPI-433 | | | | a+ | | c− | | |
| ADF-434 | ADPI-434 | b− | b− | | | | a− | | b− |
| ADF-435 | ADPI-435 | | | | a− | | | | |
| ADF-436 | ADPI-436 | | b− | | | | a− | | |
| ADF-437 | ADPI-437 | a+ | a+ | a− | | a− | a− | | b+ |
| ADF-438 | ADPI-438 | | | | | | a+ | | |
| ADF-439 | ADPI-439 | b− | b− | | | | | | |
| ADF-440 | ADPI-440.1 | a+ | b+ | | | | | | |
| ADF-440 | ADPI-440.2 | a+ | b+ | | | | | | |
| ADF-441 | ADPI-441 | b− | b− | b− | | | | | |
| ADF-442 | ADPI-442 | | | | | | a− | | a+ |
| ADF-443 | ADPI-443 | b+ | a+ | | a+ | | | a+ | a+ |
| ADF-444 | ADPI-444 | | | | | | a+ | | a− |
| ADF-445 | ADPI-445 | | | | | | a− | | |
| ADF-446 | ADPI-446 | | | | | | | | b− |
| ADF-447 | ADPI-447.1 | b+ | b+ | | | a− | a+ | | |
| ADF-447 | ADPI-447.2 | b+ | b+ | | | a− | a+ | | |
| ADF-448 | ADPI-448 | | b− | | a− | | | | |
| ADF-449 | ADPI-449 | b+ | | | | a− | | | |
| ADF-450 | ADPI-450.1 | b+ | b+ | a+ | a+ | a+ | | b+ | |
| ADF-450 | ADPI-450.2 | b+ | b+ | a+ | a+ | a+ | | b+ | |
| ADF-451 | ADPI-451 | | | | a− | a− | | | |
| ADF-452 | ADPI-452 | | | b+ | a− | a− | a+ | a− | a− |
| ADF-453 | ADPI-453 | b− | | | | | | | |
| ADF-454 | ADPI-454 | | a+ | b+ | | | a+ | | |
| ADF-455 | ADPI-455 | | | | | a+ | c− | | |
| ADF-456 | ADPI-456 | b− | a− | a+ | a− | a− | b− | a− | |
| ADF-457 | ADPI-457 | | a− | | a+ | | | | |
| ADF-458 | ADPI-458 | | | | a− | a− | | b− | |
| ADF-459 | ADPI-459 | b− | | | | | | a− | |
| ADF-460 | ADPI-460 | | | | | | c− | | |
| ADF-461 | ADPI-461 | | | b+ | a− | | | | |
| ADF-462 | ADPI-462 | a− | | | c+ | a− | a− | | |
| ADF-463 | ADPI-463 | | b− | | | | | | |
| ADF-464 | ADPI-464 | | | | | | c− | | |
| ADF-465 | ADPI-465 | | | | | | a− | | |
| ADF-466 | ADPI-466.1 | | | | | | a+ | | |
| ADF-466 | ADPI-466.2 | | | | | | a+ | | |
| ADF-467 | ADPI-467 | | b− | | | | a− | | |
| ADF-468 | ADPI-468 | | b+ | | | | a− | | |
| ADF-469 | ADPI-469.1 | | | | | | | | b− |
| ADF-469 | ADPI-469.2 | | | | | | | | b− |
| ADF-470 | ADPI-470 | a− | a− | | | | a− | | |
| ADF-471 | ADPI-471 | | a+ | a+ | | | | | a− |
| ADF-472 | ADPI-472 | | | a− | | | c− | | b+ |
| ADF-473 | ADPI-473 | | b− | | | | a− | | |
| ADF-474 | ADPI-474 | | b− | | | | | | |
| ADF-475 | ADPI-475 | | | a− | | | a+ | | |
| ADF-476 | ADPI-476 | b− | a− | b− | a+ | a− | | | |
| ADF-477 | ADPI-477 | | b− | a− | | | c− | | |
| ADF-478 | ADPI-478.1 | | | | | a− | | | b+ |
| ADF-478 | ADPI-478.2 | | | | | a− | | | b+ |
| ADF-479 | ADPI-479 | | b− | | | | a− | | |
| ADF-480 | ADPI-480 | a+ | c− | | a+ | a− | a− | | |
| ADF-481 | ADPI-481 | | | | a− | | | | |
| ADF-482 | ADPI-482 | | | | a− | | | | |
| ADF-483 | ADPI-483 | a− | | | | | | | |
| ADF-484 | ADPI-484 | | | | c− | | | | |
| ADF-485 | ADPI-485 | | | | | a− | | | b+ |
| ADF-486 | ADPI-486 | | | | | c− | a− | | |
| ADF-487 | ADPI-487 | | a+ | | a− | | | | |
| ADF-488 | ADPI-488 | | | a+ | | | a− | | |
| ADF-489 | ADPI-489 | a+ | a+ | | | | c+ | | |
| ADF-490 | ADPI-490 | | | | | a− | | | |
| ADF-491 | ADPI-491 | b+ | a+ | a+ | a+ | | | b+ | b+ |
| ADF-492 | ADPI-492 | b− | a− | | a+ | c− | | | |
| ADF-493 | ADPI-493.1 | b+ | b+ | | | c+ | | | b+ |
| ADF-493 | ADPI-493.2 | b+ | b+ | | | c+ | | | b+ |
| ADF-494 | ADPI-494 | | | | a+ | | | | |
| ADF-495 | ADPI-495 | b− | b− | | | a− | | | b− |
| ADF-496 | ADPI-496 | | | | | a− | a+ | | |
| ADF-497 | ADPI-497 | | | | | a− | | | |
| ADF-498 | ADPI-498 | | | | | c+ | | | |
| ADF-499 | ADPI-499 | | | | | a− | a− | | |
| ADF-500 | ADPI-500 | | | | | a− | | | |
| ADF-501 | ADPI-501 | | b+ | | | | | | |
| ADF-502 | ADPI-502 | | b− | | | | | | |
| ADF-503 | ADPI-503 | | b− | | | | | | |
| ADF-504 | ADPI-504 | b− | | | | | | | b− |
| ADF-505 | ADPI-505 | | | | | | | | b− |
| ADF-506 | ADPI-506 | | | b+ | a+ | c+ | a+ | | |
| ADF-507 | ADPI-507 | b+ | b+ | | | | | a+ | |
| ADF-508 | ADPI-508 | a+ | | | | a+ | a+ | | |
| ADF-509 | ADPI-509 | | | | | | a+ | | |
| ADF-510 | ADPI-510 | b+ | | | | | c+ | b+ | a+ |
| ADF-511 | ADPI-511 | | | | | | | | a+ |
| ADF-512 | ADPI-512 | | | | a− | | | | |
| ADF-513 | ADPI-513 | | | | a− | | | | |
| ADF-514 | ADPI-514 | b+ | | | a+ | | | | b+ |
| ADF-515 | ADPI-515 | a− | a+ | b− | a+ | | | | |
| ADF-516 | ADPI-516 | | | | | a− | | | |
| ADF-517 | ADPI-517 | | | | a+ | c− | a− | | |
| ADF-518 | ADPI-518 | | | | | | | | b− |
| ADF-519 | ADPI-519 | | | | | c− | | | |
| ADF-520 | ADPI-520 | | | b− | | c− | c+ | | |
| ADF-521 | ADPI-521 | | a− | | a+ | c+ | a− | | |
| ADF-522 | ADPI-522 | | | | | c− | c+ | | |
| ADF-523 | ADPI-523 | | | | | a− | c− | | |
| ADF-524 | ADPI-524 | b+ | b+ | | | | | | b+ |
| ADF-525 | ADPI-525 | | | | | a− | a− | | |
| ADF-526 | ADPI-526.1 | | | | a+ | a− | a+ | | |
| ADF-526 | ADPI-526.2 | | | | a+ | a− | a+ | | |
| ADF-527 | ADPI-527 | | | | a− | | | | a+ |
| ADF-528 | ADPI-528 | | | | | | a− | | |
| ADF-529 | ADPI-529 | | | | a− | | | | |
| ADF-530 | ADPI-530.1 | | | | | | c− | | |
| ADF-530 | ADPI-530.2 | | | | | | c− | | |
| ADF-531 | ADPI-531 | | | | | a− | a− | | |
| ADF-532 | ADPI-532 | | | | | c− | | | |
| ADF-533 | ADPI-533 | | | | | a− | | | |
| ADF-534 | ADPI-534 | | | | | a− | a− | | |
| ADF-535 | ADPI-535 | a+ | | | | a− | | | a+ |
| ADF-536 | ADPI-536 | | | | | c+ | | | |
| ADF-537 | ADPI-537 | | | | | a− | | | |
| ADF-538 | ADPI-538 | | | | | c− | | | |
| ADF-539 | ADPI-539 | | | | c+ | c− | | | |
| ADF-540 | ADPI-540 | | | | c+ | | | | |
| ADF-541 | ADPI-541 | | | | c− | a+ | | | |
| ADF-542 | ADPI-542 | | | | a− | | | | |
| ADF-543 | ADPI-543 | | | | | | a− | | |
| ADF-544 | ADPI-544 | | | | | | a− | | |
| ADF-545 | ADPI-545 | | | | c− | | | | |
| ADF-546 | ADPI-546 | | | | c− | | | | |
| ADF-547 | ADPI-547.1 | | | | | | c− | | |
| ADF-547 | ADPI-547.2 | | | | | | c− | | |
| ADF-547 | ADPI-547.3 | | | | | | c− | | |
| ADF-548 | ADPI-548.1 | | | | | a− | | | |
| ADF-548 | ADPI-548.2 | | | | | a− | | | |
| ADF-549 | ADPI-549 | | | | c+ | a+ | | | |

*HC = hippocampus, Selected HC = hippocampal samples with a post-mortem time < 4h EC = Entorhinal cortex, FC = Frontal Cortex, NC = Neocortex, AM = amygdala, Early = EC/HC vs. FC/NC/Am, ALL = all Alzheimer's disease samples vs all control samples.

The amino acid sequences of tryptic digest peptides of these ADPIs identified by tandem mass spectrometry and database searching as described in the Examples, infra are listed in Table IV, in addition to their corresponding pIs and MWs.

Table IV. ADPIs Description by Amino Acid Sequence of Tryptic Digest Peptides

TABLE IV

ADPIs description by Amino Acid Sequence of Tryptic Digest Pep-
tides

| | | Amino Acid Sequence of Tryptic | |
|---|---|---|---|
| ADF# | ADPI# | Digest peptides | SEQ ID NO: |
| ADF-1 | ADPI-1 | ALAAELNQLR, | SEQ ID NO:31 |
| | | FADLTDAAAR, | SEQ ID NO:199 |
| ADF-3 | ADPI-3.1 | LAVNMVPFPR, | SEQ ID NO:428 |
| ADF-3 | ADPI-3.2 | EIEAEIQALR, | SEQ ID NO:155 |
| ADF-3 | ADPI-3.3 | GFPGYMYTDLATIYER, | SEQ ID NO:266 |
| | | NFIAQGPYENR, | SEQ ID NO:561 |
| | | NYLSQPR, | SEQ ID NO:589 |
| | | IPQSTLSEFYPR, | SEQ ID NO:365 |
| | | QIYPPINVLPSLSR, | SEQ ID NO:611 |
| | | TPVSEDMLGR, | SEQ ID NO:708 |
| ADF-5 | ADPI-5.3 | TGQEIPVNVR, | SEQ ID NO:693 |
| | | LPDGSEIPLPPILLGR, | SEQ ID NO:491 |
| | | EGGSIPVTLTFQEATGK, | SEQ ID NO:148 |
| | | YIDENQDR, | SEQ ID NO:792 |
| | | TVFGVEPDLTR, | SEQ ID NO:714 |
| ADF-6 | ADPI-6 | LADVYQAELR, | SEQ ID NO:421 |
| | | ALAAELNQLR, | SEQ ID NO:31 |
| | | KILESLEEEIR, | SEQ ID NO:410 |
| | | LQDETNLR, | SEQ ID NO:496 |
| ADF-8 | ADPI-8 | LADVYQAELR, | SEQ ID NO:421 |
| | | KIESLEEEIR, | SEQ ID NO:411 |
| | | ALAAELNQLR, | SEQ ID NO:31 |
| ADF-9 | ADPI-9 | QLICDPSYIPDR, | SEQ ID NO:614 |
| | | TDDYLDQPCLETVNR, | SEQ ID NO:683 |
| | | FQLLEGPPESMGR, | SEQ ID NO:234 |
| | | MAGTAFDFENMK, | SEQ ID NO:531 |
| ADF-10 | ADPI-10 | ALAAELNQLR, | SEQ ID NO:31 |
| | | KIESLEEEIR, | SEQ ID NO:411 |
| | | LADVYQAELR, | SEQ ID NO:421 |
| ADF-11 | ADPI-11 | LADVYQAELR, | SEQ ID NO:421 |
| | | EAASYQEALAR, | SEQ ID NO:126 |
| ADF-12 | ADPI-12.2 | FPGQLNADLR, | SEQ ID NO:232 |
| ADF-12 | ADPI-12.1 | QLQSLTCDLESLR, | SEQ ID NO:617 |
| | | ELQEQLAR, | SEQ ID NO:173 |
| | | LADVYQAELR, | SEQ ID NO:421 |
| | | ALAAELNQLR, | SEQ ID NO:31 |
| | | KIESLEEEIR, | SEQ ID NO:410 |
| ADF-13 | ADPI-13 | TTPSYVAFTDTER, | SEQ ID NO:712 |
| | | TVTNAVVTVPAYFNDSQR, | SEQ ID NO:716 |
| | | FEELNADLFR, | SEQ ID NO:212 |
| | | VEIIANDQGNR, | SEQ ID NO:727 |
| ADF-15 | ADPI-15 | AIIESDQEQGR, | SEQ ID NO:29 |
| | | MDAEHPELR, | SEQ ID NO:533 |
| | | EPAAEIEALLGMDLVR, | SEQ ID NO:180 |
| ADF-16 | ADPI-16 | LADVYQAELR, | SEQ ID NO:421 |
| | | ALAAELNQLR, | SEQ ID NO:32 |
| ADF-22 | ADPI-22 | ELQELVQYPVEHPDK, | SEQ ID NO:172 |
| | | EVDIGIPDATGR, | SEQ ID NO:190 |
| | | KGDIFLVR, | SEQ ID NO:406 |
| ADF-23 | ADPI-23 | YYLDSLDR, | SEQ ID NO:815 |
| | | IIHEDGFSGEDVK, | SEQ ID NO:343 |
| | | LWGDSGIQECFNR, | SEQ ID NO:524 |
| | | IGAADYQPTEQDILR, | SEQ ID NO:336 |
| ADF-24 | ADPI-24 | GFPVVLDSPR, | SEQ ID NO:267 |
| | | ILIGSSFPLSGGR, | SEQ ID NO:352 |
| | | ELLGPDFGYVTR, | SEQ ID NO:167 |
| ADF-25 | ADPI-25.3 | QLFHPEQLITGK, | SEQ ID NO:613 |
| | | AVFVDLEPTVIDEVR, | SEQ ID NO:64 |
| ADF-25 | ADPI-25.2 | FASFIER, | SEQ ID NO:203 |
| | | AQLQDLNDR, | SEQ ID NO:55 |
| | | YEEEVLSR, | SEQ ID NO:787 |
| | | SAYSSYSAPVSSSLSVR, | SEQ ID NO:630 |
| | | ALYEQEIR, | SEQ ID NO:46 |
| | | SAYGGLQTSSYLMSTR, | SEQ ID NO:629 |
| ADF-26 | ADPI-26 | ELQEQLAR, | SEQ ID NO:173 |
| | | FADLTDAAAR, | SEQ ID NO:199 |
| | | EAASYQEALAR, | SEQ ID NO:125 |
| ADF-27 | ADPI-27 | LADVYQAELR, | SEQ ID NO:421 |
| | | ALAAELNQLR, | SEQ ID NO:31 |
| | | KIESLEEEIR, | SEQ ID NO:410 |
| | | DNLAQDLATVR, | SEQ ID NO:109 |
| ADF-29 | ADPI-29 | LADVYQAELR, | SEQ ID NO:421 |
| | | KIESLEEEIR, | SEQ ID NO:411 |
| | | ALAAELNQLR, | SEQ ID NO:31 |
| ADF-31 | ADPI-31 | ALAAELNQLR, | SEQ ID NO:31 |
| ADF-33 | ADPI-33.1 | FHVEEEGK, | SEQ ID NO:220 |
| ADF-35 | ADPI-35 | LYPPSAEYPDLR, | SEQ ID NO:526 |
| | | LGYILTCPSNLGTGLR, | SEQ ID NO:460 |
| ADF-37 | ADPI-37 | LDLIAQQMMPEVR, | SEQ ID NO:433 |
| | | IQMSNLMNQAR, | SEQ ID NO:373 |
| | | AEEEFNIEK, | SEQ ID NO:13 |
| ADF-39 | ADPI-39 | HIYLLPSGR, | SEQ ID NO:314 |
| | | ITWSNPPAQGAR, | SEQ ID NO:391 |
| | | VGGVQSLGGTGALR, | SEQ ID NO:735 |
| ADF-41 | ADPI-41 | NILLTNEQLESAR, | SEQ ID NO:568 |
| | | VGIPVTDENGNR, | SEQ ID NO:737 |
| | | QAITQVVVSR, | SEQ ID NO:594 |
| ADF-42 | ADPI-42 | NTGIICTIGPASR, | SEQ ID NO:581 |
| | | GDYPLEAVR, | SEQ ID NO:257 |
| | | DPVQEAWAEDVDLR, | SEQ ID NO:110 |
| | | EAEAAMFHR, | SEQ ID NO:128 |
| | | TATESFASDPILYR, | SEQ ID NO:680 |
| | | LDIDSPPITAR, | SEQ ID NO:431 |
| ADF-54 | ADPI-54 | WVVIGDENYGEGSSR, | SEQ ID NO:785 |
| | | NAVTQEFGPVPDTAR, | SEQ ID NO:557 |
| | | DGYAQILR, | SEQ ID NO:90 |
| | | IVYGHLDDPASQEIER, | SEQ ID NO:398 |
| | | EGWPLDIR, | SEQ ID NO:151 |
| ADF-56 | ADPI-56 | LEAPDADELPK, | SEQ ID NO:437 |
| | | WVVIGDENYGEGSSR, | SEQ ID NO:785 |
| | | NAVTQEFGPVPDTAR, | SEQ ID NO:557 |
| | | EGWPLDIR, | SEQ ID NO:151 |
| ADF-61 | ADPI-61 | VLDPFTIKPLDR, | SEQ ID NO:743 |
| | | ISSDLDGHPVPK, | SEQ ID NO:381 |
| | | LDNLVAILDINR, | SEQ ID NO:434 |
| | | SVPTSTVFYPSDGVATEK, | SEQ ID NO:672 |
| ADF-62 | ADPI-62 | TGAQELLR, | SEQ ID NO:691 |
| ADF-66 | ADPI-66 | VPVGDQPPDIEFQIR, | SEQ ID NO:759 |
| | | IEGSGDQIDTYELSGGAR, | SEQ ID NO:328 |
| ADF-67 | ADPI-67 | DDGSWEVIEGYR, | SEQ ID NO:76 |
| | | NLNLIVSYGR, | SEQ ID NO:572 |
| ADF-70 | ADPI-70 | VPVGDQPPDIIEFQIR, | SEQ ID NO:759 |
| | | GISPVPTINLR, | SEQ ID NO:276 |
| ADF-72 | ADPI-72 | TGAQELLR, | SEQ ID NO:690 |
| | | EPGLQIWR, | SEQ ID NO:181 |
| | | HVVPNEVVVQR, | SEQ ID NO:320 |
| | | QTQVSVLPEGGETPLFK, | SEQ ID NO:621 |
| | | YIETDPANR, | SEQ ID NO:794 |
| | | VVEHPEFLK, | SEQ ID NO:770 |
| ADF-77 | ADPI-77 | LADVYQAELR, | SEQ ID NO:421 |
| | | KIESLEEEIR, | SEQ ID NO:411 |
| | | ALAAELNQLR, | SEQ ID NO:31 |
| ADF-78 | ADPI-78.3 | LADVYQAELR, | SEQ ID NO:421 |
| | | ALAAELNQLR, | SEQ ID NO:32 |
| ADF-78 | ADPI-78.2 | ISEQFTAMFR, | SEQ ID NO:378 |
| | | FPGQLNADLR, | SEQ ID NO:231 |
| | | LAVNMVPFPR, | SEQ TD NO:427 |
| | | INVYYNEATGGNYVPR, | SEQ ID NO:361 |
| | | AVLVDLEPGTMDSVR, | SEQ ID NO:66 |
| | | YLTVAAVFR, | SEQ ID NO:800 |
| ADF-79 | ADPI-79 | ELEEETNAFNIR, | SEQ ID NO:163 |
| | | EFLSELQR, | SEQ ID NO:144 |
| | | SLFDYHDTR, | SEQ ID NO:649 |
| ADF-81 | ADPI-81.2 | LADVYQAELR, | SEQ ID NO:421 |
| | | KIESLEEEIR, | SEQ ID NO:411 |
| | | ALAAELNQLR, | SEQ ID NO:31 |
| ADF-82 | ADPI-82 | PYQYPALTPEQK, | SEQ ID NO:592 |
| | | ALTFSYGR, | SEQ ID NO:43 |
| | | LQSIGTENTEENR, | SEQ ID NO:499 |
| ADF-85 | ADPI-85 | QEYDESGPSIVHR, | SEQ ID NO:599 |
| | | DSYVGDEAQSK, | SEQ ID NO:113 |
| | | SYELPDGQVITIGNER, | SEQ ID NO:674 |
| ADF-87 | ADPI-87.1 | LIIWDSYTTNK, | SEQ ID NO:466 |
| | | IYAMHWGTDSR, | SEQ ID NO:400 |
| ADF-88 | ADPI-88 | VDIIENQVMDFR, | SEQ ID NO:724 |
| | | LLLEFTDTSYEEK, | SEQ ID NO:481 |
| | | FSWFAGEK, | SEQ ID NO:239 |
| | | LKPQYLEELPGQLK, | SEQ ID NO:470 |
| ADF-90 | ADPI-90 | LADVYQAELR, | SEQ ID NO:421 |
| | | KIESLEEEIR, | SEQ ID NO:411 |
| | | ALAAELNQLR, | SEQ ID NO:31 |
| ADF-91 | ADPI-91.1 | MIEEAGAIISTR, | SEQ ID NO:538 |
| | | VLEVPPVVYSR, | SEQ ID NO:745 |
| | | LMDEVAGIVAAR, | SEQ ID NO:487 |
| ADF-91 | ADPI-91.2 | DDNGVPFVR, | SEQ ID NO:78 |
| | | ALQASALNAWR, | SEQ ID NO:41 |
| | | LSQIGVENTEENR. | SEQ ID NO:509 |
| ADF-92 | ADPI-92 | VTLTSEEEAR, | SEQ ID NO:765 |
| | | GEMMDLQHGSLFLR, | SEQ ID NO:261 |
| | | LVIITAGAR, | SEQ ID NO:518 |
| ADF-94 | ADPI-94 | EYYFAITMER, | SEQ ID NO:196 |
| | | INFDSNSAYR, | SEQ ID NO:357 |
| ADF-95 | ADPI-95 | GQVFDVGPR, | SEQ ID NO:291 |
| ADF-97 | ADPI-97 | NVNQSLLELHIK, | SEQ ID NO:586 |
| | | ELGDHVTNLR, | SEQ ID NO:165 |
| ADF-98 | ADPI-98 | YALYDATYETK, | SEQ ID NO:786 |
| ADF-101 | ADPI-1O1 | NIILEEGK, | SEQ ID NO:566 |
| | | YALYDATYETK, | SEQ ID NO:786 |
| ADF-102 | ADPI-102.1 | TTGIVETIIIPTFK, | SEQ ID NO:710 |
| | | IGAADYQPTEQDILR, | SEQ ID NO:336 |
| | | IIHEDGFSGEDVK, | SEQ ID NO:343 |
| ADF-103 | ADPI-103 | ATAVMPDGQFK, | SEQ ID NO:59 |
| | | QITVNDLPVGR, | SEQ ID NO:610 |
| ADF-105 | ADPI-105.2 | LYPEGLAQLAR, | SEQ ID NO:525 |
| | | FFEHEVK, | SEQ ID NO:214 |
| | | LLFEGAGSNPGDK, | SEQ ID NO:478 |
| | | NVADYYPEYK, | SEQ ID NO:583 |
| ADF-105 | ADPI-105.1 | LADVYQAELR, | SEQ ID NO:421 |
| | | ALAAELNQLR, | SEQ ID NO:31 |
| | | KIESLEEEIR, | SEQ ID NO:410 |
| | | DNLAQDLATVR, | SEQ ID NO:108 |
| | | LQDETNLR, | SEQ ID NO:496 |
| ADF-107 | ADPI-107 | QDEHGFISR, | SEQ ID NO:596 |
| | | VLGDVIEVHGK, | SEQ ID NO:748 |
| | | APSWFDTGLSEMR, | SEQ ID NO:52 |
| ADF-108 | ADPI-108 | IVVVTAGVR, | SEQ ID NO:397 |
| | | SADTLWDIQK, | SEQ D NO:628 |
| | | SLADELALVDVLEDK, | SEQ D NO:645 |
| | | LIAPVAEEEATVPNNK, | SEQ ID NO:462 |
| ADF-109 | ADPI-109 | GNYLVDVDGNR, | SEQ ID NO:287 |
| ADF-111 | ADPI-111.1 | LADVYQAELR, | SEQ ID NO:421 |
| | | KIESLEEEIR, | SEQ ID NO:410 |
| | | EAASYQEALAR, | SEQ ID NO:125 |
| | | DNLAQDLATVR, | SEQ ID NO:109 |
| ADF-112 | ADPI-112 | DFQSGQHVTVR, | SEQ ID NO:83 |
| | | LFADAEEEQR, | SEQ ID NO:445 |
| ADF-113 | ADPI-113 | EAYPGDVFYLHSR, | SEQ ID NO:133 |
| | | ILGADTSVDLEETGR, | SEQ ID NO:351 |
| | | QMSLLLR, | SEQ ID NO:618 |
| | | TGAIVDVPVGEELLGR, | SEQ ID NO:689 |
| | | TGTAEMSSILEER, | SEQ ID NO:694 |
| | | AVDSLVPIGR, | SEQ ID NO:61 |
| ADF-115 | ADPI-115 | DFQSGQHVIVR, | SEQ ID NO:83 |
| | | LFADAEEEQR, | SEQ ID NO:445 |
| | | YVGESEANIR, | SEQ ID NO:812 |
| ADF-117 | ADPI-117.1 | LGAEVYHTLK, | SEQ ID NO:454 |
| | | IEEELGDEAR, | SEQ ID NO:327 |
| ADF-119 | ADPI-119 | LADVYQAELR, | SEQ ID NO:421 |
| | | ALAAIELNQLR, | SEQ ID NO:31 |
| | | KIIESLEEEIR, | SEQ TD NO:410 |
| | | EAASYQEALAR, | SEQ ID NO:126 |
| ADF-120 | ADPI-120 | LADVYQAELR, | SEQ ID NO:421 |
| | | EAASYQEALAR, | SEQ ID NO:126 |
| ADF-121 | ADPI-121 | IAVGSDADLVIWDPDSVK, | SEQ ID NO:324 |
| | | DHGVNSFLVYMAFK, | SEQ ID NO:92 |
| | | MDENQFVAVTSTNAAK, | SEQ ID NO:534 |
| | | VFNLYPR, | SEQ ID NO:732 |
| | | GLYDGPVCEVSVTPK, | SEQ ID NO:282 |
| | | IVLEDGTLHVTEGSGR, | SEQ ID NO:395 |
| | | DNFTLIPEGTNGTEER, | SEQ ID NO:107 |
| ADF-124 | ADPI-124 | DVLTGQEFDVR, | SEQ ID NO:118 |
| | | LVGAIVYYDGQHNIDAR, | SEQ ID NO:516 |
| | | ALEHFPMLQK, | SEQ ID NO:35 |
| ADF-125 | ADPI-125 | LADVYQAELR, | SEQ ID NO:421 |
| | | ALAAELNQLR, | SEQ ID NO:31 |
| | | KTESLEEETR, | SEQ ID NO:410 |
| | | EAASYQEALAR, | SEQ ID NO:126 |
| ADF-126 | ADPI-126 | FPGQLNADLR, | SEQ ID NO:232 |
| ADF-127 | ADPI-127 | AMDTLGIIEYGDK, | SEQ ID NO:47 |
| | | EYQLNDSAK, | SEQ ID NO:195 |
| | | IIHEDGFSGEDVK, | SEQ ID NO:343 |
| | | IGAADYQPTEQDTLR, | SEQ ID NO:336 |
| ADF-128 | ADPI-128 | GSAPPGPVPEGSIR, | SEQ ID NO:292 |
| ADF-129 | ADPI-129 | SSYYVVSGNDPAAEEPSR, | SEQ ID NO:666 |
| | | NVDVEFFK, | SEQ ID NO:585 |
| ADF-130 | ADPI-130 | LADVYQAELR, | SEQ ID NO:421 |
| | | KTESLEEEIR, | SEQ ID NO:411 |
| | | ALAAELNQLR, | SEQ ID NO:31 |
| ADF-131 | ADPI-131 | AILVDLEPGTMDSVR, | SEQ ID NO:30 |
| | | FPGQLNADLR, | SEQ ID NO:231 |
| | | LAVNMVPFPR, | SEQ ID NO:427 |
| | | ISVYYNEATGGK, | SEQ ID NO:384 |
| ADF-132 | ADPI-132 | ALAAELNQLR, | SEQ ID NO:31 |
| | | KIESLEEEIR, | SEQ ID NO:411 |
| | | LADVYQAELR, | SEQ ID NO:421 |
| ADF-133 | ADPI-133 | LEEGPPVTTVLTR, | SEQ ID NO:440 |
| ADF-138 | ADPI-138 | LTFDSSFSPNTGK, | SEQ ID NO:511 |
| | | WTEYGLTFTEK, | SEQ ID NO:783 |
| | | VTQSNFAVGYK, | SEQ ID NO:767 |
| | | SENGLEFTSSGSANTETTK, | SEQ ID NO:637 |
| ADF-139 | ADPI-139 | ILVLDDTNHER, | SEQ ID NO:353 |
| | | AGQVFLEELGNHK, | SEQ ID NO:24 |
| | | APGAEEYAQQDVLK, | SEQ ID NO:49 |
| | | LSPTDNLPR, | SEQ ID NO:508 |
| ADF-140 | ADPI-140 | ISEQFTAMFR, | SEQ ID NO:378 |
| | | TAVCDTPPR, | SEQ ID NO:682 |
| ADF-141 | ADPI-141 | LGDLYEEEMR, | SEQ ID NO:456 |
| | | VELQELNDR, | SEQ ID NO:728 |
| | | ITEIYEGTSEIQR, | SEQ ID NO:386 |
| | | AEGDSWVLNGTK, | SEQ ID NO:14 |
| ADF-143 | ADPI-143.1 | LFSGDVVLTAR, | SEQ ID NO:450 |
| | | GQAAVQQLQAEGLSPR, | SEQ ID NO:290 |
| ADF-143 | ADPI-143.2 | LTFDSSFSPNTGK, | SEQ ID NO:511 |
| | | WTEYGLTFTEK, | SEQ ID NO:783 |
| | | VTQSNFAVGYK, | SEQ ID NO:767 |
| | | YQTDPDACFSAK, | SEQ ID NO:809 |
| | | SENGLEFTSSGSANTETTK, | SEQ ID NO:636 |
| | | LTLSALLDGK, | SEQ ID NO:514 |
| ADF-144 | ADPI-144 | LADVYQAELR, | SEQ ID NO:421 |
| | | KIESLEEEIR, | SEQ ID NO:411 |
| | | ALAAELNQLR, | SEQ ID NO:31 |
| ADF-146 | ADPI-146.1 | MVMTLTFGDVVAVR, | SEQ ID NO:554 |
| | | LTNSQNFDEYMK, | SEQ ID NO:515 |
| | | ALGVGFATR, | SEQ ID NO:36 |
| ADF-146 | ADPI-146.2 | MVMTLTFGDVVAVR, | SEQ ID NO:554 |
| | | LTNSQNFDEYMK, | SEQ ID NO:515 |
| | | ALGVGFATR, | SEQ ID NO:36 |
| ADF-148 | ADPI-148 | ITIPVQTFSNLQIR, | SEQ ID NO:387 |
| | | ALAAELNQLR, | SEQ ID NO:32 |
| | | LADVYQAELR, | SEQ ID NO:421 |
| ADF-149 | ADPI-149.1 | LADVYQAELR, | SEQ ID NO:421 |
| | | KIESLEEEILR, | SEQ ID NO:411 |
| | | ALAAELNQLR, | SEQ ID NO:31 |
| ADF-150 | ADPI-150 | LADVYQAELR, | SEQ ID NO:421 |
| | | KIESLEBEIR, | SEQ ID NO:411 |
| | | ALAAELNQLR, | SEQ ID NO:31 |
| ADF-151 | ADPI-151.1 | FPGQLNADLR, | SEQ ID NO:232 |
| ADF-151 | ADPI-151.2 | LADVYQAELR, | SEQ ID NO:421 |
| | | ALAAELNQLR, | SEQ ID NO:31 |
| | | QEADEATLAR, | SEQ ID NO:598 |
| | | KIESLEEEIR, | SEQ ID NO:411 |
| ADF-152 | ADPI-152 | LADVYQAELR, | SEQ ID NO:421 |
| | | ITIPVQTFSNLQIR, | SEQ ID NO:387 |
| | | ALAAELNQLR, | SEQ ID NO:31 |
| | | KIESLEEEIR, | SEQ ID NO:410 |
| | | LEAENNLAAYR, | SEQ ID NO:436 |
| | | LEEEGQSLKDEMAR, | SEQ ID NO:438 |
| | | EAASYQEALAR, | SEQ ID NO:126 |
| ADF-153 | ADPI-153.1 | IPVGPETLGR, | SEQ ID NO:368 |
| ADF-153 | ADPI-153.2 | SLYASSPGGVYATR, | SEQ ID NO:653 |
| | | VELQELINDR, | SEQ ID NO:728 |
| ADF-153 | ADPI-153.3 | MAGTAFDFEINMKR, | SEQ ID NO:532 |
| | | TDDYLDQPCLETVNR, | SEQ ID NO:683 |
| | | FLMANGQLVK, | SEQ ID NO:223 |
| | | FQLLEGPPESMGR, | SEQ ID NO:234 |
| | | MAGTAFDFENMK, | SEQ ID NO:530 |
| | | FLVFVANFDENDPK, | SEQ ID NO:225 |
| | | DWNVDLIPK, | SEQ ID NO:121 |
| | | TFEGVDPQTTSMR, | SEQ ID NO:688 |
| | | KQNDVFGEAEQ, | SEQ ID NO:417 |
| ADF-154 | ADPI-154 | LADVYQAELR, | SEQ ID NO:421 |
| | | KIESLEEEIR, | SEQ ID NO:411 |
| | | ALAAELNQLR, | SEQ IDNO:31 |
| ADF-155 | ADPI-155 | TTPSYVAFTDTER, | SEQ ID NO:712 |
| | | AQIHDLVLVGGSTR, | SEQ ID NO:54 |
| | | DAGVIAGLNVLR, | SEQ ID NO:72 |
| | | IINEPTAAAIAYGLDR, | SEQ ID NO:345 |
| ADF-156 | ADPI-156.1 | FADLTDAAAR, | SEQ ID NO:198 |
| ADF-156 | ADPI-156.2 | SAYSSYSAPVSSSLSVR, | SEQ ID NO:630 |
| | | FASFIER, | SEQ ID NO:204 |
| | | ALYEQEIR, | SEQ TD NO:46 |
| ADF-157 | ADPI-157 | GFCLPPHCSR, | SEQ ID NO:262 |
| | | GTGGVDTAAVGGVFDVSNAD, | SEQ ID NO:295 |
| | | R, | SEQ TD NO:443 |
| | | LEQGQAIDDLMPAQK, | SEQ ID NO:754 |
| | | VLTPELYAELR, | |
| ADF-159 | ADPI-159.2 | QLQSLTCDLESLR, | SEQ ID NO:617 |
| | | DNLAQDLATVR, | SEQ ID NO:109 |
| ADF-159 | ADPI-159.1 | AVFPSIVGR, | SEQ ID NO:63 |
| | | SYELPDGQVITIGNER, | SEQ ID NO:675 |
| | | EITALAPSTMK, | SEQ ID NO:158 |
| ADF-160 | ADPI-160 | IVIGMDVAASEFYR, | SEQ ID NO:393 |
| | | LGAEVYHTLK, | SEQ ID NO:454 |
| | | AAVPSGASTGIYEALELR, | SEQ ID NO:8 |
| | | IEEELGDEAR, | SEQ ID NO:327 |
| ADF-162 | ADPI-162.1 | QEYDESGPSIVHR, | SEQ ID NO:599 |
| | | SYELPDGQVITIGNER, | SEQ ID NO:675 |
| ADF-162 | ADPI-162.2 | AQLQDLNDR, | SEQ ID NO:55 |
| | | FASFIER, | SEQ ID NO:204 |
| | | ALYEQEIIR, | SEQ ID NO:46 |
| ADF-162 | ADPI-162.3 | QLQSLTCDLESLR, | SEQ ID NO:617 |
| | | LAD VYQAELR, | SEQ 11) NO:421 |
| | | ALAAELNQLR, | SEQ ID NO:31 |
| | | HLQEYQDLLNYK, | SEQ ID NO:317 |
| | | KIESLEEEIR, | SEQ ID NO:410 |
| | | DNLAQDLATVR, | SEQ ID NO:109 |
| ADF-163 | ADPI-163 | LADVYQAELR, | SEQ ID NO:421 |
| | | ALAAELNQLR, | SEQ ID NO:31 |
| | | IIESLEEBIIR, | SEQ ID NO:329 |
| | | LEEEGQSLKDEMAR, | SEQ ID NO:438 |
| | | KIIESLEEEIR, | SEQ ID NO:411 |
| ADF-165 | ADPI-165 | DGDSVMVLPTIPEEEAK, | SEQ ID NO:87 |
| | | DINAYNCEEPTEK, | SEQ ID NO:94 |
| | | VVFVFGPDK, | SEQ ID NO:771 |
| | | VVFVFGPDKI( | SEQ ID NO:773 |
| | | ELATLLGMLDPAEK, | SEQ ID NO:161 |
| | | LPFPUDDR, | SEQ ID NO:493 |
| | | DFTPVCTTELGR, | SEQ ID NO:85 |
| | | VATPVDWK, | SEQ ID NO:720 |
| ADF-172 | ADPI-172 | VGAHAGEYGAEALER, | SEQ ID NO:734 |
| | | MFLSFPTTK, | SEQ ID NO:537 |
| ADF-173 | ADPI-173.1 | ISEQTYQLSR, | SEQ ID NO:380 |
| | | VEQDLAMGTDAEGEK, | SEQ ID NO:729 |
| | | DIMEDTIEDK, | SEQ ID NO:93 |
| | | SQLLILDR, | SEQ ID NO:661 |
| ADF-173 | ADPI-173.2 | ISEQTYQLSR, | SEQ ID NO:380 |
| | | VEQDLAMGTDAEGEK, | SEQ ID NO:729 |
| | | DIMEDTIEDK, | SEQ ID NO:93 |
| | | SQLLILDR, | SEQ ID NO:661 |
| ADF-175 | ADPI-175.1 | IFGVTTLDIVR, | SEQ ID NO:331 |
| | | AGAGSATLSMAYAGAR, | SEQ ID NO:21 |
| | | VDFPQDQLTALTGR, | SEQ ID NO:723 |
| ADF-175 | ADPI-175.2 | GALQNIIPASTGAAK, | SEQ ID NO:250 |
| | | LISWYDNEFGYSNR, | SEQ ID NO:467 |
| | | VVDLMAHMASK, | SEQ ID NO:769 |
| | | LVINGNPITTFQER, | SEQ ID NO:520 |
| ADF-176 | ADPI-176 | ISEQTYQLSR, | SEQ ID NO:380 |
| | | DIMEDTIEDK, | SEQ ID NO:93 |
| | | HIAEVSQEVTR, | SEQ ID NO:312 |
| | | SQLLILDR, | SEQ ID NO:660 |
| | | SSASFSTTAVSAR, | SEQ ID NO:662 |
| ADF-182 | ADPI-182 | LTFDSSFSPNTGK, | SEQ ID NO:511 |
| | | VTQSNFAVGYK, | SEQ ID NO:767 |
| | | YQIDPDACFSAK, | SEQ ID NO:809 |
| | | SENGLEFTSSGSANTETTK, | SEQ ID NO:637 |
| ADF-183 | ADPI-183 | QELSETEK, | SEQ ID NO:601 |
| | | MPFPVNHGASSEDTLLK, | SEQ ID NO:545 |
| | | MQLKPMEINPEMLNK, | SEQ ID NO:547 |
| | | LGFEDGSVLK, | SEQ ID NO:457 |
| ADF-188 | ADPI-188.1 | NPDDITNEEYGEFYK, | SEQ ID NO:578 |
| | | GVVDSEDLPLMSR, | SEQ ID NO:298 |
| | | TKPIWTR, | SEQ ID NO:702 |
| | | HFSVEGQLEFR, | SEQ ID NO:308 |
| | | EDQTEYLEER, | SEQ ID NO:137 |
| ADF-189 | ADPI-189.2 | YLYEIAR, | SEQ ID NO:801 |
| | | AVMDDFAAFVEK, | SEQ ID NO:68 |
| | | DVFLGMIFLYEYAR, | SEQ ID NO:115 |
| | | FQNALLVR, | SEQ ID NO:236 |
| | | KVPQVSTPTLVEVSR, | SEQ ID NO:418 |
| ADF-191 | ADPI-191 | WSLQSEAHR, | SEQ ID NO:782 |
| ADF-193 | ADPI-193 | YLYEIAR, | SEQ ID NO:801 |
| | | AVMDDFAXFVEK, | SEQ ID NO:68 |
| | | FQNALLVR, | SEQ ID NO:236 |
| | | DVFLGMFLYEYAR, | SEQ ID NO:115 |
| | | VPQVSTPTLVEVSR, | SEQ ID NO:758 |
| ADF-194 | ADPI-194.2 | LQHGSILGFPK, | SEQ ID NO:498 |
| | | DDGSWEVIEGYR, | SEQ ID NO:76 |
| | | MVEGFFDR, | SEQ ID NO:551 |
| | | IIAEGANGPTTPEADK, | SEQ ID NO:342 |
| | | EDDPNFFK, | SEQ ID NO:134 |
| ADF-196 | ADPI-196.1 | MIGGPILPSER, | SEQ ID NO:539 |
| | | EENEGVYNGSWGGR, | SEQ ID NO:140 |
| ADF-196 | ADPI-196.2 | QDGSVDFGR, | SEQ ID NO:597 |
| ADF-196 | ADPI-196.3 | DNDGWLTSDPR, | SEQ ID NO:105 |
| | | QDGSVDFGR, | SEQ ID NO:597 |
| | | EDGGGWWYNR, | SEQ ID NO:135 |
| ADF-196 | ADPI-196.4 | DAQELYAAGENR, | SEQ ID NO:74 |
| | | SELSGNFEK, | SEQ ID NO:634 |
| | | SLYHDISGDTSGDYR, | SEQ ID NO:654 |
| | | NTPAFFAER, | SEQ ID NO:582 |
| ADF-202 | ADPI-202 | LSEQTYQLSR, | SEQ ID NO:380 |
| | | DIMEDTTEDK, | SEQ ID NO:93 |
| | | HIAEVSQEVTR, | SEQ ID NO:312 |
| | | SQLLIILDR, | SEQ ID NO:660 |
| | | SSASFSTTAVSAR, | SEQ ID NO:662 |
| ADF-204 | ADPI-204 | GFDEYMK, | SEQ ID NO:263 |
| | | ELGVGIALR, | SEQ ID NO:166 |
| ADF-208 | ADPI-208 | ELGDHVTNLR, | SEQ ID NO:165 |
| ADF-209 | ADPI-209 | LNLEAINYMAADGDFK, | SEQ ID NO:490 |
| | | LPDGYEFK, | SEQ ID NO:492 |
| ADF-216 | ADPI-216 | LQAVTDDHIR, | SEQ ID NO:495 |
| ADF-217 | ADPI-217 | LLVVYPWTQR, | SEQ ID NO:486 |
| | | VLGAFSDGLAHLDNLK, | SEQ ID NO:747 |
| | | EFTPPVQAAYQK, | SEQ ID NO:146 |
| ADF-220 | ADPI-220 | ESISVSSEQLAQFR, | SEQ ID NO:187 |
| | | GGPFSDSYR, | SEQ ID NO:270 |
| ADF-223 | ADPI-223 | LEEGPPVTTVLTR, | SEQ ID NO:439 |
| | | TREEIQEVR, | SEQ ID NO:709 |
| ADF-228 | ADPI-228 | APDFVFYAPR, | SEQ ID NO:48 |
| ADF-229 | ADPI-229 | YPQFISR, | SEQ ID NO:805 |
| | | GNYLVDVDGNR, | SEQ ID NO:287 |
| ADF-230 | NDPI-230.1 | FPGQLNADLR, | SEQ ID NO:231 |
| | | YLTVAAVFR, | SEQ ID NO:800 |
| | | ISEQFTAMFR, | SEQ ID NO:378 |
| ADF-230 | ADPI-230.2 | LADVYQAELR, | SEQ ID NO:421 |
| | | KIESLEBEIR, | SEQ ID NO:411 |
| | | ALAAELNQLR, | SEQ ID NO:31 |
| ADF-232 | ADPI-232.1 | ISEQFTAMFR, | SEQ ID NO:378 |
| | | FPGQLNADLR, | SEQ ID NO:232 |
| ADF-232 | ADPI-232.2 | VPAFLSAAEVEEHLR, | SEQ ID NO:757 |
| | | EAVLYVDSQEAALK, | SEQ ID NO:131 |
| ADF-236 | ADPI-236.2 | APSTYGGGLSVSSR, | SEQ ID NO:51 |
| ADF-236 | ADPI-236.3 | NGSGTLDLEEFLR, | SEQ ID NO 565 |
| | | SLDADEFR, | SEQ ID NO:647 |
| | | GASGIQGLAR, | SEQ ID NO:251 |
| ADF-237 | ADPI-237.1 | EQMAISGGFIR, | SEQ ID NO:185 |
| ADF-237 | ADPI-237.2 | AILVDLEPGTMDSVR, | SEQ ID NO:30 |
| | | INVYYNEAAGNK, | SEQ ID NO:359 |
| ADF-240 | ADPI-240 | IYGLGSLALYEK, | SEQ ID NO:402 |
| | | HLLIGVSSDR, | SEQ ID NO:316 |
| ADF-243 | ADPI-243 | ALYDFEPENEGELGFK, | SEQ ID NO:45 |
| | | RLDFDYK, | SEQ ID NO:626 |
| | | QAVQILQQVTVR, | SEQ ID NO:595 |
| | | TIEYLQPNPASR, | SEQ ID NO:699 |
| ADF-245 | ADPI-245 | ILDQGEDFPASEMTR, | SEQ ID NO:349 |
| ADF-250 | ADPI-250 | HQAFEAELSANQSR, | SEQ ID NO:318 |
| | | LFGAAEVQR, | SEQ ID NO:449 |
| | | KHQALQAEIAGHEPR, | SEQ ID NO:409 |
| ADF-251 | ADPI-251 | ELPTAIFDYVEFTR, | SEQ ID NO:171 |
| | | LLEAQSHFR, | SEQ ID NO:476 |
| | | SSEETESAFR, | SEQ ID NO:663 |
| | | SQLLGSAHEVQR, | SEQ ID NO:659 |
| ADF-252 | ADPI-252.2 | AVFDETYPDPVR, | SEQ ID NO:62 |
| ADF-252 | ADPI-252.1 | AENYDIPSADR, | SEQ ID NO:16 |
| | | ALPAVQQNNLDEDLIR, | SEQ ID NO:37 |
| | | LAGTQPLEVLEAVQR, | SEQ ID NO:423 |
| | | NEEDAAELVALAQAVNAR, | SEQ ID NO:559 |
| ADF-253 | ADPI-253 | FLSQIESDR, | SEQ ID NO:224 |
| | | HIDLVEGDEGR, | SEQ ID NO:313 |
| ADF-254 | ADPI-254 | FLSQIESDR, | SEQ ID NO:224 |
| | | HIDLVEGDEGR, | SEQ ID NO:313 |
| ADF-255 | ADPI-255 | FLSQIESDR, | SEQ ID NO:224 |
| | | HIDLVEGDEGR, | SEQ ID NO:313 |
| | | MLPTFVR, | SEQ ID NO:543 |
| ADF-256 | ADPI-256 | MLPSFVR, | SEQ ID NO:542 |
| | | HIDLVEGDEGR, | SEQ ID NO:313 |
| | | FLSQIESDR, | SEQ ID NO:224 |
| ADF-257 | ADPI-257.1 | EIEAEIQALR, | SEQ ID NO:155 |
| ADF-257 | ADPI-257.2 | IPQSTLSEFYPR, | SEQ ID NO:365 |
| | | QIYPPINVLPSLSR, | SEQ ID NO:612 |
| | | NFIAQGPYENR, | SEQ ID NO:561 |
| ADF-258 | ADPI-258 | YFDLGLPNIR, | SEQ ID NO:789 |
| | | TIEAEAAHGTVTR, | SEQ ID NO:698 |
| | | NILGGTVFR, | SEQ ID NO:567 |
| ADF-259 | ADPI-259.2 | TIISYIDEQFER, | SEQ ID NO:700 |
| ADF-259 | ADPI-259.1 | IYQIYEGTSQIQR, | SEQ ID NO:403 |
| | | TGEYPVPIAR, | SEQ ID NO:692 |
| ADF-260 | ADPI-260 | ILVLDDTNHER, | SEQ ID NO:353 |
| | | GAFSEEYKR, | SEQ ID NO:248 |
| | | AGQVFLEELGNHK, | SEQ ID NO:24 |
| | | APGAEEYAQQDVLK, | SEQ ID NO:49 |
| | | LSPTDNLPR, | SEQ ID NO:507 |
| | | GEEVGELSR, | SEQ ID NO:259 |
| | | LYSLGNGR, | SEQ ID NO:527 |
| ADF-261 | ADPI-261 | ALYDFEPENEGELGFK, | SEQ ID NO:45 |
| | | RLDFDYK, | SEQ ID NO:626 |
| | | QAVQILQQVTVR, | SEQ ID NO:595 |
| | | TIEYLQPNIPASR, | SEQ ID NO:699 |
| ADF-262 | ADPI-262 | LADVYQAELR, | SEQ ID NO:421 |
| | | ALAAELNQLR, | SEQ ID NO:31 |
| | | KIESLEEEIR, | SEQ ID NO:410 |
| | | EAASYQEALAR, | SEQ ID NO:126 |
| ADF-263 | ADPI-263 | VISFEEQVASIR, | SEQ ID NO:740 |
| ADF-264 | ADPI-264 | VADPAYLPTQQDVLR, | SEQ ID NO:718 |
| | | VSAFENPYVDAIK, | SEQ ID NO:762 |
| ADF-265 | ADPI-265 | DDNGVPFVR, | SEQ ID NO:78 |
| | | ALQASALNAWR, | SEQ ID NO:41 |
| | | LSQIGVENTEENR. | SEQ ID NO:509 |
| ADF-266 | ADPI-266 | IINNTENLVR, | SEQ ID NO:346 |
| | | FGTINIVHPK, | SEQ ID NO:218 |
| | | DDLLGFALR, | SEQ ID NO:77 |
| | | GVGISVLEMSHR, | SEQ ID NO:296 |
| ADF-267 | ADPI-267 | QAVQILQQVTVR, | SEQ ID NO:595 |
| | | TIEYLQPNPASR, | SEQ ID NO:699 |
| ADF-268 | ADPI-268.2 | IAEFAFEYAR, | SEQ ID NO:323 |
| ADF-268 | ADPI-268.1 | GTGGVDTAAVGGVFDVSNAD, | SEQ ID NO:295 |
| | | R, | SEQ ID NO:426 |
| | | LAVEALSSLDGDLAGR, | SEQ ID NO:443 |
| | | LEQGQAIDDLMPAQK, | |
| ADF-269 | ADPI-269 | FVTVQTISGTGALR, | SEQ ID NO:244 |
| | | NMGLYGER, | SEQ ID NO:574 |
| | | KMNLGVGAYR, | SEQ ID NO:415 |
| | | DAGMQLQGYR, | SEQ ID NO:71 |
| | | IAAAILNTPDLR, | SEQ ID NO:321 |
| ADF-270 | ADPI-270 | LADVYQAELR, | SEQ ID NO:421 |
| | | KIESLEEEIR, | SEQ ED NO:411 |
| | | ALAAELNQLR, | SEQ ID NO:31 |
| ADF-271 | ADPI-271 | LAD VYQAELR, | SEQ ID NO:421 |
| | | KIESLEEEIR, | SEQ ID NO:411 |
| | | ALAAELNQLR, | SEQ ID NO:31 |
| ADF-272 | ADPI-272 | QLLLTADDR, | SEQ ID NO:615 |
| | | PYQYPALTPEQK, | SEQ ID NO:593 |
| ADF-273 | ADPI-273 | TTGIVETHFTFK, | SEQ ID NO:710 |
| | | MFDVGGQR, | SEQ ID NO:535 |
| ADF-274 | ADPI-274 | TTGIVETHFTFK, | SEQ ID NO:710 |
| | | IGAADYQPTEQDILR, | SEQ ID NO:336 |
| | | IIHEDGFSGEDVK, | SEQ ID NO:343 |
| ADF-275 | ALDPI-275 | LADVYQAELR, | SEQ ID NO:421 |
| | | KIESLEEEIR, | SEQ ID NO:411 |
| | | ALAAELNQLR, | SEQ ID NO:31 |
| ADF-276 | ADPI-276 | LQSWLYSSR, | SEQ ID NO:501 |
| | | ITSAYLQDIENAYK, | SEQ ID NO:390 |
| ADF-277 | ADPI-277 | LADVYQAELR, | SEQ ID NO:421 |
| | | EAASYQEALAR, | SEQ ID NO:126 |
| | | KIESLEEEIR, | SEQ ID NO:410 |
| ADF-278 | ADPI-278.1 | LMDEVAGIVAAR, | SEQ ID NO:487 |
| | | VLEVPPVVYSR, | SEQ ID NO:745 |
| ADF-278 | ADPI-278.2 | DDNGVPFVR, | SEQ ID NO:78 |
| | | LSQIGVENTEENIR, | SEQ ID NO:510 |
| ADF-279 | ADPI-279.1 | SLAEGYFDAAGR, | SEQ ID NO:646 |
| ADF-279 | ADPI-279.2 | SLAEGYFDAAGR, | SEQ ID NO:646 |
| ADF-280 | ADPI-280 | LLDELTLEGVAR, | SEQ ID NO:473 |
| | | ELEDLVR, | SEQ ID NO:162 |
| ADF-281 | ADPI-281 | DYAVSTVPVADGLHLK, | SEQ ID NO:122 |
| | | DENATLDGGDVLFTGR, | SEQ ID NO:79 |
| ADF-282 | ADPI-282 | LLDELTLEGVAR, | SEQ ID NO:473 |
| | | ELEDLVR, | SEQ ID NO:162 |
| ADF-283 | ADPI-283 | AILVDLEPGTMDSVR, | SEQ ID NO:30 |
| | | LAVNMVPFPR, | SEQ ID NO:428 |
| ADF-284 | ADPI-284 | TLVMAVYDFDR, | SEQ ID NO:704 |
| | | VFVGYNSTGAELR, | SEQ ID NO:733 |
| | | VPYSELGGK, | SEQ ID NO:760 |
| ADF-285 | ADPI-285 | YPENFFLLR, | SEQ ID NO:803 |
| | | AHQVVEDGYEFFAK, | SEQ ID NO:28 |
| ADF-286 | ADPI-286 | LISWYDNEFGYSNIR, | SEQ ID NO:467 |
| | | LVINGNPITIFQER, | SEQ ID NO:520 |
| ADF-287 | ADPI-287 | GHWFLSPR, | SEQ ID NO:273 |
| | | ISWAMEDSDLER, | SEQ ED NO:385 |
| ADF-288 | ADPI-288 | SPSTGLYDNLEK, | SEQ ID NO:657 |
| | | LLDELTLEGVAR, | SEQ ID NO:474 |
| ADF-289 | ADPI-289 | GALQNIIPASTGAAK, | SEQ ID NO:250 |
| | | LVINGNPITIFQER, | SEQ ID NO:520 |
| | | LISWYDNEFGYSNR, | SEQ ID NO:467 |
| ADF-290 | ADPI-290 | EHHFEAIALVEK, | SEQ ID NO:153 |
| | | FFGNSWAETYR, | SEQ ID NO:216 |
| | | ALQAAYGASAPSVTSAALR, | SEQ ID NO:40 |
| ADF-291 | ADPI-291 | GALQNIIPASTGAAK, | SEQ ID NO:250 |
| | | LISWYDNEFGYSNR, | SEQ ID NO:468 |
| ADF-292 | ADPI-292 | VDFPQDQLTALTGR, | SEQ ID NO:723 |
| | | AGAGSATLSMAYAGAR, | SEQ ID NO:20 |
| | | HGVYNPNK, | SEQ ID NO:311 |
| | | VNVPVIGGHAGK, | SEQ ID NO:756 |
| ADF-293 | ADPI-293 | EVGVYEALKDDSWLK, | SEQ ID NO:192 |
| | | VIVVGNPANTNCLTASK, | SEQ ID NO:741 |
| | | DVIATDKEDVAFK, | SEQ ID NO:116 |
| | | EVGVYEALK, | SEQ ID NO:191 |
| | | FVEGLPINDFSR, | SEQ ID NO:242 |
| ADF-294 | ADPI-294 | EIIDLVLDR, | SEQ ID NO:157 |
| | | AVFVDLEPTVIDEVR, | SEQ ID NO:64 |
| ADF-295 | ADPI-295 | AILVDLEPGTMDSVR, | SEQ ID NO:30 |
| | | INVYYNEAAGNK, | SEQ ID NO:358 |
| | | LAVNMVPFPR, | SEQ ID NO:427 |
| | | GHYTEGAELVDSVLDVVR, | SEQ ID NO:274 |
| ADF-296 | ADPI-296 | FFEHFIEGGR, | SEQ ID NO:215 |
| | | LLTSGYLQR, | SEQ ID NO:485 |
| | | IQQEIAVQNPLVSER, | SEQ ID NO:375 |
| ADF-297 | ADPI-297 | FPGQLNADLR, | SEQ ID NO:231 |
| | | LAVNMVPFPR, | SEQ ID NO:427 |
| | | INVYYNEATGGNYVPR, | SEQ ID NO:361 |
| | | AVLVDLEPGTMDSVR, | SEQ ID NO:67 |
| ADF-298 | ADPI-298 | AILVDLEPGTMDSVR, | SEQ ID NO:30 |
| | | INVYYNEAAGNK, | SEQ ID NO:358 |
| | | FPGQLNADLR, | SEQ ID NO:231 |
| | | LAVNMVPFPR, | SEQ ID NO:427 |
| | | GHYTEGAELVDSVLDVVR, | SEQ ID NO:274 |
| ADF-299 | ADPI-299 | LVIITAGAR, | SEQ ID NO:519 |
| ADF-300 | ADPI-300 | LDASLVIAGVR, | SEQ ID NO:429 |
| ADF-301 | ADPI-301 | NILLTNEQLESAR, | SEQ ID NO:569 |
| ADF-302 | ADPI-302 | LTFDSSFSPNTGK, | SEQ ID NO:512 |
| ADF-303 | ADPI-303 | GAGTDDHTLIR, | SEQ ID NO:249 |
| | | FITIFGTR, | SEQ ID NO:222 |
| | | YMTISGFQIEETIDR, | SEQ ID NO:802 |
| | | SEIDLFNIR, | SEQ ID NO:632 |
| ADF-304 | ADPI-304 | LIGTAVPQR, | SEQ ID NO:465 |
| | | DYNPLLAR, | SEQ ID NO:123 |
| ADF-305 | ADPI-305 | AASDIAMTELPPTHPIR, | SEQ ID NO:7 |
| | | YLAEFATGNDR, | SEQ ID NO:799 |
| ADF-306 | ADPI-306 | IQLVEEELDR, | SEQ ID NO:371 |
| | | IQVLQQQADDAEER, | SEQ ID NO:376 |
| | | KLVIIEGDLER, | SEQ IDNO:413 |
| ADF-307 | ADPI-307.1 | EKAEGDVAALNR, | SEQ ID NO:159 |
| | | IQALQQQADEAEDR, | SEQ ID NO:369 |
| ADF-307 | ADPI-307.2 | YLAEFATGNDR, | SEQ ID NO:798 |
| | | HLIPAANTGESK, | SEQ ID NO:315 |
| ADF-308 | ADPI-308 | THSDQFLVAFK, | SEQ ID NO:696 |
| | | HLLIGVSSDR, | SEQ ID NO:316 |
| | | IYGLGSLALYEK, | SEQ ID NO:402 |
| ADF-309 | ADPI-309 | IQLVEEELDR, | SEQ ID NO:371 |
| | | EDRYEEEIK, | SEQ ID NO:138 |
| | | ETAEADVASLNR, | SEQ ID NO:188 |
| | | LVIIESDLER, | SEQ ID NO:517 |
| ADF-310 | ADPI-310 | GDNFETVTWLSDSEVVR, | SEQ ID NO:256 |
| | | VAVHHAEAEALAR, | SEQ ID NO:721 |
| | | VLPGHFNTFGAER, | SEQ ID NO:750 |
| ADF-311 | ADPI-311 | FDGILGMAYPR, | SEQ ID NO:206 |
| | | YYTVFDR, | SEQ ID NO:816 |
| ADF-312 | ADPI-312 | DLPAIQPR, | SEQ ID NO:102 |
| | | IGSTIFGER, | SEQ ID NO:341 |
| ADF-313 | ADPI-313 | LQVSQQEDITK, | SEQ ID NO:503 |
| | | SLLVTELGSSR, | SEQ ID NO:652 |
| ADF-314 | ADPI-314 | GAEIVADTFR, | SEQ ID NO:247 |
| | | ALQDLGLR, | SEQ ID NO:42 |
| ADF-315 | ADPI-315 | GEEDWLYER, | SEQ ID NO:258 |
| ADF-316 | ADPI-316.2 | NNTVGLIQLNRPK, | SEQ ID NO:577 |
| | | LFYSTFATDDR, | SEQ ID NO:453 |
| ADF-316 | ADPI-316.1 | LATQSNEITIPVTFESR, | SEQ ID NO:425 |
| | | GPSWDPFR, | SEQ ID NO:288 |
| | | LFDQAFGLPR, | SEQ ID NO:446 |
| ADF-317 | ADPI-317 | EQMAISGGFIR, | SEQ ID NO:184 |
| | | TLVMLDEQGEQLER, | SEQ ID NO:705 |
| ADF-318 | ADPI-318 | DIPNENELQFQIK, | SEQ ID NO:95 |
| | | GLEISGTFTHR, | SEQ ID NO:280 |
| | | IQPGNIPNYTLSLK, | SEQ ID NO:374 |
| ADF-319 | ADPI-319 | NVTVTDVDIVFSK, | SEQ ID NO:587 |
| | | VDLVDESGYVSGYK, | SEQ ID NO:725 |
| ADF-320 | ADPI-320.1 | SVGYKPDFVGFEIPDK, | SEQ ID NO:671 |
| | | VFIPHGLIMDR, | SEQ ID NO:731 |
| ADF-320 | ADPI-320.2 | LPFPIIDDR, | SEQ ID NO:493 |
| | | VVFVFGPDK, | SEQ ID NO:772 |
| ADF-321 | ADPI-321 | PMTLGYWNIR, | SEQ ID NO:590 |
| | | LLLEYTDSSYEEK, | SEQ ID NO:482 |
| | | EDILENQFMDSR, | SEQ ID NO:136 |
| ADF-322 | ADPI-322 | VYEVATFYTMYNIR, | SEQ ID NO:780 |
| | | AAAVLPVLDLAQR, | SEQ ID NO:2 |
| ADF-323 | ADPI-323 | THLAPYSDELR, | SEQ ID NO:695 |
| | | DEPPQSPWDR, | SEQ ID NO:80 |
| | | DYVSQFEGSALGK, | SEQ ID NO:124 |
| | | VQPYLDDFQK, | SEQ ID NO:761 |
| AIDF-324 | ADPI-324 | FNVWDTAGQEK, | SEQ ID NO:228 |
| | | NLQYYDISAK, | SEQ ID NO:573 |
| ADF-325 | ADPI-325 | ELVDDSINNVR, | SEQ ID NO:175 |
| | | VFEVMLATDR, | SEQ ID NO:730 |
| AIDF-326 | ADPI-326 | NNIAMALEVTYR, | SEQ ID NO:575 |
| ADF-327 | ADPI-327 | IPNQFQSDPPAPSDK, | SEQ ID NO:364 |
| | | AALEGTATYR, | SEQ ID NO:5 |
| | | EADYVAQATR, | SEQ ID NO:127 |
| ADF-328 | ADPI-328 | DFLLQQTMLR, | SEQ ID NO:82 |
| ADF-329 | ADPI-329 | DFLLQQTMLR, | SEQ ID NO:81 |
| | | SLDFYTR, | SEQ ID NO:648 |
| ADF-330 | ADPI-330 | ATEPVIAFYFK, | SEQ ID NO:60 |
| | | EVQQGEEFER, | SEQ ID NO:193 |
| ADF-331 | ADPI-331 | QGGLGPMNIPLVSDPK, | SEQ ID NO:604 |
| | | QITVNDLPVGR, | SEQ ID NO:609 |
| | | LVQAFQFTDK, | SEQ ID NO:522 |
| | | IGHPAPNFK, | SEQ ID NO:339 |
| ADF-332 | ADPI-332 | TDPSILGGMIVR, | SEQ ID NO:684 |
| | | FSPLTTNLINLLAENGR, | SEQ ID NO:238 |
| | | SFLSQGQVLK, | SEQ ID NO:642 |
| ADF-333 | ADPI-333.1 | LYTLVLTDPDAPSR, | SEQ ID NO:528 |
| ADF-333 | ADPI-333.2 | QITVNIDLPVGR, | SEQ ID NO:609 |
| | | LVQAFQFTDK, | SEQ ID NO:523 |
| ADF-334 | ADPI-334 | QDEHGFISR, | SEQ ID NO:596 |
| | | APSWFDTGLSEMR, | SEQ ID NO:52 |
| | | HFSPEELK, | SEQ ID NO:306 |
| | | VLGDVIEVHGK, | SEQ ID NO:748 |
| ADF-335 | ADPI-335 | YLAEFATGNDR, | SEQ ID NO:799 |
| ADF-336 | ADPI-336 | NDFTEEEEAQVR, | SEQ ID NO:558 |
| ADF-337 | ADPI-337 | QDEHGFISR, | SEQ ID NO:596 |
| | | APSWFDTGLSEMR, | SEQ ID NO:52 |
| | | HESPEELK, | SEQ ID NO:306 |
| | | VLGDVIEVHGK, | SEQ ID NO:748 |
| ADF-338 | ADPI-338 | TEGDGVYTLNDKK, | SEQ ID NO:686 |
| | | TEGDGVYTLNDK, | SEQ ID NO:685 |
| | | TEGDGVYTLNNEK, | SEQ ID NO:687 |
| ADF-339 | ADPI-339 | LNGTDPEDVIR, | SEQ ID NO:489 |
| | | FTDEEVDELYR, | SEQ ID NO:240 |
| | | GNFNYIEFTR, | SEQ ID NO:286 |
| | | EAFNMIDQNR, | SEQ ID NO:129 |
| ADF-340 | ADPI-340 | EAFSLFDK, | SEQ ID NO:130 |
| | | DTDSEEEIR, | SEQ ID NO:114 |
| ADF-341 | ADPI-341 | TVEGAGSIAAATGFVK, | SEQ ID NO:713 |
| | | EGVVHGVATVAEK, | SEQ ID NO:150 |
| | | EQVTNVGGAVVTGVTAVAQK, | SEQ ID NO:186 |
| ADF-342 | ADPI-342 | HEWQVNGLDDIK, | SEQ ID NO:305 |
| | | YALYDATYETK, | SEQ ID NO:786 |
| | | LGGNVVVSLEGKPL, | SEQ ID NO:458 |
| ADF-343 | ADPI-343 | YALYDATYETK, | SEQ ID NO:786 |
| ADF-344 | ADPI-344 | AILVDLEPGTMIDSVR, | SEQ ID NO:30 |
| | | INVYYNEAAGNK, | SEQ ID NO:359 |
| ADF-345 | ADPI-345 | FEDENFILK, | SEQ ID NO:209 |
| | | IIPGFMCQGGDFTR, | SEQ ID NO:347 |
| | | EGMNIVEAMER, | SEQ ID NO:149 |
| | | VSFELFADK, | SEQ ID NO:763 |
| ADF-346 | ADPI-346 | EFIEGVSQFSVK, | SEQ ID NO:143 |
| | | VIDIFDTDGNGEVDFK, | SEQ ID NO:738 |
| | | DGYISNGELFQVLK, | SEQ ID NO:91 |
| ADF-347 | ADPI-347 | AQAELVGTADEATR, | SEQ ID NO:53 |
| ADF-348 | ADPI-348 | GNIDISSGTVLSDYVGSGPPK, | SEQ ID NO:285 |
| | | LYTLVLTDPDAPSR, | SEQ ID NO:528 |
| ADF-349 | ADPI-349 | AILVDLEPGTMDSVR, | SEQ ID NO:30 |
| | | INVYYNEAAGNK, | SEQ ID NO:359 |
| ADF-350 | ADPI-350 | EFEEDLTGIDDR, | SEQ ID NO:142 |
| | | MLVNENFEEYLR, | SEQ ID NO:544 |
| ADF-351 | ADPI-351 | DREGFFTNGLTLGAK, | SEQ ID NO:112 |
| | | EGFFTNGLTLGAK, | SEQ ID NO:147 |
| | | SQGGEPTYNVAVGR, | SEQ ID NO:658 |
| ADF-352 | ADPI-352 | LGVEFDETTADDR, | SEQ ID NO:459 |
| ADF-353 | ADPI-353 | LLVVYPWTQR, | SEQ ID NO:486 |
| | | EFTPPVQAAYQK, | SEQ ID NO:146 |
| | | VVAGVANALAHK, | SEQ ID NO:768 |
| | | VLGAFSDGLAHLDNLK, | SEQ ID NO:746 |
| | | LHVDPENFR, | SEQ ID NO:461 |
| ADF-354 | ADPI-354 | ILDVLEEIPK, | SEQ ID NO:350 |
| ADF-355 | ADPI-355 | VGAHAGEYGAEALER, | SEQ ID NO:734 |
| | | MFLSFPTTK, | SEQ ID NO:537 |
| ADF-356 | ADPI-356 | FLSQIESDR, | SEQ ID NO:224 |
| | | HIDLVEGDEGR, | SEQ ID NO:313 |
| | | GDFIALDLGGSSFR, | SEQ ID NO:254 |
| ADF-357 | ADPI-357 | DIPNENELQFQIK, | SEQ ID NO:95 |
| | | GLEISGTFTHR, | SEQ ID NO:280 |
| ADF-358 | ADPI-358 | VIFLENYR, | SEQ ID NO:739 |
| | | GLAGLGDVAEVR, | SEQ ID NO:278 |
| ADF-359 | ADPI-359 | TGAQELLR, | SEQ ID NO:690 |
| | | HVVPNEVVVQR, | SEQ ID NO:320 |
| | | EPGLQIWR, | SEQ ID NO:181 |
| | | QTQVSVLPEGGETPLFK, | SEQ ID NO:621 |
| | | VEHPEFLK, | SEQ ID NO:726 |
| ADF-360 | ADPI-360 | NAVTQEFGPVPDTAR, | SEQ ID NO:557 |
| | | EGWPLDIR, | SEQ ID NO:151 |
| | | WVVIGDENYGEGSSR, | SEQ ID NO:785 |
| ADF-361 | ADPI-361 | QLLTLSSELSQAR, | SEQ ID NO:616 |
| | | APDFVFYAPR, | SEQ ID NO:48 |
| ADF-362 | ADPI-362 | EFSDLFR, | SEQ ID NO:145 |
| | | DFQSGQHVIVR, | SEQ ID NO:83 |
| | | NFSGAELEGLVR, | SEQ ID NO:562 |
| | | LFADAEEEQR, | SEQ ID NO:444 |
| | | YVGESEANIR, | SEQ ID NO:813 |
| ADF-363 | ADPI-363 | SPLTYGVGVLNR, | SEQ ID NO:656 |
| | | ETAPTSAYSSPAR, | SEQ ID NO:189 |
| | | ELSDQAGSEFENSDVR, | SEQ ID NO:174 |
| | | TPTTILLTPER, | SEQ ID NO:707 |
| ADF-364 | ADPI-364 | TVNVVQFEPSK, | SEQ ID NO:715 |
| | | YPLFEGQETGK, | SEQ ID NO:804 |
| ADF-365 | ADPI-365.2 | ELLQESALIR, | SEQ ID NO:169 |
| | | GVVDSEDIPLNLSR, | SEQ ID NO:297 |
| ADF-365 | ADPI-365.1 | SPLTYGVGVLNR, | SEQ ID NO:656 |
| | | RFHSFGYAAR, | SEQ ID NO:404 |
| | | ELSDQAGSEFENSDVR, | SEQ ID NO:174 |
| | | TPTTILLTPER, | SEQ ID NO:707 |
| ADF-366 | ADPI-366 | VFNLYPR, | SEQ ID NO:732 |
| | | DNFTLTPEGTNGTEER, | SEQ ID NO:107 |
| ADF-367 | ADPI-367.2 | LASFYER, | SEQ ID NO:424 |
| | | VGHSELVGEIIR, | SEQ ID NO:736 |
| | | DDFLQQNGYTPYDR, | SEQ ID NO:75 |
| | | HFTEFVPLR, | SEQ ID NO:309 |
| ADF-367 | ADPI-367.1 | TTPSYVAFTDTER, | SEQ ID NO:712 |
| | | TVTNAVVTVPAYFNDSQR, | SEQ ID NO:716 |
| | | STAGDTHLGGEDFDNR, | SEQ ID NO:667 |
| | | FEELNADLFR, | SEQ ID NO:212 |
| | | VEIIANIDQGNR, | SEQ ID NO:727 |
| ADF-368 | ADPI-368.1 | AQIHDLVLVGGSTR, | SEQ ID NO:54 |
| | | DAGVIAGLNVLR, | SEQ ID NO:72 |
| | | ATAGDTHLGGEDFDNR, | SEQ ID NO:58 |
| | | IINEPTAAAIAYGLDR, | SEQ ID NO:345 |
| ADF-368 | ADPI-368.2 | STPEYFAIER, | SEQ ID NO:669 |
| | | SEIDLLNIR, | SEQ ID NO:633 |
| | | SLHQAIEGDTSGDFLK, | SEQ ID NO:651 |
| | | TTGKPIEASIR, | SEQ ID NO:711 |
| | | DLEADIIGDTSGHFQK, | SEQ ID NO:99 |
| ADF-369 | ADPI-369.1 | ISEQTYQLSR, | SEQ ID NO:380 |
| | | SQLLILDR, | SEQ ID NO:661 |
| ADF-369 | ADPI-369.2 | NVDLSTFYQNR, | SEQ ID NO:584 |
| | | LRPESALAQAQK, | SEQ ID NO:505 |
| ADF-370 | ADPI-370 | LQVAGEITTGPR, | SEQ ID NO:502 |
| ADF-371 | ADPI-371 | LVNEVTEFAK, | SEQ ID NO:521 |
| | | DVFLGMFLYEYAR, | SEQ ID NO:115 |
| | | FQNALLVR, | SEQ ID NO:236 |
| | | VPQVSTPTLVEVSR, | SEQ ID NO:758 |
| ADF-372 | ADPI-372 | LVNEVTEFAK, | SEQ ID NO:521 |
| | | DVFLGMFLYBYAR, | SEQ ID NO:115 |
| | | FQNALLVR, | SEQ ID NO:236 |
| | | VPQVSTPTLVEVSR, | SEQ ID NO:758 |
| ADF-373 | AIDPI-373 | ISEQTYQLSR, | SEQ ID NO:380 |
| | | SQLLILDR, | SEQ ID NO:661 |
| AIDF-374 | ADPI-374 | SEVIGDGNQIEIEIPPTR, | SEQ ID NO:639 |
| | | ASEGPAFFPGR, | SEQ ID NO:57 |
| ADF-375 | ADPI-375 | VFNLYPR, | SEQ ID NO:732 |
| | | MVIPGGIDVHTR, | SEQ ID NO:553 |
| ADF-376 | ADPI-376 | ISEQTYQLSR, | SEQ ID NO:380 |
| | | SSASFSTTAVSAR, | SEQ ID NO:662 |
| | | SQLLILDR, | SEQ ID NO:660 |
| ADF-377 | ADPI-377 | ILVLDDTNHER, | SEQ ID NO:353 |
| | | LYSLGNGR, | SEQ ID NO:527 |
| ADF-378 | ADPI-378 | NLFSQTLSLGSQK, | SEQ ID NO:570 |
| | | ELEDLVR, | SEQ ID NO:162 |
| | | LLDELTLEGVAR, | SEQ ID NO:473 |
| ADF-379 | ADPI-379 | FFSEYEK, | SEQ ID NO:217 |
| | | LLSAEFLEQHYDR, | SEQ ID NO:484 |
| | | LLGELLLDR, | SEQ ID NO:480 |
| ADF-380 | ADPI-380 | LFSGDVVLTAR, | SEQ ID NO:450 |
| | | VVNVSSIMSVR, | SEQ ID NO:776 |
| | | SETITEEELVGLMNK, | SEQ ID NO:638 |
| ADF-381 | ADPI-381 | IFTSIGEDYDER, | SEQ ID NO:334 |
| | | DLQNVMTLR, | SEQ ID NO:104 |
| | | FDAGELITQR, | SEQ ID NO:205 |
| ADF-382 | ADPI-382 | KNGIPLELR, | SEQ ID NO:416 |
| | | NGIPLELR, | SEQ ID NO:564 |
| | | TAMDQALQWLEDK, | SEQ ID NO:679 |
| ADF-383 | ADPI-383 | LQSSDGEIFEVDVEIAK, | SEQ ID NO:500 |
| | | NDFTEEEEAQVR, | SEQ ID NO:558 |
| ADF-384 | ADPI-384 | AENFFILR, | SEQ ID NO:15 |
| | | ELLLQPVTISR, | SEQ ID NO:168 |
| | | VLIEGSINSVR, | SEQ ID NO:749 |
| ADF-385 | ADPI-385 | SSEIEQAVQSLDR, | SEQ ID NO:664 |
| | | ALAVGGLGSIIR, | SEQ ID NO:34 |
| ADF-386 | ADPI-386 | LELFLPEEYPMAAPK, | SEQ ID NO:442 |
| | | LLAEPVPGIK, | SEQ ID NO:471 |
| ADF-387 | ADPI-387 | AYELALYLR, | SEQ ID NO:69 |
| | | EGFFTNGLTLGAK, | SEQ ID NO:147 |
| | | SQGGEPTYNVAVGR, | SEQ ID NO:658 |
| ADF-388 | ADPI-388 | LIEEVHAVVTVR, | SEQ ID NO:463 |
| | | FILIQNR, | SEQ ID NO:221 |
| ADF-389 | ADPI-389 | FEEFLQR, | SEQ ID NO:211 |
| | | VYYDLTR, | SEQ IDNO:781 |
| | | QILLPFR, | SEQ ID NO:608 |
| ADF-390 | ADPI-390 | IDIIPNPQER, | SEQ ID NO:326 |
| | | NPDDITQEEYGEFYK, | SEQ ID NO:579 |
| ADF-391 | ADPI-391 | ELVVTQLGYDTR, | SEQ ID NO:178 |
| ADF-392 | ADPI-392 | DAGVIAGLNVLR, | SEQ ID NO:72 |
| | | IINEPTAAAIAYGLDR, | SEQ ID NO:345 |
| ADF-393 | ADPI-393 | FEELNADLFR, | SEQ ID NO:213 |
| ADF-394 | ADPI-394 | FDLTGIPPAPR, | SEQ ID NO:207 |
| | | VEIIANDQGNR, | SEQ ID NO:727 |
| ADF-395 | ADPI-395 | GGAEQFMEETER, | SEQ ID NO:268 |
| ADF-396 | ADPI-396 | FPFAANSR, | SEQ ID NO:230 |
| | | NLGLEELGIELDPR, | SEQ ID NO:571 |
| | | SEEQLKEEGIFYK, | SEQ ID NO:631 |
| ADF-397 | ADPI-397 | SEMPPVQFK, | SEQ ID NO:635 |
| | | SVAQQASLTEQR, | SEQ ID NO:670 |
| ADF-398 | ADPI-398 | EMNDAATFYTNR, | SEQ ID NO:179 |
| ADF-399 | ADPI-399.2 | TIPIDGNFFTYTR, | SEQ ID NO:701 |
| | | IFVEESIYDEFVR, | SEQ ID NO:335 |
| ADF-399 | ADPI-399.1 | AGTGVDNVDLEAATR, | SEQ ID NO:25 |
| | | DLPLLLFR, | SEQ ID NO:103 |
| | | QHVTEAFQFHF, | SEQ ID NO:606 |
| | | GGIVDEGALLR, | SEQ ID NO:269 |
| ADF-400 | ADPI-400 | FAAYFQQGDMESNGK, | SEQ ID NO:197 |
| | | AVLHVALR, | SEQ ID NO:65 |
| | | HFVALSTNTTK, | SEQ ID NO:310 |
| | | TNYTEGR, | SEQ ID NO:362 |
| ADF-401 | ADPI-401 | GSGNLEAIHIIK, | SEQ ID NO:293 |
| | | GATQQILDEAER, | SEQ ID NO:252 |
| | | LIEEVMIGEDK, | SEQ ID NO:464 |
| ADF-402 | ADPI-402 | IVLEDGTLHVTEGSGR, | SEQ ID NO:395 |
| | | DNFTLIPEGTNGTEER, | SEQ ID NO:107 |
| | | MDENQFVAVTSTNAAK, | SEQ ID NO:534 |
| ADF-403 | ADPI-403 | GDYPLEAVR, | SEQ ID NO:257 |
| | | LDIDSPPITAR, | SEQ ID NO:431 |
| ADF-404 | AIDPI-404.1 | MPLFEHYTR, | SEQ ID NO:546 |
| | | LFEASIETGDR, | SEQ ID NO:448 |
| | | AAGIDEQENWHEGK, | SEQ ID NO:3 |
| ADF-404 | ADPI-404.2 | MIGGPILPSER, | SEQ ID NO:539 |
| | | EENEGVYNGSWGGR, | SEQ ID NO:140 |
| | | IWAIDIPAPK, | SEQ ID NO:399 |
| AIDF-405 | ADPI-405 | NSSYFVEWIPNNVK, | SEQ ID NO:580 |
| | | AILVDLEPGTMDSVR, | SEQ ID NO:30 |
| | | TAVCDIPPR, | SEQ ID NO:681 |
| | | ISVYYNIEATGGK, | SEQ ID NO:383 |
| | | ISEQFTAMFR, | SEQ ID NO:378 |
| | | LAVNMVPFPR, | SEQ ID NO:428 |
| ADF-406 | ADPI-406 | STLMDTLFNTK, | SEQ ID NO:668 |
| | | TAAELLQSQGSQAGGSQTLK, | SEQ ID NO:678 |
| ADF-407 | ADPI-407 | AVLVDLEPGTMDSVR, | SEQ ID NO:66 |
| | | INVYYNEATGGK, | SEQ ID NO:360 |
| ADF-408 | ADPI-408 | TGQEIPVNVR, | SEQ ID NO:693 |
| | | TVFGVEPDLTR, | SEQ ID NO:714 |
| | | EGGSIPVTLTFQEATGK, | SEQ ID NO:148 |
| ADF-409 | ADPI-409 | TVFGVEPDLTR, | SEQ ID NO:714 |
| ADF-410 | ADPI-410 | EIIDLVLDR, | SEQ ID NO:157 |
| | | AVFVDLEPTVIDEVR, | SEQ ID NO:64 |
| ADF-411 | ADPI-411 | LADVYQAELR, | SEQ ID NO:421 |
| | | KIESLEEEIR, | SEQ ID NO:411 |
| | | ALAAELNQLR, | SEQ ID NO:31 |
| ADF-412 | ADPI-4122 | LADVYQAELR, | SEQ ID NO:421 |
| | | EAASYQEALAR, | SEQ ID NO:126 |
| ADF-412 | ADPI-412.1 | VALVYGQMNEPPGAR, | SEQ ID NO:719 |
| | | IMNVIGEPIDER, | SEQ ID NO:355 |
| | | IGLFGGAGVGK, | SEQ ID NO:340 |
| | | IPVGPETLGR, | SEQ ID NO:368 |
| ADF-413 | ADPI-413 | EAYPGDVFYLHSR, | SEQ ID NO:133 |
| | | ILGADTSVDLEETGR, | SEQ ID NO:351 |
| | | TGAIVDVPVGEELLGR, | SEQ ID NO:689 |
| | | AVDSLVPIGR, | SEQ ID NO:61 |
| ADF-414 | ADPI-414 | IMNVIGEPIDER, | SEQ ID NO:356 |
| ADF-415 | ADPI-415 | EIEAEIQALR, | SEQ ID NO:155 |
| ADF-416 | ADPI-416 | SLFNYHDTR, | SEQ ID NO:650 |
| | | SYELQESNVR, | SEQ ID NO:676 |
| | | AAAQLLQSQAQQSGAQQTK, | SEQ ID NO:1 |
| ADF-417 | ADPI-417 | AHGGYSVFAGVGER, | SEQ ID NO:27 |
| | | VALVYGQMNEPPGAR, | SEQ ID NO:719 |
| | | IMNVIGEPIDER, | SEQ ID NO:355 |
| | | IGLFGGAGVGK, | SEQ ID NO:340 |
| | | FTQAGSEVSALLGR, | SEQ ID NO:241 |
| | | IPVGPETLGR, | SEQ ID NO:367 |
| | | TIAMDGTEGLVR, | SEQ ID NO:697 |
| ADF-418 | ADPI-418 | IVIGMDVAASEFYR, | SEQ ID NO:393 |
| | | LGAEVYHTLK, | SEQ ID NO:455 |
| ADF-419 | ADPI-419 | AAVPSGASTGIYEALELR, | SEQ ID NO:8 |
| | | VVIGMDVAASEFFR, | SEQ ID NO:775 |
| ADF-420 | ADPI-420 | NGDGTIDFR, | SEQ ID NO:563 |
| ADF-421 | ADPI-421 | HEWQVNGLDDIK, | SEQ ID NO:305 |
| | | YALYDATYETK, | SEQ ID NO:786 |
| | | QIIVEEAK, | SEQ ID NO:607 |
| ADF-422 | ADPI-422 | AVFDETYPDPVR, | SEQ ID NO:62 |
| ADF-423 | ADPI-423 | GSPLVVISQGK, | SEQ ID NO:294 |
| | | VFNLYPR, | SEQ ID NO:732 |
| | | GLYDGPVCEVSVTPK, | SEQ ID NO:282 |
| | | IVLEDGTLHVTEGSGR, | SEQ ID NO:396 |
| ADF-424 | ADPI-424 | ISEQFTAMFR, | SEQ ID NO:378 |
| | | LAVNMVPFPR, | SEQ ID NO:427 |
| | | INVYYNEATGGNYVPR, | SEQ ID NO:361 |
| | | YLTVAAVFR, | SEQ ID NO:800 |
| ADF-425 | ADPI-425 | ILGADTSVDLEETGR, | SEQ ID NO:351 |
| | | VLSIGDGIAR, | SEQ ID NO:752 |
| ADF-426 | ADPI-426.2 | SFENSLGINVPR, | SEQ ID NO:640 |
| ADF-426 | ADPI-426.1 | EAYPGDVFYLHSR, | SEQ ID NO:133 |
| | | AVDSLVPIGR, | SEQ ID NO:61 |
| | | ILGADTSVDLEETGR, | SEQ ID NO:351 |
| ADF-427 | ADPI-427 | TGAIVDVPVGEELLGR, | SEQ ID NO:689 |
| | | AVDSLVPIGR, | SEQ ID NO:61 |
| ADF-428 | ADPI-428 | LLEGEENR, | SEQ ID NO:477 |
| | | LADVYQAELR, | SEQ ID NO:421 |
| | | EAASYQEALAR, | SEQ ID NO:125 |
| | | DNLAQDLATVR, | SEQ ID NO:109 |
| ADF-429 | ADPI-429 | AAVPSGASTGIYEALELR, | SEQ ID NO:8 |
| | | IGAEVYHNLK, | SEQ ID NO:337 |
| | | VVIGMDVAASEFFR, | SEQ ID NO:774 |
| ADF-430 | ADPI-430 | FVIDVGYER, | SEQ ID NO:243 |
| | | NVVLSGGSTMFR, | SEQ ID NO:588 |
| | | KFVIDVGYER, | SEQ ID NO:405 |
| ADF-431 | ADPI-431 | VWDYETGDFER, | SEQ ID NO:779 |
| | | EWIPRPPEK, | SEQ ID NO:194 |
| ADF-432 | ADPI-432 | FEDYLNAESR, | SEQ ID NO:210 |
| | | DVTNNVHYENYR, | SEQ ID NO:119 |
| ADF-433 | ADPI-433 | FEDYLNAESR, | SEQ ID NO:210 |
| | | DVTNNVHYENYR, | SEQ ID NO:119 |
| ADF-434 | ADPI-434 | IVFLEEASQQEK, | SEQ ID NO:392 |
| | | VKPIVTPR, | SEQ ID NO:742 |
| | | FLYLGDDR, | SEQ ID NO:226 |
| ADF-435 | ADPI-435 | YYGAQTVR, | SEQ ID NO:814 |
| | | YYGAQTVR, | SEQ ID NO:814 |
| | | IYELAAGGTAVGTGLNTR, | SEQ ID NO:401 |
| ADF-436 | ADPI-436 | KLEELELDEQQR, | SEQ ID NO:412 |
| | | DVKPSNILVNSR, | SEQ ID NO:117 |
| ADF-437 | ADPI-437 | LADVYQAELR, | SEQ ID NO:421 |
| | | KIESLEEEIR, | SEQ ID NO:411 |
| | | ALAAELNQLR, | SEQ ID NO:31 |
| ADF-438 | ADPI-438 | GISEETTTGVHNLYK, | SEQ ID NO:275 |
| | | IILLAEGR, | SEQ ID NO:344 |
| | | YPQLLPGIR, | SEQ ID NO:806 |
| ADF-439 | ADPI-439 | AEAGDNLGALVR, | SEQ ID NO:11 |
| | | YEEIDNAPEER, | SEQ ID NO:788 |
| ADF-440 | ADPI-440.1 | GYSFTTTAER, | SEQ ID NO:303 |
| ADF-440 | ADPI-440.2 | LADVYQAELR, | SEQ ID NO:421 |
| | | EAASYQEALAR, | SEQ ID NO:126 |
| | | ALAAELNQLR, | SEQ ID NO:31 |
| ADF-441 | ADPI-441 | APGFAHLAGLDK, | SEQ ID NO:50 |
| | | TYLQALPYFDR, | SEQ ID NO:717 |
| | | LDELEELLTNNR, | SEQ ID NO:430 |
| | | MFEFYER, | SEQ ID NO:536 |
| ADF-442 | ADPI-442 | LADVYQAELR, | SEQ ID NO:421 |
| | | KIESLEEEIR, | SEQ ID NO:411 |
| ADF-443 | ADPI-443 | ALAAELNQLR, | SEQ ID NO:32 |
| ADF-444 | ADPI-444 | DIVEAHYR, | SEQ ID NO:96 |
| | | KGYFEDR, | SEQ ID NO:408 |
| ADF-445 | ADPI-445 | GTGGVDTAAVGGVFDVSNAD, | SEQ ID NO:295 |
| | | R, | SEQ ID NO:426 |
| | | LAVEALSSLDGDLAGR, | SEQ ID NO:753 |
| | | VLTPELYAELR, | |
| ADF-446 | ADPI-446 | YIEEAIEK, | SEQ ID NO:793 |
| | | DIVEAHYR, | SEQ ID NO:96 |
| | | KGYFEDR, | SEQ ID NO:407 |
| | | QVYMSLPQGEK, | SEQ ID NO:624 |
| ADF-447 | ADPI-447.1 | ELPTAFDYVEFTR, | SEQ ID NO:171 |
| | | SSLSSAQADFNQLAELDR, | SEQ ID NO:665 |
| ADF-447 | ADPI-447.2 | LADVYQAELR, | SEQ ID NO:421 |
| | | ALAAELNQLR, | SEQ ID NO:31 |
| | | KIESLEEEIR, | SEQ ID NO:410 |
| | | EAASYQEALAR, | SEQ ID NO:126 |
| ADF-448 | ADPI-448 | LYPPSAEYPDLR, | SEQ ID NO:526 |
| | | SGYFDER, | SEQ ID NO:644 |
| | | SFLIWVNEEDHTR, | SEQ ID NO:641 |
| ADF-449 | ADPI-449 | LADVYQAELR, | SEQ ID NO:421 |
| | | ALAAELNQLR, | SEQ ID NO:32 |
| ADF-450 | ADPI-450.1 | AGFAGDDAPR, | SEQ ID NO:22 |
| | | GYSFTTTAER, | SEQ ID NO:303 |
| | | SYELPDGQVITIGNER, | SEQ ID NO:674 |
| ADF-450 | ADPI-450.2 | LADVYQAELR, | SEQ ID NO 421 |
| | | ALAAELNQLR, | SEQ ID NO:31 |
| | | KIESLEEEIR, | SEQ ID NO:410 |
| | | EAASYQEALAR, | SEQ ID NO:125 |
| | | DNLAQDLATVR, | SEQ ID NO:109 |
| ADF-451 | ADPI-451 | LEEGPPVTTVLTR, | SEQ ID NO:439 |
| | AHGFTFTR, | SEQ ID NO:26 | |
| ADF-452 | ADPI-452 | AGQVFLEELGNHK, | SEQ ID NO:24 |
| | | LYSLGNGR, | SEQ ID NO:527 |
| ADF-453 | ADPI-453 | SMLEVNYPMENGIVR, | SEQ ID NO:655 |
| | | GYAFNHSADFETVR, | SEQ ID NO:301 |
| | | DLMVGDEASELR, | SEQ ID NO:101 |
| ADF-454 | ADPI-454 | LQFPLPTAQR, | SEQ ID NO :497 |
| | | EEFGAEPELAVSAPGR, | SEQ ID NO:139 |
| ADF-455 | ADPI-455 | ISEQFTAMFR, | SEQ ID NO:379 |
| ADF-456 | ADPI-456 | EIIDLVLDR, | SEQ ID NO:157 |
| | | AVFVDLEPTVIDEVR, | SEQ ID NO:64 |
| | | QLFHPEQLITGK, | SEQ ID NO:613 |
| ADF-457 | ADPI-457 | FHQLDIDDLQSIR, | SEQ ID NO:219 |
| | | GQAAVQQLQAEGLSPR, | SEQ ID NO:289 |
| | | LFSGDVVLTAR, | SEQ ID NO:450 |
| | | EGWPSSAYGVTK, | SEQ ID NO:152 |
| | | VVNVSSIMSVR, | SEQ ID NO:776 |
| ADF-458 | ADPI-458 | EIIDLVLDR, | SEQ ID NO:157 |
| | | AVFVDLEPTVIDEVR, | SEQ ID NO:64 |
| | | QLFHPEQLITGK, | SEQ ID NO:613 |
| ADF-459 | ADPI-459 | LTFDTTFSPNTGK, | SEQ ID NO:513 |
| ADF-460 | ADPI-460 | QDEHGFISR, | SEQ ID NO:596 |
| | | HFSPEELK, | SEQ ID NO:307 |
| | | APSWFDTGLSEMR, | SEQ ID NO:52 |
| ADF-461 | ADPI-461 | KGDIFLVR, | SEQ ID NO:406 |
| | | EVDIGIPDATGR, | SEQ ID NO:190 |
| ADF-462 | ADPI-462 | ISEQTYQLSR, | SEQ ID NO:380 |
| | | SQLLILDR, | SEQ ID NO:661 |
| ADF-463 | ADPI-463 | ISEQTYQLSR, | SEQ ID NO:380 |
| | | SSASFSTTAVSAR, | SEQ ID NO:662 |
| | | SQLLILDR, | SEQ ID NO:660 |
| ADF-464 | ADPI-464 | IFNLYPR, | SEQ ID NO:332 |
| | | MADLHAVPR, | SEQ ID NO:529 |
| ADF-465 | ADPI-465 | AFPEHLYQR, | SEQ ID NO:19 |
| | | IFNLYPR, | SEQ IDNO:333 |
| ADF-466 | ADPI-466.1 | LFTPDVLR, | SEQ ID NO:451 |
| | | ALTSQLTDEELAQGR, | SEQ ID NO:44 |
| | | YIYIMGIQER, | SEQ ID NO:797 |
| ADF-466 | ADPI-466.2 | ELLSGPNR, | SEQ ID NO:170 |
| | | IDAMHGVVGPYVK, | SEQ ID NO:325 |
| | | ADNFEYSDPVDGSISR, | SEQ ID NO:9 |
| ADF-467 | ADPI-467 | GMYDGPVYEVPATPK, | SEQ ID NO:284 |
| ADF-468 | ADPI-468 | GMYDGPVFDLTTTPK, | SEQ ID NO:283 |
| | | ISVGSDSDLVIWDPDAVK, | SEQ ID NO:382 |
| | | IFNLYPR, | SEQ ID NO:332 |
| ADF-469 | ADPI-469.2 | FAEAFEAIPR, | SEQ ID NO:200 |
| | | LFVTNDAATILR, | SEQ ID NO:452 |
| ADF-469 | ADPI-469.1 | FSELTAEK, | SEQ ID NO:237 |
| | | IADGYEQAAR, | SEQ ID NO:322 |
| | | QQISLATQMVR, | SEQ ID NO:619 |
| | | DFSHPQMPK, | SEQ ID NO:84 |
| | | LDVTSVEDYK, | SEQ ID NO:435 |
| ADF-470 | ADPI-470 | ELVNNLAEIYGR, | SEQ ID NO:176 |
| | | LFEAEEQDLFR, | SEQ ID NO:447 |
| ADF-471 | ADPI-471 | IQDWYDK, | SEQ ID NO:370 |
| | | MTLDDFR, | SEQ ID NO:550 |
| ADF-472 | ADPI-472 | EIEAEIQALR, | SEQ ID NO:155 |
| ADF-473 | ADPI-473 | ISEQTYQLSR, | SEQ ID NO:380 |
| | | SQLLILDR, | SEQ ID NO:661 |
| ADF-474 | ADPI-474 | GDYPLEAVR, | SEQ ID NO:257 |
| | | LDIDSPPITAR, | SEQ ID NO:431 |
| | | TATESFASDPILYR, | SEQ ID NO:680 |
| ADF-475 | ADPI-475 | FLVFVANFDENDPK, | SEQ ID NO:225 |
| | | FQLLEGPPESMGR, | SEQ ID NO:235 |
| ADF-476 | ADPI-476 | DIMEDTIEDK, | SEQ ID NO:93 |
| | | SQLLILDR, | SEQ ID NO:661 |
| ADF-477 | ADPI-477 | EPVVTLEGHTK, | SEQ ID NO:182 |
| | | AAPEASGTPSSDAVSR, | SEQ ID NO:6 |
| ADF-478 | ADPI-478.1 | IAVGSDADLVIWDPDSVK, | SEQ ID NO:324 |
| | | IVLEDGTLHVTEGSGR, | SEQ ID NO:395 |
| | | VFNLYPR, | SEQ ID NO:732 |
| | | MVIPGGIDVHTR, | SEQ ID NO:553 |
| ADF-478 | ADPI-478.2 | IAVGSDADLVIWDPDSVK, | SEQ ID NO:324 |
| | | IVLEDGTLHVTEGSGR, | SEQ ID NO:395 |
| | | VFNLYPR, | SEQ ID NO:732 |
| | | MVIPGGIDVHTR, | SEQ ID NO:553 |
| ADF-479 | ADPI-479 | ELEEETNAFNR, | SEQ ID NO:163 |
| | | EFLSELQR, | SEQ ID NO:144 |
| ADF-480 | ADPI-480 | EIIDLVLDR, | SEQ ID NO:157 |
| | | AVFVDLEPTVIDEVR, | SEQ ID NO:64 |
| ADF-481 | ADPI-481 | MVSSYVGENAEFER, | SEQ 1D NO:555 |
| | | DGSVAIASKPR, | SEQ ID NO:89 |
| | | FYTDPVEAVK, | SEQ ID NO:246 |
| | | YGDLANWMIPGK, | SEQ ID NO:791 |
| ADF-482 | ADPI-482 | LIEEVMIGEDK, | SEQ ID NO:464 |
| | | GATQQILDEAER, | SEQ ID NO:252 |
| ADF-483 | ADPI-483 | HALIIYDDLSK, | SEQ ID NO:304 |
| | | ILGADTSVDLEETGR, | SEQ ID NO:351 |
| | | TGAIVDVPVGEELLGR, | SEQ ID NO:689 |
| | | VLSIGDGIAR, | SEQ ID NO:751 |
| | | TGTAEMSSILEER, | SEQ ID NO:694 |
| | | AVDSLVPIGR, | SEQ ID NO:61 |
| ADF-484 | ADPI-484 | ILGADTSVDLEETGR, | SEQ ID NO:351 |
| | | AVDSLVPIGR, | SEQ ID NO:61 |
| | | TGAIVDVPVGEELLGR, | SEQ ID NO:689 |
| ADF-485 | ADPI-485 | ELEEEVNNFQK, | SEQ ID NO:164 |
| | | SYELQESNVR, | SEQ ID NO:676 |
| ADF-486 | ADPI-486 | ALAAELNQLR, | SEQ ID NO:31 |
| | | LADVYQAELR, | SEQ ID NO:422 |
| ADF-487 | ADPI-487 | RWLAEWESR, | SEQ ID NO:625 |
| | | IPLAEWESR, | SEQ ID NO:363 |
| ADF-488 | ADPI-488 | ITQYLDAGGIPR, | SEQ ID NO:389 |
| | | FYFENLWSR, | SEQ ID NO:245 |
| ADF-489 | ADPI-489 | LADVYQAELR, | SEQ ID NO:421 |
| | | ALAAELNQLR, | SEQ ID NO:31 |
| | | KIESLEEEIR, | SEQ ID NO:410 |
| | | EAASYQEALAR, | SEQ ID NO:126 |
| ADF-490 | ADPI-490 | MLLYTEVTR, | SEQ ID NO:541 |
| | | DLGTESQIFISR, | SEQ ID NO:100 |
| ADF-491 | ADPI-491 | EAASYQEALAR, | SEQ ID NO:126 |
| ADF-492 | ADPI-492 | ADTLGELDLER, | SEQ ID NO:10 |
| | | GLAPEQPVTLR, | SEQ ID NO:279 |
| ADF-493 | ADPI-493.1 | AGFAGDDAPR, | SEQ ID NO:22 |
| | | SYELPDGQVITIGNER, | SEQ ID NO:674 |
| | | GYSFTTTAER, | SEQ ID NO:302 |
| | | QEYDESGPSIVHR, | SEQ ID NO:600 |
| ADF-493 | ADPI-493.2 | ALAAELNQLR, | SEQ ID NO:31 |
| | | HLQEYQDLLNVK, | SEQ ID NO:317 |
| | | KIESLEEEIR, | SEQ ID NO:410 |
| | | EAASYQEALAR, | SEQ ID NO:125 |
| | | DNLAQDLATVR, | SEQ ID NO:109 |
| ADF-494 | ADPI-494 | AEAGDNLGALVR, | SEQ ID NO:11 |
| | | YEEIDNAPEER, | SEQ ID NO:788 |
| ADF-495 | ADPI-495 | GKPVPTQGSR, | SEQ ID NO:277 |
| | | LYSLGNGR, | SEQ ID NO:527 |
| ADF-496 | ADPI-496 | SGYFDER, | SEQ ID NO:643 |
| | | IPTPVIHTK, | SEQ ID NO:366 |
| | | GWEFMWNER, | SEQ ID NO:300 |
| ADF-497 | ADPI-497 | ISAEGGEQVER, | SEQ ID NO:377 |
| | | ALAEEVEQVHR, | SEQ ID NO:33 |
| ADF-498 | ADPI-498 | LYPEGLAQLAR, | SEQ ID NO:525 |
| | | LLFEGAGSNPGDK, | SEQ ID NO:479 |
| ADF-499 | ADPI-499 | QFLSETEK, | SEQ ID NO:602 |
| ADF-500 | ADPI-500 | VFNLYPR, | SEQ ID NO:732 |
| | | DNFTLIPEGTNGTEER, | SEQ ID NO:107 |
| ADF-501 | ADPI-501 | FPAFGFGAR, | SEQ ID NO:229 |
| | | NNLNPSWEPFR, | SEQ ID NO:576 |
| | | DIVQFVPFR, | SEQ ID NO:98 |
| | | LLDGDDGPLR, | SEQ ID NO:475 |
| ADF-502 | ADPI-502 | YFVEAGAMAVR, | SEQ ID NO:790 |
| | | YINENLIVNTDELGR, | SEQ ID NO:796 |
| | | AFHNEAQVNPER, | SEQ ID NO:17 |
| | | EQLAIAEFAR, | SEQ ID NO:183 |
| ADF-503 | ADPI-503 | LLNLYPR, | SEQ ID NO:483 |
| | | MSVIWER, | SEQ ID NO:548 |
| ADF-504 | ADPI-504 | LLNLYPR, | SEQ ID NO:483 |
| | | MSVIWER, | SEQ ID NO:548 |
| ADF-505 | ADPI-505 | FANLNEQAAR, | SEQ ID NO:201 |
| ADF-506 | ADPI-506 | EDGGGWWYNR, | SEQ ID NO:135 |
| | | DNDGWLTSDPR, | SEQ ID NO:106 |
| | | QDGSVDFGR, | SEQ ID NO:597 |
| ADF-507 | ADPI-507 | EIEAEIQALR, | SEQ ID NO:154 |
| | | LRDDTEAAIR, | SEQ ID NO:504 |
| ADF-508 | ADPI-508 | LADVYQAELR, | SEQ ID NO:421 |
| | | KIESLEEEIR, | SEQ ID NO:411 |
| | | ALAAELNQLR, | SEQ ID NO:31 |
| ADF-509 | ADPI-509 | ILVLDDTNHER, | SEQ ID NO:354 |
| ADF-510 | ADPI-510 | ALAAELNQLR, | SEQ ID NO:31 |
| | | LADVYQAELR, | SEQ ID NO:421 |
| | | KIESLEEEIR, | SEQ ID NO:410 |
| | | EAASYQEALAR, | SEQ ID NO:126 |
| ADF-51 1 | ADPI-511 | AYTNFDAER, | SEQ ID NO:70 |
| | | TNQELQEINR, | SEQ ID NO:706 |
| | | AEDGSVIDYELTDQDAR, | SEQ ID NO:12 |
| | | SYSPYDMLESIR, | SEQ ID NO:677 |
| ADF-512 | ADPI-512 | ELVDDSINNVR, | SEQ ID NO:175 |
| | | VFEVMLATDR, | SEQ ID NO:730 |
| ADF-513 | ADPI-513 | DPVQEAWAEDVDLR, | SEQ ID NO:110 |
| | | LDIDSPPITAR, | SEQ ID NO:431 |
| | | TATESFASDPILYR, | SEQ ID NO:680 |
| ADF-514 | ADPI-514 | DLPLLLFR, | SEQ ID NO:103 |
| | | QHVTEAFQFHF, | SEQ ID NO:606 |
| | | TLGILGLGR, | SEQ ID NO:703 |
| | | GGIVDEGALLR, | SEQ ID NO:269 |
| ADF-515 | ADPI-515 | MVEGFFDR, | SEQ ID NO:551 |
| | | GFIGPGIDVPAPDMSTGER, | SEQ ID NO:264 |
| | | DIVHSGLAYTMER, | SEQ ID NO:97 |
| ADF-516 | ADPI-516 | GLVYETSVLDPDEGIR, | SEQ ID NO:281 |
| | | VVPGYGHAVLR, | SEQ ID NO:777 |
| ADF-517 | ADPI-517 | LADVYQAELR, | SEQ ID NO:421 |
| | | KIESLEEEIR, | SEQ ID NO:411 |
| | | ALAAELNQLR, | SEQ ID NO:31 |
| ADF-518 | ADPI-518 | GSPLVVISQGK, | SEQ ID NO:294 |
| | | IAVGSDADLVIWDPDSVK, | SEQ ID NO:324 |
| | | MDENQFVAVTSTNAAK, | SEQ ID NO:534 |
| | | MVIPGGIDVHTR, | SEQ ID NO:552 |
| | | IVLEDGTLHVTEGSGR, | SEQ ID NO:395 |
| | | DNFTLIPEGTNGTEER, | SEQ ID NO:107 |
| ADF-519 | ADPI-519 | AILVDLEPGTMDSVR, | SEQ ID NO:30 |
| | | LAVNMVPFPR, | SEQ ID NO:428 |
| | | ISEQFTAMFR, | SEQ ID NO:378 |
| ADF-520 | ADPI-520 | ISEQFTAMFR, | SEQ ID NO:378 |
| | | LAVNMVPFPR, | SEQ ID NO:427 |
| | | AVLVDLEPGTMDSVR, | SEQ ID NO:66 |
| | | INVYYNEATGGK, | SEQ ID NO:360 |
| ADF-521 | ADPI-521 | ALAAELNQLR, | SEQ ID NO:31 |
| | | KIESLEEEIR, | SEQ ID NO:410 |
| | | LEAENNLAAYR, | SEQ ID NO:436 |
| | | EAASYQEALAR, | SEQ ID NO:126 |
| ADF-522 | ADPI-522 | EIEAEIQALR, | SEQ ID NO:155 |
| ADF-523 | ADPI-523 | IGGIGTVPVGR, | SEQ ID NO:338 |
| | | LPLQDVYK, | SEQ ID NO:494 |
| | | QTVAVGVIK, | SEQ ID NO:622 |
| ADF-524 | ADPI-524 | LADVYQAELR, | SEQ ID NO:421 |
| | | KIESLEEEIR, | SEQ ID NO:411 |
| | | ALAAELNQLR, | SEQ ID NO:31 |
| ADF-525 | ADPI-525 | ALAAELNQLR, | SEQ ID NO:31 |
| | | EAASYQEALAR, | SEQ ID NO:126 |
| | | KIESLEEEIR, | SEQ ID NO:410 |
| ADF-526 | ADPI-526.1 | KLVILEGELER, | SEQ ID NO:414 |
| | | IQLVEEELDR, | SEQ ID NO:372 |
| ADF-526 | ADPI-526.2 | LADVYQAELR, | SEQ TD NO:421 |
| | | KIESLEEEIR, | SEQ ID NO:411 |
| | | ALAAIELNQLR, | SEQ ID NO:31 |
| ADF-527 | ADPI-527 | FEDENFILK, | SEQ ID NO:209 |
| | | VNPTVFFDIAVDGEPLGR, | SEQ ID NO:755 |
| | | IIPGFMCQGGDFTR, | SEQ ID NO:347 |
| | | EGMNIVEAMER, | SEQ ID NO:149 |
| | | VSFELFADK, | SEQ ID NO:763 |
| ADF-528 | ADPI-528 | GVVDSEDLPLNISR, | SEQ ID NO:299 |
| ADF-529 | ADPI-529 | YQFNYYEAK, | SEQ ID NO:808 |
| | | LDASLVIAGVR, | SEQ ID NO:429 |
| ADF-530 | ADPI-530.1 | AILVDLEPGTMDSVR, | SEQ ID NO:30 |
| | | LAVNMVPFPR, | SEQ ID NO:428 |
| | | ISEQFTAMFR, | SEQ ID NO:378 |
| ADF-530 | ADPI-530.2 | LADVYQAELR, | SEQ ID NO:421 |
| | | EAASYQEALAR, | SEQ ID NO:126 |
| | | ALAAELNQLR, | SEQ ID NO:31 |
| ADF-531 | ADPI-531 | VTILGHVQR, | SEQ ID NO:764 |
| | | GGTPSAFDR, | SEQ ID NO:272 |
| | | ELVVTQLGYDTR, | SEQ ID NO:177 |
| ADF-532 | ADPI-532 | DPVQEAWAEDVDLR, | SEQ ID NO:110 |
| | | LDIDSPPITAR, | SEQ ID NO:431 |
| | | TATESFASDPILYR, | SEQ ID NO:680 |
| ADF-533 | ADPI-533 | IVIGMDVAASEFYR, | SEQ ID NO:394 |
| ADF-534 | ADPI-534 | AGQVFLEELGNHK, | SEQ ID NO:24 |
| | | LYSLGNGR, | SEQ ID NO:527 |
| ADF-535 | ADPI-535 | GDYPLEAVR, | SEQ ID NO:257 |
| | | DPVQEAWAEDVDLR, | SEQ ID NO:111 |
| ADF-536 | ADPI-536 | YTLNFEAAQK, | SEQ ID NO:810 |
| | | LLASDAGLYR, | SEQ ID NO:472 |
| ADF-537 | ADPI-537 | LADVYQAELR, | SEQ ID NO:421 |
| | | KIESLEEEIR, | SEQ ID NO:411 |
| | | ALAAELNQLR, | SEQ ID NO:31 |
| ADF-538 | ADPI-538 | VLEAELLVLR, | SEQ ID NO:744 |
| | | FASFIER, | SEQ ID NO:204 |
| ADF-539 | ADPI-539 | LADVYQAELR, | SEQ ID NO:421 |
| | | EAASYQEALAR, | SEQ ID NO:126 |
| | | KIESLEEEIR, | SEQ ID NO:410 |
| ADF-540 | ADPI-540 | IGAADYQPTEQDILR, | SEQ ID NO:336 |
| ADF-541 | ADPI-541 | NEQDAYAINSYTR, | SEQ ID NO:560 |
| | | EAYMGNVLQGGEGQAPTR, | SEQ ID NO:132 |
| ADF-542 | ADPI-542 | VVYTGTYDTEGVTPTK, | SEQ ID NO:778 |
| ADF-543 | ADPI-543 | LTFDSSFSPNTGK, | SEQ ID NO:511 |
| | | WTEYGLTFTEK, | SEQ ID NO:784 |
| ADF-544 | ADPI-544 | ALPESLGQHALR, | SEQ ID NO:38 |
| | | DENATLDGGDVLFTGR, | SEQ ID NO:79 |
| | | DYAVSTVPVADGLHLK, | SEQ ID NO:122 |
| ADF-545 | ADPI-545 | TTPSYVAFTDTER, | SEQ ID NO:712 |
| | | FEELNADLFR, | SEQ ID NO:213 |
| ADF-546 | ADPI-546 | QGIQFYTQLK, | SEQ ID NO:605 |
| ADF-547 | ADPI-547.2 | ALAAELNQLR, | SEQ ID NO:32 |
| ADF-547 | ADPI-547.3 | MTLDDFR, | SEQ ID NO:550 |
| ADF-547 | ADPI-547.1 | FPGQLNADLR, | SEQ ID NO:231 |
| | | ISEQFTAMFR, | SEQ ID NO:379 |
| ADF-548 | ADPI-548.2 | EIEQEAAVELSQLR, | SEQ ID NO:156 |
| ADF-548 | ADPI-548.1 | LTFDSSFSPNTGK, | SEQ ID NO:511 |
| | | SENGLEFTSSGSANTETTK, | SEQ ID NO:637 |
| | | WTEYGLTFTEK, | SEQ ID NO:783 |
| ADF-549 | ADPI-549 | VIFLENYR, | SEQ ID NO:739 |
| | | GLAGLGDVAEVR, | SEQ ID NO:278 |

Those skilled in the art will understand, based upon the present description, that a given ADPI can be described according to the data provided for that ADPI in Table IV or V. The ADPI is a protein comprising a peptide sequence described for that ADPI (preferably comprising a plurality of, more preferably all of, the peptide sequences described for that ADPI) and has a pI of about the value stated for that ADPI (preferably within about 10%, more preferably within about 5% still more preferably within about 1% of the stated value) and has a MW of about the value stated for that ADPI (preferably within about 10%, more preferably within about 5%, still more preferably within about 1% of the stated value).

In one embodiment, brain tissue from one brain region of a subject is analyzed for quantitative detection of one or more of the following ADPIs: ADPI-1, ADPI-3.1, ADPI-3.2, ADPI-3.3, ADPI-5.3, ADPI-6, ADPI-8, ADPI-9, ADPI-10, ADPI-11, ADPI-12.1, ADPI-12.2, ADPI-13, ADPI-15, ADPI-16, ADPI-22, ADPI-23, ADPI-24, ADPI-25.3, ADPI-25.2, ADPI-26, ADPI-27, ADPI-29, ADPI-31, ADPI-33.1, ADPI-35, ADPI-37, ADPI-39, ADPI-41, ADPI-42, ADPI-54, ADPI-56, ADPI-61, ADPI-62, ADPI-66, ADPI-67, ADPI-68, ADPI-70, ADPI-72, ADPI-77, ADPI-78.2, ADPI-78.3, ADPI-79, ADPI-81.2, ADPI-82, ADPI-85, ADPI-87.1, ADPI-88, ADPI-90, ADPI-91.1, ADPI-91.2, ADPI-92, ADPI-94, ADPI-95, ADPI-97, ADPI-98, ADPI-101, ADPI-102.1, ADPI-103, ADPI-105.2, ADPI-105.1, ADPI-107, ADPI-108, ADPI-109, ADPI-111.1, ADPI-112, ADPI-113, ADPI-115, ADPI-117.1, ADPI-119, ADPI-120, ADPI-121, ADPI-124, ADPI-125, ADPI-126, ADPI-126, ADPI-126, ADPI-127, ADPI-128, ADPI-129, ADPI-130, ADPI-131, ADPI-132, ADPI-133, ADPI-138, ADPI-139, ADPI-140, ADPI-141, ADPI-142.2, ADPI-142.1, ADPI-143.1, ADPI-143.2, ADPI-144, ADPI-146.2, ADPI-146.1, ADPI-148, ADPI-149.1, ADPI-150, ADPI-151.2, ADPI-151.1, ADPI-152, ADPI-153.1, ADPI-153.2, ADPI-153.3, ADPI-154, ADPI-155, ADPI-156.2, ADPI-156.1, ADPI-157, ADPI-159.1, ADPI-159.2, ADPI-160, ADPI-162.2, ADPI-162.3, ADPI-162.1, ADPI-163, ADPI-165, ADPI-172, ADPI-173.1, ADPI-173.2, ADPI-175.2, ADPI-175.1, ADPI-176, ADPI-182, ADPI-183, ADPI-188.1, ADPI-189.2, ADPI-191, ADPI-193, ADPI-194.2, ADPI-196.2, ADPI-196.3, ADPI-196.1, ADPI-196.4, ADPI-202, ADPI-204, ADPI-208, ADPI-209, ADPI-216, ADPI-217, ADPI-220, ADPI-223, ADPI-228, ADPI-229, ADPI-230.1, ADPI-230.2, ADPI-232.1, ADPI-232.2, ADPI-236.2, ADPI-236.3, ADPI-237.2, ADPI-237.1, ADPI-240, ADPI-243, ADPI-245, ADPI-250, ADPI-251, ADPI-252.1, ADPI-252.2, ADPI-253, ADPI-254, ADPI-255, ADPI-256, ADPI-257.1, ADPI-257.2, ADPI-258, ADPI-259.1, ADPI-259.2, ADPI-260, ADPI-261, ADPI-262, ADPI-263, ADPI-264, ADPI-265, ADPI-266, ADPI-267, ADPI-268.1, ADPI-268.2, ADPI-269, ADPI-270, ADPI-271, ADPI-272, ADPI-273, ADPI-274, ADPI-275, ADPI-276, ADPI-277, ADPI-278.1, ADPI-278.2, ADPI-279.1, ADPI-279.2, ADPI-280, ADPI-281, ADPI-282, ADPI-283, ADPI-284, ADPI-285, ADPI-286, ADPI-287, ADPI-288, ADPI-289, ADPI-290, ADPI-291, ADPI-292, ADPI-293, ADPI-294, ADPI-295, ADPI-296, ADPI-297, ADPI-298, ADPI-299, ADPI-300, ADPI-301, ADPI-302, ADPI-303, ADPI-304, ADPI-305, ADPI-306, ADPI-307.1, ADPI-307.2, ADPI-308, ADPI-309, ADPI-311, ADPI-312, ADPI-313, ADPI-314, ADPI-315, ADPI-316.1, ADPI-316.2, ADPI-317, ADPI-318, ADPI-319, ADPI-320.1, ADPI-320.2, ADPI-321, ADPI-322, ADPI-323, ADPI-324, ADPI-325, ADPI-326, ADPI-327, ADPI-328, ADPI-329, ADPI-330, ADPI-331, ADPI-332, ADPI-333.1, ADPI-333.2, ADPI-334, ADPI-335, ADPI-336, ADPI-337, ADPI-338, ADPI-339, ADPI-340, ADPI-341, ADPI-342, ADPI-343, ADPI-344, ADPI-345, ADPI-346, ADPI-347, ADPI-348, ADPI-349, ADPI-350, ADPI-351, ADPI-352, ADPI-353, ADPI-354, ADPI-355, ADPI-356, ADPI-357, ADPI-358, ADPI-359, ADPI-360, ADPI-361, ADPI-362, ADPI-363, ADPI-364, ADPI-365.1, ADPI-365.2, ADPI-366, ADPI-367.1, ADPI-367.2, ADPI-368.1, ADPI-368.2, ADPI-369.1, ADPI-369.2, ADPI-370, ADPI-371, ADPI-372, ADPI-373, ADPI-374, ADPI-375, ADPI-376, ADPI-377, ADPI-378, ADPI-379, ADPI-380, ADPI-381, ADPI-382, ADPI-383, ADPI-384, ADPI-386, ADPI-387, ADPI-388, ADPI-389, ADPI-390, ADPI-391, ADPI-392, ADPI-393, ADPI-394, ADPI-395, ADPI-396, ADPI-397, ADPI-398, ADPI-399.1, ADPI-399.2, ADPI-400, ADPI-401, ADPI-402, ADPI-403, ADPI-404.1, ADPI-404.2, ADPI-405, ADPI-406, ADPI-407, ADPI-408, ADPI-409, ADPI-410, ADPI-411, ADPI-412.1, ADPI-412.2, ADPI-413, ADPI-414, ADPI-415, ADPI-416, ADPI-417, ADPI-418, ADPI-419, ADPI-420, ADPI-421, ADPI-422, ADPI-423, ADPI-424, ADPI-425, ADPI-426.1, ADPI-426.2, ADPI-427, ADPI-428, ADPI-429, ADPI-430, ADPI-431, ADPI-432, ADPI-433, ADPI-434, ADPI-435, ADPI-435, ADPI-436, ADPI-437, ADPI-438, ADPI-439, ADPI-440.1, ADPI-440.2, ADPI-441, ADPI-442, ADPI-443, ADPI-444, ADPI-445, ADPI-446, ADPI-447.1, ADPI-447.2, ADPI-448, ADPI-449, ADPI-450.1, ADPI-450.2, ADPI-451, ADPI-452, ADPI-453, ADPI-454, ADPI-455, ADPI-456, ADPI-457, ADPI-458, ADPI-459, ADPI-460, ADPI-461, ADPI-462, ADPI-463, ADPI-464, ADPI-465, ADPI-466.1, ADPI-466.2, ADPI-467, ADPI-468, ADPI-469.1, ADPI-469.2, ADPI-470, ADPI-471, ADPI-472, ADPI-473, ADPI-474, ADPI-475, ADPI-476, ADPI-477, ADPI-478.1, ADPI-478.2, ADPI-479, ADPI-480, ADPI-481, ADPI-482, ADPI-483, ADPI-484, ADPI-485, ADPI-486, ADPI-487, ADPI-488, ADPI-489, ADPI-490, ADPI-491, ADPI-492, ADPI-493.1, ADPI-493.2, ADPI-494, ADPI-495, ADPI-496, ADPI-497, ADPI-498, ADPI-499, ADPI-500, ADPI-501, ADPI-502, ADPI-503, ADPI-504, ADPI-505, ADPI-506, ADPI-507, ADPI-508, ADPI-509, ADPI-510, ADPI-511, ADPI-512, ADPI-513, ADPI-514, ADPI-515, ADPI-516, ADPI-517, ADPI-518, ADPI-519, ADPI-520, ADPI-521, ADPI-522, ADPI-523, ADPI-524, ADPI-525, ADPI-526.1, ADPI-526.2, ADPI-527, ADPI-528, ADPI-529, ADPI-530.1, ADPI-530.2, ADPI-531, ADPI-532, ADPI-533, ADPI-534, ADPI-535, ADPI-536, ADPI-537, ADPI-538, ADPI-539, ADPI-540, ADPI-541, ADPI-542, ADPI-543, ADPI-544, ADPI-545, ADPI-546, ADPI-547.1, ADPI-547.2, ADPI-547.3, ADPI-548.1, ADPI-548.2, ADPI-549 or any suitable combination of them, wherein an altered abundance of the ADPI or ADPIs (or any suitable combination of them) in the brain tissue from one brain region of the subject relative to brain tissue from the same region in a subject or subjects free from Alzheimer's disease (e.g., a control sample or a previously determined reference range) indicates the presence of Alzheimer's disease.

In yet a further embodiment, brain tissue from a subject is analyzed for quantitative detection of one or more ADPIs and one or more previously known biomarkers of Alzheimer's disease. In accordance with this embodiment, the abundance of each ADPI and known biomarker relative to a control or reference range indicates whether a subject has Alzheimer's disease.

In another embodiment, brain tissue from a subject is analyzed for quantitative detection of one or more of the following ADPIs: ADPI-1, ADPI-6, ADPI-8, ADPI-9, ADPI-10, ADPI-16, ADPI-26, ADPI-27, ADPI-31, ADPI-67, ADPI-77, ADPI-82, ADPI-90, ADPI-98, ADPI-119, ADPI-120, ADPI-124, ADPI-130, ADPI-132, ADPI-148, ADPI-150, ADPI-152, ADPI-159.1, ADPI-159.2, ADPI-162.2, ADPI-162.3, ADPI-162.1, ADPI-163, ADPI-204, ADPI-237.2, ADPI-237.1, ADPI-268.1, ADPI-268.2, ADPI-270, ADPI-271, ADPI-275, ADPI-284, ADPI-294, ADPI-297, ADPI-318, ADPI-328, ADPI-338, ADPI-346, ADPI-382, ADPI-384, ADPI-411, ADPI-413, ADPI-419, ADPI-427, ADPI-443, ADPI-450.1, ADPI-450.2, ADPI-452, ADPI-456, ADPI-458, ADPI-491, ADPI-507, ADPI-510 or any suitable combination of them, wherein an altered abundance of the ADPI or ADPIs (or any suitable combination of them) in the brain tissue from any region of the brain of the subject relative to brain tissue from a subject or subjects free from Alzheimer's disease (e.g., a control sample or a previously determined reference range) indicates the presence of Alzheimer's disease.

In yet a further embodiment, brain tissue from a subject is analyzed for quantitative detection of one or more ADPIs and one or more previously known biomarkers of Alzheimer's disease. In accordance with this embodiment, the abundance of each ADPI and known biomarker relative to a control or reference range indicates whether a subject has Alzheimer's disease.

In one embodiment, brain tissue from a subject is analyzed for quantitative detection of one or more of the following ADPIs: ADPI-1, ADPI-6, ADPI-8, ADPI-9, ADPI-10, ADPI-12.1, ADPI-12.2, ADPI-23, ADPI-25.3, ADPI-25.2, ADPI-26, ADPI-27, ADPI-29, ADPI-31, ADPI-67, ADPI-77, ADPI-79, ADPI-85, ADPI-90, ADPI-91.1, ADPI-91.2, ADPI-102.1, ADPI-103, ADPI-119, ADPI-120, ADPI-121, ADPI-124, ADPI-132, ADPI-142.2, ADPI-142.1, ADPI-144, ADPI-148, ADPI-149.1, ADPI-150, ADPI-151.2, ADPI-151.1, ADPI-152, ADPI-154, ADPI-155, ADPI-157, ADPI-159.1, ADPI-159.2, ADPI-160, ADPI-162.2, ADPI-162.3, ADPI-162.1, ADPI-163, ADPI-165, ADPI-173.1, ADPI-173.2, ADPI-175.2, ADPI-175.1, ADPI-176, ADPI-193, ADPI-202, ADPI-204, ADPI-250, ADPI-251, ADPI-260, ADPI-262, ADPI-266, ADPI-270, ADPI-271, ADPI-272, ADPI-275, ADPI-280, ADPI-282, ADPI-283, ADPI-286, ADPI-288, ADPI-295, ADPI-300, ADPI-302, ADPI-304, ADPI-319, ADPI-321, ADPI-331, ADPI-335, ADPI-336, ADPI-337, ADPI-355, ADPI-364, ADPI-366, ADPI-367.1, ADPI-367.2, ADPI-368.1, ADPI-368.2, ADPI-373, ADPI-375, ADPI-376, ADPI-381, ADPI-387, ADPI-390, ADPI-399.1, ADPI-399.2, ADPI-400, ADPI-403, ADPI-404.1, ADPI-404.2, ADPI-405, ADPI-410, ADPI-412.1, ADPI-412.2, ADPI-414, ADPI-415, ADPI-419, ADPI-423, ADPI-429, ADPI-434, ADPI-437, ADPI-442, ADPI-443, ADPI-444, ADPI-446, ADPI-452, ADPI-456, ADPI-469.1, ADPI-469.2, ADPI-471, ADPI-472, ADPI-478.1, ADPI-478.2, ADPI-485, ADPI-491, ADPI-493.1, ADPI-493.2, ADPI-495, ADPI-504, ADPI-505, ADPI-510, ADPI-511, ADPI-514, ADPI-518, ADPI-524, ADPI-527, ADPI-535 or any suitable combination of them, wherein an altered abundance of the ADPI or ADPIs (or any suitable combination of them) in the brain tissue from the subject relative to brain tissue from a subject or subjects free from Alzheimer's disease (e.g., a control sample or a previously determined reference range) is an early indicator for the presence of Alzheimer's disease.

In yet a further embodiment, brain tissue from a subject is analyzed for quantitative detection of one or more ADPIs and one or more previously known biomarkers of Alzheimer's disease. In accordance with this embodiment, the abundance of each ADPI and known biomarker relative to a control or reference range indicates whether a subject has Alzheimer's disease.

Preferably, the abundance of a ADPI is normalized to an Expression Reference Protein Isoform (ERPI). ERPIs can be identified by partial amino acid sequence characterization of ERFs, which are described above, and which may be accomplished using e.g. the methods and apparatus of the Preferred Technology. The partial amino acid sequences of ERPIs are presented in Table VI.

Table V. Expression Reference Protein Isoforms

TABLE V

| | | Amino Acid Sequence of | |
|---|---|---|---|
| | | Tryptic Digest | |
| ADF# | ADPI# | peptides | SEQ ID NO: |
| ERF-1 | ERPI-1 | YQETFNVIER | SEQ ID NO:807 |
| ERF-2 | ERPI-2 | SVTEQGAELSNEER, | SEQ ID NO:673 |
| | | FKIETELR | SEQ ID NO:160 |
| ERF-3 | ERPI-3 | QSPVDIDTHTAK, | SEQ ID NO:620 |
| | | GGPLDGTYR, | SEQ ID NO:271 |
| | | GGPLDGTYR, | SEQ ID NO:271 |
| ERF-4 | ERPI-4 | DAGYEFDICFTSVQK, | SEQ ID NO:73 |
| | | ALPFWNEEIVPQIK | SEQ ID NO:39 |
| ERF-5 | ERPI-5.1 | YTMGDAPDYDR | SEQ ID NO:811 |
| ERF-5 | ERPI-5.2 | AALDGTPGMIGYGMAK, | SEQ ID NO:4 |
| | | MTDSFTEQADQVTAEVGK | SEQ ID NO:549 |
| ERF-6 | ERPI-6 | IIYGGSVTGATCK, | SEQ ID NO:348 |
| | | VTNGAFTGEISPGMIK, | SEQ ID NO:766 |
| | | VTNGAFTGEISPGMIK | SEQ ID NO:766 |
| ERF-7 | ERPI-7 | DGLILTSR, | SEQ ID NO:88 |
| | | DVVICPDASLEDAK | SEQ ID NO:120 |
| ERF-8 | ERPI-8 | EEVVTVETWQEGSLK, | SEQ ID NO:141 |
| | | MLLADQGQSWK, | SEQ ID NO:540 |
| | | AFLASPEYVNLPINGNGK, | SEQ ID NO:18 |
| | | ASCLYGQLPK, | SEQ ID NO:56 |
| | | FQDGDLTLYQSNTILR, | SEQ ID NO:233 |
| | | PPYTVVYFPVR | SEQ ID NO:591 |
| ERF-9 | ERPI-9 | QTTVNDLPVGR, | SEQ ID NO:609 |
| | | LSEDYGVLK, | SEQ ID NO:506 |
| | | LSEDYGVLK | SEQ ID NO:506 |
| ERF-10 | ERPI-10 | AGLVDDFEK, | SEQ ID NO:23 |
| | | KYPYWPHQPIENL, | SEQ ID NO:419 |
| | | KYPYWPHQPIENL | SEQ ID NO:419 |
| ERF-11 | ERPI-11 | GDGPVQGIINFEQK, | SEQ ID NO:255 |
| | | HVGDLGNVTADK | SEQ ID NO:319 |
| ERF-12 | ERPI-12 | LNDFASTVR | SEQ ID NO:488 |
| ERF-13 | ERPI-13 | ITPSYVAFTPEGER | SEQ ID NO:388 |
| ERF-14 | ERPI-14 | FASEIAGVDDLGTTGR, | SEQ ID NO:202 |
| | | LEEVSPNLVR, | SEQ ID NO:441 |
| | | FEAPLFNAR, | SEQ ID NO:208 |
| | | DFYMTDSISR, | SEQ ID NO:86 |
| | | VAVTPPGLAR | SEQ ID NO:722 |
| ERF-15 | ERPI-15 | GDFCIQVGR, | SEQ ID NO:253 |
| | | FMQASEDLLK | SEQ ID NO:227 |

As shown above, the ADPIs described herein include previously unknown proteins or unknown variants of known proteins, as well as variants of known proteins where the variants were not previously known to be associated with Alzheimer's disease. For each ADPI, the present invention additionally provides: (a) a preparation comprising the isolated ADPI; (b) a preparation comprising one or more fragments of a ADPI; and (c) antibodies that bind to said ADPI, to said fragments, or both to said ADPI and to said fragments. As used herein, a ADPI is “isolated” when it is present in a preparation that is substantially free of other proteins, i.e., a preparation in which less than 10% (particularly less than 5%, more particularly less than 1%) of the total protein present is contaminating protein(s). Another protein is a protein or Protein Isoform having a significantly different pI or MW from those of the isolated ADPI, as determined by 2D electrophoresis. As used herein, a “significantly different” pI or MW is one that permits the other protein to be resolved from the ADPI on 2D electrophoresis, performed according to the Reference Protocol.

In one embodiment, an isolated protein is provided, that comprises a peptide with the amino acid sequence identified in Table IV for a ADPI, said protein having a pI and MW within 10% (particularly within 5%, more particularly within 1%) of the values identified in Table IV for that ADPI.

The ADPIs of the invention can be qualitatively or quantitatively detected by any method known to those skilled in the art, including but not limited to the Preferred Technology described herein, kinase assays, enzyme assays, binding assays and other functional assays, immunoassays, and western blotting. In one embodiment, the ADPIs are separated on a 2-D gel by virtue of their MWs. and pIs and are visualized by staining the gel. In one embodiment, the ADPIs are stained with a fluorescent dye and imaged with a fluorescence scanner. Sypro Red (Molecular Probes, Inc., Eugene, Oreg.) is a suitable dye for this purpose. Alternative dyes are described in U.S. Ser. No. 09/412,168, filed Oct. 5 1999, and incorporated herein by reference in its entirety.

Alternatively, ADPIs can be detected in an immunoassay. In one embodiment, an immunoassay is performed by contacting a sample with an anti-ADPI antibody under conditions such that immunospecific binding can occur if the ADPI is present, and detecting or measuring the amount of any immunospecific binding by the antibody. Anti-ADPI antibodies can be produced by the methods and techniques described herein; examples of such antibodies known in the art are set forth in Table VI. These antibodies shown in Table VI are already known to bind to the protein of which the ADPI is itself a family member. Particularly, the anti-ADPI antibody preferentially binds to the ADPI rather than to other isoforms of the same protein. In a particular embodiment, the anti-ADPI antibody binds to the ADPI with at least 2-fold greater affinity, more particularly at least 5-fold greater affinity, still more particularly at least 10-fold greater affinity, than to said other isoforms of the same protein. When the antibodies shown in Table VI do not display the required preferential selectivity for the target ADPI, one skilled in the art can generate additional antibodies by using the ADPI itself for the generation of such antibodies.

ADPIs can be transferred from a gel to a suitable membrane (e.g. a PVDF membrane) and subsequently probed in suitable assays that include, without limitation, competitive and non-competitive assay systems using techniques such as western blots and “sandwich” immunoassays using anti-ADPI antibodies as described herein, e.g., the antibodies identified in Table VI, or others raised against the ADPIs of interest as those skilled in the art will appreciate based on the present description. The immunoblots can be used to identify those anti-ADPI antibodies displaying the selectivity required to immuno-specifically differentiate a ADPI from other isoforms encoded by the same gene.

Table VI. Known Antibodies that Recognize ADPIs or ADPI-Related Polypeptides

TABLE VI

| ADPI# | Antibody | Manufacturer | Cat. No. |
|---|---|---|---|
| ADPI-62 | Gelsolin, plasma + | ACCURATE CHEMICAL & | YBG-4628-6210 |
| | cytoplasmic, Sheep | SCIENTIFIC CORPORATION | |
| | anti- | | |
| ADPI-68 | Gelsolin, plasma + | ACCURATE CHEMICAL & | YBG-4628-6210 |
| | cytoplasmic, Sheep | SCIENTIFIC CORPORATION | |
| | anti- | | |
| ADPI-72 | Gelsolin, plasma + | ACCURATE CHEMICAL & | YBG-4628-6210 |
| | cytoplasmic, Sheep | SCIENTIFIC CORPORATION | |
| | anti- | | |
| ADPI-78.2 | mouse anti-beta- | SANTA CRUZ | sc-5274 |
| | Tubulin (monoclonal) | BIOTECHNOLOGY, INC - | |
| | | RESEARCH ANTIBODIES | |
| | | 98/99 | |
| ADPI-108 | Lactic Dehydrogenase | ACCURATE CHEMICAL & | BYA-6019-1 |
| | (LDH) (H-subunit), | SCIENTIFIC CORPORATION | |
| | Clone: HH-17, Mab | | |
| | anti-Human | | |
| ADPI-140 | mouse anti-beta- | SANTA CRUZ | sc-5274 |
| | Tubulin (monoclonal) | BIOTECHNOLOGY, INC - | |
| | | RESEARCH ANTIBODIES | |
| | | 98/99 | |
| ADPI-141 | Monoclonal to | Abcam Ltd | ab66 |
| | Vimentin, intermediate | | |
| | filaments | | |
| ADPI-153.2 | Monoclonal to | Abcam Ltd | ab66 |
| | Vimentin, intermediate | | |
| | filaments | | |
| ADPI-175.2 | Glyceraldehyde-3- | BIODESIGN | H86504M |
| | Phosphate | INTERNATIONAL | |
| | Dehydrogenase | | |
| ADPI-188.1 | Mouse monoclonal to | Abcam Ltd | ab6536 |
| | heat shock protein 90 | | |
| | (HSP90) | | |
| ADPI-189.2 | Albumin, Human, | ACCURATE CHEMICAL & | IMS-01-026-02 |
| | Chicken anti- | SCIENTIFIC CORPORATION | |
| ADPI-193 | Albumin, Human, | ACCURATE CHEMICAL & | IMS-01-026-02 |
| | Chicken anti- | SCIENTIFIC CORPORATION | |
| ADPI-196.2 | Fibrinogen, Fibrin I, B- | ACCURATE CHEMICAL & | NYB-18C6 |
| | beta chain (Bβ 1-42), | SCIENTIFIC CORPORATION | |
| | Clone: 18C6, Mab anti- | | |
| | Human | | |
| ADPI-217 | Hemoglobin, Goat anti- | ACCURATE CHEMICAL & | BMD-J16 |
| | Human | SCIENTIFIC CORPORATION | |
| ADPI-220 | Goat anti-human | Abcam Ltd. | ab6618 |
| | Carbonic Anhydrase I | | |
| | (polyclonal) | | |
| ADPI-228 | Mouse Monoclonal | Lab Vision Corporation | MS-727-P1ABX |
| | Anti Moesin antibody | | |
| ADPI-230.1 | mouse anti-beta- | SANTA CRUZ | sc-5274 |
| | Tubulin (monoclonal) | BIOTECHNOLOGY, INC - | |
| | | RESEARCH ANTIBODIES | |
| | | 98/99 | |
| ADPI-286 | Glyceraldehyde-3- | BIODESIGN | H86504M |
| | Phosphate | INTERNATIONAL | |
| | Dehydrogenase | | |
| ADPI-289 | Glyceraldehyde-3- | BIODESIGN | H86504M |
| | Phosphate | INTERNATIONAL | |
| | Dehydrogenase | | |
| ADPI-291 | Glyceraldehyde-3- | BIODESIGN | H86504M |
| | Phosphate | INTERNATIONAL | |
| | Dehydrogenase | | |
| ADPI-297 | mouse anti-beta- | SANTA CRUZ | sc-5274 |
| | Tubulin (monoclonal) | BIOTECHNOLOGY, INC - | |
| | | RESEARCH ANTIBODIES | |
| | | 98/99 | |
| ADPI-311 | Cathepsin D (CTD19) | Abcam Ltd. | ab7433 |
| ADPI-323 | Apolipoprotein A1 | ACCURATE CHEMICAL & | ACL-20075AP |
| | (HDL), Sheep anti- | SCIENTIFIC CORPORATION | |
| | Human | | |
| ADPI-351 | Anti-Profilin affinity | immunoGlobe | IG706 |
| | purified rabbit antibody | | |
| ADPI-353 | Hemoglobin, Goat anti- | ACCURATE CHEMICAL & | BMD-J16 |
| | Human | SCIENTIFIC CORPORATION | |
| ADPI-359 | Gelsolin, plasma + | ACCURATE CHEMICAL & | YBG-4628-6210 |
| | cytoplasmic, Sheep | SCIENTIFIC CORPORATION | |
| | anti- | | |
| ADPI-371 | Albumin, Human, | ACCURATE CHEMICAL & | IMS-01-026-02 |
| | Chicken anti- | SCIENTIFIC CORPORATION | |
| ADPI-372 | Albumin, Human, | ACCURATE CHEMICAL & | IMS-01-026-02 |
| | Chicken anti- | SCIENTIFIC CORPORATION | |
| ADPI-387 | Anti-Profilin affinity | immunoGlobe | IG706 |
| | purified rabbit antibody | | |
| ADPI-390 | HSP 90 (H-114) | SANTA CRUZ | sc-7947 |
| | | BIOTECHNOLOGY, INC - | |
| | | RESEARCH ANTIBODIES | |
| | | 98/99 | |
| ADPI-399.2 | Anti-Aldehyde | Abcam Ltd | ab6192 |
| | Dehydrogenase, rabbit | | |
| | polyclonal | | |
| ADPI-419 | Monoclonal anti- | BIODESIGN | M37403M |
| | Neuron Specific | INTERNATIONAL | |
| | Enolase | | |
| ADPI-424 | mouse anti-beta- | SANTA CRUZ | sc-5274 |
| | Tubulin (monoclonal) | BIOTECHNOLOGY, INC - | |
| | | RESEARCH ANTIBODIES | |
| | | 98/99 | |
| ADPI-429 | Monoclonal anti- | BIODESIGN | M37403M |
| | Neuron Specific | INTERNATIONAL | |
| | Enolase | | |
| ADPI-471 | Monoclonal Anti- | BIODESIGN | M42107M |
| | Cytokeratin | INTERNATIONAL | |
| ADPI-506 | Fibrinogen, Fibrin I, B- | ACCURATE CHEMICAL & | NYB-18C6 |
| | beta chain (Bβ 1-42), | SCIENTIFIC CORPORATION | |
| | Clone: 18C6, Mab anti- | | |
| | Human | | |
| ADPI-511 | Rabbit anti-Annexin II | BIODESIGN | K80100R |
| | monomer | INTERNATIONAL | |
| ADPI-528 | Mouse monoclonal to | Abcam Ltd | ab6536 |
| | heat shock protein 90 | | |
| | (HSP90) | | |
| ADPI-547.3 | Monoclonal Anti- | BIODESIGN | M42107M |
| | Cytokeratin | INTERNATIONAL | |
| ADPI-547.1 | mouse anti-beta- | SANTA CRUZ | sc-5274 |
| | Tubulin (monoclonal) | BIOTECHNOLOGY, INC - | |
| | | RESEARCH ANTIBODIES | |
| | | 98/99 | |

**Further information about these antibodies can be obtained from their commercial sources at: ACCURATE CHEMICAL & SCIENTIFIC CORPORATION - http://www.accuratechemical.com/; BIODESIGN INTERNATIONAL - http://www.biodesign.com/; DAKO CORPORATION - http://www.dakousa.com; RDI RESEARCH DIAGNOSTICS, INC - http://www.researchd.com/; SANTA CRUZ BIOTECHNOLOGY, INC - http://www.scbt.com/. ImmunoGlobe - www.immunoGlobe.com; Abcam Ltd - www.abcam.co.uk

In one embodiment, binding of antibody in tissue sections can be used to detect ADPI localization or the level of one or more ADPIs. In a specific embodiment, antibody to a ADPI can be used to assay a tissue sample (e.g., a brain biopsy) from a subject for the level of the ADPI where a substantially changed level of ADPI is indicative of Alzheimer's disease. As used herein, a “substantially changed level” means a level that is increased or decreased compared with the level in a subject free from Alzheimer's disease or a reference level. If desired, the comparison can be performed with a matched sample from the same subject, taken from a portion of the body not affected by Alzheimer's disease.

Any suitable immunoassay can be used to detect a ADPI, including, without limitation, competitive and non-competitive assay systems using techniques such as western blots, radioimmunoassays, ELISAs (enzyme linked immunosorbent assays), “sandwich” immunoassays, immunoprecipitation assays, precipitin reactions, gel diffusion precipitin reactions, immunodiffusion assays, agglutination assays, complement-fixation assays, immunoradiometric assays, fluorescent immunoassays and protein A immunoassays.

For example, an ADPI can be detected in a fluid sample (e.g., brain tissue, blood, urine, or tissue homogenate) by means of a two-step sandwich assay. In the first step, a capture reagent (e.g., an anti-ADPI antibody) is used to capture the ADPI. Examples of such antibodies known in the art are set forth in Table VII. The capture reagent can optionally be immobilized on a solid phase. In the second step, a directly or indirectly labelled detection reagent is used to detect the captured ADPI. In one embodiment, the detection reagent is a lectin. A lectin can be used for this purpose that preferentially binds to the ADPI rather than to other isoforms that have the same core protein as the ADPI or to other proteins that share the antigenic determinant recognized by the antibody. In a preferred embodiment, the chosen lectin binds to the ADPI with at least 2-fold greater affinity, more preferably at least 5-fold greater affinity, still more preferably at least 10-fold greater affinity, than to said other isoforms that have the same core protein as the ADPI or to said other proteins that share the antigenic determinant recognized by the antibody. Based on the present description, a lectin that is suitable for detecting a given ADPI can readily be identified by those skilled in the art using methods well known in the art, for instance upon testing one or more lectins enumerated in Table I on pages 158-159 of Sumar et al., Lectins as Indicators of Disease-Associated Glycoforms, In: Gabius H-J & Gabius S (eds.), 1993, Lectins and Glycobiology, at pp. 158-174 (which is incorporated herein by reference in its entirety). Lectins with the desired oligosaccharide specificity can be identified, for example, by their ability to detect the ADPI in a 2D gel, in a replica of a 2D gel following transfer to a suitable solid substrate such as a nitrocellulose membrane, or in a two-step assay following capture by an antibody. In an alternative embodiment, the detection reagent is an antibody, e.g., an antibody that immunospecifically detects other post-translational modifications, such as an antibody that immunospecifically binds to phosphorylated amino acids. Examples of such antibodies include those that bind to phosphotyrosine (BD Transduction Laboratories, catalog nos.: P11230-050/P11230-150; P11120; P38820; P39020), those that bind to phosphoserine (Zymed Laboratories Inc., South San Francisco, Calif., catalog no. 61-8100) and those that bind to phosphothreonine (Zymed Laboratories Inc., South San Francisco, Calif., catalog nos. 71-8200, 13-9200).

If desired, a gene encoding an ADPI, a related gene (e.g. a gene having sequence homology), or related nucleic acid sequences or subsequences, including complementary sequences, can also be used in hybridization assays. A nucleotide encoding an ADPI, or subsequences thereof comprising at least 8 nucleotides, preferably at least 12 nucleotides, and most preferably at least 15 nucleotides can be used as a hybridization probe. Hybridization assays can be used for detection, treatment, diagnosis, or monitoring of conditions, disorders, or disease states, associated with aberrant expression of genes encoding ADPIs, or for differential diagnosis of subjects with signs or symptoms suggestive of Alzheimer's disease. In particular, such a hybridization assay can be carried out by a method comprising contacting a subject's sample containing nucleic acid with a nucleic acid probe capable of hybridizing to a DNA or RNA that encodes a ADPI, under conditions such that hybridization can occur, and detecting or measuring any resulting hybridization. Nucleotides can be used for therapy of subjects having Alzheimer's disease, as described below.

The invention also provides diagnostic kits, comprising an anti-ADPI antibody. In addition, such a kit may optionally comprise one or more of the following: (1) instructions for using the anti-ADPI antibody for diagnosis, prognosis, therapeutic monitoring or any suitable combination of these applications; (2) a labelled binding partner to the antibody; (3) a solid phase (such as a reagent strip) upon which the anti-ADPI antibody is immobilized; and (4) a label or insert indicating regulatory approval for diagnostic, prognostic or therapeutic use or any suitable combination thereof. If no labelled binding partner to the antibody is provided, the anti-ADPI antibody itself can be labelled with a detectable marker, e.g., a chemiluminescent, enzymatic, fluorescent, or radioactive moiety.

The invention also provides a kit comprising a nucleic acid probe capable of hybridizing to RNA encoding a ADPI. In a specific embodiment, a kit comprises in one or more containers a pair of primers (e.g., each in the size range of 6-30 nucleotides, more preferably 10-30 nucleotides and still more preferably 10-20 nucleotides) that under appropriate reaction conditions can prime amplification of at least a portion of a nucleic acid encoding a ADPI, such as by polymerase chain reaction (see, e.g., Innis et al., 1990, PCR Protocols, Academic Press, Inc., San Diego, Calif.), ligase chain reaction (see EP 320,308) use of Qβ replicase, cyclic probe reaction, or other methods known in the art.

Kits are also provided which allow for the detection of a plurality of ADPIs or a plurality of nucleic acids each encoding a ADPI. A kit can optionally further comprise a predetermined amount of an isolated ADPI protein or a nucleic acid encoding a ADPI, e.g., for use as a standard or control.

5.4 Statistical Techniques for Identifying ADFs and ADF Clusters

Uni-variate differential analysis tools, such as fold changes, Wilcoxon rank sum test and t-test, are useful in identifying individual ADFs or ADPIs that are diagnostically associated with Alzheimer's disease or in identifying individual ADPIs that regulate the disease process. However, those skilled in the art will appreciate that the disease process is associated with a suitable combination of ADFs or ADPIs (and to be regulated by a suitable combination of ADPIs), rather than individual ADFs and ADPIs in isolation. The strategies for discovering such suitable combinations of ADFs and ADPIs differ from those for discovering individual ADFs and ADPIs. In such cases, each individual ADF and ADPI can be regarded as one variable and the disease can be regarded as a joint, multi-variate effect caused by interaction of these variables.

The following steps can be used to identify markers from data produced by the Preferred Technology.

The first step is to identify a collection of ADFs or ADPIs that individually show significant association with Alzheimer's disease. The association between the identified individual ADFs or individual ADPIs and Alzheimer's disease need not be as highly significant when a collection of ADFs and ADPIs is used as a diagnostic as is desirable when an individual ADF or ADPI is used as a diagnostic. Any of the tests discussed above (fold changes, Wilcoxon rank sum test, etc.) can be used at this stage. Once a suitable collection of ADFs or ADPIs has been identified, a sophisticated multi-variate analysis capable of identifying clusters can then be used to estimate the significant multivariate associations with Alzheimer's disease.

Linear Discriminant Analysis (LDA) is one such procedure, which can be used to detect significant association between a cluster of variables (i.e., ADFs or ADPIs) and Alzheimer's disease. In performing LDA, a set of weights is associated with each variable (i.e., ADF or ADPI) so that the linear combination of weights and the measured values of the variables can identify the disease state by discriminating between subjects having Alzheimer's disease and subjects free from Alzheimer's disease. Enhancements to the LDA allow stepwise inclusion (or removal) of variables to optimize the discriminant power of the model. The result of the LDA is therefore a cluster of ADFs or ADPIs which can be used for diagnosis, treatment or development of pharmaceutical products. Other enhanced variations of LDA, such as Flexible Discriminant Analysis permit the use of non-linear combinations of variables to discriminate a disease state from a state in which there is no disease. The results of the discriminant analysis can be verified by post-hoc tests and also by repeating the analysis using alternative techniques such as classification trees.

A further category of ADFs or ADPIs can be identified by qualitative measures by comparing the percentage feature presence of an ADF or ADPI of one group of samples (e.g., samples from diseased subjects) with the percentage feature presence of an ADF or ADPI in another group of samples (e.g., samples from control subjects). The “percentage feature presence” of an ADF or ADPI is the percentage of samples in a group of samples in which the ADF or ADPI is detectable by the detection method of choice. For example, if an ADF is detectable in 95 percent of samples from diseased subjects, the percentage feature presence of that ADF in that sample group is 95 percent. If only 5 percent of samples from non-diseased subjects have detectable levels of the same ADF, detection of that ADF in the sample of a subject would suggest that it is likely that the subject has Alzheimer's disease.

5.5 Use in Clinical Studies

The diagnostic methods and compositions of the present invention can assist in monitoring a clinical study, e.g. to evaluate therapies for Alzheimer's disease. In one embodiment, candidate molecules are tested for their ability to restore ADF or ADPI levels in a subject having Alzheimer's disease to levels found in subjects free from Alzheimer's disease or, in a treated subject (e.g. after treatment with a cholinesterase inhibitor such as tacrine), to preserve ADF or ADPI levels at or near non-Alzheimer's disease values. The levels of one or more ADFs or ADPIs can be assayed.

In another embodiment, the methods and compositions of the present invention are used to screen individuals for entry into a clinical study to identify individuals having Alzheimer's disease; individuals already having Alzheimer's disease can then be excluded from the study or can be placed in a separate cohort for treatment or analysis. If desired, the candidates can concurrently be screened to identify individuals with Lewy Body disease and/or vascular dementia; procedures for these screens are well known in the art (Harding and Halliday, 1998, Neuropathol. Appl. Neurobiol. 24:195-201).

5.6 Purification of ADPIs

In particular aspects, the invention provides isolated mammalian ADPIs, preferably human ADPIs, and fragments thereof which comprise an antigenic determinant (i.e., can be recognized by an antibody) or which are otherwise functionally active, as well as nucleic acid sequences encoding the foregoing. “Functionally active” as used herein refers to material displaying one or more functional activities associated with a full-length (wild-type) ADPI, e.g., binding to a ADPI substrate or ADPI binding partner, antigenicity (binding to an anti-ADPI antibody), immunogenicity, enzymatic activity and the like.

In specific embodiments, the invention provides fragments of an ADPI comprising at least 5 amino acids, at least 10 amino acids, at least 50 amino acids, or at least 75 amino acids. Fragments lacking some or all of the regions of an ADPI are also provided, as are proteins (e.g., fusion proteins) comprising such fragments. Nucleic acids encoding the foregoing are provided.

Once a recombinant nucleic acid which encodes the ADPI, a portion of the ADPI, or a precursor of the ADPI is identified, the gene product can be analysed. This can be achieved by assays based on the physical or functional properties of the given product, including, for example, radioactive labelling of the product followed by analysis by gel electrophoresis, immunoassay, etc.

The ADPIs identified herein can be isolated and purified by standard methods including chromatography (e.g., ion exchange, affinity, and sizing column chromatography), centrifugation, differential solubility, or by any other standard technique for the purification of proteins.

Alternatively, once a recombinant nucleic acid that encodes the ADPI is identified, the entire amino acid sequence of the ADPI can be deduced from the nucleotide sequence of the gene coding region contained in the recombinant nucleic acid. As a result, the protein can be synthesized by standard chemical methods known in the art (e.g., see Hunkapiller et al., 1984, Nature 310:105-111).

In another alternative embodiment, native ADPIs can be purified from natural sources, by standard methods such as those described above (e.g., immunoaffinity purification).

In a preferred embodiment, ADPIs are isolated by the Preferred Technology described supra. For preparative-scale runs, a narrow-range “zoom gel” having a pH range of 2 pH units or less is preferred for the isoelectric step, according to the method described in Westermeier, 1993, Electrophoresis in Practice (VCH, Weinheim, Germany), pp. 197-209 (which is incorporated herein by reference in its entirety); this modification permits a larger quantity of a target protein to be loaded onto the gel, and thereby increases the quantity of isolated ADPI that can be recovered from the gel. When used in this way for preparative-scale runs, the Preferred Technology typically provides up to 100 ng, and can provide up to 1000 ng, of an isolated ADPI in a single run. Those of skill in the art will appreciate that a zoom gel can be used in any separation strategy which employs gel isoelectric focusing.

The invention thus provides an isolated ADPI, an isolated ADPI-related polypeptide, and an isolated derivative or fragment of a ADPI or a ADPI-related polypeptide; any of the foregoing can be produced by recombinant DNA techniques or by chemical synthetic methods.

5.7 Isolation of DNA Encoding a ADPI

Particular embodiments for the cloning of a gene encoding a ADPI, are presented below by way of example and not of limitation.

The nucleotide sequences of the present invention, including DNA and RNA, and comprising a sequence encoding a ADPI or a fragment thereof, or a ADPI-related polypeptide, may be synthesized using methods known in the art, such as using conventional chemical approaches or polymerase chain reaction (PCR) amplification. The nucleotide sequences of the present invention also permit the identification and cloning of the gene encoding a ADPI homolog or ADPI ortholog including, for example, by screening cDNA libraries, genomic libraries or expression libraries.

For example, to clone a gene encoding a ADPI by PCR techniques, anchored degenerate oligonucleotides (or a set of most likely oligonucleotides) can be designed for all ADPI peptide fragments identified as part of the same protein. PCR reactions under a variety of conditions can be performed with relevant cDNA and genomic DNAs (e.g., from a brain biopsy or from cells of the immune system) from one or more species. Also vectorette reactions can be performed on any available cDNA and genomic DNA using the oligonucleotides (which preferably are nested) as above. Vectorette PCR is a method that enables the amplification of specific DNA fragments in situations where the sequence of only one primer is known. Thus, it extends the application of PCR to stretches of DNA where the sequence information is only available at one end. (Arnold C, 1991, PCR Methods Appl. 1(1):39-42; Dyer K D, Biotechniques, 1995, 19(4):550-2). Vectorette PCR may be performed with probes that are, for example, anchored degenerate oligonucleotides (or most likely oligonucleotides) coding for ADPI peptide fragments, using as a template a genomic library or cDNA library pools.

Anchored degenerate oligonucleotides (and most likely oligonucleotides) can be designed for all ADPI peptide fragments. These oligonucleotides may be labelled and hybridised to filters containing cDNA and genomic DNA libraries. Oligonucleotides to different peptides from the same protein will often identify the same members of the library. The cDNA and genomic DNA libraries may be obtained from any suitable or desired mammalian species, for example from humans.

Nucleotide sequences comprising a nucleotide sequence encoding a ADPI or ADPI fragment of the present invention are useful, for example, for their ability to hybridise selectively with complementary stretches of genes encoding other proteins. Depending on the application, a variety of hybridisation conditions may be employed to obtain nucleotide sequences at least about 30%, 35%, 40%, 45%, 50%, 55%, 60%, 65%, 70%, 75%, 80%, 85%, 90%, 95% or 99% identical, or 100% identical, to the sequence of a nucleotide encoding a ADPI.

For a high degree of selectivity, relatively stringent conditions are used to form the duplexes, such as low salt or high temperature conditions. As used herein, “highly stringent conditions” means hybridisation to filter-bound DNA in 0.5 M NaHPO4, 7% sodium dodecyl sulfate (SDS), 1 mM EDTA at 65° C., and washing in 0.1×SSC/0.1% SDS at 68° C. (Ausubel F. M. et al., eds., 1989, Current Protocols in Molecular Biology, Vol. I, Green Publishing Associates, Inc., and John Wiley & Sons, Inc., New York, at p. 2.10.3; incorporated herein by reference in its entirety.) For some applications, less stringent conditions for duplex formation are required. As used herein “moderately stringent conditions” means washing in 0.2×SSC/0.1% SDS at 42° C. (Ausubel et al., 1989, supra). Hybridization conditions can also be rendered more stringent by the addition of increasing amounts of formamide, to destabilize the hybrid duplex. Thus, particular hybridisation conditions can be readily manipulated, and will generally be chosen depending on the desired results. In general, convenient hybridisation temperatures in the presence of 50% formamide are: 42° C. for a probe which is 95 to 100% identical to the fragment of a gene encoding a ADPI, 37° C. for 90 to 95% identity and 32° C. for 70 to 90% identity.

In the preparation of genomic libraries, DNA fragments are generated, some of which will encode parts or the whole of an ADPI. Any suitable method for preparing DNA fragments may be used in the present invention. For example, the DNA may be cleaved at specific sites using various restriction enzymes. Alternatively, one may use DNAse in the presence of manganese to fragment the DNA, or the DNA can be physically sheared, as for example, by sonication. The DNA fragments can then be separated according to size by standard techniques, including but not limited to agarose and polyacrylamide gel electrophoresis, column chromatography and sucrose gradient centrifugation. The DNA fragments can then be inserted into suitable vectors, including but not limited to plasmids, cosmids, bacteriophages lambda or T4, and yeast artificial chromosome (YAC). (See, e.g., Sambrook et al., 1989, Molecular Cloning, A Laboratory Manual, 2d Ed., Cold Spring Harbor Laboratory Press, Cold Spring Harbor, New York; Glover, D. M. (ed.), 1985, DNA Cloning: A Practical Approach, MRL Press, Ltd., Oxford, U. K. Vol. I, II; Ausubel F. M. et al., eds., 1989, Current Protocols in Molecular Biology, Vol. I, Green Publishing Associates, Inc., and John Wiley & sons, Inc., New York). The genomic library may be screened by nucleic acid hybridization to labeled probe (Benton and Davis, 1977, Science 196:180; Grunstein and Hogness, 1975, Proc. Natl. Acad. Sci. U.S.A. 72:3961).

Based on the present description, the genomic libraries may be screened with labelled degenerate oligonucleotide probes corresponding to the amino acid sequence of any peptide of the ADPI using optimal approaches well known in the art. Any probe used is at least 10 nucleotides, at least 15 nucleotides, at least 20 nucleotides, at least 25 nucleotides, at least 30 nucleotides, at least 40 nucleotides, at least 50 nucleotides, at least 60 nucleotides, at least 70 nucleotides, at least 80 nucleotides, or at least 100 nucleotides. Preferably a probe is 10 nucleotides or longer, and more preferably 15 nucleotides or longer.

In Table IV above, some ADPIs disclosed herein correspond to variants of previously identified proteins encoded by genes whose sequences are publicly known. To screen such a gene, any probe may be used that is complementary to the gene or its complement; preferably the probe is 10 nucleotides or longer, more preferably 15 nucleotides or longer. The SWISS-PROT and trEMBL databases (held by the Swiss Institute of Bioinformatics (SIB) and the European Bioinformatics Institute (EBI) which are available at http://www.expasy.ch/) and the GenBank database (held by the National Institute of Health (NIH) which is available at http://www.ncbi.nlm.nih.gov/) provide protein sequences comprising the amino acid sequences listed for the ADPIs in Tables IV and V under the following accession numbers and each sequence is incorporated herein by reference:

Table VII. Nucleotide Sequences Encoding ADPIs, ADPI-Related Proteins or ERPIs

TABLE VII

| ADF# | ADPI# | ACCESSION |
|---|---|---|
| ADF-1 | ADPI-1 | P14136 |
| ADF-3 | ADPI-3.1 | AF141349.1 |
| ADF-3 | ADPI-3.2 | P07197 |
| ADF-3 | ADPI-3.3 | P21281 |
| ADF-5 | ADPI-5.3 | 8922699 |
| ADF-6 | ADPI-6 | P14136 |
| ADF-8 | ADPI-8 | P14136 |
| ADF-9 | ADPI-9 | P31150 |
| ADF-10 | ADPI-10 | P14136 |
| ADF-11 | ADPI-11 | P14136 |
| ADF-12 | ADPI-12.1 | P14136 |
| ADF-12 | ADPI-12.2 | U47634.1 |
| ADF-13 | ADPI-13 | P11142 |
| ADF-15 | ADPI-15 | Q12765 |
| ADF-16 | ADPI-16 | P14136 |
| ADF-22 | ADPI-22 | P55072 |
| ADF-23 | ADPI-23 | P29777 |
| ADF-24 | ADPI-24 | Q9Y2J8 |
| ADF-25 | ADPI-25.3 | P04687 |
| ADF-25 | ADPI-25.2 | P07196 |
| ADF-26 | ADPI-26 | P14136 |
| ADF-27 | ADPI-27 | P14136 |
| ADF-29 | ADPI-29 | P14136 |
| ADF-31 | ADPI-31 | P14136 |
| ADF-33 | ADPI-33.1 | P00558 |
| ADF-35 | ADPI-35 | P12532 |
| ADF-37 | ADPI-37 | P36543 |
| ADF-39 | ADPI-39 | P17174 |
| ADF-41 | ADPI-41 | NOVEL |
| ADF-42 | ADPI-42 | 2117873 |
| ADF-54 | ADPI-54 | Q99798 |
| ADF-56 | ADPI-56 | Q99798 |
| ADF-61 | ADPI-61 | P29401 |
| ADF-62 | ADPI-62 | P06396 |
| ADF-66 | ADPI-66 | Q05193 |
| ADF-67 | ADPI-67 | P00367 |
| ADF-68 | ADPI-68 | P06396 |
| ADF-70 | ADPI-70 | Q05193 |
| ADF-72 | ADPI-72 | P06396 |
| ADF-77 | ADPI-77 | P14136 |
| ADF-78 | ADPI-78.2 | P04350 |
| ADF-78 | ADPI-78.3 | P14136 |
| ADF-79 | ADPI-79 | D86957.1 |
| ADF-81 | ADPI-81.2 | P14136 |
| ADF-82 | ADPI-82 | P04075 |
| ADF-85 | ADPI-85 | X04098.1 |
| ADF-87 | ADPI-87.1 | P11016 |
| ADF-88 | ADPI-88 | P21266 |
| ADF-90 | ADPI-90 | P14136 |
| ADF-91 | ADPI-91.1 | O00154 |
| ADF-91 | ADPI-91.2 | P09972 |
| ADF-92 | ADPI-92 | P00338 |
| ADF-94 | ADPI-94 | 7328935 |
| ADF-95 | ADPI-95 | P28482 |
| ADF-97 | ADPI-97 | P02794 |
| ADF-98 | ADPI-98 | P23528 |
| ADF-101 | ADPI-101 | P23528 |
| ADF-102 | ADPI-102.1 | P29777 |
| ADF-103 | ADPI-103 | Q06830 |
| ADF-105 | ADPI-105.2 | P12953 |
| ADF-105 | ADPI-105.1 | P14136 |
| ADF-107 | ADPI-107 | P02511 |
| ADF-108 | ADPI-108 | P07195 |
| ADF-109 | ADPI-109 | P80404 |
| ADF-111 | ADPI-111.1 | P14136 |
| ADF-112 | ADPI-112 | P46459 |
| ADF-113 | ADPI-113 | P25705 |
| ADF-115 | ADPI-115 | P46459 |
| ADF-117 | ADPI-117.1 | 1732416 |
| ADF-119 | ADPI-119 | P14136 |
| ADF-120 | ADPI-120 | P14136 |
| ADF-121 | ADPI-121 | Q16555 |
| ADF-124 | ADPI-124 | 1389721 |
| ADF-125 | ADPI-125 | P14136 |
| ADF-126 | ADPI-126 | AF141349.1 |
| ADF-126 | ADPI-126 | P12277 |
| ADF-126 | ADPI-126 | P14136 |
| ADF-127 | ADPI-127 | P29777 |
| ADF-128 | ADPI-128 | P78417 |
| ADF-129 | ADPI-129 | P46821 |
| ADF-130 | ADPI-130 | P14136 |
| ADF-131 | ADPI-131 | P07437 |
| ADF-132 | ADPI-132 | P14136 |
| ADF-133 | ADPI-133 | P08559 |
| ADF-138 | ADPI-138 | P21796 |
| ADF-139 | ADPI-139 | P09543 |
| ADF-140 | ADPI-140 | P04350 |
| ADF-141 | ADPI-141 | P08670 |
| ADF-142 | ADPI-142.2 | P09972 |
| ADF-142 | ADPI-142.1 | P16219 |
| ADF-143 | ADPI-143.1 | P16152 |
| ADF-143 | ADPI-143.2 | P21796 |
| ADF-144 | ADPI-144 | P14136 |
| ADF-146 | ADPI-146.2 | O15540 |
| ADF-146 | ADPI-146.1 | U81235.1 |
| ADF-148 | ADPI-148 | P14136 |
| ADF-149 | ADPI-149.1 | P14136 |
| ADF-150 | ADPI-150 | P14136 |
| ADF-151 | ADPI-151.2 | P14136 |
| ADF-151 | ADPI-151.1 | U47634.1 |
| ADF-152 | ADPI-152 | P14136 |
| ADF-153 | ADPI-153.2 | P08670 |
| ADF-153 | ADPI-153.1 | P06576 |
| ADF-153 | ADPI-153.3 | P31150 |
| ADF-154 | ADPI-154 | P14136 |
| ADF-155 | ADPI-155 | M11717.1 |
| ADF-156 | ADPI-156.2 | P07196 |
| ADF-156 | ADPI-156.1 | P14136 |
| ADF-157 | ADPI-157 | P12277 |
| ADF-159 | ADPI-159.1 | P02571 |
| ADF-159 | ADPI-159.2 | P14136 |
| ADF-160 | ADPI-160 | 1732416 |
| ADF-162 | ADPI-162.2 | P07196 |
| ADF-162 | ADPI-162.3 | P14136 |
| ADF-162 | ADPI-162.1 | X04098.1 |
| ADF-163 | ADPI-163 | P14136 |
| ADF-165 | ADPI-165 | P30041 |
| ADF-172 | ADPI-172 | P01922 |
| ADF-173 | ADPI-173.1 | 3041875 |
| ADF-173 | ADPI-173.2 | 3041875 |
| ADF-175 | ADPI-175.2 | P04406 |
| ADF-175 | ADPI-175.1 | P40926 |
| ADF-176 | ADPI-176 | 3041875 |
| ADF-182 | ADPI-182 | P21796 |
| ADF-183 | ADPI-183 | P09936 |
| ADF-188 | ADPI-188.1 | P07900 |
| ADF-189 | ADPI-189.2 | P02768 |
| ADF-191 | ADPI-191 | Q16658 |
| ADF-193 | ADPI-193 | P02768 |
| ADF-194 | ADPI-194.2 | P00367 |
| ADF-196 | ADPI-196.2 | P02675 |
| ADF-196 | ADPI-196.3 | P02675 |
| ADF-196 | ADPI-196.1 | P49419 |
| ADF-196 | ADPI-196.4 | P50995 |
| ADF-202 | ADPI-202 | 3041875 |
| ADF-204 | ADPI-204 | Q01469 |
| ADF-208 | ADPI-208 | P02794 |
| ADF-209 | ADPI-209 | P09382 |
| ADF-216 | ADPI-216 | P30043 |
| ADF-217 | ADPI-217 | P02023 |
| ADF-220 | ADPI-220 | P00915 |
| ADF-223 | ADPI-223 | P08559 |
| ADF-228 | ADPI-228 | P26038 |
| ADF-229 | ADPI-229 | P80404 |
| ADF-230 | ADPI-230.1 | P04350 |
| ADF-230 | ADPI-230.2 | P14136 |
| ADF-232 | ADPI-232.1 | P05217 |
| ADF-232 | ADPI-232.2 | Q14894 |
| ADF-236 | ADPI-236.2 | 4321795 |
| ADF-236 | ADPI-236.3 | Q13938 |
| ADF-237 | ADPI-237.2 | 7441369 |
| ADF-237 | ADPI-237.1 | P13795 |
| ADF-240 | ADPI-240 | P36542 |
| ADF-243 | ADPI-243 | Q99962 |
| ADF-245 | ADPI-245 | P30040 |
| ADF-250 | ADPI-250 | 1805280 |
| ADF-251 | ADPI-251 | 1805280 |
| ADF-252 | ADPI-252.1 | P22314 |
| ADF-252 | ADPI-252.2 | P49588 |
| ADF-253 | ADPI-253 | P19367 |
| ADF-254 | ADPI-254 | P19367 |
| ADF-255 | ADPI-255 | P19367 |
| ADF-256 | ADPI-256 | P19367 |
| ADF-257 | ADPI-257.1 | P07197 |
| ADF-257 | ADPI-257.2 | P21281 |
| ADF-258 | ADPI-258 | P48735 |
| ADF-259 | ADPI-259.1 | P11310 |
| ADF-259 | ADPI-259.2 | Q15019 |
| ADF-260 | ADPI-260 | P09543 |
| ADF-261 | ADPI-261 | Q99962 |
| ADF-262 | ADPI-262 | P14136 |
| ADF-263 | ADPI-263 | 5410300 |
| ADF-264 | ADPI-264 | P50148 |
| ADF-265 | ADPI-265 | P09972 |
| ADF-266 | ADPI-266 | Q9Y617 |
| ADF-267 | ADPI-267 | Q99962 |
| ADF-268 | ADPI-268.1 | P12277 |
| ADF-268 | ADPI-268.2 | P50213 |
| ADF-269 | ADPI-269 | P00505 |
| ADF-270 | ADPI-270 | P14136 |
| ADF-271 | ADPI-271 | P14136 |
| ADF-272 | ADPI-272 | P04075 |
| ADF-273 | ADPI-273 | P04898 |
| ADF-274 | ADPI-274 | P29777 |
| ADF-275 | ADPI-275 | P14136 |
| ADF-276 | ADPI-276 | O95299 |
| ADF-277 | ADPI-277 | P14136 |
| ADF-278 | ADPI-278.1 | O00154 |
| ADF-278 | ADPI-278.2 | P09972 |
| ADF-279 | ADPI-279.1 | 5174445 |
| ADF-279 | ADPI-279.2 | 5174445 |
| ADF-280 | ADPI-280 | 5225320 |
| ADF-281 | ADPI-281 | O94760 |
| ADF-282 | ADPI-282 | 5225320 |
| ADF-283 | ADPI-283 | P07437 |
| ADF-284 | ADPI-284 | P21579 |
| ADF-285 | ADPI-285 | P08129 |
| ADF-286 | ADPI-286 | P04406 |
| ADF-287 | ADPI-287 | Q92561 |
| ADF-288 | ADPI-288 | 5225320 |
| ADF-289 | ADPI-289 | P04406 |
| ADF-290 | ADPI-290 | O43488 |
| ADF-291 | ADPI-291 | P04406 |
| ADF-292 | ADPI-292 | P40926 |
| ADF-293 | ADPI-293 | P40925 |
| ADF-294 | ADPI-294 | AF081484.1 |
| ADF-295 | ADPI-295 | 7441369 |
| ADF-296 | ADPI-296 | 6841176, |
| | | 8923114 |
| ADF-297 | ADPI-297 | P04350 |
| ADF-298 | ADPI-298 | 631483 |
| ADF-299 | ADPI-299 | P00338 |
| ADF-300 | ADPI-300 | 11094293 |
| ADF-301 | ADPI-301 | AK022287.1 |
| ADF-302 | ADPI-302 | P21796 |
| ADF-303 | ADPI-303 | P08758 |
| ADF-304 | ADPI-304 | 10800412 |
| ADF-305 | ADPI-305 | P42655 |
| ADF-306 | ADPI-306 | P12324 |
| ADF-307 | ADPI-307.1 | P07226 |
| ADF-307 | ADPI-307.2 | P42655 |
| ADF-308 | ADPI-308 | P36542 |
| ADF-309 | ADPI-309 | P10469 |
| ADF-311 | ADPI-311 | P07339 |
| ADF-312 | ADPI-312 | 6005842 |
| ADF-313 | ADPI-313 | P29218 |
| ADF-314 | ADPI-314 | O95865 |
| ADF-315 | ADPI-315 | 8922498 |
| ADF-316 | ADPI-316.1 | P04792 |
| ADF-316 | ADPI-316.2 | P30084 |
| ADF-317 | ADPI-317 | P13795 |
| ADF-318 | ADPI-318 | P21851 |
| ADF-319 | ADPI-319 | 5902018 |
| ADF-320 | ADPI-320.1 | P00492 |
| ADF-320 | ADPI-320.2 | P30041 |
| ADF-321 | ADPI-321 | P28161 |
| ADF-322 | ADPI-322 | P19404 |
| ADF-323 | ADPI-323 | P02647 |
| ADF-324 | ADPI-324 | P17080 |
| ADF-325 | ADPI-325 | P22061 |
| ADF-326 | ADPI-326 | P24539 |
| ADF-327 | ADPI-327 | 9622095 |
| ADF-328 | ADPI-328 | Q04760 |
| ADF-329 | ADPI-329 | Q04760 |
| ADF-330 | ADPI-330 | P00568 |
| ADF-331 | ADPI-331 | Q06830 |
| ADF-332 | ADPI-332 | P48047 |
| ADF-333 | ADPI-333.1 | P30086 |
| ADF-333 | ADPI-333.2 | Q06830 |
| ADF-334 | ADPI-334 | P02511 |
| ADF-335 | ADPI-335 | P42655 |
| ADF-336 | ADPI-336 | P34991 |
| ADF-337 | ADPI-337 | P02511 |
| ADF-338 | ADPI-338 | P00738 |
| ADF-339 | ADPI-339 | P19105 |
| ADF-340 | ADPI-340 | P02593 |
| ADF-341 | ADPI-341 | P37840 |
| ADF-342 | ADPI-342 | Q9Y281 |
| ADF-343 | ADPI-343 | P23528 |
| ADF-344 | ADPI-344 | 7441369 |
| ADF-345 | ADPI-345 | P05092 |
| ADF-346 | ADPI-346 | P06705 |
| ADF-347 | ADPI-347 | P30049 |
| ADF-348 | ADPI-348 | P30086 |
| ADF-349 | ADPI-349 | 7441369 |
| ADF-350 | ADPI-350 | P09455 |
| ADF-351 | ADPI-351 | P35080 |
| ADF-352 | ADPI-352 | P05413 |
| ADF-353 | ADPI-353 | P02023 |
| ADF-354 | ADPI-354 | Q16718 |
| ADF-355 | ADPI-355 | P01922 |
| ADF-356 | ADPI-356 | P19367 |
| ADF-357 | ADPI-357 | P21851 |
| ADF-358 | ADPI-358 | P11216 |
| ADF-359 | ADPI-359 | P06396 |
| ADF-360 | ADPI-360 | Q99798 |
| ADF-361 | ADPI-361 | 7328175 |
| ADF-362 | ADPI-362 | P46459 |
| ADF-363 | ADPI-363 | 7512974 |
| ADF-364 | ADPI-364 | P41250 |
| ADF-365 | ADPI-365.1 | 7512974 |
| ADF-365 | ADPI-365.2 | Q12931 |
| ADF-366 | ADPI-366 | Q16555 |
| ADF-367 | ADPI-367.1 | P11142 |
| ADF-367 | ADPI-367.2 | P38606 |
| ADF-368 | ADPI-368.1 | P08107 |
| ADF-368 | ADPI-368.2 | P08133 |
| ADF-369 | ADPI-369.1 | 3041875 |
| ADF-369 | ADPI-369.2 | 7662673 |
| ADF-370 | ADPI-370 | 4507007 |
| ADF-371 | ADPI-371 | P02768 |
| ADF-372 | ADPI-372 | P02768 |
| ADF-373 | ADPI-373 | 3041875 |
| ADF-374 | ADPI-374 | 7768938 |
| ADF-375 | ADPI-375 | Q16555 |
| ADF-376 | ADPI-376 | 3041875 |
| ADF-377 | ADPI-377 | P09543 |
| ADF-378 | ADPI-378 | 5225320 |
| ADF-379 | ADPI-379 | 6523827 |
| ADF-380 | ADPI-380 | P16152 |
| ADF-381 | ADPI-381 | P35232 |
| ADF-382 | ADPI-382 | P30712 |
| ADF-383 | ADPI-383 | P34991 |
| ADF-384 | ADPI-384 | O15509 |
| ADF-386 | ADPI-386 | Q16781 |
| ADF-387 | ADPI-387 | P35080 |
| ADF-388 | ADPI-388 | P53680 |
| ADF-389 | ADPI-389 | Q02218 |
| ADF-390 | ADPI-390 | P08238 |
| ADF-391 | ADPI-391 | Q01813 |
| ADF-392 | ADPI-392 | P08107 |
| ADF-393 | ADPI-393 | P11142 |
| ADF-394 | ADPI-394 | P54652 |
| ADF-395 | ADPI-395 | Q99832 |
| ADF-396 | ADPI-396 | P09622 |
| ADF-397 | ADPI-397 | 7239381 |
| ADF-398 | ADPI-398 | P40123 |
| ADF-399 | ADPI-399.2 | P00352 |
| ADF-399 | ADPI-399.1 | O43175 |
| ADF-400 | ADPI-400 | P06744 |
| ADF-401 | ADPI-401 | P78371 |
| ADF-402 | ADPI-402 | Q16555 |
| ADF-403 | ADPI-403 | P14618 |
| ADF-404 | ADPI-404.1 | 4335941 |
| ADF-404 | ADPI-404.2 | P49419 |
| ADF-405 | ADPI-405 | AF141349.1 |
| ADF-406 | ADPI-406 | Q14141 |
| ADF-407 | ADPI-407 | P05217 |
| ADF-408 | ADPI-408 | 8922699 |
| ADF-409 | ADPI-409 | 8922699 |
| ADF-410 | ADPI-410 | AF081484.1 |
| ADF-411 | ADPI-411 | P14136 |
| ADF-412 | ADPI-412.1 | P06576 |
| ADF-412 | ADPI-412.2 | P14136 |
| ADF-413 | ADPI-413 | P25705 |
| ADF-414 | ADPI-414 | P06576 |
| ADF-415 | ADPI-415 | P07197 |
| ADF-416 | ADPI-416 | 8922712 |
| ADF-417 | ADPI-417 | P06576 |
| ADF-418 | ADPI-418 | 1732416 |
| ADF-419 | ADPI-419 | P06733 |
| ADF-420 | ADPI-420 | 4104814 |
| ADF-421 | ADPI-421 | Q9Y281 |
| ADF-422 | ADPI-422 | P49588 |
| ADF-423 | ADPI-423 | Q16555 |
| ADF-424 | ADPI-424 | P04350 |
| ADF-425 | ADPI-425 | P25705 |
| ADF-426 | ADPI-426.1 | P25705 |
| ADF-426 | ADPI-426.2 | Q16851 |
| ADF-427 | ADPI-427 | P25705 |
| ADF-428 | ADPI-428 | P14136 |
| ADF-429 | ADPI-429 | P06733 |
| ADF-430 | ADPI-430 | 4090868 |
| ADF-431 | ADPI-431 | P43034 |
| ADF-432 | ADPI-432 | Q16181 |
| ADF-433 | ADPI-433 | Q16181 |
| ADF-434 | ADPI-434 | Q9Y2T3 |
| ADF-435 | ADPI-435 | P07954 |
| ADF-436 | ADPI-436 | Q02750 |
| ADF-437 | ADPI-437 | P14136 |
| ADF-438 | ADPI-438 | P23526 |
| ADF-439 | ADPI-439 | P49411 |
| ADF-440 | ADPI-440.1 | P02571 |
| ADF-440 | ADPI-440.2 | P14136 |
| ADF-441 | ADPI-441 | O75306 |
| ADF-442 | ADPI-442 | P14136 |
| ADF-443 | ADPI-443 | P14136 |
| ADF-444 | ADPI-444 | P15104 |
| ADF-445 | ADPI-445 | P12277 |
| ADF-446 | ADPI-446 | P15104 |
| ADF-447 | ADPI-447.1 | 1805280 |
| ADF-447 | ADPI-447.2 | P14136 |
| ADF-448 | ADPI-448 | P12532 |
| ADF-449 | ADPI-449 | P14136 |
| ADF-450 | ADPI-450.1 | P02571 |
| ADF-450 | ADPI-450.2 | P14136 |
| ADF-451 | ADPI-451 | P08559 |
| ADF-452 | ADPI-452 | P09543 |
| ADF-453 | ADPI-453 | O15142 |
| ADF-454 | ADPI-454 | P51570 |
| ADF-455 | ADPI-455 | AF141349.1 |
| ADF-456 | ADPI-456 | P04687 |
| ADF-457 | ADPI-457 | P16152 |
| ADF-458 | ADPI-458 | P04687 |
| ADF-459 | ADPI-459 | P45880 |
| ADF-460 | ADPI-460 | P02511 |
| ADF-461 | ADPI-461 | P55072 |
| ADF-462 | ADPI-462 | 3041875 |
| ADF-463 | ADPI-463 | 3041875 |
| ADF-464 | ADPI-464 | Q14195 |
| ADF-465 | ADPI-465 | Q14194 |
| ADF-466 | ADPI-466.1 | P23368 |
| ADF-466 | ADPI-466.2 | P36871 |
| ADF-467 | ADPI-467 | Q14194 |
| ADF-468 | ADPI-468 | Q14195 |
| ADF-469 | ADPI-469.1 | P48643 |
| ADF-469 | ADPI-469.2 | P50990 |
| ADF-470 | ADPI-470 | 6942004 |
| ADF-471 | ADPI-471 | P35527 |
| ADF-472 | ADPI-472 | P07197 |
| ADF-473 | ADPI-473 | 3041875 |
| ADF-474 | ADPI-474 | P14618 |
| ADF-475 | ADPI-475 | P31150 |
| ADF-476 | ADPI-476 | 3041875 |
| ADF-477 | ADPI-477 | P31146 |
| ADF-478 | ADPI-478.1 | Q16555 |
| ADF-478 | ADPI-478.2 | Q16555 |
| ADF-479 | ADPI-479 | Q92599 |
| ADF-480 | ADPI-480 | P04687 |
| ADF-481 | ADPI-481 | P55809 |
| ADF-482 | ADPI-482 | P78371 |
| ADF-483 | ADPI-483 | P25705 |
| ADF-484 | ADPI-484 | P25705 |
| ADF-485 | ADPI-485 | 8922712 |
| ADF-486 | ADPI-486 | P14136 |
| ADF-487 | ADPI-487 | P31930 |
| ADF-488 | ADPI-488 | 5915912 |
| ADF-489 | ADPI-489 | P14136 |
| ADF-490 | ADPI-490 | P50395 |
| ADF-491 | ADPI-491 | P14136 |
| ADF-492 | ADPI-492 | 9800545 |
| ADF-493 | ADPI-493.1 | P02571 |
| ADF-493 | ADPI-493.2 | P14136 |
| ADF-494 | ADPI-494 | P49411 |
| ADF-495 | ADPI-495 | P09543 |
| ADF-496 | ADPI-496 | P12532 |
| ADF-497 | ADPI-497 | 5725224 |
| ADF-498 | ADPI-498 | Q02547 |
| ADF-499 | ADPI-499 | P09936 |
| ADF-500 | ADPI-500 | Q16555 |
| ADF-501 | ADPI-501 | O95741 |
| ADF-502 | ADPI-502 | P17987 |
| ADF-503 | ADPI-503 | 8671168 |
| ADF-504 | ADPI-504 | 8886025 |
| ADF-505 | ADPI-505 | Q16352 |
| ADF-506 | ADPI-506 | P02675 |
| ADF-507 | ADPI-507 | P07197 |
| ADF-508 | ADPI-508 | P14136 |
| ADF-509 | ADPI-509 | P09543 |
| ADF-510 | ADPI-510 | P14136 |
| ADF-511 | ADPI-511 | P07355 |
| ADF-512 | ADPI-512 | P22061 |
| ADF-513 | ADPI-513 | P14618 |
| ADF-514 | ADPI-514 | O43175 |
| ADF-515 | ADPI-515 | P00367 |
| ADF-516 | ADPI-516 | O75390 |
| ADF-517 | ADPI-517 | P14136 |
| ADF-518 | ADPI-518 | Q16555 |
| ADF-519 | ADPI-519 | AF141349.1 |
| ADF-520 | ADPI-520 | P05217 |
| ADF-521 | ADPI-521 | P14136 |
| ADF-522 | ADPI-522 | P07197 |
| ADF-523 | ADPI-523 | P04720 |
| ADF-524 | ADPI-524 | P14136 |
| ADF-525 | ADPI-525 | P14136 |
| ADF-526 | ADPI-526.1 | P06468 |
| ADF-526 | ADPI-526.2 | P14136 |
| ADF-527 | ADPI-527 | P05092 |
| ADF-528 | ADPI-528 | P07900 |
| ADF-529 | ADPI-529 | 11094293 |
| ADF-530 | ADPI-530.1 | P07437 |
| ADF-530 | ADPI-530.2 | P14136 |
| ADF-531 | ADPI-531 | Q01813 |
| ADF-532 | ADPI-532 | P14618 |
| ADF-533 | ADPI-533 | 1732416 |
| ADF-534 | ADPI-534 | P09543 |
| ADF-535 | ADPI-535 | P14618 |
| ADF-536 | ADPI-536 | P13611 |
| ADF-537 | ADPI-537 | P14136 |
| ADF-538 | ADPI-538 | P07196 |
| ADF-539 | ADPI-539 | P14136 |
| ADF-540 | ADPI-540 | P29777 |
| ADF-541 | ADPI-541 | P24752 |
| ADF-542 | ADPI-542 | 7512834 |
| ADF-543 | ADPI-543 | P21796 |
| ADF-544 | ADPI-544 | O94760 |
| ADF-545 | ADPI-545 | P11142 |
| ADF-546 | ADPI-546 | Q02252 |
| ADF-547 | ADPI-547.3 | P35527 |
| ADF-547 | ADPI-547.1 | P04350 |
| ADF-547 | ADPI-547.2 | P14136 |
| ADF-548 | ADPI-548.1 | P21796 |
| ADF-548 | ADPI-548.2 | P47985 |
| ADF-549 | ADPI-549 | P11216 |

For ADPI-41, the partial sequence information derived from tandem mass spectrometry was not found to be described as a transcribed protein in any known public database. ADPI-41 is therefore listed as ‘NOVEL’ in Table VII. ADPI-41 has been cloned, and is further described below. For any ADPI, degenerate probes, or probes taken from the sequences described above by accession number may be used for screening. In the case of degenerate probes, they can be constructed from the partial amino sequence information obtained from tandem mass spectra of tryptic digest peptides of the ADPI. To screen such a gene, any probe may be used that is complementary to the gene or its complement; preferably the probe is 10 nucleotides or longer, more preferably 15 nucleotides or longer.

When a library is screened, clones with insert DNA encoding the ADPI of interest or a fragment thereof will hybridise to one or more members of the corresponding set of degenerate oligonucleotide probes (or their complement). Hybridisation of such oligonucleotide probes to genomic libraries is carried out using methods known in the art. For example, hybridisation with one of the above-mentioned degenerate sets of oligonucleotide probes, or their complement (or with any member of such a set, or its complement) can be performed under highly stringent or moderately stringent conditions as defined above, or can be carried out in 2×SSC, 1.0% SDS at 50° C. and washed using the washing conditions described supra for highly stringent or moderately stringent hybridisation.

In yet another aspect of the invention, clones containing nucleotide sequences encoding the entire ADPI, a fragment of an ADPI, an ADPI-related polypeptide, or a fragment of an ADPI-related polypeptide or any of the foregoing may also be obtained by screening expression libraries. For example, DNA from the relevant source is isolated and random fragments are prepared and ligated into an expression vector (e.g., a bacteriophage, plasmid, phagemid or cosmid) such that the inserted sequence in the vector is capable of being expressed by the host cell into which the vector is then introduced. Various screening assays can then be used to select for the expressed ADPI or ADPI-related polypeptides. In one embodiment, the various anti-ADPI antibodies of the invention can be used to identify the desired clones using methods known in the art. See, for example, Harlow and Lane, 1988, Antibodies: A Laboratory Manual, Cold Spring Harbor Laboratory Press, Cold Spring Harbor, N.Y., Appendix IV. Colonies or plaques from the library are brought into contact with the antibodies to identify those clones that bind antibody.

In an embodiment, colonies or plaques containing DNA that encodes a ADPI, a fragment of a ADPI, a ADPI-related polypeptide, or a fragment of a ADPI-related polypeptide can be detected using DYNA Beads according to Olsvick et al., 29th ICAAC, Houston, Tex. 1989, incorporated herein by reference. Anti-ADPI antibodies are crosslinked to tosylated DYNA Beads M280, and these antibody-containing beads are then contacted with colonies or plaques expressing recombinant polypeptides. Colonies or plaques expressing a ADPI or ADPI-related polypeptide are identified as any of those that bind the beads.

Alternatively, the anti-ADPI antibodies can be nonspecifically immobilized to a suitable support, such as silica or Celite® resin. This material is then used to adsorb to bacterial colonies expressing the ADPI protein or ADPI-related polypeptide as described herein.

In another aspect, PCR amplification may be used to isolate from genomic DNA a substantially pure DNA (i.e., a DNA substantially free of contaminating nucleic acids) encoding the entire ADPI or a part thereof. Preferably such a DNA is at least 95% pure, more preferably at least 99% pure. Oligonucleotide sequences, degenerate or otherwise, that correspond to peptide sequences of ADPIs disclosed herein can be used as primers.

PCR can be earned out, e.g., by use of a Perkin-Elmer Cetus thermal cycler and Taq polymerase (Gene Amp® or AmpliTaq DNA polymerase). One can choose to synthesize several different degenerate primers, for use in the PCR reactions. It is also possible to vary the stringency of hybridisation conditions used in priming the PCR reactions, to allow for greater or lesser degrees of nucleotide sequence similarity between the degenerate primers and the corresponding sequences in the DNA. After successful amplification of a segment of the sequence encoding an ADPI, that segment may be molecularly cloned and sequenced, and utilized as a probe to isolate a complete genomic clone. This, in turn, will permit the determination of the gene's complete nucleotide sequence, the analysis of its expression, and the production of its protein product for functional analysis, as described infra.

The gene encoding an ADPI can also be identified by mRNA selection by nucleic acid hybridisation followed by in vitro translation. In this procedure, fragments are used to isolate complementary mRNAs by hybridisation. Such DNA fragments may represent available, purified DNA encoding a ADPI of another species (e.g., mouse, human). Immunoprecipitation analysis or functional assays (e.g., aggregation ability in vitro; binding to receptor) of the in vitro translation products of the isolated products of the isolated mRNAs identifies the mRNA and, therefore, the complementary DNA fragments that contain the desired sequences. In addition, specific mRNAs may be selected by adsorption of polysomes isolated from cells to immobilized antibodies that specifically recognize a ADPI. A radiolabelled cDNA encoding a ADPI can be synthesized using the selected mRNA (from the adsorbed polysomes) as a template. The radiolabelled mRNA or cDNA may then be used as a probe to identify the DNA fragments encoding an ADPI from among other genomic DNA fragments.

Alternatives to isolating genomic DNA encoding an ADPI include, but are not limited to, chemically synthesizing the gene sequence itself from a known sequence or making cDNA to the mRNA which encodes the ADPI. For example, RNA for cDNA cloning of the gene encoding a ADPI can be isolated from cells which express the ADPI. Those skilled in the art will understand from the present description that other methods may be used and are within the scope of the invention.

Any suitable eukaryotic cell can serve as the nucleic acid source for the molecular cloning of the gene encoding an ADPI. The nucleic acid sequences encoding the ADPI can be isolated from vertebrate, mammalian, primate, human, porcine, bovine, feline, avian, equine, canine or murine sources. The DNA may be obtained by standard procedures known in the art from cloned DNA (e.g., a DNA “library”), by chemical synthesis, by cDNA cloning, or by the cloning of genomic DNA, or fragments thereof, purified from the desired cell. (See, e.g., Sambrook et al., 1989, Molecular Cloning, A Laboratory Manual, 2d Ed., Cold Spring Harbor Laboratory Press, Cold Spring Harbor, N.Y.; Glover, D. M. (ed.), 1985, DNA Cloning: A Practical Approach, MRL Press, Ltd., Oxford, U. K. Vol. I, II.) Clones derived from genomic DNA may contain regulatory and intron DNA regions in addition to coding regions; clones derived from cDNA will contain only exon sequences.

The identified and isolated gene or cDNA can then be inserted into any suitable cloning vector. A large number of vector-host systems known in the art may be used. As those skilled in the art will appreciate, the vector system chosen should be compatible with the host cell used. Such vectors include, but are not limited to, bacteriophages such as lambda derivatives, plasmids such as PBR322 or pUC plasmid derivatives or the Bluescript vector (Stratagene) or modified viruses such as adenoviruses, adeno-associated viruses or retroviruses. The insertion into a cloning vector can be accomplished, for example, by ligating the DNA fragment into a cloning vector which has complementary cohesive termini. However, if the complementary restriction sites used to fragment the DNA are not present in the cloning vector, the ends of the DNA molecules may be enzymatically modified. Alternatively, any site desired may be produced by ligating nucleotide sequences (linkers) onto the DNA termini; these ligated linkers may comprise specific chemically synthesized oligonucleotides encoding restriction endonuclease recognition sequences. In an alternative method, the cleaved vector and the gene encoding a ADPI may be modified by homopolymeric tailing. Recombinant molecules can be introduced into host cells via transformation, transfection, infection, electroporation, etc., so that many copies of the gene sequence are generated.

In specific embodiments, transformation of host cells with recombinant DNA molecules that incorporate the isolated gene encoding the ADPI, cDNA, or synthesized DNA sequence enables generation of multiple copies of the gene. Thus, the gene may be obtained in large quantities by growing transformants, isolating the recombinant DNA molecules from the transformants and, when necessary, retrieving the inserted gene from the isolated recombinant DNA.

The nucleotide sequences of the present invention include nucleotide sequences encoding amino acid sequences with substantially the same amino acid sequences as native ADPI, nucleotide sequences encoding amino acid sequences with functionally equivalent amino acids, nucleotide sequences encoding ADPIs, fragments of ADPIs, ADPI-related polypeptides, or fragments of ADPI-related polypeptides.

In a specific embodiment, an isolated nucleic acid molecule encoding a ADPI-related polypeptide can be created by introducing one or more nucleotide substitutions, additions or deletions into the nucleotide sequence of a ADPI such that one or more amino acid substitutions, additions or deletions are introduced into the encoded protein. Standard techniques known to those of skill in the art can be used to introduce mutations, including, for example, site-directed mutagenesis and PCR-mediated mutagenesis. Preferably, conservative amino acid substitutions are made at one or more predicted non-essential amino acid residues. A “conservative amino acid substitution” is one in which the amino acid residue is replaced with an amino acid residue having a side chain with a similar charge. Families of amino acid residues having side chains with similar charges have been defined in the art. These families include amino acids with basic side chains (e.g., lysine, arginine, histidine), acidic side chains (e.g., aspartic acid, glutamic acid), uncharged polar side chains (e.g., glycine, asparagine, glutamine, serine, threonine, tyrosine, cysteine), nonpolar side chains (e.g., alanine, valine, leucine, isoleucine, proline, phenylalanine, methionine, tryptophan), beta-branched side chains (e.g., threonine, valine, isoleucine) and aromatic side chains (e.g., tyrosine, phenylalanine, tryptophan, histidine). Alternatively, mutations can be introduced randomly along all or part of the coding sequence, such as by saturation mutagenesis, and the resultant mutants can be screened for biological activity to identify mutants that retain activity. Following mutagenesis, the encoded protein can be expressed and the activity of the protein can be determined.

5.8 Expression of DNA Encoding ADPIs

The nucleotide sequence coding for a ADPI, a ADPI analogue, a ADPI-related peptide, or a fragment or other derivative of any of the foregoing, can be inserted into an appropriate expression vector, i.e., a vector which contains the necessary elements for the transcription and translation of the inserted protein-coding sequence. The necessary transcriptional and translational signals can also be supplied by the native gene encoding the ADPI or its flanking regions, or the native gene encoding the ADPI-related polypeptide or its flanking regions. A variety of host-vector systems may be utilized in the present invention to express the protein-coding sequence. These include but are not limited to mammalian cell systems infected with virus (e.g., vaccinia virus, adenovirus, etc.); insect cell systems infected with virus (e.g., baculovirus); microorganisms such as yeast containing yeast vectors; or bacteria transformed with bacteriophage, DNA, plasmid DNA, or cosmid DNA. The expression elements of vectors vary in their strengths and specificities. Depending on the host-vector system utilized, any one of a number of suitable transcription and translation elements may be used. In specific embodiments, a nucleotide sequence encoding a human gene (or a nucleotide sequence encoding a functionally active portion of a human ADPI) is expressed. In yet another embodiment, a fragment of an ADPI comprising a domain of the ADPI is expressed.

Any of the methods previously described for the insertion of DNA fragments into a vector may be used to construct expression vectors containing a chimeric gene consisting of appropriate transcriptional and translational control signals and the protein coding sequences. These methods may include in vitro recombinant DNA and synthetic techniques and in vivo recombinants (genetic recombination). Expression of nucleic acid sequence encoding an ADPI or fragment thereof may be regulated by a second nucleic acid sequence so that the ADPI or fragment is expressed in a host transformed with the recombinant DNA molecule. For example, expression of an ADPI may be controlled by any promoter or enhancer element known in the art. Promoters which may be used to control the expression of the gene encoding a ADPI or a ADPI-related polypeptide include, but are not limited to, the SV40 early promoter region (Bernoist and Chambon, 1981, Nature 290:304-310), the promoter contained in the 3′ long terminal repeat of Rous sarcoma virus (Yamamoto, et al., 1980, Cell 22:787-797), the herpes thymidine kinase promoter (Wagner et al., 1981, Proc. Natl. Acad. Sci. U.S.A. 78:1441-1445), the regulatory sequences of the metallothionein gene (Brinster et al., 1982, Nature 296:39-42), the tetracycline (Tet) promoter (Gossen et al., 1995, Proc. Nat. Acad. Sci. USA 89:5547-5551); prokaryotic expression vectors such as the β-lactamase promoter (Villa-Kamaroff, et al., 1978, Proc. Natl. Acad. Sci. U.S.A. 75:3727-3731), or the tac promoter (DeBoer, et al., 1983, Proc. Natl. Acad. Sci. U.S.A. 80:21-25; see also “Useful proteins from recombinant bacteria” in Scientific American, 1980, 242:74-94); plant expression vectors comprising the nopaline synthetase promoter region (Herrera-Estrella et al., Nature 303:209-213) or the cauliflower mosaic virus 35S RNA promoter (Gardner, et al., 1981, Nucl. Acids Res. 9:2871), and the promoter of the photosynthetic enzyme ribulose biphosphate carboxylase (Herrera-Estrella et al., 1984, Nature 310:115-120); promoter elements from yeast or other fungi such as the Gal 4 promoter, the ADC (alcohol dehydrogenase) promoter, PGK (phosphoglycerol kinase) promoter, alkaline phosphatase promoter, and the following animal transcriptional control regions, which exhibit tissue specificity and have been utilized in transgenic animals: elastase I gene control region which is active in pancreatic acinar cells (Swift et al., 1984, Cell 38:639-646; Ornitz et al., 1986, Cold Spring Harbor Symp. Quant. Biol. 50:399-409; MacDonald, 1987, Hepatology 7:425-515); insulin gene control region which is active in pancreatic beta cells (Hanahan, 1985, Nature 315:115-122), immmunoglobulin gene control region which is active in lymphoid cells (Grosschedl et al., 1984, Cell 38:647-658; Adames et al., 1985, Nature 318:533-538; Alexander et al., 1987, Mol. Cell. Biol. 7:1436-1444), mouse mammary tumor virus control region which is active in testicular, breast, lymphoid and mast cells (Leder et al., 1986, Cell 45:485-495), albumin gene control region which is active in liver (Pinkert et al., 1987, Genes and Devel. 1:268-276), alpha-fetoprotein gene control region which is active in liver (Krumlauf et al., 1985, Mol. Cell. Biol. 5:1639-1648; Hammer et al., 1987, Science 235:53-58; alpha 1-antitrypsin gene control region which is active in the liver (Kelsey et al., 1987, Genes and Devel. 1:161-171), beta-globin gene control region which is active in myeloid cells (Mogram et al., 1985, Nature 315:338-340; Kollias et al., 1986, Cell 46:89-94; myelin basic protein gene control region which is active in oligodendrocyte cells in the brain (Readhead et al., 1987, Cell 48:703-712); myosin light chain-2 gene control region which is active in skeletal muscle (Sani, 1985, Nature 314:283-286); neuronal-specific enolase (NSE) which is active in neuronal cells (Morelli et al., 1999, Gen. Virol. 80:571-83); brain-derived neurotrophic factor (BDNF) gene control region which is active in neuronal cells (Tabuchi et al., 1998, Biochem. Biophysic. Res. Com. 253:818-823); glial fibrillary acidic protein (GFAP) promoter which is active in astrocytes (Gomes et al., 1999, Braz J Med Biol Res 32(5):619-631; Morelli et al., 1999, Gen. Virol. 80:571-83) and gonadotropic releasing hormone gene control region which is active in the hypothalamus (Mason et al., 1986, Science 234:1372-1378).

In a specific embodiment, a vector is used that comprises a promoter operably linked to a ADPI-encoding nucleic acid, one or more origins of replication, and, optionally, one or more selectable markers (e.g., an antibiotic resistance gene).

In a specific embodiment, an expression construct is made by subcloning a ADPI or a ADPI-related polypeptide coding sequence into the EcoRI restriction site of each of the three pGEX vectors (Glutathione S-Transferase expression vectors; Smith and Johnson, 1988, Gene 7:31-40). This allows for the expression of the ADPI product or ADPI-related polypeptide from the subclone in the correct reading frame.

In mammalian host cells, a number of viral-based expression systems may be utilized. In cases where an adenovirus is used as an expression vector, the ADPI coding sequence or ADPI-related polypeptide coding sequence may be ligated to an adenovirus transcription/translation control complex, e.g., the late promoter and tripartite leader sequence. This chimeric gene may then be inserted in the adenovirus genome by in vitro or in vivo recombination. Insertion in a non-essential region of the viral genome (e.g., region E1 or E3) will result in a recombinant virus that is viable and capable of expressing the antibody molecule in infected hosts. (e.g., see Logan & Shenk, 1984, Proc. Natl. Acad. Sci. USA 81:355-359). Specific initiation signals may also be required for efficient translation of inserted antibody coding sequences. These signals include the ATG initiation codon and adjacent sequences. Furthermore, the initiation codon must be in phase with the reading frame of the desired coding sequence to ensure translation of the entire insert. These exogenous translational control signals and initiation codons can be of a variety of origins, both natural and synthetic. The efficiency of expression may be enhanced by the inclusion of appropriate transcription enhancer elements, transcription terminators, etc. (see Bittner et al., 1987, Methods in Enzymol. 153:51-544).

Expression vectors containing inserts of a gene encoding a ADPI or a ADPI-related polypeptide can be identified, for example, by three general approaches: (a) nucleic acid hybridisation, (b) presence or absence of “marker” gene functions, and (c) expression of inserted sequences. In the first approach, the presence of a gene encoding a ADPI inserted in an expression vector can be detected by nucleic acid hybridisation using probes comprising sequences that are homologous to an inserted gene encoding a ADPI. In the second approach, the recombinant vector/host system can be identified and selected based upon the presence or absence of certain “marker” gene functions (e.g., thymidine kinase activity, resistance to antibiotics, transformation phenotype, occlusion body formation in baculovirus, etc.) caused by the insertion of a gene encoding a ADPI in the vector. For example, if the gene encoding the ADPI is inserted within the marker gene sequence of the vector, recombinants containing the gene encoding the ADPI insert can be identified by the absence of the marker gene function. In the third approach, recombinant expression vectors can be identified by assaying the gene product (i.e., ADPI) expressed by the recombinant. Such assays can be based, for example, on the physical or functional properties of the ADPI in in vitro assay systems, e.g., binding with anti-ADPI antibody.

In addition, a host cell strain may be chosen which modulates the expression of the inserted sequences, or modifies and processes the gene product in the specific fashion desired. Expression from certain promoters can be elevated in the presence of certain inducers; thus, expression of the genetically engineered ADPI or ADPI-related polypeptide may be controlled. Furthermore, different host cells have characteristic and specific mechanisms for the translational and post-translational processing and modification (e.g., glycosylation, phosphorylation of proteins). Appropriate cell lines or host systems can be chosen to ensure the desired modification and processing of the foreign protein expressed. For example, expression in a bacterial system will produce an unglycosylated product and expression in yeast will produce a glycosylated product. Eukaryotic host cells which possess the cellular machinery for proper processing of the primary transcript, glycosylation, and phosphorylation of the gene product may be used. Such mammalian host cells include but are not limited to CHO, VERY, BHK, Hela, COS, MDCK, 293, 3T3, WI38, and in particular, neuronal cell lines such as, for example, SK-N-AS, SK-N-FI, SK-N-DZ human neuroblastomas (Sugimoto et al., 1984, J. Natl. Cancer Inst. 73: 51-57), SK-N-SH human neuroblastoma (Biochim. Biophys. Acta, 1982, 704: 450-460), Daoy human cerebellar medulloblastoma (He et al., 1992, Cancer Res. 52: 1144-1148) DBTRG-05 MG glioblastoma cells (Kruse et al., 1992, In Vitro Cell. Dev. Biol. 28A: 609-614), IMR-32 human neuroblastoma (Cancer Res., 1970, 30: 2110-2118), 1321N1 human astrocytoma (Proc. Natl Acad. Sci. USA, 1977, 74: 4816), MOG-G-CCM human astrocytoma (Br. J. Cancer, 1984, 49: 269), U87MG human glioblastoma-astrocytoma (Acta Pathol. Microbiol. Scand., 1968, 74: 465-486), A172 human glioblastoma (Olopade et al., 1992, Cancer Res. 52: 2523-2529), C6 rat glioma cells (Benda et al., 1968, Science 161: 370-371), Neuro-2a mouse neuroblastoma (Proc. Natl. Acad. Sci. USA, 1970, 65: 129-136), NB41A3 mouse neuroblastoma (Proc. Natl. Acad. Sci. USA, 1962, 48: 1184-1190), SCP sheep choroid plexus (Bolin et al., 1994, J. Virol. Methods 48: 211-221), G355-5, PG-4 Cat normal astrocyte (Haapala et al., 1985, J. Virol. 53: 827-833), Mpf ferret brain (Trowbridge et al., 1982, In Vitro 18: 952-960), and normal cell lines such as, for example, CTX TNA2 rat normal cortex brain (Radany et al., 1992, Proc. Natl. Acad. Sci. USA 89: 6467-6471) such as, for example, CRL7030 and Hs578Bst. Furthermore, different vector/host expression systems may effect processing reactions to different extents.

For long-term, high-yield production of recombinant proteins, stable expression is preferred. For example, cell lines which stably express the differentially expressed or pathway gene protein may be engineered. Rather than using expression vectors which contain viral origins of replication, host cells can be transformed with DNA controlled by appropriate expression control elements (e.g., promoter, enhancer, sequences, transcription terminators, polyadenylation sites, etc.), and a selectable marker. Following the introduction of the foreign DNA, engineered cells may be allowed to grow for 1-2 days in an enriched medium, and then are switched to a selective medium. The selectable marker in the recombinant plasmid confers resistance to the selection and allows cells to stably integrate the plasmid into their chromosomes and grow to form foci, which in turn can be cloned and expanded into cell lines. This method may advantageously be used to engineer cell lines which express the differentially expressed or pathway gene protein. Such engineered cell lines may be particularly useful in screening and evaluation of compounds that affect the endogenous activity of the differentially expressed or pathway gene protein.

A number of selection systems may be used, including but not limited to the herpes simplex virus thymidine kinase (Wigler, et al., 1977, Cell 11:223), hypoxanthine-guanine phosphoribosyltransferase (Szybalska & Szybalski, 1962, Proc. Natl. Acad. Sci. USA 48:2026), and adenine phosphoribosyltransferase (Lowy, et al., 1980, Cell 22:817) genes can be employed in tk-, hgprt- or aprt-cells, respectively. Also, antimetabolite resistance can be used as the basis of selection for dhfr, which confers resistance to methotrexate (Wigler, et al., 1980, Natl. Acad. Sci. USA 77:3567; O'Hare, et al., 1981, Proc. Natl. Acad. Sci. USA 78:1527); gpt, which confers resistance to mycophenolic acid (Mulligan & Berg, 1981, Proc. Natl. Acad. Sci. USA 78:2072); neo, which confers resistance to the aminoglycoside G-418 (Colberre-Garapin, et al., 1981, J. Mol. Biol. 150:1); and hygro, which confers resistance to hygromycin (Santerre, et al., 1984, Gene 30:147) genes.

In other embodiments, the ADPI, fragment, analogue, or derivative may be expressed as a fusion, or chimeric protein product (comprising the protein, fragment, analogue, or derivative joined via a peptide bond to a heterologous protein sequence). For example, the polypeptides of the present invention may be fused with the constant domain of immunoglobulins (IgA, IgE, IgG, IgM), or portions thereof (CH1, CH2, CH3, or any combination thereof and portions thereof) resulting in chimeric polypeptides. Such fusion proteins may facilitate purification, increase half-life in vivo, and enhance the delivery of an antigen across an epithelial barrier to the immune system. An increase in the half-life in vivo and facilitated purification has been shown for chimeric proteins consisting of the first two domains of the human CD4-polypeptide and various domains of the constant regions of the heavy or light chains of mammalian immunoglobulins. See, e.g., EP 394,827; Traunecker et al., Nature, 331:84-86 (1988). Enhanced delivery of an antigen across the epithelial barrier to the immune system has been demonstrated for antigens (e.g., insulin) conjugated to an FcRn binding partner such as IgG or Fc fragments (see, e.g., PCT publications WO 96/22024 and WO 99/04813).

Nucleic acids encoding an ADPI, a fragment of an ADPI, an ADPI-related polypeptide, or a fragment of an ADPI-related polypeptide can be fused to an epitope tag (e.g., the hemagglutinin (“HA”) tag or flag tag) to aid in detection and purification of the expressed polypeptide. For example, a system described by Janknecht et al. allows for the ready purification of non-denatured fusion proteins expressed in human cell lines (Janknecht et al., 1991, Proc. Natl. Acad. Sci. USA 88:8972-897).

An ADPI fusion protein can be made by ligating the appropriate nucleic acid sequences encoding the desired amino acid sequences to each other by methods known in the art, in the proper coding frame, and expressing the chimeric product by methods commonly known in the art. Alternatively, a ADPI fusion protein may be made by protein synthetic techniques, e.g., by use of a peptide synthesizer.

Both cDNA and genomic sequences can be cloned and expressed.

5.9 Domain Structure of ADPIs

Domains of some of the ADPIs provided by the present invention are known in the art and have been described in the scientific literature. Moreover, domains of a ADPI can be identified using techniques known to those of skill in the art. For example, one or more domains of a ADPI can be identified by using one or more of the following programs: ProDom, TMpred, and SAPS. ProDom compares the amino acid sequence of a polypeptide to a database of compiled domains (see, e.g., http://www.toulouse.inra.fr/prodom.html; Corpet F., Gouzy J. & Kahn D., 1999, Nucleic Acids Res., 27:263-267). TMpred predicts membrane-spanning regions of a polypeptide and their orientation. This program uses an algorithm that is based on the statistical analysis of TMbase, a database of naturally occuring transmembrane proteins (see, e.g., http://www.ch.embnet.org/software/TMPRED_form.html; Hofmann & Stoffel. (1993) “TMbase-A database of membrane spanning proteins segments.” Biol. Chem. Hoppe-Seyler 347,166). The SAPS program analyses polypeptides for statistically significant features like charge-clusters, repeats, hydrophobic regions, compositional domains (see, e.g., Brendel et al., 1992, Proc. Natl. Acad. Sci. USA 89: 2002-2006). Thus, based on the present description, those skilled in the art can identify domains of a ADPI having enzymatic or binding activity, and further can identify nucleotide sequences encoding such domains. These nucleotide sequences can then be used for recombinant expression of a ADPI fragment that retains the enzymatic or binding activity of the ADPI.

Based on the present description, those skilled in the art can identify domains of a ADPI having enzymatic or binding activity, and further can identify nucleotide sequences encoding such domains. These nucleotide sequences can then be used for recombinant expression of ADPI fragments that retain the enzymatic or binding activity of the ADPI.

In one embodiment, a ADPI has an amino acid sequence sufficiently similar to an identified domain of a known polypeptide. As used herein, the term “sufficiently similar” refers to a first amino acid or nucleotide sequence which contains a sufficient number of identical or equivalent (e.g., with a similar side chain) amino acid residues or nucleotides to a second amino acid or nucleotide sequence such that the first and second amino acid or nucleotide sequences have or encode a common structural domain or common functional activity or both.

An ADPI domain can be assessed for its function using techniques well known to those of skill in the art. For example, a domain can be assessed for its kinase activity or for its ability to bind to DNA using techniques known to the skilled artisan. Kinase activity can be assessed, for example, by measuring the ability of a polypeptide to phosphorylate a substrate. DNA binding activity can be assessed, for example, by measuring the ability of a polypeptide to bind to a DNA binding element in an electromobility shift assay. In a preferred embodiment, the function of a domain of an ADPI is determined using an assay described in one or more of the references identified in Table VIII, infra.

5.10 Production of Antibodies to ADPIs

According to the invention a ADPI, ADPI analogue, ADPI-related protein or a fragment or derivative of any of the foregoing may be used as an immunogen to generate antibodies which immunospecifically bind such an immunogen. Such immunogens can be isolated by any convenient means, including the methods described above. Antibodies of the invention include, but are not limited to polyclonal, monoclonal, bispecific, humanized or chimeric antibodies, single chain antibodies, Fab fragments and F(ab′) fragments, fragments produced by a Fab expression library, anti-idiotypic (anti-Id) antibodies, and epitope-binding fragments of any of the above. The term “antibody” as used herein refers to immunoglobulin molecules and immunologically active portions of immunoglobulin molecules, i.e., molecules that contain an antigen binding site that specifically binds an antigen. The immunoglobulin molecules of the invention can be of any class (e.g., IgG, IgE, IgM, IgD and IgA ) or subclass of immunoglobulin molecule.

In one embodiment, antibodies that recognize gene products of genes encoding ADPIs may be prepared. For example, antibodies that recognize these ADPIs and/or their isoforms include antibodies recognizing ADPI-62, ADPI-68, ADPI-72, ADPI-78.2, ADPI-108, ADPI-140, ADPI-141, ADPI-153.2, ADPI-175.2, ADPI-188.1, ADPI-189.2, ADPI-193, ADPI-196.2, ADPI-217, ADPI-220, ADPI-228, ADPI-230.1, ADPI-286, ADPI-289, ADPI-291, ADPI-297, ADPI-311, ADPI-323, ADPI-351, ADPI-353, ADPI-359, ADPI-371, ADPI-372, ADPI-387, ADPI-390, ADPI-399.2, ADPI-419, ADPI-424, ADPI-429, ADPI-471, ADPI-506, ADPI-511, ADPI-528, ADPI-547.3, ADPI-547.1. Certain antibodies are already known and can be purchased from commercial sources as shown in Table VII above. In another embodiment, methods known to those skilled in the art are used to produce antibodies that recognize an ADPI, an ADPI analogue, an ADPI-related polypeptide, or a derivative or fragment of any of the foregoing.

In one embodiment of the invention, antibodies to a specific domain of a ADPI are produced. In a specific embodiment, hydrophilic fragments of a ADPI are used as immunogens for antibody production.

In the production of antibodies, screening for the desired antibody can be accomplished by techniques known in the art, e.g. ELISA (enzyme-linked immunosorbent assay). For example, to select antibodies that recognize a specific domain of a ADPI, one may assay generated hybridomas for a product which binds to a ADPI fragment containing such domain. For selection of an antibody that specifically binds a first ADPI homologue but which does not specifically bind to (or binds less avidly to) a second ADPI homologue, one can select on the basis of positive binding to the first ADPI homologue and a lack of binding to (or reduced binding to) the second ADPI homologue. Similarly, for selection of an antibody that specifically binds a ADPI but which does not specifically bind to (or binds less avidly to) a different isoform of the same protein (such as a different glycoform having the same core peptide as the ADPI), one can select on the basis of positive binding to the ADPI and a lack of binding to (or reduced binding to) the different isoform (e.g., a different glycoform). Thus, the present invention provides an antibody (particularly a monoclonal antibody) that binds with greater affinity (particularly at least 2-fold, more particularly at least 5-fold still more particularly at least 10-fold greater affinity) to a ADPI than to a different isoform or isoforms (e.g., glycoforms) of the ADPI.

Polyclonal antibodies which may be used in the methods of the invention are heterogeneous populations of antibody molecules derived from the sera of immunized animals. Unfractionated immune serum can also be used. Various procedures known in the art may be used for the production of polyclonal antibodies to a ADPI, a fragment of a ADPI, a ADPI-related polypeptide, or a fragment of a ADPI-related polypeptide. In a particular embodiment, rabbit polyclonal antibodies to an epitope of a ADPI or a ADPI-related polypeptide can be obtained. For example, for the production of polyclonal or monoclonal antibodies, various host animals can be immunized by injection with the native or a synthetic (e.g., recombinant) version of a ADPI, a fragment of a ADPI, a ADPI-related polypeptide, or a fragment of a ADPI-related polypeptide, including but not limited to rabbits, mice, rats, etc. Isolated ADPIs suitable for such immunization may be obtained by the use of discovery techniques, such as the preferred technology described herein. If the ADPI is purified by gel electrophoresis, the ADPI can be used for immunization with or without prior extraction from the polyacrylamide gel. Various adjuvants may be used to enhance the immunological response, depending on the host species, including, but not limited to, complete or incomplete Freund's adjuvant, a mineral gel such as aluminium hydroxide, surface active substance such as lysolecithin, pluronic polyol, a polyanion, a peptide, an oil emulsion, keyhole limpet hemocyanin, dinitrophenol, and an adjuvant such as BCG (bacille Calmette-Guerin) or corynebacterium parvum. Additional adjuvants are also well known in the art.

For preparation of monoclonal antibodies (mAbs) directed toward an ADPI, a fragment of an ADPI, an ADPI-related polypeptide, or a fragment of an ADPI-related polypeptide, any technique that provides for the production of antibody molecules by continuous cell lines in culture may be used. For example, the hybridoma technique originally developed by Kohler and Milstein (1975, Nature 256:495-497), as well as the trioma technique, the human B-cell hybridoma technique (Kozbor et al., 1983, Immunology Today 4:72), and the EBV-hybridoma technique to produce human monoclonal antibodies (Cole et al., 1985, in Monoclonal Antibodies and Cancer Therapy, Alan R. Liss, Inc., pp. 77-96). Such antibodies may be of any immunoglobulin class including IgG, IgM, IgE, IgA, IgD and any subclass thereof. The hybridoma producing the mAbs of the invention may be cultivated in vitro or in vivo. In an additional embodiment of the invention, monoclonal antibodies can be produced in germ-free animals utilizing known technology (PCT/US90/02545, incorporated herein by reference).

The monoclonal antibodies include but are not limited to human monoclonal antibodies and chimeric monoclonal antibodies (e.g., human-mouse chimeras). Humanized antibodies are antibody molecules from non-human species having one or more complementarily determining regions (CDRs) from the non-human species and a framework region from a human immunoglobulin molecule. (See, e.g., Queen, U.S. Pat. No. 5,585,089, which is incorporated herein by reference in its entirety.) Chimeric and humanized monoclonal antibodies can be produced by recombinant DNA techniques known in the art, for example using methods described in PCT Publication No. WO 87/02671; European Patent Application 184,187; European Patent Application 171,496; European Patent Application 173,494; PCT Publication No. WO 86/01533; U.S. Pat. No. 4,816,567; European Patent Application 125,023; Better et al., 1988, Science 240:1041-1043; Liu et al., 1987, Proc. Natl. Acad. Sci. USA 84:3439-3443; Liu et al., 1987, J. Immunol. 139:3521-3526; Sun et al., 1987, Proc. Natl. Acad. Sci. USA 84:214-218; Nishimura et al., 1987, Canc. Res. 47:999-1005; Wood et al., 1985, Nature 314:446-449; and Shaw et al., 1988, J. Natl. Cancer Inst. 80:1553-1559; Morrison, 1985, Science 229:1202-1207; Oi et al., 1986, Bio/Techniques 4:214; U.S. Pat. No. 5,225,539; Jones et al., 1986, Nature 321:552-525; Verhoeyan et al. (1988) Science 239:1534; and Beidler et al., 1988, J. Immunol. 141:4053-4060.

Completely human antibodies are particularly desirable for therapeutic treatment of human subjects. Such antibodies can be produced using transgenic mice which are incapable of expressing endogenous immunoglobulin heavy and light chains genes, but which can express human heavy and light chain genes. The transgenic mice are immunized in the normal fashion with selected antigens, e.g., all or a portion of a ADPI of the invention. Monoclonal antibodies directed against the antigen can be obtained using conventional hybridoma technology. The human immunoglobulin transgenes harbored by the transgenic mice rearrange during B cell differentiation, and subsequently undergo class switching and somatic mutation. Thus, using such a technique, it is possible to produce therapeutically useful IgG, IgA, IgM and IgE antibodies. For an overview of this technology for producing human antibodies, see Lonberg and Huszar (1995, Int. Rev. Immunol. 13:65-93). For a detailed discussion of this technology for producing human antibodies and human monoclonal antibodies and protocols for producing such antibodies, see, e.g., U.S. Pat. Nos. 5,625,126; 5,633,425; 5,569,825; 5,661,016; and 5,545,806. In addition, companies such as Abgenix, Inc. (Freemont, Calif.) and Genpharm (San Jose, Calif.) can be engaged to provide human antibodies directed against a selected antigen using technology similar to that described above.

Completely human antibodies which recognize a selected epitope can be generated using a technique referred to as “guided selection.” In this approach a selected non-human monoclonal antibody, e.g., a mouse antibody, is used to guide the selection of a completely human antibody recognizing the same epitope. (Jespers et al. (1994) Biotechnology 12:899-903).

The antibodies of the present invention can also be generated using various phage display methods known in the art. In phage display methods, functional antibody domains are displayed on the surface of phage particles which carry the polynucleotide sequences encoding them. In a particular, such phage can be utilized to display antigen binding domains expressed from a repertoire or combinatorial antibody library (e.g., human or murine). Phage expressing an antigen binding domain that binds the antigen of interest can be selected or identified with antigen, e.g., using labeled antigen or antigen bound or captured to a solid surface or bead. Phage used in these methods are typically filamentous phage including fd and M13 binding domains expressed from phage with Fab, Fv or disulfide stabilized Fv antibody domains recombinantly fused to either the phage gene III or gene VIII protein. Phage display methods that can be used to make the antibodies of the present invention include those disclosed in Brinkman et al., J. Immunol. Methods 182:41-50 (1995); Ames et al., J. Immunol. Methods 184:177-186 (1995); Kettleborough et al., Eur. J. Immunol. 24:952-958 (1994); Persic et al., Gene 187 9-18 (1997); Burton et al., Advances in Immunology 57:191-280 (1994); PCT Application No. PCT/GB91/01134; PCT Publications WO 90/02809; WO 91/10737; WO 92/01047; WO 92/18619; WO 93/11236; WO 95/15982; WO 95/20401; and U.S. Pat. Nos. 5,698,426; 5,223,409; 5,403,484; 5,580,717; 5,427,908; 5,750,753; 5,821,047; 5,571,698; 5,427,908; 5,516,637; 5,780,225; 5,658,727; 5,733,743 and 5,969,108; each of which is incorporated herein by reference in its entirety.

As described in the above references, after phage selection, the antibody coding regions from the phage can be isolated and used to generate whole antibodies, including human antibodies, or any other desired antigen binding fragment, and expressed in any desired host, including mammalian cells, insect cells, plant cells, yeast, and bacteria, e.g., as described in detail below. For example, techniques to recombinantly produce Fab, Fab′ and F(ab′)2 fragments can also be employed using methods known in the art such as those disclosed in PCT publication WO 92/22324; Mullinax et al., BioTechniques 12(6):864-869 (1992); and Sawai et al., AJRI 34:26-34 (1995); and Better et al., Science 240:1041-1043 (1988) (said references incorporated by reference in their entireties).

Examples of suitable techniques which can be used to produce single-chain Fvs and antibodies against ADPIs of the present invention include those described in U.S. Pat. Nos. 4,946,778 and 5,258,498; Huston et al., Methods in Enzymology 203:46-88 (1991); Shu et al., PNAS 90:7995-7999 (1993); and Skerra et al., Science 240:1038-1040 (1988).

The invention further provides for the use of bispecific antibodies, which can be made by methods known in the art. Traditional production of full length bispecific antibodies is based on the co-expression of two immunoglobulin heavy chain-light chain pairs, where the two chains have different specificities (Milstein et al., 1983, Nature 305:537-539). Because of the random assortment of immunoglobulin heavy and light chains, these hybridomas (quadromas) produce a potential mixture of 10 different antibody molecules, of which only one has the correct bispecific structure. Purification of the correct molecule, which is usually done by affinity chromatography steps, is rather cumbersome, and the product yields are low. Similar procedures are disclosed in WO 93/08829, published May 13, 1993, and in Traunecker et al., 1991, EMBO J. 10:3655-3659.

According to a different and more preferred approach, antibody variable domains with the desired binding specificities (antibody-antigen combining sites) are fused to immunoglobulin constant domain sequences. The fusion preferably is with an immunoglobulin heavy chain constant domain, comprising at least part of the hinge, CH2, and CH3 regions. It is preferred to have the first heavy-chain constant region (CH1) containing the site necessary for light chain binding, present in at least one of the fusions. DNAs encoding the immunoglobulin heavy chain fusions and, if desired, the immunoglobulin light chain, are inserted into separate expression vectors, and are co-transfected into a suitable host organism. This provides for great flexibility in adjusting the mutual proportions of the three polypeptide fragments in embodiments when unequal ratios of the three polypeptide chains used in the construction provide the optimum yields. It is, however, possible to insert the coding sequences for two or all three polypeptide chains in one expression vector when the expression of at least two polypeptide chains in equal ratios results in high yields or when the ratios are of no particular significance.

In a preferred embodiment of this approach, the bispecific antibodies are composed of a hybrid immunoglobulin heavy chain with a first binding specificity in one arm, and a hybrid immunoglobulin heavy chain-light chain pair (providing a second binding specificity) in the other arm. It was found that this asymmetric structure facilitates the separation of the desired bispecific compound from unwanted immunoglobulin chain combinations, as the presence of an immunoglobulin light chain in only one half of the bispecific molecule provides for a facile way of separation. This approach is disclosed in WO 94/04690 published Mar. 3, 1994. For further details for generating bispecific antibodies see, for example, Suresh et al., Methods in Enzymology, 1986, 121:210.

The invention provides functionally active fragments, derivatives or analogues of the anti-ADPI immunoglobulin molecules. Functionally active means that the fragment, derivative or analogue is able to elicit anti-anti-idiotype antibodies (i.e., tertiary antibodies) that recognize the same antigen that is recognized by the antibody from which the fragment, derivative or analogue is derived. Specifically, in a preferred embodiment the antigenicity of the idiotype of the immunoglobulin molecule may be enhanced by deletion of framework and CDR sequences that are C-terminal to the CDR sequence that specifically recognizes the antigen. To determine which CDR sequences bind the antigen, synthetic peptides containing the CDR sequences can be used in binding assays with the antigen by any suitable binding assay known in the art.

The present invention provides antibody fragments such as, but not limited to, F(ab′)2 fragments and Fab fragments. Antibody fragments which recognize specific epitopes may be generated by known techniques. F(ab′)2 fragments consist of the variable region, the light chain constant region and the CH1 domain of the heavy chain and are generated by pepsin digestion of the antibody molecule. Fab fragments are generated by reducing the disulfide bridges of the F(ab′)2 fragments. The invention also provides heavy chain and light chain dimers of the antibodies of the invention, or any minimal fragment thereof such as Fvs or single chain antibodies (SCAs) (e.g., as described in U.S. Pat. No. 4,946,778; Bird, 1988, Science 242:423-42; Huston et al., 1988, Proc. Natl. Acad. Sci. USA 85:5879-5883; and Ward et al., 1989, Nature 334:544-54), or any other molecule with the same specificity as the antibody of the invention. Single chain antibodies are formed by linking the heavy and light chain fragments of the Fv region via an amino acid bridge, resulting in a single chain polypeptide. Techniques for the assembly of functional Fv fragments in E. coli may be used (Skerra et al., 1988, Science 242:1038-1041).

In other embodiments, the invention provides fusion proteins of the immunoglobulins of the invention (or functionally active fragments thereof), for example in which the immunoglobulin is fused via a covalent bond (e.g., a peptide bond), at either the N-terminus or the C-terminus to an amino acid sequence of another protein (or portion thereof, preferably at least 10, 20 or 50 amino acid portion of the protein) that is not the immunoglobulin. Preferably the immunoglobulin, or fragment thereof, is covalently linked to the other protein at the N-terminus of the constant domain. As stated above, such fusion proteins may facilitate purification, increase half-life in vivo, and enhance the delivery of an antigen across an epithelial barrier to the immune system.

The immunoglobulins of the invention include analogues and derivatives that are either modified, i.e., by the covalent attachment of any type of molecule as long as such covalent attachment that does not impair immunospecific binding. For example, but not by way of limitation, the derivatives and analogues of the immunoglobulins include those that have been further modified, e.g., by glycosylation, acetylation, pegylation, phosphorylation, amidation, derivatization by known protecting/blocking groups, proteolytic cleavage, linkage to a cellular ligand or other protein, etc. Any of numerous chemical modifications may be carried out by known techniques, including, but not limited to specific chemical cleavage, acetylation, formylation, etc. Additionally, the antilog or derivative may contain one or more non-classical or unnatural amino acids.

The foregoing antibodies can be used in methods known in the art relating to the localization and activity of the ADPIs of the invention, e.g., for imaging these proteins, measuring levels thereof in appropriate physiological samples, in diagnostic methods, etc.

5.11 Expression of Antibodies

The antibodies of the invention can be produced by any suitable method known in the art for the synthesis of antibodies, in particular, by chemical synthesis or by recombinant expression, and are preferably produced by recombinant expression techniques.

Recombinant expression of antibodies, or fragments, derivatives or analogues thereof, requires construction of a nucleic acid that encodes the antibody. If the nucleotide sequence of the antibody is known, a nucleic acid encoding the antibody may be assembled from chemically synthesized oligonucleotides (e.g., as described in Kutmeier et al., 1994, BioTechniques 17:242), which, briefly, involves the synthesis of overlapping oligonucleotides containing portions of the sequence encoding antibody, annealing and ligation of those oligonucleotides, and then amplification of the ligated oligonucleotides by PCR.

Alternatively, the nucleic acid encoding the antibody may be obtained by cloning the antibody. If a clone containing the nucleic acid encoding the particular antibody is not available, but the sequence of the antibody molecule is known, a nucleic acid encoding the antibody may be obtained from a suitable source (e.g., an antibody cDNA library, or cDNA library generated from any tissue or cells expressing the antibody) by PCR amplification using synthetic primers hybridisable to the 3′ and 5′ ends of the sequence or by cloning using an oligonucleotide probe specific for the particular gene sequence.

If an antibody molecule that specifically recognizes a particular antigen is not available (or a source for a cDNA library for cloning a nucleic acid encoding such an antibody), antibodies specific for a particular antigen may be generated by any method known in the art, for example, by immunizing an animal, such as a rabbit, to generate polyclonal antibodies or, more preferably, by generating monoclonal antibodies. Alternatively, a clone encoding at least the Fab portion of the antibody may be obtained by screening Fab expression libraries (e.g., as described in Huse et al., 1989, Science 246:1275-1281) for clones of Fab fragments that bind the specific antigen or by screening antibody libraries (See, e.g., Clackson et al., 1991, Nature 352:624; Hane et al., 1997 Proc. Natl. Acad. Sci. USA 94:4937).

Once a nucleic acid encoding at least the variable domain of the antibody molecule is obtained, it may be introduced into a vector containing the nucleotide sequence encoding the constant region of the antibody molecule (see, e.g., PCT Publication WO 86/05807; PCT Publication WO 89/01036; and U.S. Pat. No. 5,122,464). Vectors containing the complete light or heavy chain for co-expression with the nucleic acid to allow the expression of a complete antibody molecule are also available. Then, the nucleic acid encoding the antibody can be used to introduce the nucleotide substitution(s) or deletion(s) necessary to substitute (or delete) the one or more variable region cysteine residues participating in an intrachain disulfide bond with an amino acid residue that does not contain a sulfhydyl group. Such modifications can be carried out by any method known in the art for the introduction of specific mutations or deletions in a nucleotide sequence, for example, but not limited to, chemical mutagenesis, in vitro site directed mutagenesis (Hutchinson et al., 1978, J. Biol. Chem. 253:6551), PCT based methods, etc.

In addition, techniques developed for the production of “chimeric antibodies” (Morrison et al., 1984, Proc. Natl. Acad. Sci. 81:851-855; Neuberger et al., 1984, Nature 312:604-608; Takeda et al., 1985, Nature 314:452-454) by splicing genes from a mouse antibody molecule of appropriate antigen specificity together with genes from a human antibody molecule of appropriate biological activity can be used. As described supra, a chimeric antibody is a molecule in which different portions are derived from different animal species, such as those having a variable region derived from a murine mAb and a human antibody constant region, e.g., humanized antibodies.

Once a nucleic acid encoding an antibody molecule of the invention has been obtained, the vector for the production of the antibody molecule may be produced by recombinant DNA technology using techniques well known in the art. Thus, methods for preparing the protein of the invention by expressing nucleic acid containing the antibody molecule sequences are described herein. Methods which are well known to those skilled in the art can be used to construct expression vectors containing an antibody molecule coding sequences and appropriate transcriptional and translational control signals. These methods include, for example, in vitro recombinant DNA techniques, synthetic techniques, and in vivo genetic recombination. See, for example, the techniques described in Sambrook et al. (1990, Molecular Cloning, A Laboratory Manual, 2d Ed., Cold Spring Harbor Laboratory, Cold Spring Harbor, N.Y.) and Ausubel et al. (eds., 1998, Current Protocols in Molecular Biology, John Wiley & Sons, NY).

The expression vector is transferred to a host cell by conventional techniques and the transfected cells are then cultured by conventional techniques to produce an antibody of the invention.

The host cells used to express a recombinant antibody of the invention may be either bacterial cells such as Escherichia coli, or, preferably, eukaryotic cells, especially for the expression of whole recombinant antibody molecule. In particular, mammalian cells such as Chinese hamster ovary cells (CHO), in conjunction with a vector such as the major intermediate early gene promoter element from human cytomegalovirus is an effective expression system for antibodies (Foecking et al., 1986, Gene 45:101; Cockett et al., 1990, Bio/Technology 8:2).

A variety of host-expression vector systems may be utilized to express an antibody molecule of the invention. Such host-expression systems represent vehicles by which the coding sequences of interest may be produced and subsequently purified, but also represent cells which may, when transformed or transfected with the appropriate nucleotide coding sequences, express the antibody molecule of the invention in situ. These include but are not limited to microorganisms such as bacteria (e.g., E. coli, B. subtilis) transformed with recombinant bacteriophage DNA, plasmid DNA or cosmid DNA expression vectors containing antibody coding sequences; yeast (e.g., Saccharomyces, Pichia) transformed with recombinant yeast expression vectors containing antibody coding sequences; insect cell systems infected with recombinant virus expression vectors (e.g., baculovirus) containing the antibody coding sequences; plant cell systems infected with recombinant virus expression vectors (e.g., cauliflower mosaic virus, CaMV; tobacco mosaic virus, TMV) or transformed with recombinant plasmid expression vectors (e.g., Ti plasmid) containing antibody coding sequences; or mammalian cell systems (e.g., COS, CHO, BHK, HEK 293, 3T3 cells) harbouring recombinant expression constructs containing promoters derived from the genome of mammalian cells (e.g., metallothionein promoter) or from mammalian viruses (e.g., the adenovirus late promoter; the vaccinia virus 7.5K promoter).

In bacterial systems, a number of expression vectors may be advantageously selected depending upon the use intended for the antibody molecule being expressed. For example, when a large quantity of such a protein is to be produced, for the generation of pharmaceutical compositions comprising an antibody molecule, vectors which direct the expression of high levels of fusion protein products that are readily purified may be desirable. Such vectors include, but are not limited, to the E. coli expression vector pUR278 (Ruther et al., 1983, EMBO J. 2:1791), in which the antibody coding sequence may be ligated individually into the vector in frame with the lac Z coding region so that a fusion protein is produced; pIN vectors (Inouye & Inouye, 1985, Nucleic Acids Res. 13:3101-3109; Van Heeke & Schuster, 1989, J. Biol. Chem. 24:5503-5509); and the like. pGEX vectors may also be used to express foreign polypeptides as fusion proteins with glutathione S-transferase (GST). In general, such fusion proteins are soluble and can easily be purified from lysed cells by adsorption and binding to a matrix glutathione-agarose beads followed by elution in the presence of free glutathione. The pGEX vectors are designed to include thrombin or factor Xa protease cleavage sites so that the cloned target gene product can be released from the GST moiety.

In an insect system, Autographa californica nuclear polyhedrosis virus (AcNPV) is used as a vector to express foreign genes. The virus grows in Spodoptera frugiperda cells. The antibody coding sequence may be cloned individually into non-essential regions (for example the polyhedrin gene) of the virus and placed under control of an AcNPV promoter (for example the polyhedrin promoter). In mammalian host cells, a number of viral-based expression systems (e.g., an adenovirus expression system) may be utilized.

As discussed above, a host cell strain may be chosen based on the present description which modulates the expression of the inserted sequences, or modifies and processes the gene product in the specific fashion desired. Such modifications (e.g., glycosylation) and processing (e.g., cleavage) of protein products may be important for the function of the protein.

For long-term, high-yield production of recombinant antibodies, stable expression is preferred. For example, cells lines that stably express an antibody of interest can be produced by transfecting the cells with an expression vector comprising the nucleotide sequence of the antibody and the nucleotide sequence of a selectable (e.g., neomycin or hygromycin), and selecting for expression of the selectable marker. Such engineered cell lines may be particularly useful in screening and evaluation of compounds that interact directly or indirectly with the antibody molecule.

The expression levels of the antibody molecule can be increased by vector amplification (for a review, see Bebbington and Hentschel, The use of vectors based on gene amplification for the expression of cloned genes in mammalian cells in DNA cloning, Vol.3. (Academic Press, New York, 1987)). When a marker in the vector system expressing antibody is amplifiable, an increase in the level of inhibitor present in culture of host cell will increase the number of copies of the marker gene. Since the amplified region is associated with the antibody gene, production of the antibody will also increase (Crouse et al., 1983, Mol. Cell. Biol. 3:257).

The host cell may be co-transfected with two expression vectors of the invention, the first vector encoding a heavy chain derived polypeptide and the second vector encoding a light chain derived polypeptide. The two vectors may contain identical selectable markers which enable equal expression of heavy and light chain polypeptides. Alternatively, a single vector may be used which encodes both heavy and light chain polypeptides. In such situations, the light chain should be placed before the heavy chain to avoid an excess of toxic free heavy chain (Proudfoot, 1986, Nature 322:52; Kohler, 1980, Proc. Natl. Acad. Sci. USA 77:2197). The coding sequences for the heavy and light chains may comprise cDNA or genomic DNA.

Once the antibody molecule of the invention has been recombinantly expressed, it may be purified by any method known in the art for purification of an antibody molecule, for example, by chromatography (e.g., ion exchange chromatography, affinity chromatography such as with protein A or specific antigen, and sizing column chromatography), centrifugation, differential solubility, or by any other standard technique for the purification of proteins.

Alternatively, any fusion protein may be readily purified by utilizing an antibody specific for the fusion protein being expressed. For example, a system described by Janknecht et al. allows for the ready purification of non-denatured fusion proteins expressed in human cell lines (Janknecht et al., 1991, Proc. Natl. Acad. Sci. USA 88:8972-897). In this system, the gene of interest is subcloned into a vaccinia recombination plasmid such that the open reading frame of the gene is translationally fused to an amino-terminal tag consisting of six histidine residues. The tag serves as a matrix binding domain for the fusion protein. Extracts from cells infected with recombinant vaccinia virus are loaded onto Ni2+ nitriloacetic acid-agarose columns and histidine-tagged proteins are selectively eluted with imidazole-containing buffers.

5.12 Conjugated Antibodies

In a preferred embodiment, anti-ADPI antibodies or fragments thereof are conjugated to a diagnostic or a therapeutic moiety. The antibodies can be used, for example, for diagnosis or to determine the efficacy of a given treatment regimen. Detection can be facilitated by coupling the antibody to a detectable substance. Examples of detectable substances include various enzymes, prosthetic groups, fluorescent materials, luminescent materials, bioluminescent materials, radioactive nuclides, positron emitting metals (for use in positron emission tomography), and nonradioactive paramagnetic metal ions. See generally U.S. Pat. No. 4,741,900 for metal ions which can be conjugated to antibodies for use as diagnostics according to the present invention. Suitable enzymes include horseradish peroxidase, alkaline phosphatase, beta-galactosidase, or acetylcholinesterase; suitable prosthetic groups include streptavidin, avidin and biotin; suitable fluorescent materials include umbelliferone, fluorescein, fluorescein isothiocyanate, rhodamine, dichlorotriazinylamine fluorescein, dansyl chloride and phycoerythrin; suitable luminescent materials include luminol; suitable bioluminescent materials include luciferase, luciferin, and aequorin; and suitable radioactive nuclides include ¹²⁵I, ¹³¹I, ¹¹¹In and ⁹⁹Tc.

Anti-ADPI antibodies or fragments thereof can be conjugated to a therapeutic agent or drug moiety to modify a given biological response. The therapeutic agent or drug moiety is not to be construed as limited to classical chemical therapeutic agents. For example, the drug moiety may be a protein or polypeptide possessing a desired biological activity. Such proteins may include, for example, a toxin such as abrin, ricin A, pseudomonas exotoxin, or diphtheria toxin; a protein such as tumor necrosis factor, α-interferon, β-interferon, nerve growth factor, platelet derived growth factor, tissue plasminogen activator, a thrombotic agent or an anti-angiogenic agent, e.g., angiostatin or endostatin; or, a biological response modifier such as a lymphokine, interleukin-1 (IL-1), interleukin-2 (IL-2), interleukin-6 (IL-6), granulocyte macrophage colony stimulating factor (GM-CSF), granulocyte colony stimulating factor (G-CSF), nerve growth factor (NGF) or other growth factor.

Techniques for conjugating such therapeutic moiety to antibodies are well known, see, e.g., Arnon et al., “Monoclonal Antibodies For Immunotargeting of Drugs In Cancer Therapy”, in Monoclonal Antibodies And Cancer Therapy, Reisfeld et al. (eds.), pp. 243-56 (Alan R. Liss, Inc. 1985); Hellstrom et al., “Antibodies For Drug Delivery”, in Controlled Drug Delivery (2nd Ed.), Robinson et al. (eds.), pp. 623-53 (Marcel Dekker, Inc. 1987); Thorpe, “Antibody Carriers of Cytotoxic Agents In Cancer Therapy: A Review”, in Monoclonal Antibodies '84: Biological And Clinical Applications, Pinchera et al. (eds.), pp. 475-506 (1985); “Analysis, Results, And Future Prospective of The Therapeutic Use of Radiolabeled Antibody In Cancer Therapy”, in Monoclonal Antibodies For Cancer Detection And Therapy, Baldwin et al. (eds.), pp. 303-16 (Academic Press 1985), and Thorpe et al., “The Preparation And Cytotoxic Properties of Antibody-Toxin Conjugates”, Immunol. Rev., 62:119-58 (1982). These references are incorporated herein in their entirety.

Alternatively, an antibody can be conjugated to a second antibody to form an antibody heteroconjugate as described by Segal in U.S. Pat. No. 4,676,980.

An antibody with or without a therapeutic moiety conjugated to it can be used as a therapeutic that is administered alone or in combination with cytotoxic factor(s) and/or cytokine(s).

5.13 Diagnosis of Alzheimer's Disease

In accordance with the present invention, suitable test samples, e.g., of brain tissue, obtained from a subject suspected of having or known to have Alzheimer's disease can be used for diagnosis. In one embodiment, an altered abundance of one or more ADFs or ADPIs (or any combination of them) in a test sample relative to a control sample (from a subject or subjects free from Alzheimer's disease) or a previously determined reference range indicates the presence of Alzheimer's disease; ADFs and ADPIs suitable for this purpose are identified in Tables I and III, respectively, as described in detail above. In another embodiment, the relative abundance of one or more ADFs or ADPIs (or any combination of them) in a test sample compared to a control sample or a previously determined reference range indicates a subtype of Alzheimer's disease (e.g., familial or sporadic Alzheimer's disease). In yet another embodiment, the relative abundance of one or more ADFs or ADPIs (or any combination of them) in a test sample relative to a control sample or a previously determined reference range indicates the degree or severity of Alzheimer's disease. In any of the aforesaid methods, detection of one or more ADPIs described herein may optionally be combined with detection of one or more additional biomarkers for Alzheimer's disease including, but not limited to apolipoprotein E (ApoE), amyloid -peptides (A), tau and neural thread protein (NTP). Any suitable method in the art can be employed to measure the level of ADFs and ADPIs, including but not limited to the Preferred Technology described herein, kinase assays, immunoassays to detect and/or visualize the ADPIs (e.g., Western blot, immunoprecipitation followed by sodium dodecyl sulfate polyacrylamide gel electrophoresis, immunocytochemistry, etc.). In cases where an ADPI has a known function, an assay for that function may be used to measure ADPI expression. In a further embodiment, an altered abundance of mRNA encoding one or more ADPIs identified in Table III (or any combination of them) in a test sample relative to a control sample or a previously determined reference range indicates the presence of Alzheimer's disease. Any suitable hybridisation assay can be used to detect ADPI expression by detecting and/or visualizing mRNA encoding the ADPI (e.g., Northern assays, dot blots, in situ hybridisation, etc.).

In another embodiment of the invention, labelled antibodies, derivatives and analogues thereof, which specifically bind to an ADPI can be used for diagnostic purposes, e.g., to detect, diagnose, or monitor Alzheimer's disease. Preferably, Alzheimer's disease is detected in an animal, more preferably in a mammal and most preferably in a human.

5.14 Screening Assays

The invention provides methods for identifying agents (e.g., chemical compounds, proteins, or peptides) that bind to an ADPI or have a stimulatory or inhibitory effect on the expression or activity of an ADPI. The invention also provides methods of identifying agents, candidate compounds or test compounds that bind to a ADPI-related polypeptide or a ADPI fusion protein or have a stimulatory or inhibitory effect on the expression or activity of a ADPI-related polypeptide or a ADPI fusion protein. Examples of agents, candidate compounds or test compounds include, but are not limited to, nucleic acids (e.g., DNA and RNA), carbohydrates, lipids, proteins, peptides, peptidomimetics, small molecules and other drugs. Agents can be obtained using any of the numerous suitable approaches in combinatorial library methods known in the art, including: biological libraries; spatially addressable parallel solid phase or solution phase libraries; synthetic library methods requiring deconvolution; the “one-bead one-compound” library method; and synthetic library methods using affinity chromatography selection. The biological library approach is limited to peptide libraries, while the other four approaches are applicable to peptide, non-peptide oligomer or small molecule libraries of compounds (Lam, 1997, Anticancer Drug Des. 12:145; U.S. Pat. Nos. 5,738,996; and 5,807,683, each of which is incorporated herein in its entirety by reference).

Examples of methods for the synthesis of molecular libraries can be found in the art, for example in: DeWitt et al., 1993, Proc. Natl. Acad. Sci. USA 90:6909; Erb et al., 1994, Proc. Natl. Acad. Sci. USA 91:11422; Zuckermann et al., 1994, J. Med. Chem. 37:2678; Cho et al., 1993, Science 261:1303; Carrell et al., 1994, Angew. Chem. Int. Ed. Engl. 33:2059; Carell et al., 1994, Angew. Chem. Int. Ed. Engl. 33:2061; and Gallop et al., 1994, J. Med. Chem. 37:1233, each of which is incorporated herein in its entirety by reference.

Libraries of compounds may be presented, e.g., presented in solution (e.g., Houghten, 1992, Bio/Techniques 13:412-421), or on beads (Lam, 1991, Nature 354:82-84), chips (Fodor, 1993, Nature 364:555-556), bacteria (U.S. Pat. No. 5,223,409), spores (U.S. Pat. Nos. 5,571,698; 5,403,484; and 5,223,409), plasmids (Cull et al., 1992, Proc. Natl. Acad. Sci. USA 89:1865-1869) or phage (Scott and Smith, 1990, Science 249:386-390; Devlin, 1990, Science 249:404-406; Cwirla et al., 1990, Proc. Natl. Acad. Sci. USA 87:6378-6382; and Felici, 1991, J. Mol. Biol. 222:301-310), each of which is incorporated herein in its entirety by reference.

In one embodiment, agents that interact with (i.e., bind to) an ADPI, an ADPI fragment (e.g. a functionally active fragment), an ADPI-related polypeptide, a fragment of an ADPI-related polypeptide, or an ADPI fusion protein are identified in a cell-based assay system. In accordance with this embodiment, cells expressing an ADPI, a fragment of an ADPI, an ADPI-related polypeptide, a fragment of an ADPI-related polypeptide, or an ADPI fusion protein are contacted with a candidate compound or a control compound and the ability of the candidate compound to interact with the ADPI is determined. If desired, this assay may be used to screen a plurality (e.g. a library) of candidate compounds. The cell, for example, can be of prokaryotic origin (e.g., E. coli) or eukaryotic origin (e.g., yeast or mammalian). Further, the cells can express the ADPI, fragment of the ADPI, ADPI-related polypeptide, a fragment of the ADPI-related polypeptide, or a ADPI fusion protein endogenously or be genetically engineered to express the ADPI, fragment of the ADPI, ADPI-related polypeptide, a fragment of the ADPI-related polypeptide, or an ADPI fusion protein. In some embodiments, the ADPI, fragment of the ADPI, ADPI-related polypeptide, a fragment of the ADPI-related polypeptide, or an ADPI fusion protein or the candidate compound is labelled, for example with a radioactive label (such as ³²P, ³⁵S or ¹²⁵I) or a fluorescent label (such as fluorescein isothiocyanate, rhodamine, phycoerythrin, phycocyanin, allophycocyanin, o-phthaldehyde or fluorescamine) to enable detection of an interaction between a ADPI and a candidate compound. The ability of the candidate compound to interact directly or indirectly with an ADPI, an fragment of an ADPI, an ADPI-related polypeptide, a fragment of an ADPI-related polypeptide, or an ADPI fusion protein can be determined by methods known to those of skill in the art. For example, the interaction between a candidate compound and an ADPI, a fragment of an ADPI, an ADPI-related polypeptide, a fragment of an ADPI-related polypeptide, or an ADPI fusion protein can be determined by flow cytometry, a scintillation assay, immunoprecipitation or western blot analysis.

In another embodiment, agents that interact with (i.e., bind to) an ADPI, an ADPI fragment (e.g., a functionally active fragment) an ADPI-related polypeptide, a fragment of an ADPI-related polypeptide, or an ADPI fusion protein are identified in a cell-free assay system. In accordance with this embodiment, a native or recombinant ADPI or fragment thereof, or a native or recombinant ADPI-related polypeptide or fragment thereof, or an ADPI-fusion protein or fragment thereof, is contacted with a candidate compound or a control compound and the ability of the candidate compound to interact with the ADPI or ADPI-related polypeptide, or ADPI fusion protein is determined. If desired, this assay may be used to screen a plurality (e.g. a library) of candidate compounds. Preferably, the ADPI, ADPI fragment, ADPI-related polypeptide, fragment of a ADPI-related polypeptide, or ADPI-fusion protein is first immobilized, by, for example, contacting the ADPI, ADPI fragment, ADPI-related polypeptide, fragment of a ADPI-related polypeptide, or ADPI fusion protein with an immobilized antibody which specifically recognizes and binds it, or by contacting a purified preparation of the ADPI, ADPI fragment, ADPI-related polypeptide, fragment of an ADPI-related polypeptide, or ADPI fusion protein with a surface designed to bind proteins. The ADPI, ADPI fragment, ADPI-related polypeptide, fragment of a ADPI-related polypeptide, or ADPI fusion protein may be partially or completely purified (e.g., partially or completely free of other polypeptides) or part of a cell lysate. Further, the ADPI, ADPI fragment, ADPI-related polypeptide, fragment of a ADPI-related polypeptide may be a fusion protein comprising the ADPI or a biologically active portion thereof, or ADPI-related polypeptide and a domain such as glutathionine-S-transferase. Alternatively, the ADPI, ADPI fragment, ADPI-related polypeptide, fragment of a ADPI-related polypeptide or ADPI fusion protein can be biotinylated using techniques well known to those of skill in the art (e.g., biotinylation kit, Pierce Chemicals; Rockford, Ill.). The ability of the candidate compound to interact with a ADPI, ADPI fragment, ADPI-related polypeptide, fragment of an ADPI-related polypeptide, or ADPI fusion protein can be can be determined by methods known to those of skill in the art.

In another embodiment, a cell-based assay system is used to identify agents that bind to or modulate the activity of a protein, such as an enzyme, or a biologically active portion thereof, which is responsible for the production or degradation of a ADPI or is responsible for the post-translational modification of a ADPI. In a primary screen, a plurality (e.g., a library) of compounds are contacted with cells that naturally or recombinantly express: (i) an ADPI, an isoform of an ADPI, an ADPI homologue, an ADPI-related polypeptide, an ADPI fusion protein, or a biologically active fragment of any of the foregoing; and (ii) a protein that is responsible for processing of the ADPI, ADPI isoform, ADPI homologue, ADPI-related polypeptide, ADPI fusion protein, or fragment in order to identify compounds that modulate the production, degradation, or post-translational modification of the ADPI, ADPI isoform, ADPI homologue, ADPI-related polypeptide, ADPI fusion protein or fragment. If desired, compounds identified in the primary screen can then be assayed in a secondary screen against cells naturally or recombinantly expressing the specific ADPIs of interest. The ability of the candidate compound to modulate the production, degradation or post-translational modification of a ADPI, isoform, homologue, ADPI-related polypeptide, or ADPI fusion protein can be determined by methods known to those of skill in the art, including without limitation, flow cytometry, a scintillation assay, immunoprecipitation and western blot analysis.

In another embodiment, agents that competitively interact with (i.e., bind to) an ADPI, ADPI fragment, ADPI-related polypeptide, a fragment of an ADPI-related polypeptide, or an ADPI fusion protein are identified in a competitive binding assay. In accordance with this embodiment, cells expressing an ADPI, ADPI fragment, ADPI-related polypeptide, a fragment of an ADPI-related polypeptide, or an ADPI fusion protein are contacted with a candidate compound and a compound known to interact with the ADPI, ADPI fragment, ADPI-related polypeptide, a fragment of an ADPI-related polypeptide or an ADPI fusion protein; the ability of the candidate compound to competitively interact with the ADPI, ADPI fragment, ADPI-related polypeptide, fragment of a ADPI-related polypeptide, or ADPI fusion protein is then determined. Alternatively, agents that competitively interact with (i.e., bind to) a ADPI, ADPI fragment, ADPI-related polypeptide or fragment of a ADPI-related polypeptide are identified in a cell-free assay system by contacting a ADPI, ADPI fragment, ADPI-related polypeptide, fragment of a ADPI-related polypeptide, or a ADPI fusion protein with a candidate compound and a compound known to interact with the ADPI, ADPI-related polypeptide or ADPI fusion protein. As stated above, the ability of the candidate compound to interact with a ADPI, ADPI fragment, ADPI-related polypeptide, a fragment of a ADPI-related polypeptide, or a ADPI fusion protein can be determined by methods known to those of skill in the art. These assays, whether cell-based or cell-free, can be used to screen a plurality (e.g., a library) of candidate compounds.

In another embodiment, agents that modulate (i.e., upregulate or downregulate) the expression of a ADPI, or a ADPI-related polypeptide are identified by contacting cells (e.g., cells of prokaryotic origin or eukaryotic origin) expressing the ADPI, or ADPI-related polypeptide with a candidate compound or a control compound (e.g., phosphate buffered saline (PBS)) and determining the expression of the ADPI, ADPI-related polypeptide, or ADPI fusion protein, mRNA encoding the ADPI, or mRNA encoding the ADPI-related polypeptide. The level of expression of a selected ADPI, ADPI-related polypeptide, mRNA encoding the ADPI, or mRNA encoding the ADPI-related polypeptide in the presence of the candidate compound is compared to the level of expression of the ADPI, ADPI-related polypeptide, mRNA encoding the ADPI, or mRNA encoding the ADPI-related polypeptide in the absence of the candidate compound (e.g., in the presence of a control compound). The candidate compound can then be identified as a modulator of the expression of the ADPI, or a ADPI-related polypeptide based on this comparison. For example, when expression of the ADPI or mRNA is significantly greater in the presence of the candidate compound than in its absence, the candidate compound is identified as a stimulator of expression of the ADPI or mRNA. Alternatively, when expression of the ADPI or mRNA is significantly less in the presence of the candidate compound than in its absence, the candidate compound is identified as an inhibitor of the expression of the ADPI or mRNA. The level of expression of a ADPI or the mRNA that encodes it can be determined by methods known to those of skill in the art based on the present description. For example, mRNA expression can be assessed by Northern blot analysis or RT-PCR, and protein levels can be assessed by western blot analysis.

In another embodiment, agents that modulate the activity of an ADPI, or an ADPI-related polypeptide are identified by contacting a preparation containing the ADPI or ADPI-related polypeptide, or cells (e.g., prokaryotic or eukaryotic cells) expressing the ADPI or ADPI-related polypeptide with a test compound or a control compound and determining the ability of the test compound to modulate (e.g., stimulate or inhibit) the activity of the ADPI or ADPI-related polypeptide. The activity of an ADPI or an ADPI-related polypeptide can be assessed by detecting induction of a cellular signal transduction pathway of the ADPI or ADPI-related polypeptide (e.g., intracellular Ca2+, diacylglycerol, IP3, etc.), detecting catalytic or enzymatic activity of the target on a suitable substrate, detecting the induction of a reporter gene ( e.g., a regulatory element that is responsive to a ADPI or a ADPI-related polypeptide and is operably linked to a nucleic acid encoding a detectable marker, e.g., luciferase), or detecting a cellular response, for example, cellular differentiation, or cell proliferation as the case may be, based on the present description, techniques known to those of skill in the art can be used for measuring these activities (see, e.g., U.S. Pat. No. 5,401,639, which is incorporated in its entirety herein by reference). The candidate agent can then be identified as a modulator of the activity of a ADPI or ADPI-related polypeptide by comparing the effects of the candidate compound to the control compound. Suitable control compounds include phosphate buffered saline (PBS) and normal saline (NS).

In another embodiment, agents that modulate (i.e., upregulate or downregulate) the expression, activity or both the expression and activity of a ADPI or ADPI-related polypeptide are identified in an animal model. Examples of suitable animals include, but are not limited to, mice, rats, rabbits, monkeys, guinea pigs, dogs and cats. Preferably, the animal used represent a model of Alzheimer's disease (e.g., (e.g., animals that express human familial Alzheimer's disease (FAD) -amyloid precursor (APP), animals that over-express human wild-type APP, animals that over-express -amyloid 1-42 (A), animals that express FAD presenillin-1 (PS-1). See, e.g., Higgins, LS, 1999, Molecular Medicine Today 5:274-276.). In accordance with this embodiment, the test compound or a control compound is administered (e.g., orally, rectally or parenterally such as intraperitoneally or intravenously) to a suitable animal and the effect on the expression, activity or both expression and activity of the ADPI or ADPI-related polypeptide is determined. Changes in the expression of an ADPI or ADPI-related polypeptide can be assessed by any suitable method described above, based on the present description.

In yet another embodiment, an ADPI or ADPI-related polypeptide is used as a “bait protein” in a two-hybrid assay or three hybrid assay to identify other proteins that bind to or interact with a ADPI or ADPI-related polypeptide (see, e.g., U.S. Pat. No. 5,283,317; Zervos et al. (1993) Cell 72:223-232; Madura et al. (1993) J. Biol. Chem. 268:12046-12054; Bartel et al. (1993) Bio/Techniques 14:920-924; Iwabuchi et al. (1993) Oncogene 8:1693-1696; and PCT Publication No. WO 94/10300). As those skilled in the art will appreciate, such binding proteins are also likely to be involved in the propagation of signals by the ADPIs of the invention as, for example, upstream or downstream elements of a signaling pathway involving the ADPIs of the invention.

As those skilled in the art will appreciate, Table VIII enumerates scientific publications describing suitable assays for detecting or quantifying enzymatic or binding activity of an ADPI, an ADPI analogue, an ADPI-related polypeptide, or a fragment of any of the foregoing. Each such reference is hereby incorporated in its entirety. In a preferred embodiment, an assay referenced in Table VIII is used in the screens and assays described herein, for example, to screen for or to identify an agent that modulates the activity of (or that modulates both the expression and activity of) an ADPI, ADPI analogue, or ADPI-related polypeptide, a fragment of any of the foregoing or an ADPI fusion protein.

Table VIII. Known Scientific Assays for ADPIs.

TABLE VIII

| ADPI# | Scientific Assay Reference |
|---|---|
| ADPI-109 | “omega-Fluoromethyl analogues of omega-amino acids as irreversible |
| ADPI-229 | inhibitors of 4-aminobutyrate:2-oxoglutarate aminotransferase.” J Neurochem |
| | 1981 Nov 37:5 1341-4 |
| | “4-Aminobutyrate aminotransferase. Different susceptibility to inhibitors, |
| | microenvironment of the cofactor binding site and distance of the catalytic |
| | sites.” Eur J Biochem 1982 126:3 507-11 |
| ADPI-108 | “Kinetic and crystallographic studies on the active site Arg289Lys mutant of |
| | flavocytochrome b2 (yeast L-lactate dehydrogenase)”, (2000), 39(12), pp3266-75 |
| ADPI-139 | “Determination of the Structure of the N-terminal Splice Region of the Cyclic |
| ADPI-260 | AMP-specific Phosphodiesterase RD1 (RNPDE4A1) by 1H NMR and |
| ADPI-377 | Identification of the Membrane Association Domain Using Chimeric |
| ADPI-452 | Constructs.” J Biol Chem 1996, 271: 16703-16711. |
| ADPI-495 | “The effect of Met−−>Leu mutations on calmodulin's ability to activate cyclic |
| ADPI-509 | nucleotide phosphodiesterase.” J Biol Chem 1994, 269: 15546-15552. |
| ADPI-534 | |
| ADPI-399.2 | “Cytosolic aldehyde dehydrogenase (ALDH1) variants found in alcohol |
| | flushers” (1989), Ann Hum Genet. 53 pp1-7 |
| | “Studies of the esterase activity of cytosolic aldehyde dehydrogenase with |
| | resorufin acetate as substrate” (1997), Biochem J, 332, pp701-708. |
| | “Hepatic lipid peroxidation and aldehyde dehydrogenase activity in alcoholic |
| | and non-alcholic liver disease” (1989) Alcohol Alcohol, 24(2), pp121-8 |
| ADPI-183 | “Capillary electrophoresis assay for ubiquitin carboxyl-terminal hydrolases with |
| ADPI-499 | chemically synthesized ubiquitin-valine as substrate.” Anal Biochem 1997 May |
| | 1; 247(2): 305-9 |
| ADPI-175.2 | “Alteration of glyceraldehyde-3-phosphate dehydrogenase activity and |
| ADPI-286 | messenger mRNA content by androgen in human prostate carcinoma cells”, |
| ADPI-289 | (1995), Cancer Res. 55(19, pp4234-6 |
| ADPI-291 | “Increased membrane activity of glyceraldehyde 3-phosphate dehydrogenase in |
| | erythrocytes of patients with homozygous sickle cell anaemia”, (1992), Clin |
| | Chim Acta 209(3), pp189-95 |
| | “Heterogeneity of glyceraldehyde-3-phosphate dehydrogenase from human |
| | brain” (1988), 954(3), pp309-24 |
| ADPI-95 | “A single amino acid substitution makes ERK2 susceptible to pyridinyl |
| | imidazole inhibitors of p38 MAP kinase.” Protein Sci 1998 Nov 7: 11 2249-55 |
| ADPI-466.2 | “Purification and partial characterization of the phosphoglucomutase isozymes |
| | from human placenta” (1990), Prep. Biochem. 20(3-4), pp219-240. |
| | “Isoenzymes of human red blood cells: isolation and kinetic properties”, |
| | (1989), Prep Biochem. 19(3), pp251-271 |
| | “Human erythrocyte phosphoglucomutase: comparison of the properties of |
| | PGM1 and PGM2 isoenzymes” (1984), Biochimie, 66(9-10), pp617-623 |
| ADPI-390 | “Hsp90 Chaperone Activity Requires the Full-length Protein and Interaction |
| | among Its Multiple Domains” (2000) J. Biol. Chem., 275(42), pp32499-32507. |

This invention further provides novel agents identified by the above-described screening assays and uses thereof for treatments as described herein.

5.15 Therapeutic Uses of ADPIs

The invention provides for treatment or prevention of various diseases and disorders by administration of a therapeutic agent. Such agents include but are not limited to: ADPIs, ADPI analogues, ADPI-related polypeptides and derivatives (including fragments) thereof; antibodies to the foregoing; nucleic acids encoding ADPIs, ADPI analogues, ADPI-related polypeptides and fragments thereof; antisense nucleic acids to a gene encoding a ADPI or ADPI-related polypeptide; and modulator (e.g., agonists and antagonists) of a gene encoding a ADPI or ADPI-related polypeptide. An important feature of the present invention is the identification of genes encoding ADPIs involved in Alzheimer's disease. Alzheimer's disease can be treated (e.g. to ameliorate symptoms or to retard onset or progression) or prevented by administration of a therapeutic compound that promotes function or expression of one or more ADPIs that are decreased in the brain tissue of subjects having Alzheimer's disease, or by administration of a therapeutic compound that reduces function or expression of one or more ADPIs that are increased in the brain tissue of subjects having Alzheimer's disease.

In one embodiment, one or more antibodies each specifically binding to a ADPI are administered alone or in combination with one or more additional therapeutic compounds or treatments. Examples of such therapeutic compounds or treatments include, but are not limited to, acetylcholinesterase inhibitors e.g. Tacrine (Cognex®), Donepezil (Aricept®), Rivastigmine (Exelon®).

Preferably, a biological product such as an antibody is allogeneic to the subject to which it is administered. In a preferred embodiment, a human ADPI or a human ADPI-related polypeptide, a nucleotide sequence encoding a human ADPI or a human ADPI-related polypeptide, or an antibody to a human ADPI or a human ADPI-related polypeptide, is administered to a human subject for therapy (e.g. to ameliorate symptoms or to retard onset or progression) or prophylaxis.

5.15.1 Treatment and Prevention of Alzheimer's Disease

Alzheimer's disease can be treated or prevented by administration to a subject suspected of having or known to have Alzheimer's disease or to be at risk of developing Alzheimer's disease of an agent that modulates (i.e., increases or decreases) the level or activity (i.e., function) of one or more ADPIs—or the level of one or more ADFs—that are differentially present in the brain tissue of subjects having Alzheimer's disease compared with brain tissue of subjects free from Alzheimer's disease. In one embodiment, Alzheimer's disease is treated by administering to a subject suspected of having or known to have Alzheimer's disease or to be at risk of developing Alzheimer's disease an agent that upregulates (i.e., increases) the level or activity (i.e., function) of one or more ADPIs—or the level of one or more ADFs—that are decreased in the brain tissue of subjects having Alzheimer's disease. In another embodiment, an agent is administered that downregulates the level or activity (i.e., function) of one or more ADPIs—or the level of one or more ADFs—that are increased in the brain tissue of subjects having Alzheimer's disease. Examples of such a compound include but are not limited to: ADPIs, ADPI fragments and ADPI-related polypeptides; nucleic acids encoding an ADPI, an ADPI fragment and an ADPI-related polypeptide (e.g., for use in gene therapy); and, for those ADPIs or ADPI-related polypeptides with enzymatic activity, compounds or molecules known to modulate that enzymatic activity. Other compounds that can be used, e.g., ADPI agonists, can be identified using in vitro assays, as defined or described above or earlier.

Alzheimer's disease is also treated or prevented by administration to a subject suspected of having or known to have Alzheimer's disease or to be at risk of developing Alzheimer's disease of a compound that downregulates the level or activity of one or more ADPIs—or the level of one or more ADFs—that are increased in the brain tissue of subjects having Alzheimer's disease. In another embodiment, a compound is administered that upregulates the level or activity of one or more ADPIs—or the level of one or more ADFs—that are decreased in the brain tissue of subjects having Alzheimer's disease. Examples of such a compound include, but are not limited to, ADPI antisense oligonucleotides, ribozymes, antibodies directed against ADPIs, and compounds that inhibit the enzymatic activity of a ADPI. Other useful compounds e.g., ADPI antagonists and small molecule ADPI antagonists, can be identified using in vitro assays.

In a preferred embodiment, therapy or prophylaxis is tailored to the needs of an individual subject. Thus, in specific embodiments, compounds that promote the level or function of one or more ADPIs, or the level of one or more ADFs, are therapeutically or prophylactically administered to a subject suspected of having or known to have Alzheimer's disease, in whom the levels or functions of said one or more ADPIs, or levels of said one or more ADFs, are absent or are decreased relative to a control or normal reference range. In further embodiments, compounds that promote the level or function of one or more ADPIs, or the level of one or more ADFs, are therapeutically or prophylactically administered to a subject suspected of having or known to have Alzheimer's disease in whom the levels or functions of said one or more ADPIs, or levels of said one or more ADFs, are increased relative to a control or to a reference range. In further embodiments, compounds that decrease the level or function of one or more ADPIs, or the level of one or more ADFs, are therapeutically or prophylactically administered to a subject suspected of having or known to have Alzheimer's disease in whom the levels or functions of said one or more ADPIs, or levels of said one or more ADFs, are increased relative to a control or to a reference range. In further embodiments, compounds that decrease the level or function of one or more ADPIs, or the level of one or more ADFs, are therapeutically or prophylactically administered to a subject suspected of having or known to have Alzheimer's disease in whom the levels or functions of said one or more ADPIs, or levels of said one or more ADFs, are decreased relative to a control or to a reference range. The change in ADPI function or level, or ADF level, due to the administration of such compounds can be readily detected, e.g., by obtaining a sample (e.g., a sample of brain tissue, blood or urine or a tissue sample such as biopsy tissue) and assaying in vitro the levels of said ADFs or the levels or activities of said ADPIs, or the levels of mRNAs encoding said ADPIs. or any combination of the foregoing. Such assays can be performed before and after the administration of the compound as described herein.

The compounds of the invention include but are not limited to any compound, e.g., a small organic molecule, protein, peptide, antibody, nucleic acid, etc. that restores the Alzheimer's disease ADPI or ADF profile towards normal with the proviso that such compounds do not include Tacrine (Cognex®), Donepezil (Aricept®), Rivastigmine (Exelon®).

5.15.2 Gene Therapy

In another embodiment, nucleic acids comprising a sequence encoding a ADPI, a ADPI fragment, ADPI-related polypeptide or fragment of a ADPI-related polypeptide, are administered to promote ADPI function by way of gene therapy. Gene therapy refers to the administration of an expressed or expressible nucleic acid to a subject. In this embodiment, the nucleic acid produces its encoded polypeptide and the polypeptide mediates a therapeutic effect by promoting ADPI function.

Any suitable methods for gene therapy available in the art can be used according to the present invention.

For general reviews of the methods of gene therapy, see Goldspiel et al., 1993, Clinical Pharmacy 12:488-505; Wu and Wu, 1991, Biotherapy 3:87-95; Tolstoshev, 1993, Ann. Rev. Pharmacol. Toxicol. 32:573-596; Mulligan, 1993, Science 260:926-932; and Morgan and Anderson, 1993, Ann. Rev. Biochem. 62:191-217; May, 1993, TIBTECH 11(5):155-215. Methods commonly known in the art of recombinant DNA technology which can be used in the present invention are described in Ausubel et al. (eds.), 1993, Current Protocols in Molecular Biology, John Wiley & Sons, NY; and Kriegler, 1990, Gene Transfer and Expression, A Laboratory Manual, Stockton Press, NY.

In a particular aspect, the compound comprises a nucleic acid encoding a ADPI or fragment or chimeric protein thereof, said nucleic acid being part of an expression vector that expresses a ADPI or fragment or chimeric protein thereof in a suitable host. In particular, such a nucleic acid has a promoter operably linked to the ADPI coding region, said promoter being inducible or constitutive (and, optionally, tissue-specific). In another particular embodiment, a nucleic acid molecule is used in which the ADPI coding sequences and any other desired sequences are flanked by regions that promote homologous recombination at a desired site in the genome, thus providing for intrachromosomal expression of the ADPI nucleic acid (Koller and Smithies, 1989, Proc. Natl. Acad. Sci. USA 86:8932-8935; Zijlstra et al., 1989, Nature 342:435-438).

Delivery of the nucleic acid into a subject may be direct, in which case the subject is directly exposed to the nucleic acid or nucleic acid-carrying vector; this approach is known as in vivo gene therapy. Alternatively, delivery of the nucleic acid into the subject may be indirect, in which case cells are first transformed with the nucleic acid in vitro and then transplanted into the subject, known as “ex vivo gene therapy”.

In another embodiment, the nucleic acid is directly administered in vivo, where it is expressed to produce the encoded product. This can be accomplished by any of numerous methods known in the art, e.g., by constructing it as part of an appropriate nucleic acid expression vector and administering it so that it becomes intracellular, e.g., by infection using a defective or attenuated retroviral or other viral vector (see U.S. Pat. No. 4,980,286); by direct injection of naked DNA; by use of microparticle bombardment (e.g., a gene gun; Biolistic, Dupont); by coating with lipids, cell-surface receptors or transfecting agents; by encapsulation in liposomes, microparticles or microcapsules; by administering it in linkage to a peptide which is known to enter the nucleus; or by administering it in linkage to a ligand subject to receptor-mediated endocytosis (see, e.g., Wu and Wu, 1987, J. Biol. Chem. 262:4429-4432), which can be used to target cell types specifically expressing the receptors. In another embodiment, a nucleic acid-ligand complex can be formed in which the ligand comprises a fusogenic viral peptide to disrupt endosomes, allowing the nucleic acid to avoid lysosomal degradation. In yet another embodiment, the nucleic acid can be targeted in vivo for cell specific uptake and expression, by targeting a specific receptor (see, e.g., PCT Publications WO 92/06180 dated Apr. 16, 1992 (Wu et al.); WO 92/22635 dated Dec. 23, 1992 (Wilson et al.); WO92/20316 dated Nov. 26, 1992 (Findeis et al.); WO93/14188 dated Jul. 22, 1993 (Clarke et al.), WO 93/20221 dated Oct. 14, 1993 (Young)). Alternatively, the nucleic acid can be introduced intracellularly and incorporated within host cell DNA for expression, by homologous recombination (Koller and Smithies, 1989, Proc. Natl. Acad. Sci. USA 86:8932-8935; Zijlstra et al., 1989, Nature 342:435-438).

In a further embodiment, a viral vector that contains a nucleic acid encoding a ADPI is used. For example, a retroviral vector can be used (see Miller et al., 1993, Meth. Enzymol. 217:581-599). These retroviral vectors have been modified to delete retroviral sequences that are not necessary for packaging of the viral genome and integration into host cell DNA. The nucleic acid encoding the ADPI to be used in gene therapy is cloned into the vector, which facilitates delivery of the gene into a subject. More detail about retroviral vectors can be found in Boesen et al., 1994, Biotherapy 6:291-302, which describes the use of a retroviral vector to deliver the mdrl gene to hematopoietic stem cells in order to make the stem cells more resistant to chemotherapy. Other references illustrating the use of retroviral vectors in gene therapy are: Clowes et al., 1994, J. Clin. Invest. 93:644-651; Kiem et al., 1994, Blood 83:1467-1473; Salmons and Gunzberg, 1993, Human Gene Therapy 4:129-141; and Grossman and Wilson, 1993, Curr. Opin. in Genetics and Devel. 3:110-114.

Adenoviruses are other viral vectors that can be used in gene therapy. Adenoviruses are especially attractive vehicles for delivering genes to respiratory epithelia. Adenoviruses naturally infect respiratory epithelia where they cause a mild disease. Other targets for adenovirus-based delivery systems are liver, the central nervous system, endothelial cells, and muscle. Adenoviruses have the advantage of being capable of infecting non-dividing cells. Kozarsky and Wilson, 1993, Current Opinion in Genetics and Development 3:499-503 present a review of adenovirus-based gene therapy. Bout et al., 1994, Human Gene Therapy 5:3-10 demonstrated the use of adenovirus vectors to transfer genes to the respiratory epithelia of rhesus monkeys. Other instances of the use of adenoviruses in gene therapy can be found in Rosenfeld et al., 1991, Science 252:431-434; Rosenfeld et al., 1992, Cell 68:143-155; Mastrangeli et al., 1993, J. Clin. Invest. 91:225-234; PCT Publication WO94/12649; and Wang, et al., 1995, Gene Therapy 2:775-783.

Adeno-associated virus (AAV) has also been proposed for use in gene therapy (Walsh et al., 1993, Proc. Soc. Exp. Biol. Med. 204:289-300; U.S. Pat. No. 5,436,146).

Another suitable approach to gene therapy involves transferring a gene to cells in tissue culture by such methods as electroporation, lipofection, calcium phosphate mediated transfection, or viral infection. Usually, the method of transfer includes the transfer of a selectable marker to the cells. The cells are then placed under selection to isolate those cells that have taken up and are expressing the transferred gene. Those cells are then delivered to a subject.

In this embodiment, the nucleic acid is introduced into a cell prior to administration in vivo of the resulting recombinant cell. Such introduction can be carried out by any method known in the art, including but not limited to transfection, electroporation, microinjection, infection with a viral or bacteriophage vector containing the nucleic acid sequences, cell fusion, chromosome-mediated gene transfer, microcell-mediated gene transfer, spheroplast fusion, etc. Numerous techniques are known in the art for the introduction of foreign genes into cells (see, e.g., Loeffler and Behr, 1993, Meth. Enzymol. 217:599-618; Cohen et al., 1993, Meth. Enzymol. 217:618-644; Cline, 1985, Pharmac. Ther. 29:69-92) and may be used in accordance with the present invention, provided that the necessary developmental and physiological functions of the recipient cells are not disrupted. The technique should provide for the stable transfer of the nucleic acid to the cell, so that the nucleic acid is expressible by the cell and preferably heritable and expressible by its cell progeny.

The resulting recombinant cells can be delivered to a subject by various methods known in the art. In a preferred embodiment, epithelial cells are injected, e.g., subcutaneously. In another embodiment, recombinant skin cells may be applied as a skin graft onto the subject. Recombinant blood cells (e.g., hematopoietic stem or progenitor cells) are preferably administered intravenously. The amount of cells envisioned for use depends on the desired effect, the condition of the subject, etc., and can be determined by one skilled in the art.

Cells into which a nucleic acid can be introduced for purposes of gene therapy encompass any desired, available cell type, and include but are not limited to neuronal cells, glial cells (e.g., oligodendrocytes or astrocytes), epithelial cells, endothelial cells, keratinocytes, fibroblasts, muscle cells, hepatocytes; blood cells such as T lymphocytes, B lymphocytes, monocytes, macrophages, neutrophils, eosinophils, megakaryocytes, granulocytes; various stem or progenitor cells, in particular hematopoietic stem or progenitor cells, e.g., as obtained from bone marrow, umbilical cord blood, peripheral blood or fetal liver.

In a preferred embodiment, the cell used for gene therapy is autologous to the subject that is treated.

In an embodiment in which recombinant cells are used in gene therapy, a nucleic acid encoding a ADPI is introduced into the cells such that it is expressible by the cells or their progeny, and the recombinant cells are then administered in vivo for therapeutic effect. In a specific embodiment, stem or progenitor cells are used. Any stem or progenitor cells which can be isolated and maintained in vitro can be used in accordance with this embodiment of the present invention (see e.g. PCT Publication WO 94/08598, dated Apr. 28, 1994; Stemple and Anderson, 1992, Cell 71:973-985; Rheinwald, 1980, Meth. Cell Bio. 21A:229; and Pittelkow and Scott, 1986, Mayo Clinic Proc. 61:771).

In another embodiment, the nucleic acid to be introduced for purposes of gene therapy may comprise an inducible promoter operably linked to the coding region, such that expression of the nucleic acid is controllable by controlling the presence or absence of the appropriate inducer of transcription.

Direct injection of a DNA coding for a ADPI may also be performed according to, for example, the techniques described in U.S. Pat. No. 5,589,466. These techniques involve the injection of “naked DNA”, i.e., isolated DNA molecules in the absence of liposomes, cells, or any other material besides a suitable carrier. The injection of DNA encoding a protein and operably linked to a suitable promoter results in the production of the protein in cells near the site of injection and the elicitation of an immune response in the subject to the protein encoded by the injected DNA. In a preferred embodiment, naked DNA comprising (a) DNA encoding a ADPI and (b) a promoter are injected into a subject to elicit an immune response to the ADPI.

5.15.3 Inhibition of ADPIs to Treat Alzheimer's Disease

In one embodiment of the invention, Alzheimer's disease is treated or prevented by administration of a compound that antagonizes (inhibits) the level(s) and/or function(s) of one or more ADPIs which are elevated in the brain tissue of subjects having Alzheimer's disease as compared with brain tissue of subjects free from Alzheimer's disease. Compounds useful for this purpose include but are not limited to anti-ADPI antibodies (and fragments and derivatives containing the binding region thereof), ADPI antisense or ribozyme nucleic acids, and nucleic acids encoding dysfunctional ADPIs that are used to “knockout” endogenous ADPI function by homologous recombination (see, e.g., Capecchi, 1989, Science 244:1288-1292). Other compounds that inhibit ADPI function can be identified by use of known in vitro assays, e.g., assays for the ability of a test compound to inhibit binding of a ADPI to another protein or a binding partner, or to inhibit a known ADPI function. Preferably such inhibition is assayed in vitro or in cell culture, but genetic assays may also be employed. The Preferred Technology can also be used to detect levels of the ADPI before and after the administration of the compound. Preferably, suitable in vitro or in vivo assays are utilized to determine the effect of a specific compound and whether its administration is indicated for treatment of the affected tissue, as described in more detail below.

In a particular embodiment, a compound that inhibits a ADPI function is administered therapeutically or prophylactically to a subject in whom an increased brain tissue level or functional activity of the ADPI (e.g., greater than the normal level or desired level) is detected as compared with brain tissue of subjects free from Alzheimer's disease or a predetermined reference range. Methods standard in the art can be employed to measure the increase in a ADPI level or function, as outlined above. Preferred ADPI inhibitor compositions include small molecules, i.e., molecules of 1000 daltons or less. Such small molecules can be identified by the screening methods described herein.

5.15.4 Antisense Regulation of ADPIs

In a further embodiment, ADPI expression is inhibited by use of ADPI antisense nucleic acids. The present invention provides the therapeutic or prophylactic use of nucleic acids comprising at least six nucleotides that are antisense to a gene or cDNA encoding a ADPI or a portion thereof. As used herein, a ADPI “antisense” nucleic acid refers to a nucleic acid capable of hybridizing by virtue of some sequence complementarity to a portion of an RNA (preferably mRNA) encoding a ADPI. The antisense nucleic acid may be complementary to a coding and/or noncoding region of an mRNA encoding a ADPI. Such antisense nucleic acids have utility as compounds that inhibit ADPI expression, and can be used in the treatment or prevention of Alzheimer's disease.

The antisense nucleic acids of the invention are double-stranded or single-stranded oligonucleotides, RNA or DNA or a modification or derivative thereof, and can be directly administered to a cell or produced intracellularly by transcription of exogenous, introduced sequences.

The invention further provides pharmaceutical compositions comprising a therapeutically effective amount of a ADPI antisense nucleic acid, and a pharmaceutically-acceptable carrier, vehicle or diluent.

In another embodiment, the invention provides methods for inhibiting the expression of a ADPI nucleic acid sequence in a prokaryotic or eukaryotic cell comprising providing the cell with an effective amount of a composition comprising a ADPI antisense nucleic acid of the invention.

ADPI antisense nucleic acids and their uses are described in detail below.

5.15.5 ADPI Antisense Nucleic Acids

The ADPI antisense nucleic acids are of at least six nucleotides and are preferably oligonucleotides ranging from 6 to about 50 oligonucleotides. In specific aspects, the oligonucleotide is at least 10 nucleotides, at least 15 nucleotides, at least 100 nucleotides, or at least 200 nucleotides. The oligonucleotides can be DNA or RNA or chimeric mixtures or derivatives or modified versions thereof and can be single-stranded or double-stranded. The oligonucleotide can be modified at the base moiety, sugar moiety, or phosphate backbone. The oligonucleotide may include other appended groups such as peptides; agents that facilitate transport across the cell membrane (see, e.g., Letsinger et al., 1989, Proc. Natl. Acad. Sci. USA 86:6553-6556; Lemaitre et al., 1987, Proc. Natl. Acad. Sci. 84:648-652; PCT Publication No. WO 88/09810, published Dec. 15, 1988) or blood-brain barrier (see, e.g., PCT Publication No. WO 89/10134, published Apr. 25, 1988); hybridization-triggered cleavage agents (see, e.g., Krol et al., 1988, BioTechniques 6:958-976) or intercalating agents (see, e.g., Zon, 1988, Pharm. Res. 5:539-549).

In a particular aspect of the invention, a ADPI antisense oligonucleotide is provided, preferably of single-stranded DNA. The oligonucleotide may be modified at any position on its structure with substituents generally known in the art.

The ADPI antisense oligonucleotide may comprise any suitable of the following modified base moieties, e.g. 5-fluorouracil, 5-bromouracil, 5-chlorouracil, 5-iodouracil, hypoxanthine, xantine, 4-acetylcytosine, 5-(carboxyhydroxyhmethyl) uracil, 5-carboxymethylaminomethyl-2-thiouridine, 5-carboxymethylaminomethyluracil, dihydrouracil, beta-D-galactosylqueosine, inosine, N6-isopentenyladenine, 1-methylguanine, 1-methylinosine, 2,2-dimethylguanine, 2-methyladenine, 2-methylguanine, 3-methylcytosine, 5-methylcytosine, N6-adenine, 7-methylguanine, 5-methylaminomethyluracil, 5-methoxyaminomethyl-2-thiouracil, beta-D-mannosylqueosine, 5 -methoxycarboxymethyluracil, 5-methoxyuracil, 2-methylthio-N6-isopentenyladenine, uracil-5-oxyacetic acid (v), wybutoxosine, pseudouracil, queosine, 2-thiocytosine, 5-methyl-2-thiouracil, 2-thiouracil, 4-thiouracil, 5-methyluracil, uracil-5-oxyacetic acid methylester, uracil-5-oxyacetic acid (v), 5-methyl-2-thiouracil, 3-(3-amino-3-N-2-carboxypropyl) uracil, (acp3)w, 2,6-diaminopurine, and other base analogs.

In another embodiment, the oligonucleotide comprises at least one modified sugar moiety, e.g., one of the following sugar moieties: arabinose, 2-fluoroarabinose, xylulose, and hexose.

In yet another embodiment, the oligonucleotide comprises at least one of the following modified phosphate backbones: a phosphorothioate, a phosphorodithioate, a phosphoramidothioate, a phosphoramidate, a phosphordiamidate, a methylphosphonate, an alkyl phosphotriester, a formacetal, or an analogue of formacetal.

In yet another embodiment, the oligonucleotide is an α-anomeric oligonucleotide. An α-anomeric oligonucleotide forms specific double-stranded hybrids with complementary RNA in which, contrary to the usual β-units, the strands run parallel to each other (Gautier et al., 1987, Nucl. Acids Res. 15:6625-6641).

The oligonucleotide may be conjugated to another molecule, e.g., a peptide, hybridization triggered cross-linking agent, transport agent, or hybridization-triggered cleavage agent.

Oligonucleotides of the invention may be synthesized by standard methods known in the art, e.g., by use of an automated DNA synthesizer (such as are commercially available from Biosearch, Applied Biosystems, etc.). As examples, phosphorothioate oligonucleotides may be synthesized by the method of Stein et al. (1988, Nucl. Acids Res. 16:3209), and methylphosphonate oligonucleotides can be prepared by use of controlled pore glass polymer supports (Sarin et al., 1988, Proc. Natl. Acad. Sci. USA 85:7448-7451).

In another embodiment, the ADPI antisense nucleic acid of the invention is produced intracellularly by transcription from an exogenous sequence. For example, a vector can be introduced in vivo such that it is taken up by a cell, within which cell the vector or a portion thereof is transcribed, producing an antisense nucleic acid (RNA) of the invention. Such a vector would contain a sequence encoding the ADPI antisense nucleic acid. Such a vector can remain episomal or become chromosomally integrated, as long as it can be transcribed to produce the desired antisense RNA. Such vectors can be constructed by recombinant DNA technology standard in the art. Vectors can be plasmid, viral, or others known in the art, used for replication and expression in mammalian cells. Expression of the sequence encoding the ADPI antisense RNA can be by any promoter known in the art to act in mammalian, preferably human, cells. Such promoters can be inducible or constitutive. Examples of such promoters are outlined above.

The antisense nucleic acids of the invention comprise a sequence complementary to at least a portion of an RNA transcript of a gene encoding a ADPI, preferably a human gene encoding a ADPI. However, absolute complementarity, although preferred, is not required. A sequence “complementary to at least a portion of an RNA,” as referred to herein, means a sequence having sufficient complementarity to be able to hybridise under stringent conditions (e.g., highly stringent conditions comprising hybridisation in 7% sodium dodecyl sulfate (SDS), 1 mM EDTA at 65° C. and washing in 0.1×SSC/0.1% SDS at 68° C., or moderately stringent conditions comprising washing in 0.2×SSC/0.1% SDS at 42° C.) with the RNA, forming a stable duplex; in the case of double-stranded ADPI antisense nucleic acids, a single strand of the duplex DNA may thus be tested, or triplex formation may be assayed. The ability to hybridise will depend on both the degree of complementarity and the length of the antisense nucleic acid. Generally, the longer the hybridising nucleic acid, the more base mismatches with an RNA encoding a ADPI it may contain and still form a stable duplex (or triplex, as the case may be). One skilled in the art can ascertain a tolerable degree of mismatch by use of standard procedures to determine the melting point of the hybridised complex.

5.15.6 Therapeutic Use of ADPI Antisense Nucleic Acids

The ADPI antisense nucleic acids can be used to treat or prevent Alzheimer's disease when the target ADPI is overexpressed in the brain tissue of subjects suspected of having or suffering from Alzheimer's disease. In a preferred embodiment, a single-stranded DNA antisense ADPI oligonucleotide is used.

Cell types which express or over-express RNA encoding a ADPI can be identified by various methods known in the art. Such cell types include but are not limited to leukocytes (e.g., neutrophils, macrophages, monocytes) and resident cells (e.g., astrocytes, glial cells, neuronal cells, and ependymal cells). Such methods include, but are not limited to, hybridisation with a ADPI-specific nucleic acid (e.g., by Northern hybridisation, dot blot hybridisation, in situ hybridisation), observing the ability of RNA from the cell type to be translated in vitro into a ADPI, immunoassay, etc. In a preferred aspect, primary tissue from a subject can be assayed for ADPI expression prior to treatment, e.g., by immunocytochemistry or in situ hybridisation.

Pharmaceutical compositions of the invention, comprising an effective amount of a ADPI antisense nucleic acid in a pharmaceutically acceptable carrier, vehicle or diluent can be administered to a subject having Alzheimer's disease.

The amount of ADPI antisense nucleic acid which will be effective in the treatment of Alzheimer's disease can be determined by standard clinical techniques.

In a specific embodiment, pharmaceutical compositions comprising one or more ADPI antisense nucleic acids are administered via liposomes, microparticles, or microcapsules. In various embodiments of the invention, such compositions may be used to achieve sustained release of the ADPI antisense nucleic acids.

5.15.7 Inhibitory Ribozyme and Triple Helix Approaches

In another embodiment, symptoms of Alzheimer's disease may be ameliorated by decreasing the level of a ADPI or ADPI activity by using gene sequences encoding the ADPI in conjunction with well-known gene “knock-out,” ribozyme or triple helix methods to decrease gene expression of a ADPI. In this approach ribozyme or triple helix molecules are used to modulate the activity, expression or synthesis of the gene encoding the ADPI, and thus to ameliorate the symptoms of Alzheimer's disease. Such molecules may be designed to reduce or inhibit expression of a mutant or non-mutant target gene. Techniques for the production and use of such molecules are well known to those of skill in the art.

Ribozyme molecules designed to catalytically cleave gene mRNA transcripts encoding a ADPI can be used to prevent translation of target gene mRNA and, therefore, expression of the gene product. (See, e.g., PCT International Publication WO90/11364, published Oct. 4, 1990; Sarver et al., 1990, Science 247:1222-1225).

Ribozymes are enzymatic RNA molecules capable of catalyzing the specific cleavage of RNA. (For a review, see Rossi, 1994, Current Biology 4, 469-471). The mechanism of ribozyme action involves sequence specific hybridisation of the ribozyme molecule to complementary target RNA, followed by an endonucleolytic cleavage event. The composition of ribozyme molecules must include one or more sequences complementary to the target gene mRNA, and must include the well known catalytic sequence responsible for mRNA cleavage. For this sequence, see, e.g., U.S. Pat. No. 5,093,246, which is incorporated herein by reference in its entirety.

While ribozymes that cleave mRNA at site specific recognition sequences can be used to destroy mRNAs encoding a ADPI, the use of hammerhead ribozymes is preferred. Hammerhead ribozymes cleave mRNAs at locations dictated by flanking regions that form complementary base pairs with the target mRNA. The sole requirement is that the target mRNA have the following sequence of two bases: 5′-UG-3′. The construction and production of hammerhead ribozymes is well known in the art and is described more fully in Myers, 1995, Molecular Biology and Biotechnology: A Comprehensive Desk Reference, VCH Publishers, New York, (see especially FIG. 4, page 833) and in Haseloff and Gerlach, 1988, Nature, 334, 585-591, each of which is incorporated herein by reference in its entirety.

Preferably the ribozyme is engineered so that the cleavage recognition site is located near the 5′ end of the mRNA encoding the ADPI, i.e., to increase efficiency and minimize the intracellular accumulation of non-functional mRNA transcripts.

The ribozymes of the present invention also include RNA endoribonucleases (hereinafter “Cech-type ribozymes”) such as the one that occurs naturally in Tetrahymena thermophila (known as the IVS, or L-19 IVS RNA) and that has been extensively described by Thomas Cech and collaborators (Zaug, et al., 1984, Science, 224, 574-578; Zaug and Cech, 1986, Science, 231, 470-475; Zaug, et al., 1986, Nature, 324, 429-433; published International patent application No. WO 88/04300 by University Patents Inc.; Been and Cech, 1986, Cell, 47, 207-216). The Cech-type ribozymes have an eight base pair active site which hybridizes to a target RNA sequence whereafter cleavage of the target RNA takes place. The invention encompasses those Cech-type ribozymes which target eight base-pair active site sequences that are present in the gene encoding the ADPI.

As in the antisense approach, the ribozymes can be composed of modified oligonucleotides (e.g., for improved stability, targeting, etc.) and should be delivered to cells that express the ADPI in vivo. A preferred method of delivery involves using a DNA construct “encoding” the ribozyme under the control of a strong constitutive pol III or pol II promoter, so that transfected cells will produce sufficient quantities of the ribozyme to destroy endogenous mRNA encoding the ADPI and inhibit translation. Because ribozymes, unlike antisense molecules, are catalytic, a lower intracellular concentration is required for efficacy.

Endogenous ADPI expression can also be reduced by inactivating or “knocking out” the gene encoding the ADPI, or the promoter of such a gene, using targeted homologous recombination (e.g., see Smithies, et al., 1985, Nature 317:230-234; Thomas and Capecchi, 1987, Cell 51:503-512; Thompson et al., 1989, Cell 5:313-321; and Zijlstra et al., 1989, Nature 342:435-438, each of which is incorporated by reference herein in its entirety). For example, a mutant gene encoding a non-functional ADPI (or a completely unrelated DNA sequence) flanked by DNA homologous to the endogenous gene (either the coding regions or regulatory regions of the gene encoding the ADPI) can be used, with or without a selectable marker and/or a negative selectable marker, to transfect cells that express the target gene in vivo. Insertion of the DNA construct, via targeted homologous recombination, results in inactivation of the target gene. Such approaches are particularly suited in the agricultural field where modifications to ES (embryonic stem) cells can be used to generate animal offspring with an inactive target gene (e.g., see Thomas and Capecchi, 1987 and Thompson, 1989, supra). However, this approach can be adapted for use in humans provided the recombinant DNA constructs are directly administered or targeted to the required site in vivo using appropriate viral vectors.

Alternatively, the endogenous expression of a gene encoding a ADPI can be reduced by targeting deoxyribonucleotide sequences complementary to the regulatory region of the gene (i.e., the gene promoter and/or enhancers) to form triple helical structures that prevent transcription of the gene encoding the ADPI in target cells in the body. (See generally, Helene, 1991, Anticancer Drug Des., 6(6), 569-584; Helene, et al., 1992, Ann. N.Y. Acad. Sci., 660, 27-36; and Maher, 1992, Bioassays 14(12), 807-815).

Nucleic acid molecules to be used in triplex helix formation for the inhibition of transcription in the present invention should be single stranded and composed of deoxynucleotides. The base composition of these oligonucleotides must be designed to promote triple helix formation via Hoogsteen base pairing rules, which generally require sizeable stretches of either purines or pyrimidines to be present on one strand of a duplex. Nucleotide sequences may be pyrimidine-based, which will result in TAT and CGC+ triplets across the three associated strands of the resulting triple helix. The pyrimidine-rich molecules provide base complementarity to a purine-rich region of a single strand of the duplex in a parallel orientation to that strand. In addition, nucleic acid molecules may be chosen that are purine-rich, for example, contain a stretch of G residues. These molecules will form a triple helix with a DNA duplex that is rich in GC pairs, in which the majority of the purine residues are located on a single strand of the targeted duplex, resulting in GGC triplets across the three strands in the triplex.

Alternatively, the potential sequences that can be targeted for triple helix formation may be increased by creating a so called “switchback” nucleic acid molecule. Switchback molecules are synthesized in an alternating 5′-3′, 3′-5′ manner, such that they base pair with first one strand of a duplex and then the other, eliminating the necessity for a sizeable stretch of either purines or pyrimidines to be present on one strand of a duplex.

In one embodiment, wherein the antisense, ribozyme, or triple helix molecules described herein are utilized to inhibit mutant gene expression, it is possible that the technique may so efficiently reduce or inhibit the transcription (triple helix) or translation (antisense, ribozyme) of mRNA produced by normal gene alleles of a ADPI that the situation may arise wherein the concentration of ADPI present may be lower than is necessary for a normal phenotype. In such cases, to ensure that substantially normal levels of activity of a gene encoding a ADPI are maintained, gene therapy may be used to introduce into cells nucleic acid molecules that encode and express the ADPI that exhibit normal gene activity and that do not contain sequences susceptible to whatever antisense, ribozyme, or triple helix treatments are being utilized. Alternatively, in instances whereby the gene encodes an extracellular protein, normal ADPI can be co-administered in order to maintain the requisite level of ADPI activity.

Antisense RNA and DNA, ribozyme, and triple helix molecules of the invention may be prepared by any method known in the art for the synthesis of DNA and RNA molecules, as discussed above. These include techniques for chemically synthesizing oligodeoxyribonucleotides and oligoribonucleotides well known in the art such as for example solid phase phosphoramidite chemical synthesis. Alternatively, RNA molecules may be generated by in vitro and in vivo transcription of DNA sequences encoding the antisense RNA molecule. Such DNA sequences may be incorporated into a wide variety of vectors that incorporate suitable RNA polymerase promoters such as the T7 or SP6 polymerase promoters. Alternatively, antisense cDNA constructs that synthesize antisense RNA constitutively or inducibly, depending on the promoter used, can be introduced stably into cell lines.

5.16 Assays for Therapeutic or Prophylactic Compounds

The present invention also provides assays for use in discovery of pharmaceutical products in order to identify or verify the efficacy of compounds for treatment or prevention of Alzheimer's disease. Agents can be assayed for their ability to restore ADF or ADPI levels in a subject having Alzheimer's disease towards levels found in subjects free from Alzheimer's disease or to produce similar changes in experimental animal models of Alzheimer's disease. Compounds able to restore ADF or ADPI levels in a subject having Alzheimer's disease towards levels found in subjects free from Alzheimer's disease or to produce similar changes in experimental animal models of Alzheimer's disease can be used as lead compounds for further drug discovery, or used therapeutically. ADF and ADPI expression can be assayed by the Preferred Technology, immunoassays, gel electrophoresis followed by visualization, detection of ADPI activity, or any other method taught herein or known to those skilled in the art. Such assays can be used to screen candidate drugs, in clinical monitoring or in drug development, where abundance of an ADF or ADPI can serve as a surrogate marker for clinical disease.

In various embodiments, in vitro assays can be carried out with cells representative of cell types involved in a subject's disorder, to determine if a compound has a desired effect upon such cell types.

Compounds for use in therapy can be tested in suitable animal model systems prior to testing in humans, including but not limited to rats, mice, chicken, cows, monkeys, rabbits, etc. For in vivo testing, prior to administration to humans, any animal model system known in the art may be used. It is also apparent to the skilled artisan that, based upon the present disclosure, transgenic animals can be produced with “knock-out” mutations of the gene or genes encoding one or more ADPIs. A “knock-out” mutation of a gene is a mutation that causes the mutated gene to not be expressed, or expressed in an aberrant form or at a low level, such that the activity associated with the gene product is nearly or entirely absent. Preferably, the transgenic animal is a mammal, more preferably, the transgenic animal is a mouse.

In one embodiment, test compounds that modulate the expression of a ADPI are identified in non-human animals (e.g., mice, rats, monkeys, rabbits, and guinea pigs), preferably non-human animal models for Alzheimer's disease, expressing the ADPI. In accordance with this embodiment, a test compound or a control compound is administered to the animals, and the effect of the test compound on expression of one or more ADPIs is determined. A test compound that alters the expression of a ADPI (or a plurality of ADPIs) can be identified by comparing the level of the selected ADPI or ADPIs (or mRNA(s) encoding the same) in an animal or group of animals treated with a test compound with the level of the ADPI(s) or mRNA(s) in an animal or group of animals treated with a control compound. Techniques known to those of skill in the art can be used to determine the mRNA and protein levels, for example, in situ hybridisation. The animals may or may not be sacrificed to assay the effects of a test compound.

In another embodiment, test compounds that modulate the activity of an ADPI or a biologically active portion thereof are identified in non-human animals (e.g., mice, rats, monkeys, rabbits, and guinea pigs), preferably non-human animal models for Alzheimer's disease, expressing the ADPI. In accordance with this embodiment, a test compound or a control compound is administered to the animals, and the effect of a test compound on the activity of an ADPI is determined. A test compound that alters the activity of an ADPI (or a plurality of ADPIs) can be identified by assaying animals treated with a control compound and animals treated with the test compound. The activity of the ADPI can be assessed by detecting induction of a cellular second messenger of the ADPI (e.g., intracellular Ca2+, diacylglycerol, IP3, etc.), detecting catalytic or enzymatic activity of the ADPI or binding partner thereof, detecting the induction of a reporter gene (e.g., a regulatory element that is responsive to a ADPI of the invention operably linked to a nucleic acid encoding a detectable marker, such as luciferase or green fluorescent protein), or detecting a cellular response (e.g., cellular differentiation or cell proliferation). Techniques known to those of skill in the art can be utilized to detect changes in the activity of a ADPI (see, e.g., U.S. Pat. No. 5,401,639, which is incorporated herein in its entirety by reference).

In yet another embodiment, test compounds that modulate the level or expression of a ADPI (or plurality of ADPIs) are identified in human subjects having Alzheimer's disease, most preferably those having severe Alzheimer's disease. In accordance with this embodiment, a test compound or a control compound is administered to the human subject, and the effect of a test compound on ADPI expression is determined by analysing the expression of the ADPI or the mRNA encoding the same in a biological sample (e.g., brain tissue, cerebrospinal fluid, serum, plasma, or urine). A test compound that alters the expression of a ADPI can be identified by comparing the level of the ADPI or mRNA encoding the same in a subject or group of subjects treated with a control compound to that in a subject or group of subjects treated with a test compound. Alternatively, alterations in the expression of a ADPI can be identified by comparing the level of the ADPI or mRNA encoding the same in a subject or group of subjects before and after the administration of a test compound. Any suitable techniques known to those of skill in the art can be used to obtain the biological sample and analyse the mRNA or protein expression. For example, the Preferred Technology described herein can be used to assess changes in the level of a ADPI.

In another embodiment, test compounds that modulate the activity of a ADPI (or plurality of ADPIs) are identified in human subjects having Alzheimer's disease, most preferably those with severe Alzheimer's disease. In this embodiment, a test compound or a control compound is administered to the human subject, and the effect of a test compound on the activity of an ADPI is determined. A test compound that alters the activity of an ADPI can be identified by comparing biological samples from subjects treated with a control compound to samples from subjects treated with the test compound. Alternatively, alterations in the activity of an ADPI can be identified by comparing the activity of an ADPI in a subject or group of subjects before and after the administration of a test compound. The activity of the ADPI can be assessed by detecting in a biological sample (e.g., brain tissue, cerebrospinal fluid, serum, plasma, or urine) induction of a cellular signal transduction pathway of the ADPI (e.g., intracellular Ca2+, diacylglycerol, IP3, etc.), catalytic or enzymatic activity of the ADPI or a binding partner thereof, or a cellular response, for example, cellular differentiation, or cell proliferation. Techniques known to those of skill in the art can be used to detect changes in the induction of a second messenger of an ADPI or changes in a cellular response. For example, RT-PCR can be used to detect changes in the induction of a cellular second messenger.

In a particular embodiment, an agent that changes the level or expression of an ADPI towards levels detected in control subjects (e.g., humans free from Alzheimer's disease) is selected for further testing or therapeutic use. In another preferred embodiment, a test compound that changes the activity of an ADPI towards the activity found in control subjects (e.g., humans free from Alzheimer's disease) is selected for further testing or therapeutic use.

In another embodiment, test compounds that reduce the severity of one or more symptoms associated with Alzheimer's disease are identified in human subjects having Alzheimer's disease, most preferably subjects with severe Alzheimer's disease. In accordance with this embodiment, a test compound or a control compound is administered to the subjects, and the effect of a test compound on one or more symptoms of Alzheimer's disease is determined. A test compound that reduces one or more symptoms can be identified by comparing the subjects treated with a control compound to the subjects treated with the test compound. Techniques known to physicians familiar with Alzheimer's disease can be used to determine whether a test compound reduces one or more symptoms associated with Alzheimer's disease. For example, a test compound that improves cognitive ability in a subject having Alzheimer's disease will be beneficial for treating subjects having Alzheimer's disease.

In a preferred embodiment, an agent that reduces the severity of one or more symptoms associated with Alzheimer's disease in a human having Alzheimer's disease is selected for further testing or therapeutic use.

5.17 Therapeutic and Prophylactic Compositions and Their Use

The invention provides methods of treatment comprising administering to a subject an effective amount of an agent of the invention. In a preferred aspect, the compound is substantially purified (e.g., substantially free from substances that limit its effect or produce undesired side-effects). The subject is preferably an animal, including but not limited to animals such as cows, pigs, horses, chickens, cats, dogs, etc., and is preferably a mammal, and most preferably human. In a specific embodiment, a non-human mammal is the subject.

Formulations and methods of administration that can be employed when the compound comprises a nucleic acid are described above; additional appropriate formulations and routes of administration are described below.

Various delivery systems are known and can be used to administer a compound of the invention, e.g., encapsulation in liposomes, microparticles, microcapsules, recombinant cells capable of expressing the compound, receptor-mediated endocytosis (see, e.g., Wu and Wu, 1987, J. Biol. Chem. 262:4429-4432), construction of a nucleic acid as part of a retroviral or other vector, etc. Methods of introduction can be enteral or parenteral and include but are not limited to intradermal, intramuscular, intraperitoneal, intravenous, subcutaneous, intranasal, epidural, and oral routes. The compounds may be administered by any convenient route, for example by infusion or bolus injection, by absorption through epithelial or mucocutaneous linings (e.g., oral mucosa, rectal and intestinal mucosa, etc.) and may be administered together with other biologically active agents. Administration can be systemic or local. In addition, it may be desirable to introduce the pharmaceutical compositions of the invention into the central nervous system by any suitable route, including intraventricular and intrathecal injection; intraventricular injection may be facilitated by an intraventricular catheter, for example, attached to a reservoir, such as an Ommaya reservoir. Pulmonary administration can also be employed, e.g., by use of an inhaler or nebulizer, and formulation with an aerosolizing agent.

In a specific embodiment, it may be desirable to administer the pharmaceutical compositions of the invention locally to the area in need of treatment; this may be achieved, for example, and not by way of limitation, by local infusion during surgery, topical application, e.g., by injection, by means of a catheter, or by means of an implant, said implant being of a porous, non-porous, or gelatinous material, including membranes, such as sialastic membranes, or fibers. In one embodiment, administration can be by direct injection into direct injection into cerebrospinal fluid or at the site (or former site) of neurodegeneration or to CNS tissue.

In another embodiment, the compound can be delivered in a vesicle, in particular a liposome (see Langer, 1990, Science 249:1527-1533; Treat et al., in Liposomes in the Therapy of Infectious Disease and Cancer, Lopez-Berestein and Fidler (eds.), Liss, New York, pp. 353-365 (1989); Lopez-Berestein, ibid., pp. 317-327; see generally ibid.)

In yet another embodiment, the compound can be delivered in a controlled release system. In one embodiment, a pump may be used (see Langer, supra; Sefton, 1987, CRC Crit. Ref. Biomed. Eng. 14:201; Buchwald et al., 1980, Surgery 88:507; Saudek et al., 1989, N. Engl. J. Med. 321:574). In another embodiment, polymeric materials can be used (see Medical Applications of Controlled Release, Langer and Wise (eds.), CRC Pres., Boca Raton, Fla. (1974); Controlled Drug Bioavailability, Drug Product Design and Performance, Smolen and Ball (eds.), Wiley, New York (1984); Ranger and Peppas, J., 1983, Macromol. Sci. Rev. Macromol. Chem. 23:61; see also Levy et al., 1985, Science 228:190; During et al., 1989, Ann. Neurol. 25:351; Howard et al., 1989, J. Neurosurg. 71:105). In yet another embodiment, a controlled release system can be placed in proximity of the therapeutic target, i.e., the brain, thus requiring only a fraction of the systemic dose (see, e.g., Goodson, in Medical Applications of Controlled Release, supra, vol. 2, pp. 115-138 (1984)).

Other suitable controlled release systems are discussed in the review by Langer (1990, Science 249:1527-1533).

In another embodiment where the compound of the invention is a nucleic acid encoding a protein, the nucleic acid can be administered in vivo to promote expression of its encoded protein, by constructing it as part of an appropriate nucleic acid expression vector and administering it so that it becomes intracellular, e.g., by use of a retroviral vector (see U.S. Pat. No. 4,980,286), or by direct injection, or by use of microparticle bombardment (e.g., a gene gun; Biolistic, Dupont), or coating with lipids or cell-surface receptors or transfecting agents, or by administering it in linkage to a homeobox-like peptide which is known to enter the nucleus (see e.g., Joliot et al., 1991, Proc. Natl. Acad. Sci. USA 88:1864-1868), etc. Alternatively, a nucleic acid can be introduced intracellularly and incorporated within host cell DNA for expression, by homologous recombination.

The present invention also provides pharmaceutical compositions. Such compositions comprise a therapeutically effective amount of an agent, and a pharmaceutically acceptable carrier. In a particular embodiment, the term “pharmaceutically acceptable” means approved by a regulatory agency of the Federal or a state government or listed in the U.S. Pharmacopeia or other generally recognized pharmacopeia for use in animals, and more particularly in humans. The term “carrier” refers to a diluent, adjuvant, excipient, or vehicle with which the therapeutic is administered. Such pharmaceutical carriers can be sterile liquids, such as water and oils, including those of petroleum, animal, vegetable or synthetic origin, such as peanut oil, soybean oil, mineral oil, sesame oil and the like. Water is a preferred carrier when the pharmaceutical composition is administered intravenously. Saline solutions and aqueous dextrose and glycerol solutions can also be employed as liquid carriers, particularly for injectable solutions. Suitable pharmaceutical excipients include starch, glucose, lactose, sucrose, gelatin, malt, rice, flour, chalk, silica gel, sodium stearate, glycerol monostearate, talc, sodium chloride, dried skim milk, glycerol, propylene, glycol, water, ethanol and the like. The composition, if desired, can also contain minor amounts of wetting or emulsifying agents, or pH buffering agents. These compositions can take the form of solutions, suspensions, emulsion, tablets, pills, capsules, powders, sustained-release formulations and the like. The composition can be formulated as a suppository, with traditional binders and carriers such as triglycerides. Oral formulation can include standard carriers such as pharmaceutical grades of mannitol, lactose, starch, magnesium stearate, sodium saccharine, cellulose, magnesium carbonate, etc. Examples of suitable pharmaceutical carriers are described in “Remington's Pharmaceutical Sciences” by E. W. Martin. Such compositions will contain a therapeutically effective amount of the compound, preferably in purified form, together with a suitable amount of carrier so as to provide the form for proper administration to the subject. The formulation should suit the mode of administration.

In a preferred embodiment, the composition is formulated in accordance with routine procedures as a pharmaceutical composition adapted for intravenous administration to human beings. Typically, compositions for intravenous administration are solutions in sterile isotonic aqueous buffer. Where necessary, the composition may also include a solubilizing agent and a local anaesthetic such as lidocaine to ease pain at the site of the injection. Generally, the ingredients are supplied either separately or mixed together in unit dosage form, for example, as a dry lyophilized powder or water free concentrate in a hermetically sealed container such as an ampoule or sachette indicating the quantity of active agent. Where the composition is to be administered by infusion, it can be dispensed with an infusion bottle containing sterile pharmaceutical grade water or saline. Where the composition is administered by injection, an ampoule of sterile water for injection or saline can be provided so that the ingredients may be mixed prior to administration.

The compounds of the invention can be formulated as neutral or salt forms. Pharmaceutically acceptable salts include those formed with free amino groups such as those derived from hydrochloric, phosphoric, acetic, oxalic, tartaric acids, etc., and those formed with free carboxyl groups such as those derived from sodium, potassium, ammonium, calcium, ferric hydroxides, isopropylamine, triethylamine, 2-ethylamino ethanol, histidine, procaine, etc.

The amount of the compound of the invention which will be effective in the treatment of Alzheimer's disease can be determined by standard clinical techniques based on the present description. In addition, in vitro assays may optionally be employed to help identify optimal dosage ranges. The precise dose to be employed in the formulation will also depend on the route of administration, and the seriousness of the disease or disorder, and should be decided according to the judgment of the practitioner and each subject's circumstances. However, suitable dosage ranges for intravenous administration are generally about 20-500 micrograms of active compound per kilogram body weight. Suitable dosage ranges for intranasal administration are generally about 0.01 pg/kg body weight to 1 mg/kg body weight. Effective doses may be extrapolated from dose-response curves derived from in vitro or animal model test systems.

Suppositories generally contain active ingredient in the range of 0.5% to 10% by weight; oral formulations preferably contain 10% to 95% active ingredient.

The invention also provides a pharmaceutical pack or kit comprising one or more containers filled with one or more of the ingredients of the pharmaceutical compositions of the invention. Optionally associated with such container(s) can be a notice in the form prescribed by a governmental agency regulating the manufacture, use or sale of pharmaceuticals or biological products, which notice reflects (a) approval by the agency of manufacture, use or sale for human administration, (b) directions for use, or both.

The invention will now be described further with reference to the following non-limited examples.

### 6. EXAMPLE 1

### Identification of Proteins Differentially Expressed in the Brain Tissue of Alzheimer's Disease Patients

Using the following exemplary and non-limiting procedure, proteins from a total of 37 brain tissue samples from subjects having Alzheimer's disease and 39 brain tissue samples from control subjects were separated by isoelectric focusing followed by SDS-PAGE and analysed, see table IX for the details of patient numbers for each anatomical region studied. Parts 6.1.1 to 6.1.14 (inclusive) of the procedure set forth below are hereby designated as the “Reference Protocol” and are presented for purposes of setting forth the standard procedures that the artisan may follow in the practice of the invention.

6.1 Materials and Methods

6.1.1 Sample Preparation

Tissue samples used in this study were taken post mortem from anatomically defined regions including hippocampus, entorhinal cortex, frontal cortex, neocortex and amygdala, from subjects having Alzheimer's disease and control subjects. Samples were selected with the minimum possible post-mortem interval (PMI) and similar age of the control and Alzheimer's patients as summarised below. ps Table IX. Summary of samples used.

TABLE IX
Summary of samples used.

| | Alzheimer's disease | Control |
|---|---|---|
| Anatomical region | patients | subjects |
| All Hippocampus | 18 | 20 |
| Selected Hippocampus | 9 | 11 |
| (post-mortem delay of less than 4h) | | |
| Frontal Cortex | 10 | 10 |
| Neocortex | 3 | 3 |
| Entorhinal Cortex | 3 | 3 |
| Amygdala | 3 | 3 |

Samples were stored at minus 80° C. throughout. For cryostat sectioning, samples were mounted on cork disks with OCT. Sections of each block were stained with haematoxylin and eosin (H&E) and then evaluated by a pathologist to confirm the diagnosis and presence of characteristic features of Alzheimer's disease in the diseased samples. These included: neurofibrillary tangles in the cortical neurons and neuritic (amyloid) plaques. An approximate estimate on the percentage of target neurons (cortical vs pyramidal) remaining was carried out. The brain samples were mounted on cork discs and supported in OCT Embedding Matrix (CellPath plc, UK). Seven frozen sections approximately 5 um each were cut on a cryostat and were transferred directly to eppendorf tubes (1.5 ml) containing 200 ul of lysis buffer 8M urea (BDH 452043w), 4% CHAPS (Sigma C3023), 65 mM dithiotheitol (DTT), 2% (v/v) Resolytes 3.5-10 (BDH 44338 2x). Eppendorf tubes were vortexed for about 30 seconds, briefly centrifuged, frozen and stored at −70° C. Approximately 75-120 μg of total protein were taken forward to 2-dimensional gel electrophoresis.

6.1.2 Isoelectric Focusing

Isoelectric focusing (IEF), was performed using the Immobiline™ DryStrip Kit (Pharmacia BioTech), following the procedure described in the manufacturer's instructions, see Instructions for Immobiline™ DryStrip Kit, Pharmacia, 18-1038-63, Edition AB (incorporated herein by reference in its entirety). Immobilized pH Gradient (IPG) strips (18 cm, pH 3-10 non-linear strips; Pharmacia 17-1235-01) were rehydrated overnight at 20° C. with the 370 microl. of sample prepared in 6.1.1. The reswelled IPG strips were then tranferred to a Multiphor II Electrophoresis Unit (Pharmacia 18-1018-06), covered with mineral oil (Pharmacia 17-3335-01) and subjected to first dimension isoelectric focussing using a Pharmacia EPS3500XL power supply (19-3500-01) according to the following profile:

Initial voltage=300V for 2 hrs

Linear Ramp from 300V to 3500V over 3 hrs

Hold at 3500V for 19 hrs

For all stages of the process, the current limit was set to 10 mA for 12 gels, and the power limit to 5 W. The temperature was held constant at 20° C. throughout the run.

6.1.3 Gel Equilibration and SDS-PAGE

After the final 19 hr step, the strips were immediately removed and immersed for 10 mins at 20° C. in a solution of the following composition: 6M urea; 2% (w/v) DTT; 2% (w/v) SDS; 30% (v/v) glycerol (Fluka 49767); 0.05M Tris/HCl, pH 6.8 (Sigma T-1503). After removal from the solution, the strips were loaded onto supported gels for SDS-PAGE according to Hochstrasser et al., 1988, Analytical Biochemistry 173: 412-423 (incorporated herein by reference in its entirety), with modifications as specified below.

6.1.4 Preparation of Supported Gels

The gels were cast between two glass plates of the following dimensions: 23 cm wide×24 cm long (back plate); 23 cm wide×24 cm long with a 2 cm deep notch in the central 19 cm (front plate). To promote covalent attachment of SDS-PAGE gels, the back plate was treated with a 0.4% solution of γ-methacryl-oxypropyltrimethoxysilane in ethanol (BindSilane™; Pharmacia 17-1330-01). The front plate was treated with (RepelSilane™ Pharmacia 17-1332-01) to reduce adhesion of the gel. Excess reagent was removed by washing with water, and the plates were allowed to dry. At this stage, both as identification for the gel, and as a marker to identify the coated face of the plate, an adhesive bar-code was attached to the back plate in a position such that it would not come into contact with the gel matrix.

The dried plates were assembled into a casting box with a capacity of 13 gel sandwiches. The front and back plates of each sandwich were spaced by means of 1 mm thick spacers, 2.5 cm wide. The sandwiches were interleaved with acetate sheets to facilitate separation of the sandwiches after gel polymerisation. Casting was then carried out according to Hochstrasser et al., op. cit.

A 9-16% linear polyacrylamide gradient was cast, extending up to a point 2 cm below the level of the notch in the front plate, using the Angelique gradient casting system (Large Scale Biology). Stock solutions were as follows. Acrylamide (40% in water) was from Serva (10677). The cross-linking agent was PDA (BioRad 161-0202), at a concentration of 2.6% (w/w) of the total starting monomer content. The gel buffer was 0.375M Tris/HCl, pH 8.8. The polymerisation catalyst was 0.05% (v/v) TEMED (BioRad 161-0801), and the initiator was 0.1% (w/v) APS (BioRad 161-0700). No SDS was included in the gel and no stacking gel was used. The cast gels were allowed to polymerise at 20° C. overnight, and then stored at 4° C. in sealed polyethylene bags with 6 ml of gel buffer, and were used within 4 weeks.

6.1.5 SDS-PAGE

A solution of 0.5% (w/v) agarose (Fluka 05075) was prepared in running buffer (0.025M Tris, 0.192M glycine (Fluka 50050), 0.1% (w/v) SDS), supplemented also by a trace of bromophenol blue. The agarose suspension was heated to 70° C. with stirring, until the agarose had dissolved. The top of the supported 2nd D gel was filled with the agarose solution, and the equilibrated IPG strip was placed into the agarose, and tapped gently with a palette knife until the IPG strip was intimately in contact with the 2nd D gel. The gels were placed in the 2nd D running tank, as described by Amess et al., 1995, Electrophoresis 16: 1255-1267 (incorporated herein by reference in its entirety). The tank was filled with running buffer (as above) until the level of the buffer just exceeded the top of the slab gel, which promoted efficient cooling of the active gel area. Running buffer was added to the top buffer compartments formed by the gels, and then voltage was applied immediately to the gels using a Consort E-833 power supply. The gels were run at 10 mA/gel for 10 mins. The power limit was set to 150 W for a tank containing 6 gels, and the voltage limit was set to 600V. After 10 mins, the gels were then run at 30 mA/gel, with the same voltage and power limits as before, until the bromophenol blue line was 0.5 cm from the bottom of the gel. The temperature of the buffer was held constant at 16° C. throughout the run.

6.1.6 Staining

Upon completion of the electrophoresis run, the gels were immediately removed from the tank for fixation. The top plate of the gel cassette was carefully removed, leaving the gel bonded to the bottom plate. The bottom plate with its attached gel was then placed into a staining apparatus, which can accommodate 12 gels. The gels were completely immersed in fixative solution of 40% (v/v) ethanol (BDH 28719), 10% (v/v) acetic acid (BDH 100016X), 50% (v/v) water (MilliQ-Millipore), which was continuously circulated over the gels. After an overnight incubation at room temperature, the fixative solution was drained from the tank, and the gels were primed by immersion in 7.5% (v/v) acetic acid, 0.05% (w/v) SDS, 92.5% (v/v) water for 30 mins. The priming solution was then drained, and the gels were stained by complete immersion for 4 hours in a staining solution of Sypro Red (Molecular Probes, Inc., Eugene, Oreg.). Alternative dyes which can be used for this purpose are described in U.S. patent application Ser. No. 09/412168, filed Oct. 5, 1999, and incorporated herein by reference in its entirety.

6.1.7 Imaging of the Gel

A computer-readable output was produced by imaging the fluorescently stained gels with the Apollo 3 scanner (Oxford Glycosciences, Oxford, UK) described in section 5.2, supra. This scanner has a gel carrier with four integral fluorescent markers (Designated M1, M2, M3, M4) that are used to correct the image geometry and are a quality control feature to confirm that the scanning has been performed correctly.

For scanning, the gels were removed from the stain, rinsed with water and allowed to air dry briefly, before they were scanned. After imaging, the gels were sealed in polyethylene bags containing a small volume of staining solution, and then stored at 4° C.

6.1.8 Digital Analysis of the Data

The data were processed as described in U.S. Pat. No. 6,064,754, (published as WO 98/23950) at Sections 5.4 and 5.5 (incorporated herein by reference), as set forth more particularly below.

The output from the scanner was first processed using the MELANIE® II 2D PAGE analysis program (Release 2.2, 1997, BioRad Laboratories, Hercules, Calif., Cat. #170-7566) to autodetect the registration points, M1, M2, M3 and M4; to autocrop the images (i.e., to eliminate signals originating from areas of the scanned image lying outside the boundaries of the gel, e.g. the reference frame); to filter out artifacts due to dust; to detect and quantify features; and to create image files in GIF format. Features were detected using the following parameters:

Smooths=2

Laplacian threshold 50

Partials threshold 1

Saturation=100

Peakedness=0

Minimum Perimeter=10

6.1.9 Assignment of pI and MW Values

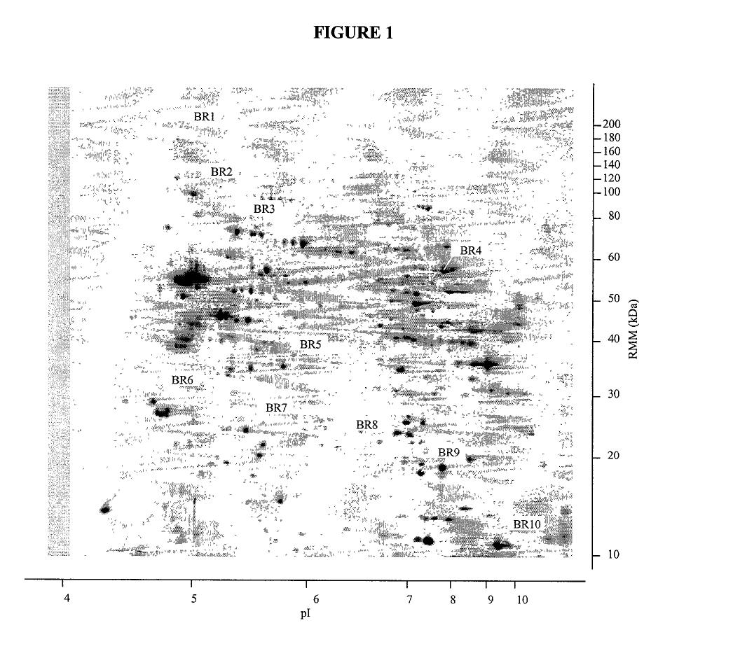

Landmark identification was used to determine the pI and MW of features detected in the images. Ten landmark features, designated BR1 to BR10, were identified in a standard brain tissue image. These landmark features are identified in FIG. 1 and were assigned the pI and/or MW values identified in Table X.

Table X. Landmark Features Used in this Study

TABLE X

| BR# | pI | MW (Da) |
|---|---|---|
| BR1 | 4.84 | 176396 |
| BR2 | 4.96 | 94107 |
| BR3 | 5.34 | 69139 |
| BR4 | 7.80 | 54509 |
| BR5 | 5.74 | 34187 |
| BR6 | 4.60 | 29905 |
| BR7 | 5.42 | 23589 |
| BR8 | 6.19 | 21003 |
| BR9 | 7.42 | 18398 |
| BR10 | 9.39 | 10912 |

As many of these landmarks as possible were identified in each gel image of the dataset. Each feature in the study gels was then assigned a pI value by linear interpolation or extrapolation (using the MELANIE®-II software) to the two nearest landmarks, and was assigned a MW value by linear interpolation or extrapolation (using the MELANIE®-II software) to the two nearest landmarks.

6.1.10 Matching With Primary Master Image

Images were edited to remove gross artefacts such as dust, to reject images which had gross abnormalities such as smearing of protein features, or were of too low a loading or overall image intensity to allow identification of more than the most intense features, or were of too poor a resolution to allow accurate detection of features. Images were then compared by pairing with one common image from the whole sample set. This common image, the “primary master image”, was selected on the basis of protein load (maximum load consistent with maximum feature detection), and general image quality. Additionally, the primary master image was chosen to be an image which appeared to be generally representative of all those to be included in the analysis. (This process by which a primary master gel was judged to be representative of the study gels was rechecked by the method described below and in the event that the primary master gel was seen to be unrepresentative, it was rejected and the process repeated until a representative primary master gel was found.)

Each of the remaining study gel images was individually matched to the primary master image such that common protein features were paired between the primary master image and each individual study gel image as described below.

6.1.11 Cross-matching Between Samples

To facilitate statistical analysis of large numbers of samples for purposes of identifying features that are differentially expressed, the geometry of each study gel was adjusted for maximum alignment between its pattern of protein features, and that of the primary master, as follows. Each of the study gel images was individually transformed into the geometry of the primary master image using a multi-resolution warping procedure. This procedure corrects the image geometry for the distortions brought about by small changes in the physical parameters of the electrophoresis separation process from one sample to another. The observed changes are such that the distortions found are not simple geometric distortions, but rather a smooth flow, with variations at both local and global scale.

The fundamental principle in multi-resolution modelling is that smooth signals may be modelled as an evolution through ‘scale space’, in which details at successively finer scales are added to a low resolution approximation to obtain the high resolution signal. This type of model is applied to the flow field of vectors (defined at each pixel position on the reference image) and allows flows of arbitrary smoothness to be modelled with relatively few degrees of freedom. Each image is first reduced to a stack, or pyramid, of images derived from the initial image, but smoothed and reduced in resolution by a factor of 2 in each direction at every level (Gaussian pyramid) and a corresponding difference image is also computed at each level, representing the difference between the smoothed image and its progenitor (Laplacian pyramid). Thus the Laplacian images represent the details in the image at different scales.

To estimate the distortion between any 2 given images, a calculation was performed at level 7 in the pyramid (i.e. after 7 successive reductions in resolution). The Laplacian images were segmented into a grid of 16×16 pixels, with 50% overlap between adjacent grid positions in both directions, and the cross correlation between corresponding grid squares on the reference and the test images was computed. The distortion displacement was then given by the location of the maximum in the correlation matrix. After all displacements had been calculated at a particular level, they were interpolated to the next level in the pyramid, applied to the test image, and then further corrections to the displacements were calculated at the next scale.

The warping process brought about good alignment between the common features in the primary master image, and the images for the other samples. The MELANIE® II 2D PAGE analysis program was used to calculate and record approximately 500-700 matched feature pairs between the primary master and each of the other images. The accuracy of this program was significantly enhanced by the alignment of the images in the manner described above. To improve accuracy still further, all pairings were finally examined by eye in the MelView interactive editing program and residual recognizably incorrect pairings were removed. Where the number of such recognizably incorrect pairings exceeded the overall reproducibility of the Preferred Technology (as measured by repeat analysis of the same biological sample) the gel selected to be the primary master gel was judged to be insufficiently representative of the study gels to serve as a primary master gel. In that case, the gel chosen as the primary master gel was rejected, and different gel was selected as the primary master gel, and the process was repeated.

All the images were then added together to create a composite master image, and the positions and shapes of all the gel features of all the component images were super-imposed onto this composite master as described below.

Once all the initial pairs had been computed, corrected and saved, a second pass was performed whereby the original (unwarped) images were transformed a second time to the geometry of the primary master, this time using a flow field computed by smooth interpolation of the multiple tie-points defined by the centroids of the paired gel features. A composite master image was thus generated by initializing the primary master image with its feature descriptors. As each image was transformed into the primary master geometry, it was digitally summed pixel by pixel into the composite master image, and the features that had not been paired by the procedure outlined above were likewise added to the composite master image description, with their centroids adjusted to the master geometry using the flow field correction.

The final stage of processing was applied to the composite master image and its feature descriptors, which now represent all the features from all the images in the study transformed to a common geometry. The features were grouped together into linked sets or “clusters”, according to the degree of overlap between them. Each cluster was then given a unique identifying index, the molecular cluster index (MCI).

An MCI identifies a set of matched features on different images. Thus an MCI represents a protein or proteins eluting at equivalent positions in the 2D separation in different samples.

6.1.12. Construction of Profiles

After matching all component gels in the study to the final composite master image, the intensity of each feature was measured and stored. The end result of this analysis was the generation of a digital profile which contained, for each identified feature: 1) a unique identification code relative to corresponding feature within the composite master image (MCI), 2) the x, y coordinates of the features within the gel, 3) the isoelectric point (pI) of the ADFs, 4) the apparent molecular weight (MW) of the ADFs, 5) the signal value, 6) the standard deviation for each of the preceding measurements, and 7) a method of linking the MCI of each feature to the master gel to which this feature was matched. By virtue of a Laboratory Information Management System (LIMS), this MCI profile was traceable to the actual stored gel from which it was generated, so that proteins identified by computer analysis of gel profile databases could be retrieved. The LIMS also permitted the profile to be traced back to an original sample or patient.

6.1.13. Statistical Analysis of the Profiles

The statistical strategies specified below were used in the order in which they are listed to identify ADFs from the MCIs within the mastergroup.

A percentage feature presence was calculated across the control samples and the Alzheimer's disease samples for each MCI that was a potential ADF. The MCI was required to be present in at least 50% of samples from Alzheimer's disease or in at least 50% of the samples from the control group. The MCIs which fulfilled these criteria were then subjected to further analysis

The percentage feature presence for each remaining MCI was then further examined and the MCIs were divided into 2 groups—those which had at least 50% feature presence in the Alzheimer's disease sample group but were absent from all samples in the control group (designated c+), those with at least 50% feature presence in the control sample group but which were absent from all samples in the Alzheimer's disease sample group (designated c−) and those MCIs which were present in both disease and control sample groups. The MCIs that were present in both sample groups were subjected to further analysis.

A second selection strategy for the MCIs remaining from (b) was based on the fold change. A fold change representing the ratio of the average normalized protein abundances of the ADFs within an MCI, was calculated for each MCI between each the neurological disorder samples and its age-matched set of controls. A minimum threshold of 1.5 was set for the fold change to be considered significant.

For the final selection strategy the Wilcoxon Rank-Sum test was used. This test was performed between the control and the Alzheimer's disease samples for each MCI with a fold change greater than 1.5. The MCIs which recorded a p-value less than or equal to 0.05 were selected as statistically significant ADFs with 95% selectivity. The MCIs with a statistically significant change (p<0.05) were designated “b” and the MCIs which did not reach statistical significance were designated “a”. For the latter two groups a “+” or “−” sign was used to indicate a fold increase or a fold decrease respectively.

Application of these four analysis strategies allowed ADFs to be selected on the basis of: (a) feature presence in at least 50% of samples from control subjects or patients with a neurological disorders (b) qualitative differences with a chosen selectivity, (c) a significant fold change above a threshold with a chosen selectivity or (d) statistically significant changes as measured by the Wilcoxon Rank-Sum test

6.1.14 Recovery and Analysis of Selected Proteins

Proteins in ADFs were robotically excised and processed to generate tryptic digest peptides. Tryptic peptides were analysed by mass spectrometry using a PerSeptive Biosystems Voyager- DETM STR Matrix-Assisted Laser Desorption Ionization Time-of-Flight (MALDI-TOF) mass spectrometer, and selected tryptic peptides were analysed by tandem mass spectrometry (MS/MS) using a Micromass Quadrupole Time-of-Flight (Q-TOF) mass spectrometer (Micromass, Altrincham, U.K.) equipped with a nanoflowTM electrospray Z-spray source. For partial amino acid sequencing and identification of ADPIs uninterpreted tandem mass spectra of tryptic peptides were searched using the SEQUEST search program (Eng et al., 1994, J. Am. Soc. Mass Spectrom. 5:976-989), version v.C.1. Criteria for database identification included: the cleavage specificity of trypsin; and the detection of a suite of a, b and y ions in peptides returned from the database. The database searched was a database constructed of protein entries in the non-redundant database held by the National Centre for Biotechnology Information (NCBI) which is accessible at http://www.ncbi.nlm.nih.gov/. Following identification of proteins through spectral-spectral correlation using the SEQLEST program, masses detected in MALDI-TOF mass spectra were assigned to tryptic digest peptides within the proteins identified. In cases where no amino acid sequences could be identified through searching with uninterpreted MS/MS spectra of tryptic digest peptides using the SEQUEST program, tandem mass spectra of the peptides were interpreted manually, using methods known in the art. (In the case of interpretation of low-energy fragmentation mass spectra of peptide ions see Gaskell et al., 1992, Rapid Commun. Mass Spectrom. 6:658-662). The method described in PCT Application No. PCT/GB 01/04034, which is incorporated herein by reference in its entirety, was also used to interpret mass spectra.

6.2 Results

These initial experiments identified 427 features that were altered in Alzheimer's disease brain tissue as compared with normal brain tissue. Details of these ADFs are provided in Tables I and II. Each ADF was differentially present in Alzheimer's disease brain tissue as compared with normal brain tissue. For some preferred ADFs (ADF-1, ADF-3, ADF-6, ADF-8, ADF-10, ADF-11, ADF-12, ADF-23, ADF-26, ADF-27, ADF-29, ADF-31, ADF-37, ADF-42, ADF-54, ADF-56, ADF-67, ADF-77, ADF-79, ADF-85, ADF-90, ADF-91, ADF-97, ADF-102, ADF-103, ADF-119, ADF-120, ADF-121, ADF-129, ADF-131, ADF-132, ADF-139, ADF-144, ADF-148, ADF-150, ADF-152, ADF-154, ADF-155, ADF-157, ADF-159, ADF-160, ADF-162, ADF-163, ADF-165, ADF-172, ADF-173, ADF-176, ADF-183, ADF-191, ADF-193, ADF-194, ADF-202, ADF-204, ADF-209, ADF-223, ADF-232, ADF-237, ADF-250, ADF-262, ADF-266, ADF-268, ADF-270, ADF-273, ADF-275, ADF-276, ADF-277, ADF-280, ADF-282, ADF-283, ADF-284, ADF-286, ADF-288, ADF-293, ADF-295, ADF-297, ADF-303, ADF-304, ADF-305, ADF-318, ADF-319, ADF-321, ADF-324, ADF-325, ADF-328, ADF-331, ADF-332, ADF-335, ADF-337, ADF-338, ADF-342, ADF-346, ADF-351, ADF-358, ADF-362, ADF-364, ADF-366, ADF-367, ADF-368, ADF-370, ADF-373, ADF-374, ADF-376, ADF-382, ADF-387, ADF-389, ADF-395, ADF-396, ADF-398, ADF-399, ADF-400, ADF-404, ADF-405, ADF-406, ADF-410, ADF-411, ADF-415, ADF-419, ADF-423, ADF-425, ADF-429, ADF-430, ADF-431, ADF-434, ADF-437, ADF-439, ADF-440, ADF-441, ADF-446, ADF-447, ADF-449, ADF-450, ADF-452, ADF-456, ADF-461, ADF-463, ADF-467, ADF-468, ADF-469, ADF-472, ADF-473, ADF-474, ADF-477, ADF-478, ADF-479, ADF-480, ADF-491, ADF-492, ADF-493, ADF-495, ADF-501, ADF-503, ADF-504, ADF-505, ADF-506, ADF-507, ADF-510, ADF-514, ADF-515, ADF-518, ADF-520, ADF-524), the difference was highly significant (p<0.01), and for certain highly preferred ADFs (ADF-6, ADF-10, ADF-11, ADF-27, ADF-31, ADF-37, ADF-102, ADF-120, ADF-121, ADF-129, ADF-131, ADF-148, ADF-152, ADF-159, ADF-162, ADF-165, ADF-172, ADF-176, ADF-183, ADF-191, ADF-202, ADF-204, ADF-282, ADF-303, ADF-304, ADF-305, ADF-321, ADF-325, ADF-328, ADF-332, ADF-335, ADF-338, ADF-346, ADF-358, ADF-364, ADF-366, ADF-370, ADF-373, ADF-376, ADF-387, ADF-389, ADF-411, ADF-423, ADF-425, ADF-431, ADF-439, ADF-440, ADF-450, ADF-452, ADF-463, ADF-472, ADF-480, ADF-495, ADF-503, ADF-505, ADF-506, ADF-507, ADF-510, ADF-515, ADF-518, ADF-520), the difference was still more significant (p<0.001).

6.2.1 Hippocampus

6.2.1.1 All Hippocampal Samples

These initial experiments identified 132 features that were altered in the whole set of hippocampal Alzheimer's disease samples as compared with control hippocampal samples.

Details of these ADFs are provided in Table I. Each ADF was differentially present in Alzheimer's disease tissue as compared with control tissue. For some preferred ADFs, listed in Table XI(a), the fold change was greater than 1.5, and for certain highly preferred ADFs, listed in Table XI(b), the difference was significant, p<0.05.

Table XI (a). ADFs altered in Alzheimer's disease hippocampus,

TABLE XI (a)

| ADF# | pI | MW (Da) | Fold Change |
|---|---|---|---|
| ADF-305 | 4.6 | 32146 | −2.767 |
| ADF-515 | 7.52 | 49586 | −2.364 |
| ADF-427 | 8.66 | 50180 | −2.150 |
| ADF-256 | 6.91 | 136744 | −2.015 |
| ADF-300 | 9.93 | 33122 | −1.973 |
| ADF-332 | 10.02 | 21013 | −1.918 |
| ADF-255 | 6.77 | 138133 | −1.853 |
| ADF-81 | 4.83 | 42229 | −1.837 |
| ADF-483 | 7.81 | 50189 | −1.752 |
| ADF-33 | 9.07 | 42881 | −1.676 |
| ADF-297 | 5.03 | 33778 | −1.635 |
| ADF-384 | 9.43 | 16119 | −1.614 |
| ADF-16 | 5.57 | 48424 | −1.604 |
| ADF-413 | 7.59 | 50213 | −1.602 |
| ADF-401 | 6.27 | 51636 | −1.590 |
| ADF-342 | 7.51 | 14929 | −1.575 |
| ADF-240 | 9.56 | 32166 | −1.574 |
| ADF-462 | 6.88 | 66241 | −1.569 |
| ADF-67 | 7.57 | 49062 | −1.556 |
| ADF-245 | 5.82 | 26143 | −1.534 |
| ADF-193 | 5.91 | 64954 | −1.533 |
| ADF-142 | 6.53 | 39774 | −1.525 |
| ADF-470 | 6.5 | 56254 | −1.507 |
| ADF-349 | 4.73 | 13198 | 1.525 |
| ADF-429 | 7.56 | 47861 | 1.526 |
| ADF-489 | 4.96 | 46544 | 1.597 |
| ADF-412 | 5.06 | 49556 | 1.631 |
| ADF-361 | 6.32 | 81004 | 1.672 |
| ADF-480 | 4.94 | 53281 | 1.677 |
| ADF-103 | 7.14 | 20196 | 1.737 |
| ADF-5 | 5.88 | 50355 | 1.751 |
| ADF-535 | 8.78 | 55742 | 1.770 |
| ADF-437 | 4.88 | 45571 | 1.775 |
| ADF-271 | 5.01 | 39858 | 1.915 |
| ADF-151 | 4.84 | 38334 | 1.978 |
| ADF-29 | 4.9 | 39388 | 2.046 |
| ADF-390 | 5.01 | 93773 | 2.476 |
| ADF-132 | 5.06 | 40834 | 2.507 |
| ADF-355 | 9.64 | 10941 | 2.657 |
| ADF-382 | 6.36 | 22175 | 2.696 |
| ADF-508 | 4.94 | 46443 | 2.834 |
| ADF-440 | 5.31 | 44519 | 2.990 |
| ADF-338 | 5.71 | 15618 | 3.068 |

Table XI (b). ADFs altered in Alzheimer's disease hippocampus, p<0.05

TABLE XI (b)

| ADF# | pI | MW (Da) | Fold Change | p-value |
|---|---|---|---|---|
| ADF-319 | 9.97 | 24740 | −2.761 | 0.001 |
| ADF-335 | 5.13 | 17557 | −2.595 | 0.033 |
| ADF-129 | 5.58 | 15554 | −2.546 | 0.001 |
| ADF-495 | 7.46 | 43838 | −2.538 | <0.001 |
| ADF-456 | 5.05 | 35647 | −2.221 | 0.011 |
| ADF-346 | 4.56 | 14197 | −2.061 | 0.001 |
| ADF-434 | 5.46 | 45846 | −2.028 | 0.001 |
| ADF-386 | 5.94 | 12563 | −2.003 | 0.020 |
| ADF-202 | 7.14 | 63134 | −1.959 | <0.001 |
| ADF-284 | 6.28 | 36705 | −1.940 | 0.003 |
| ADF-131 | 5.13 | 33239 | −1.923 | <0.001 |
| ADF-395 | 7.82 | 53530 | −1.902 | 0.002 |
| ADF-229 | 7.77 | 46953 | −1.896 | 0.020 |
| ADF-405 | 4.83 | 50675 | −1.869 | 0.012 |
| ADF-400 | 9.14 | 52590 | −1.852 | 0.007 |
| ADF-366 | 6.34 | 73530 | −1.819 | <0.001 |
| ADF-237 | 4.67 | 25143 | −1.773 | 0.030 |
| ADF-328 | 4.94 | 21882 | −1.762 | 0.005 |
| ADF-459 | 7.67 | 31505 | −1.741 | 0.043 |
| ADF-56 | 7.45 | 91453 | −1.696 | 0.012 |
| ADF-396 | 7.15 | 53305 | −1.687 | 0.004 |
| ADF-318 | 7.15 | 24544 | −1.686 | 0.002 |
| ADF-407 | 4.9 | 50459 | −1.683 | 0.020 |
| ADF-351 | 5.91 | 10930 | −1.671 | 0.008 |
| ADF-362 | 6.81 | 76774 | −1.670 | 0.013 |
| ADF-453 | 6.54 | 41562 | −1.663 | 0.010 |
| ADF-3 | 5.53 | 51762 | −1.627 | 0.003 |
| ADF-360 | 7.36 | 92269 | −1.625 | 0.012 |
| ADF-441 | 6.56 | 43942 | −1.621 | 0.001 |
| ADF-37 | 7.57 | 30563 | −1.613 | <0.001 |
| ADF-308 | 9.64 | 32303 | −1.610 | 0.013 |
| ADF-325 | 7.24 | 22428 | −1.602 | <0.001 |
| ADF-268 | 5.32 | 39751 | −1.586 | 0.011 |
| ADF-276 | 7.51 | 39969 | −1.586 | 0.002 |
| ADF-176 | 6.94 | 63236 | −1.586 | 0.003 |
| ADF-183 | 5.42 | 23634 | −1.582 | <0.001 |
| ADF-476 | 6.45 | 54211 | −1.566 | 0.032 |
| ADF-344 | 4.77 | 14226 | −1.566 | 0.025 |
| ADF-273 | 5.7 | 39437 | −1.563 | 0.001 |
| ADF-492 | 7.5 | 44639 | −1.551 | 0.008 |
| ADF-504 | 7.47 | 59218 | −1.547 | 0.021 |
| ADF-23 | 5.22 | 38803 | −1.534 | 0.006 |
| ADF-223 | 7.08 | 42854 | −1.526 | 0.006 |
| ADF-352 | 6.12 | 10966 | −1.525 | 0.028 |
| ADF-322 | 5.89 | 22204 | −1.519 | 0.030 |
| ADF-439 | 6.84 | 44245 | −1.503 | 0.004 |
| ADF-514 | 6.44 | 52514 | 1.520 | 0.011 |
| ADF-387 | 5.33 | 11323 | 1.540 | <0.001 |
| ADF-165 | 6.42 | 24608 | 1.547 | 0.001 |
| ADF-447 | 5 | 43269 | 1.621 | 0.004 |
| ADF-258 | 8.85 | 42977 | 1.650 | 0.025 |
| ADF-449 | 4.75 | 43300 | 1.655 | 0.008 |
| ADF-303 | 4.91 | 32291 | 1.670 | <0.001 |
| ADF-97 | 5.12 | 19201 | 1.713 | 0.002 |
| ADF-257 | 5.42 | 52510 | 1.738 | 0.039 |
| ADF-419 | 6.52 | 47975 | 1.760 | 0.008 |
| ADF-144 | 4.96 | 43397 | 1.832 | 0.026 |
| ADF-262 | 5.02 | 40870 | 1.892 | 0.026 |
| ADF-204 | 6.29 | 11406 | 1.962 | 0.002 |
| ADF-90 | 5.12 | 40883 | 1.972 | 0.007 |
| ADF-275 | 4.94 | 39423 | 2.015 | 0.020 |
| ADF-98 | 6.35 | 14707 | 2.043 | 0.029 |
| ADF-77 | 5.28 | 46016 | 2.057 | 0.004 |
| ADF-27 | 5.02 | 44949 | 2.065 | 0.014 |
| ADF-8 | 4.96 | 40177 | 2.081 | 0.009 |
| ADF-154 | 4.85 | 39486 | 2.110 | 0.038 |
| ADF-491 | 5.23 | 45828 | 2.145 | 0.019 |
| ADF-119 | 4.95 | 41245 | 2.422 | 0.005 |
| ADF-524 | 4.96 | 44893 | 2.462 | 0.003 |
| ADF-150 | 5.12 | 46104 | 2.738 | 0.006 |
| ADF-493 | 5.08 | 44646 | 2.770 | 0.008 |
| ADF-26 | 5.13 | 48692 | 2.834 | 0.001 |
| ADF-163 | 5.03 | 41674 | 2.938 | 0.001 |
| ADF-510 | 5.06 | 39851 | 3.074 | <0.001 |
| ADF-450 | 5.1 | 42818 | 3.080 | <0.001 |
| ADF-1 | 5.07 | 41958 | 3.105 | 0.040 |
| ADF-120 | 5.06 | 46276 | 3.414 | <0.001 |
| ADF-162 | 5.26 | 48579 | 3.505 | <0.001 |
| ADF-31 | 5.36 | 46573 | 3.515 | 0.005 |
| ADF-159 | 5.19 | 48581 | 3.669 | <0.001 |
| ADF-270 | 4.91 | 40409 | 3.692 | 0.001 |
| ADF-148 | 5.47 | 48199 | 3.842 | <0.001 |
| ADF-152 | 5.32 | 48338 | 4.283 | <0.001 |
| ADF-443 | 4.91 | 43591 | 4.320 | 0.041 |
| ADF-6 | 5.39 | 48281 | 4.628 | <0.001 |
| ADF-411 | 5.33 | 49692 | 4.874 | 0.018 |
| ADF-10 | 5.16 | 46069 | 4.995 | <0.001 |
| ADF-507 | 5.47 | 49332 | 6.299 | 0.047 |

Partial amino acid sequences were determined for the ADPIs present in these ADFs. Details of these ADPIs are provided in Tables IV.

6.2.1.2 Selected Hippocampal Samples

These initial experiments identified 166 features that were altered in the selected set of hippocampal Alzheimer's disease samples as compared with control hippocampal samples.

Details of these ADFs are provided in Table I. Each ADF was differentially present in Alzheimer's disease tissue as compared with control tissue. For some preferred ADFs, listed in Table XII(a), the fold change was greater than 1.5, and for certain highly preferred ADFs, listed in Table XII(b), the difference was significant, p<0.05. For some preferred ADFs, listed in Table XII (c), there was a qualitative difference in ADF expression.

Table XII (a). ADFs altered in Alzheimer's disease hippocampus,

TABLE XII (a)

| ADF # | pI | MW (Da) | Fold Change |
|---|---|---|---|
| ADF-323 | 5.23 | 22730 | −2.124 |
| ADF-521 | 5.24 | 47302 | −1.931 |
| ADF-254 | 6.63 | 138817 | −1.873 |
| ADF-384 | 9.43 | 16119 | −1.811 |
| ADF-476 | 6.45 | 54211 | −1.806 |
| ADF-62 | 5.57 | 92331 | −1.785 |
| ADF-456 | 5.05 | 35647 | −1.784 |
| ADF-386 | 5.94 | 12563 | −1.784 |
| ADF-358 | 6.73 | 105628 | −1.775 |
| ADF-278 | 7.25 | 39463 | −1.754 |
| ADF-67 | 7.57 | 49062 | −1.725 |
| ADF-229 | 7.77 | 46953 | −1.717 |
| ADF-237 | 4.67 | 25143 | −1.686 |
| ADF-326 | 9.69 | 23002 | −1.685 |
| ADF-300 | 9.93 | 33122 | −1.675 |
| ADF-457 | 7.77 | 32435 | −1.655 |
| ADF-269 | 9.72 | 40657 | −1.653 |
| ADF-407 | 4.9 | 50459 | −1.643 |
| ADF-297 | 5.03 | 33778 | −1.610 |
| ADF-470 | 6.5 | 56254 | −1.600 |
| ADF-9 | 4.94 | 55118 | −1.587 |
| ADF-356 | 7.04 | 136798 | −1.582 |
| ADF-336 | 4.52 | 16749 | −1.572 |
| ADF-268 | 5.32 | 39751 | −1.568 |
| ADF-334 | 7.08 | 18540 | −1.557 |
| ADF-492 | 7.5 | 44639 | −1.530 |
| ADF-288 | 6.17 | 35958 | −1.515 |
| ADF-388 | 6.22 | 11486 | −1.514 |
| ADF-337 | 6.61 | 16325 | 1.530 |
| ADF-487 | 5.41 | 46720 | 1.537 |
| ADF-369 | 6.7 | 69857 | 1.548 |
| ADF-454 | 6.17 | 40304 | 1.564 |
| ADF-275 | 4.94 | 39423 | 1.565 |
| ADF-39 | 7.26 | 42323 | 1.570 |
| ADF-381 | 5.48 | 29061 | 1.579 |
| ADF-257 | 5.42 | 52510 | 1.588 |
| ADF-290 | 6.63 | 36052 | 1.596 |
| ADF-12 | 4.88 | 38224 | 1.601 |
| ADF-262 | 5.02 | 40870 | 1.606 |
| ADF-443 | 4.91 | 43591 | 1.625 |
| ADF-471 | 6.1 | 55319 | 1.647 |
| ADF-103 | 7.14 | 20196 | 1.663 |
| ADF-418 | 4.82 | 47830 | 1.686 |
| ADF-119 | 4.95 | 41245 | 1.696 |
| ADF-422 | 5.33 | 126111 | 1.718 |
| ADF-382 | 6.36 | 22175 | 1.748 |
| ADF-29 | 4.9 | 39388 | 1.857 |
| ADF-204 | 6.29 | 11406 | 1.870 |
| ADF-132 | 5.06 | 40834 | 1.872 |
| ADF-491 | 5.23 | 45828 | 1.884 |
| ADF-163 | 5.03 | 41674 | 1.937 |
| ADF-98 | 6.35 | 14707 | 2.124 |
| ADF-489 | 4.96 | 46544 | 2.156 |
| ADF-8 | 4.96 | 40177 | 2.388 |
| ADF-429 | 7.56 | 47861 | 2.438 |
| ADF-1 | 5.07 | 41958 | 2.533 |
| ADF-31 | 5.36 | 46573 | 2.676 |
| ADF-515 | 7.52 | 49586 | 2.821 |
| ADF-437 | 4.88 | 45571 | 3.388 |

Table XII (b). ADFs altered in Alzheimer's disease hippocampus, p<0.05

TABLE XII (b)

| ADF # | pI | MW (Da) | Fold Change | p-value |
|---|---|---|---|---|
| ADF-129 | 5.58 | 15554 | −3.950 | <0.001 |
| ADF-427 | 8.66 | 50180 | −3.744 | 0.038 |
| ADF-139 | 9.81 | 44026 | −3.061 | 0.001 |
| ADF-370 | 9.3 | 69099 | −2.791 | <0.001 |
| ADF-495 | 7.46 | 43838 | −2.721 | <0.001 |
| ADF-342 | 7.51 | 14929 | −2.682 | 0.004 |
| ADF-202 | 7.14 | 63134 | −2.664 | <0.001 |
| ADF-256 | 6.91 | 136744 | −2.601 | 0.020 |
| ADF-319 | 9.97 | 24740 | −2.587 | 0.028 |
| ADF-389 | 6.63 | 126012 | −2.535 | <0.001 |
| ADF-305 | 4.6 | 32146 | −2.501 | <0.001 |
| ADF-176 | 6.94 | 63236 | −2.421 | <0.001 |
| ADF-434 | 5.46 | 45846 | −2.415 | 0.002 |
| ADF-253 | 6.56 | 138727 | −2.397 | 0.013 |
| ADF-390 | 5.01 | 93773 | −2.349 | 0.024 |
| ADF-173 | 6.76 | 64255 | −2.308 | 0.007 |
| ADF-346 | 4.56 | 14197 | −2.299 | 0.002 |
| ADF-405 | 4.83 | 50675 | −2.237 | 0.020 |
| ADF-318 | 7.15 | 24544 | −2.198 | 0.003 |
| ADF-255 | 6.77 | 138133 | −2.192 | 0.019 |
| ADF-193 | 5.91 | 64954 | −2.096 | 0.020 |
| ADF-503 | 7.33 | 59558 | −2.046 | <0.001 |
| ADF-395 | 7.82 | 53530 | −2.025 | 0.019 |
| ADF-332 | 10.02 | 21013 | −2.000 | <0.001 |
| ADF-284 | 6.28 | 36705 | −1.975 | 0.022 |
| ADF-400 | 9.14 | 52590 | −1.975 | 0.012 |
| ADF-362 | 6.81 | 76774 | −1.955 | 0.002 |
| ADF-260 | 9.64 | 42986 | −1.954 | 0.031 |
| ADF-328 | 4.94 | 21882 | −1.868 | 0.011 |
| ADF-273 | 5.7 | 39437 | −1.829 | 0.001 |
| ADF-473 | 6.33 | 54465 | −1.814 | 0.007 |
| ADF-194 | 7.16 | 49777 | −1.806 | 0.001 |
| ADF-42 | 7.92 | 54422 | −1.778 | 0.001 |
| ADF-477 | 6.51 | 54406 | −1.765 | 0.004 |
| ADF-366 | 6.34 | 73530 | −1.763 | 0.025 |
| ADF-56 | 7.45 | 91453 | −1.755 | 0.001 |
| ADF-223 | 7.08 | 42854 | −1.741 | 0.009 |
| ADF-374 | 7.01 | 65362 | −1.728 | 0.006 |
| ADF-463 | 6.58 | 63519 | −1.719 | <0.001 |
| ADF-276 | 7.51 | 39969 | −1.718 | 0.004 |
| ADF-66 | 6.6 | 115748 | −1.709 | 0.020 |
| ADF-479 | 6.09 | 53874 | −1.704 | 0.001 |
| ADF-474 | 7.68 | 54716 | −1.693 | 0.002 |
| ADF-102 | 5.13 | 38984 | −1.679 | <0.001 |
| ADF-431 | 7.65 | 46361 | −1.678 | <0.001 |
| ADF-430 | 5.67 | 46344 | −1.673 | 0.005 |
| ADF-124 | 6.8 | 70243 | −1.670 | 0.018 |
| ADF-282 | 5.69 | 37186 | −1.666 | <0.001 |
| ADF-308 | 9.64 | 32303 | −1.650 | 0.033 |
| ADF-191 | 6.9 | 50019 | −1.636 | <0.001 |
| ADF-439 | 6.84 | 44245 | −1.630 | <0.001 |
| ADF-293 | 7.04 | 34992 | −1.630 | 0.001 |
| ADF-360 | 7.36 | 92269 | −1.627 | 0.016 |
| ADF-263 | 5.58 | 40684 | −1.623 | 0.010 |
| ADF-54 | 7.53 | 90365 | −1.615 | 0.002 |
| ADF-172 | 9.32 | 10855 | −1.613 | <0.001 |
| ADF-392 | 5.47 | 75385 | −1.610 | 0.022 |
| ADF-23 | 5.22 | 38803 | −1.602 | 0.010 |
| ADF-344 | 4.77 | 14226 | −1.598 | 0.022 |
| ADF-351 | 5.91 | 10930 | −1.581 | 0.038 |
| ADF-368 | 5.49 | 65816 | −1.569 | 0.007 |
| ADF-467 | 7.16 | 59914 | −1.567 | 0.009 |
| ADF-441 | 6.56 | 43942 | −1.548 | 0.001 |
| ADF-425 | 7.89 | 50602 | −1.546 | <0.001 |
| ADF-408 | 5.79 | 50212 | −1.545 | 0.020 |
| ADF-502 | 5.95 | 57646 | −1.542 | 0.010 |
| ADF-37 | 7.57 | 30563 | −1.540 | 0.003 |
| ADF-340 | 4.31 | 15235 | −1.536 | 0.033 |
| ADF-436 | 6.1 | 44528 | −1.535 | 0.046 |
| ADF-398 | 6.02 | 51914 | −1.534 | 0.001 |
| ADF-448 | 7.84 | 43648 | −1.517 | 0.016 |
| ADF-183 | 5.42 | 23634 | −1.504 | 0.010 |
| ADF-324 | 7.43 | 22516 | 1.417 | <0.001 |
| ADF-209 | 5.03 | 11738 | 1.553 | 0.006 |
| ADF-165 | 6.42 | 24608 | 1.566 | 0.010 |
| ADF-321 | 6.28 | 23240 | 1.605 | 0.046 |
| ADF-387 | 5.33 | 11323 | 1.663 | 0.004 |
| ADF-501 | 5.33 | 58448 | 1.692 | 0.004 |
| ADF-447 | 5 | 43269 | 1.749 | 0.008 |
| ADF-349 | 4.73 | 13198 | 1.753 | 0.040 |
| ADF-411 | 5.33 | 49692 | 1.757 | <0.001 |
| ADF-419 | 6.52 | 47975 | 1.771 | 0.027 |
| ADF-311 | 5.33 | 28896 | 1.890 | 0.010 |
| ADF-90 | 5.12 | 40883 | 1.960 | 0.026 |
| ADF-468 | 6.45 | 58723 | 1.987 | 0.007 |
| ADF-77 | 5.28 | 46016 | 2.082 | 0.022 |
| ADF-258 | 8.85 | 42977 | 2.100 | 0.018 |
| ADF-97 | 5.12 | 19201 | 2.104 | 0.014 |
| ADF-440 | 5.31 | 44519 | 2.184 | <0.001 |
| ADF-144 | 4.96 | 43397 | 2.364 | 0.003 |
| ADF-27 | 5.02 | 44949 | 2.563 | 0.040 |
| ADF-270 | 4.91 | 40409 | 2.767 | 0.021 |
| ADF-493 | 5.08 | 44646 | 2.843 | 0.014 |
| ADF-26 | 5.13 | 48692 | 2.927 | 0.006 |
| ADF-162 | 5.26 | 48579 | 3.090 | 0.013 |
| ADF-150 | 5.12 | 46104 | 3.114 | 0.033 |
| ADF-120 | 5.06 | 46276 | 3.226 | 0.012 |
| ADF-159 | 5.19 | 48581 | 3.235 | 0.012 |
| ADF-524 | 4.96 | 44893 | 3.348 | 0.004 |
| ADF-10 | 5.16 | 46069 | 3.824 | 0.030 |
| ADF-507 | 5.47 | 49332 | 4.142 | <0.001 |
| ADF-450 | 5.1 | 42818 | 4.635 | 0.001 |
| ADF-148 | 5.47 | 48199 | 5.014 | 0.016 |
| ADF-152 | 5.32 | 48338 | 5.574 | 0.001 |
| ADF-6 | 5.39 | 48281 | 6.167 | 0.002 |

Table XII (c). ADFs with a qualitative difference in expression between Alzheimer's disease samples and control samples.

TABLE XII (c)

| | | | % Feature presence | % Feature presence |
|---|---|---|---|---|
| | | | in Alzheimer's | in control samples |
| ADF # | pI | MW (Da) | disease (n = 9) | (n = 11) |
| ADF-480 | 4.94 | 53281 | 0 | 64 |
| ADF-376 | 6.52 | 65065 | 0 | 55 |

Partial amino acid sequences were determined for the ADPIs present in these ADFs. Details of these ADPIs are provided in Tables IV.

6.2.2 Frontal Cortex

These initial experiments identified 70 features that were altered in the frontal cortex Alzheimer's disease samples as compared with control frontal cortex samples.

Details of these ADFs are provided in Table I. Each ADF was differentially present in Alzheimer's disease tissue as compared with control tissue. For some preferred ADFs, listed in Table XIII (a), the fold change was greater than 1.5, and for certain highly preferred ADFs, listed in Table XIII (b), the difference was significant, p<0.05.

Table XIII (a). ADFs altered in Alzheimer's disease Frontal Cortex,

TABLE XIII (a)

| ADF # | pI | MW (Da) | Fold Change |
|---|---|---|---|
| ADF-281 | 5.4 | 37687 | −2.882 |
| ADF-278 | 7.25 | 39463 | −2.778 |
| ADF-291 | 7.79 | 36208 | −2.422 |
| ADF-475 | 4.99 | 54607 | −2.347 |
| ADF-31 | 5.36 | 46573 | −2.012 |
| ADF-133 | 6.81 | 42650 | −1.987 |
| ADF-472 | 5.24 | 54138 | −1.802 |
| ADF-127 | 5.21 | 40033 | −1.660 |
| ADF-420 | 4.85 | 16376 | −1.641 |
| ADF-366 | 6.34 | 73530 | −1.618 |
| ADF-341 | 4.68 | 15288 | −1.563 |
| ADF-336 | 4.52 | 16749 | −1.560 |
| ADF-437 | 4.88 | 45571 | −1.558 |
| ADF-477 | 6.51 | 54406 | −1.548 |
| ADF-268 | 5.32 | 39751 | −1.545 |
| ADF-329 | 5.02 | 21444 | −1.541 |
| ADF-259 | 7.16 | 42747 | −1.533 |
| ADF-471 | 6.1 | 55319 | 1.515 |
| ADF-409 | 5.69 | 50034 | 1.527 |
| ADF-353 | 7.35 | 10655 | 1.530 |
| ADF-24 | 5.47 | 77449 | 1.546 |
| ADF-220 | 7.17 | 26848 | 1.547 |
| ADF-270 | 4.91 | 40409 | 1.567 |
| ADF-98 | 6.35 | 14707 | 1.584 |
| ADF-275 | 4.94 | 39423 | 1.621 |
| ADF-488 | 7.09 | 46981 | 1.732 |
| ADF-26 | 5.13 | 48692 | 1.739 |
| ADF-315 | 6.13 | 25520 | 1.768 |
| ADF-103 | 7.14 | 20196 | 1.804 |
| ADF-67 | 7.57 | 49062 | 1.963 |
| ADF-159 | 5.19 | 48581 | 1.970 |
| ADF-300 | 9.93 | 33122 | 1.980 |
| ADF-162 | 5.26 | 48579 | 2.000 |
| ADF-338 | 5.71 | 15618 | 2.096 |
| ADF-491 | 5.23 | 45828 | 2.332 |
| ADF-450 | 5.1 | 42818 | 2.653 |
| ADF-150 | 5.12 | 46104 | 2.758 |

Table XIII (b). ADFs altered in Alzheimer's disease Frontal Cortex, p<0.05

TABLE XIII (b)

| ADF # | pI | MW (Da) | Fold Change | p-value |
|---|---|---|---|---|
| ADF-520 | 4.77 | 51522 | −3.195 | <0.001 |
| ADF-11 | 4.81 | 39554 | −3.046 | <0.001 |
| ADF-476 | 6.45 | 54211 | −2.177 | 0.023 |
| ADF-124 | 6.8 | 70243 | −1.781 | 0.030 |
| ADF-376 | 6.52 | 65065 | −1.729 | 0.004 |
| ADF-406 | 6.53 | 51024 | −1.633 | 0.009 |
| ADF-410 | 5.11 | 49646 | −1.608 | 0.006 |
| ADF-277 | 4.98 | 39146 | −1.596 | 0.003 |
| ADF-515 | 7.52 | 49586 | −1.591 | <0.001 |
| ADF-373 | 6.61 | 65116 | −1.589 | <0.001 |
| ADF-441 | 6.56 | 43942 | −1.578 | 0.005 |
| ADF-328 | 4.94 | 21882 | −1.575 | 0.021 |
| ADF-287 | 6.86 | 35923 | −1.571 | 0.015 |
| ADF-396 | 7.15 | 53305 | −1.538 | 0.011 |
| ADF-232 | 5 | 37305 | −1.503 | 0.005 |
| ADF-262 | 5.02 | 40870 | 1.599 | 0.024 |
| ADF-321 | 6.28 | 23240 | 1.719 | 0.035 |
| ADF-204 | 6.29 | 11406 | 1.747 | 0.001 |
| ADF-156 | 5.16 | 47248 | 1.764 | 0.027 |
| ADF-25 | 5.06 | 52537 | 1.771 | 0.027 |
| ADF-506 | 6.68 | 52348 | 1.819 | <0.001 |
| ADF-454 | 6.17 | 40304 | 1.879 | 0.015 |
| ADF-461 | 5.16 | 103474 | 1.890 | 0.005 |
| ADF-382 | 6.36 | 22175 | 2.114 | 0.019 |
| ADF-358 | 6.73 | 105628 | 2.179 | <0.001 |
| ADF-196 | 7.07 | 51831 | 2.182 | 0.023 |
| ADF-120 | 5.06 | 46276 | 2.481 | 0.048 |
| ADF-452 | 8.65 | 42868 | 2.516 | <0.001 |
| ADF-163 | 5.03 | 41674 | 2.680 | 0.043 |
| ADF-148 | 5.47 | 48199 | 2.914 | 0.035 |
| ADF-152 | 5.32 | 48338 | 2.987 | 0.009 |
| ADF-6 | 5.39 | 48281 | 2.991 | 0.023 |

Partial amino acid sequences were determined for the ADPIs present in these ADFs. Details of these ADPIs are provided in Tables IV.

6.2.3 Neocortex

These initial experiments identified 160 features that were altered in the neocortical Alzheimer's disease samples as compared with control neocortical samples.

Details of these ADFs are provided in Table I. Each ADF was differentially present in Alzheimer's disease tissue as compared with control tissue. For some preferred ADFs, listed in Table XIV(a), the fold change was greater than 1.5, and for certain preferred ADFs, listed in Table XIV(b), there was a qualitative difference in ADF expression.

Table XIV (a). ADFs altered in Alzheimer's disease Neocortex

TABLE IV (a)

| ADF # | pI | MW (Da) | Fold Change |
|---|---|---|---|
| ADF-298 | 5.37 | 33180 | −20.277 |
| ADF-113 | 8.39 | 50091 | −15.338 |
| ADF-343 | 9.12 | 15079 | −6.871 |
| ADF-452 | 8.65 | 42868 | −5.210 |
| ADF-300 | 9.93 | 33122 | −5.159 |
| ADF-542 | 7.68 | 16766 | −4.074 |
| ADF-67 | 7.57 | 49062 | −3.737 |
| ADF-370 | 9.3 | 69099 | −3.658 |
| ADF-109 | 8.03 | 47014 | −3.463 |
| ADF-1 | 5.07 | 41958 | −3.300 |
| ADF-292 | 9.67 | 36144 | −3.230 |
| ADF-124 | 6.8 | 70243 | −3.053 |
| ADF-291 | 7.79 | 36208 | −3.022 |
| ADF-91 | 6.95 | 39784 | −2.913 |
| ADF-257 | 5.42 | 52510 | −2.743 |
| ADF-513 | 8.34 | 55698 | −2.714 |
| ADF-132 | 5.06 | 40834 | −2.699 |
| ADF-527 | 8.1 | 13928 | −2.663 |
| ADF-482 | 6.15 | 51602 | −2.627 |
| ADF-101 | 8.99 | 15085 | −2.559 |
| ADF-98 | 6.35 | 14707 | −2.531 |
| ADF-112 | 6.94 | 76640 | −2.411 |
| ADF-413 | 7.59 | 50213 | −2.309 |
| ADF-61 | 8.02 | 65431 | −2.305 |
| ADF-393 | 5.42 | 70689 | −2.288 |
| ADF-375 | 6.12 | 63693 | −2.236 |
| ADF-308 | 9.64 | 32303 | −2.218 |
| ADF-451 | 6.59 | 42651 | −2.180 |
| ADF-412 | 5.06 | 49556 | −2.175 |
| ADF-103 | 7.14 | 20196 | −2.126 |
| ADF-341 | 4.68 | 15288 | −2.119 |
| ADF-349 | 4.73 | 13198 | −2.118 |
| ADF-142 | 6.53 | 39774 | −2.088 |
| ADF-182 | 9.36 | 32746 | −1.992 |
| ADF-458 | 5.13 | 32364 | −1.970 |
| ADF-33 | 9.07 | 42881 | −1.962 |
| ADF-294 | 5.16 | 34573 | −1.938 |
| ADF-8 | 4.96 | 40177 | −1.879 |
| ADF-342 | 7.51 | 14929 | −1.872 |
| ADF-143 | 8.99 | 32577 | −1.863 |
| ADF-77 | 5.28 | 46016 | −1.861 |
| ADF-56 | 7.45 | 91453 | −1.843 |
| ADF-269 | 9.72 | 40657 | −1.831 |
| ADF-417 | 5 | 48877 | −1.810 |
| ADF-268 | 5.32 | 39751 | −1.804 |
| ADF-313 | 5.04 | 28096 | −1.796 |
| ADF-133 | 6.81 | 42650 | −1.793 |
| ADF-409 | 5.69 | 50034 | −1.778 |
| ADF-461 | 5.16 | 103474 | −1.775 |
| ADF-339 | 4.68 | 16126 | −1.769 |
| ADF-296 | 4.76 | 34122 | −1.706 |
| ADF-432 | 9.42 | 46974 | −1.705 |
| ADF-85 | 5.71 | 41191 | −1.688 |
| ADF-138 | 9.72 | 26137 | −1.684 |
| ADF-315 | 6.13 | 25520 | −1.664 |
| ADF-390 | 5.01 | 93773 | −1.661 |
| ADF-429 | 7.56 | 47861 | −1.636 |
| ADF-337 | 6.61 | 16325 | −1.634 |
| ADF-435 | 7.25 | 45448 | −1.631 |
| ADF-345 | 7.75 | 13620 | −1.630 |
| ADF-322 | 5.89 | 22204 | −1.621 |
| ADF-448 | 7.84 | 43648 | −1.611 |
| ADF-487 | 5.41 | 46720 | −1.598 |
| ADF-512 | 6.42 | 22602 | −1.588 |
| ADF-25 | 5.06 | 52537 | −1.587 |
| ADF-415 | 5.61 | 48968 | −1.575 |
| ADF-481 | 6.18 | 53133 | −1.568 |
| ADF-88 | 5.22 | 24404 | −1.560 |
| ADF-15 | 4.64 | 50029 | −1.551 |
| ADF-42 | 7.92 | 54422 | −1.529 |
| ADF-141 | 4.66 | 41227 | −1.512 |
| ADF-360 | 7.36 | 92269 | −1.510 |
| ADF-529 | 9.71 | 33463 | −1.504 |
| ADF-188 | 4.96 | 93776 | 1.505 |
| ADF-254 | 6.63 | 138817 | 1.512 |
| ADF-260 | 9.64 | 42986 | 1.519 |
| ADF-457 | 7.77 | 32435 | 1.525 |
| ADF-492 | 7.5 | 44639 | 1.526 |
| ADF-95 | 6.65 | 40709 | 1.542 |
| ADF-237 | 4.67 | 25143 | 1.543 |
| ADF-348 | 7.36 | 12068 | 1.546 |
| ADF-16 | 5.57 | 48424 | 1.578 |
| ADF-105 | 4.81 | 38327 | 1.580 |
| ADF-494 | 6.95 | 44465 | 1.587 |
| ADF-316 | 6.05 | 25158 | 1.592 |
| ADF-156 | 5.16 | 47248 | 1.594 |
| ADF-29 | 4.9 | 39388 | 1.603 |
| ADF-162 | 5.26 | 48579 | 1.605 |
| ADF-175 | 9.55 | 36150 | 1.632 |
| ADF-306 | 4.67 | 31899 | 1.645 |
| ADF-264 | 5.53 | 40345 | 1.659 |
| ADF-514 | 6.44 | 52514 | 1.671 |
| ADF-284 | 6.28 | 36705 | 1.692 |
| ADF-285 | 5.7 | 36544 | 1.698 |
| ADF-456 | 5.05 | 35647 | 1.714 |
| ADF-372 | 5.68 | 63210 | 1.744 |
| ADF-297 | 5.03 | 33778 | 1.805 |
| ADF-450 | 5.1 | 42818 | 1.828 |
| ADF-426 | 7.68 | 50266 | 1.828 |
| ADF-353 | 7.35 | 10655 | 1.844 |
| ADF-111 | 4.87 | 43065 | 1.862 |
| ADF-515 | 7.52 | 49586 | 1.873 |
| ADF-400 | 9.14 | 52590 | 1.874 |
| ADF-217 | 7.53 | 10660 | 1.964 |
| ADF-90 | 5.12 | 40883 | 2.022 |
| ADF-189 | 5.83 | 65453 | 2.069 |
| ADF-361 | 6.32 | 81004 | 2.075 |
| ADF-154 | 4.85 | 39486 | 2.107 |
| ADF-382 | 6.36 | 22175 | 2.130 |
| ADF-155 | 5.56 | 66533 | 2.149 |
| ADF-476 | 6.45 | 54211 | 2.182 |
| ADF-22 | 5.19 | 102608 | 2.196 |
| ADF-386 | 5.94 | 12563 | 2.226 |
| ADF-220 | 7.17 | 26848 | 2.235 |
| ADF-62 | 5.57 | 92331 | 2.247 |
| ADF-403 | 7.48 | 51603 | 2.303 |
| ADF-318 | 7.15 | 24544 | 2.346 |
| ADF-277 | 4.98 | 39146 | 2.347 |
| ADF-443 | 4.91 | 43591 | 2.350 |
| ADF-172 | 9.32 | 10855 | 2.399 |
| ADF-26 | 5.13 | 48692 | 2.405 |
| ADF-6 | 5.39 | 48281 | 2.430 |
| ADF-240 | 9.56 | 32166 | 2.450 |
| ADF-371 | 5.98 | 64860 | 2.499 |
| ADF-152 | 5.32 | 48338 | 2.599 |
| ADF-506 | 6.68 | 52348 | 2.602 |
| ADF-92 | 8.98 | 34521 | 2.606 |
| ADF-526 | 4.61 | 38042 | 2.614 |
| ADF-521 | 5.24 | 47302 | 2.722 |
| ADF-480 | 4.94 | 53281 | 2.805 |
| ADF-517 | 4.86 | 40635 | 2.831 |
| ADF-9 | 4.94 | 55118 | 2.991 |
| ADF-358 | 6.73 | 105628 | 3.301 |
| ADF-159 | 5.19 | 48581 | 3.380 |
| ADF-127 | 5.21 | 40033 | 3.651 |
| ADF-433 | 9.55 | 46884 | 3.801 |
| ADF-330 | 9.53 | 21378 | 3.837 |
| ADF-148 | 5.47 | 48199 | 3.924 |
| ADF-336 | 4.52 | 16749 | 4.835 |
| ADF-81 | 4.83 | 42229 | 5.742 |
| ADF-338 | 5.71 | 15618 | 7.517 |
| ADF-491 | 5.23 | 45828 | 8.018 |

Table XIV (b). ADFs with a qualitative difference in expression between Alzheimer's disease samples and control samples.

TABLE XIV (b)

| | | | % Feature presence | % Feature presence |
|---|---|---|---|---|
| | | | in Alzheimer's | in control samples |
| ADF # | pI | MW (Da) | disease (n = 3) | (n = 3) |
| ADF-82 | 8.93 | 40442 | 0 | 100 |
| ADF-545 | 5.4 | 64958 | 0 | 67 |
| ADF-258 | 8.85 | 42977 | 0 | 67 |
| ADF-546 | 8.85 | 51770 | 0 | 67 |
| ADF-484 | 8.91 | 50220 | 0 | 67 |
| ADF-383 | 4.52 | 17290 | 0 | 67 |
| ADF-541 | 8.65 | 41532 | 0 | 67 |
| ADF-119 | 4.95 | 41245 | 0 | 67 |
| ADF-262 | 5.02 | 40870 | 0 | 67 |
| ADF-539 | 5.52 | 49807 | 67 | 0 |
| ADF-236 | 4.64 | 18906 | 67 | 0 |
| ADF-120 | 5.06 | 46276 | 67 | 0 |
| ADF-549 | 6.61 | 105070 | 100 | 0 |
| ADF-144 | 4.96 | 43397 | 67 | 0 |
| ADF-462 | 6.88 | 66241 | 100 | 0 |
| ADF-540 | 5.21 | 40933 | 67 | 0 |
| ADF-151 | 4.84 | 38334 | 67 | 0 |
| ADF-289 | 9.18 | 36921 | 67 | 0 |

Partial amino acid sequences were determined for the ADPIs present in these ADFs. Details of these ADPIs are provided in Tables IV.

6.2.4 Entorhinal Cortex

These initial experiments identified 184 features that were altered in the entorhinal cortex Alzheimer's disease samples as compared with control entorhinal cortex samples.

Details of these ADFs are provided in Table I. Each ADF was differentially present in Alzheimer's disease tissue as compared with control tissue. For some preferred ADFs, listed in Table XV (a), the fold change was greater than 1.5, and for certain preferred ADFs, listed in Table XV (b), there was a qualitative difference in ADF expression.

Table XV (a). ADFs altered in Alzheimer's disease hippocampus,

TABLE XV (a)

| ADF # | pI | MW (Da) | Fold Change |
|---|---|---|---|
| ADF-533 | 4.85 | 57995 | −48.166 |
| ADF-41 | 9.84 | 32806 | −11.481 |
| ADF-175 | 9.55 | 36150 | −7.527 |
| ADF-154 | 4.85 | 39486 | −5.929 |
| ADF-129 | 5.58 | 15554 | −5.428 |
| ADF-384 | 9.43 | 16119 | −5.204 |
| ADF-452 | 8.65 | 42868 | −5.105 |
| ADF-78 | 4.88 | 55952 | −5.006 |
| ADF-297 | 5.03 | 33778 | −4.943 |
| ADF-526 | 4.61 | 38042 | −4.168 |
| ADF-534 | 8.6 | 44086 | −4.157 |
| ADF-130 | 4.9 | 41328 | −3.846 |
| ADF-437 | 4.88 | 45571 | −3.709 |
| ADF-107 | 7.05 | 18005 | −3.520 |
| ADF-262 | 5.02 | 40870 | −3.362 |
| ADF-535 | 8.78 | 55742 | −2.960 |
| NDF-334 | 7.08 | 18540 | −2.945 |
| ADF-8 | 4.96 | 40177 | −2.913 |
| ADF-294 | 5.16 | 34573 | −2.735 |
| ADF-458 | 5.13 | 32364 | −2.641 |
| ADF-516 | 7.77 | 44058 | −2.499 |
| ADF-537 | 4.78 | 44895 | −2.495 |
| ADF-29 | 4.9 | 39388 | −2.483 |
| ADF-523 | 9.71 | 47395 | −2.446 |
| ADF-79 | 6.04 | 51058 | −2.426 |
| ADF-284 | 6.28 | 36705 | −2.403 |
| ADF-61 | 8.02 | 65431 | −2.326 |
| ADF-337 | 6.61 | 16325 | −2.256 |
| ADF-456 | 5.05 | 35647 | −2.226 |
| ADF-451 | 6.59 | 42651 | −2.185 |
| ADF-270 | 4.91 | 40409 | −2.143 |
| ADF-525 | 4.88 | 43587 | −2.140 |
| ADF-327 | 5.48 | 21128 | −2.108 |
| ADF-299 | 9.26 | 34579 | −2.108 |
| ADF-476 | 6.45 | 54211 | −2.081 |
| ADF-478 | 5.48 | 53235 | −2.069 |
| ADF-112 | 6.94 | 76640 | −2.067 |
| ADF-495 | 7.46 | 43838 | −2.032 |
| ADF-500 | 6.06 | 60911 | −2.024 |
| ADF-413 | 7.59 | 50213 | −2.006 |
| ADF-347 | 4.52 | 14033 | −1.991 |
| ADF-268 | 5.32 | 39751 | −1.981 |
| ADF-496 | 7.63 | 43211 | −1.966 |
| ADF-449 | 4.75 | 43300 | −1.944 |
| ADF-131 | 5.13 | 33239 | −1.942 |
| ADF-11 | 4.81 | 39554 | −1.877 |
| ADF-288 | 6.17 | 35958 | −1.841 |
| ADF-480 | 4.94 | 53281 | −1.830 |
| ADF-350 | 5.03 | 12536 | −1.828 |
| ADF-1 | 5.07 | 41958 | −1.818 |
| ADF-81 | 4.83 | 42229 | −1.808 |
| ADF-128 | 5.8 | 28231 | −1.801 |
| ADF-328 | 4.94 | 21882 | −1.773 |
| ADF-115 | 7.4 | 78857 | −1.767 |
| ADF-363 | 6.36 | 75388 | −1.764 |
| ADF-411 | 5.33 | 49692 | −1.744 |
| ADF-15 | 4.64 | 50029 | −1.721 |
| ADF-237 | 4.67 | 25143 | −1.715 |
| ADF-317 | 4.61 | 25514 | −1.698 |
| ADF-314 | 5.63 | 27914 | −1.685 |
| ADF-462 | 6.88 | 66241 | −1.684 |
| ADF-103 | 7.14 | 20196 | −1.658 |
| ADF-531 | 7.73 | 88666 | −1.651 |
| ADF-490 | 6.25 | 46318 | −1.647 |
| ADF-485 | 6.27 | 48105 | −1.625 |
| ADF-275 | 4.94 | 39423 | −1.618 |
| ADF-132 | 5.06 | 40834 | −1.616 |
| ADF-309 | 4.69 | 30667 | −1.612 |
| ADF-497 | 6.22 | 37984 | −1.589 |
| ADF-548 | 6.62 | 23128 | −1.585 |
| ADF-188 | 4.96 | 93776 | −1.584 |
| ADF-95 | 6.65 | 40709 | −1.584 |
| ADF-151 | 4.84 | 38334 | −1.584 |
| ADF-447 | 5 | 43269 | −1.576 |
| ADF-342 | 7.51 | 14929 | −1.574 |
| ADF-307 | 4.61 | 31610 | −1.567 |
| ADF-499 | 5.34 | 23685 | −1.564 |
| ADF-133 | 6.81 | 42650 | −1.558 |
| ADF-105 | 4.81 | 38327 | −1.547 |
| ADF-359 | 5.71 | 91189 | −1.547 |
| ADF-318 | 7.15 | 24544 | −1.540 |
| ADF-399 | 6.63 | 52447 | −1.538 |
| ADF-393 | 5.42 | 70689 | −1.530 |
| ADF-68 | 5.63 | 91712 | −1.506 |
| ADF-257 | 5.42 | 52510 | 1.505 |
| ADF-66 | 6.6 | 115748 | 1.505 |
| ADF-331 | 9.32 | 21202 | 1.529 |
| ADF-508 | 4.94 | 46443 | 1.558 |
| ADF-392 | 5.47 | 75385 | 1.564 |
| ADF-160 | 4.86 | 47558 | 1.568 |
| ADF-277 | 4.98 | 39146 | 1.588 |
| ADF-251 | 5.52 | 171859 | 1.591 |
| ADF-153 | 4.99 | 53853 | 1.618 |
| ADF-549 | 6.61 | 105070 | 1.627 |
| ADF-312 | 7.39 | 28434 | 1.641 |
| ADF-150 | 5.12 | 46104 | 1.644 |
| ADF-353 | 7.35 | 10655 | 1.656 |
| ADF-410 | 5.11 | 49646 | 1.657 |
| ADF-266 | 7.41 | 40715 | 1.669 |
| ADF-252 | 5.37 | 126296 | 1.674 |
| ADF-381 | 5.48 | 29061 | 1.694 |
| ADF-216 | 7.76 | 21455 | 1.698 |
| ADF-196 | 7.07 | 51831 | 1.707 |
| ADF-261 | 5.26 | 41426 | 1.714 |
| ADF-70 | 6.78 | 116449 | 1.745 |
| ADF-341 | 4.68 | 15288 | 1.773 |
| ADF-258 | 8.85 | 42977 | 1.778 |
| ADF-92 | 8.98 | 34521 | 1.786 |
| ADF-316 | 6.05 | 25158 | 1.834 |
| ADF-23 | 5.22 | 38803 | 1.839 |
| ADF-274 | 5.05 | 39078 | 1.868 |
| ADF-172 | 9.32 | 10855 | 1.869 |
| ADF-220 | 7.17 | 26848 | 1.870 |
| ADF-272 | 8.13 | 40619 | 1.875 |
| ADF-157 | 5.45 | 42821 | 1.879 |
| ADF-289 | 9.18 | 36921 | 1.908 |
| ADF-356 | 7.04 | 136798 | 1.927 |
| ADF-361 | 6.32 | 81004 | 1.930 |
| ADF-217 | 7.53 | 10660 | 1.955 |
| ADF-27 | 5.02 | 44949 | 2.010 |
| ADF-354 | 5.62 | 10558 | 2.016 |
| ADF-319 | 9.97 | 24740 | 2.060 |
| ADF-250 | 5.07 | 167558 | 2.094 |
| ADF-267 | 5.28 | 39907 | 2.118 |
| ADF-236 | 4.64 | 18906 | 2.138 |
| ADF-412 | 5.06 | 49556 | 2.139 |
| ADF-302 | 7.09 | 32791 | 2.148 |
| ADF-269 | 9.72 | 40657 | 2.159 |
| ADF-455 | 4.89 | 37155 | 2.217 |
| ADF-357 | 5.17 | 115959 | 2.218 |
| ADF-117 | 4.95 | 47483 | 2.296 |
| ADF-376 | 6.52 | 65065 | 2.322 |
| ADF-428 | 5.04 | 48509 | 2.360 |
| ADF-349 | 4.73 | 13198 | 2.416 |
| ADF-26 | 5.13 | 48692 | 2.427 |
| ADF-416 | 6.8 | 49576 | 2.457 |
| ADF-450 | 5.1 | 42818 | 2.513 |
| ADF-102 | 5.13 | 38984 | 2.521 |
| ADF-407 | 4.9 | 50459 | 2.614 |
| ADF-402 | 5.58 | 51111 | 2.636 |
| ADF-541 | 8.65 | 41532 | 2.728 |
| ADF-380 | 8.69 | 32065 | 2.967 |
| ADF-148 | 5.47 | 48199 | 2.986 |
| ADF-382 | 6.36 | 22175 | 3.076 |
| ADF-300 | 9.93 | 33122 | 3.143 |
| ADF-120 | 5.06 | 46276 | 3.205 |
| ADF-343 | 9.12 | 15079 | 3.277 |
| ADF-344 | 4.77 | 14226 | 3.503 |
| ADF-10 | 5.16 | 46069 | 3.518 |
| ADF-159 | 5.19 | 48581 | 3.829 |
| ADF-338 | 5.71 | 15618 | 4.002 |
| ADF-162 | 5.26 | 48579 | 4.154 |
| ADF-390 | 5.01 | 93773 | 4.290 |
| ADF-6 | 5.39 | 48281 | 4.570 |
| ADF-414 | 4.94 | 49167 | 5.162 |
| ADF-152 | 5.32 | 48338 | 5.214 |
| ADF-243 | 5.35 | 39493 | 7.183 |

Table XV (b). ADFs with a qualitative difference in expression between Alzheimer's disease samples and control samples.

TABLE XV (b)

| | | | % Feature presence | % Feature presence |
|---|---|---|---|---|
| | | | in Alzheimer's | in control samples |
| ADF # | pI | MW (Da) | disease (n = 3) | (n = 3) |
| ADF-520 | 4.77 | 51522 | 0 | 67 |
| ADF-517 | 4.86 | 40635 | 0 | 67 |
| ADF-144 | 4.96 | 43397 | 0 | 67 |
| ADF-111 | 4.87 | 43065 | 0 | 100 |
| ADF-532 | 7.9 | 56000 | 0 | 67 |
| ADF-139 | 9.81 | 44026 | 0 | 100 |
| ADF-492 | 7.5 | 44639 | 0 | 67 |
| ADF-126 | 5.15 | 42785 | 0 | 67 |
| ADF-519 | 4.84 | 53906 | 0 | 67 |
| ADF-378 | 5.66 | 38000 | 0 | 67 |
| ADF-486 | 4.77 | 47947 | 0 | 100 |
| ADF-377 | 9.59 | 44257 | 0 | 100 |
| ADF-538 | 4.73 | 74425 | 0 | 67 |
| ADF-522 | 5.7 | 46027 | 0 | 67 |
| ADF-140 | 4.58 | 15950 | 0 | 67 |
| ADF-539 | 5.52 | 49807 | 0 | 67 |
| ADF-498 | 4.77 | 38566 | 67 | 0 |
| ADF-301 | 9.79 | 33445 | 67 | 0 |
| ADF-228 | 6.41 | 74736 | 67 | 0 |
| ADF-125 | 4.84 | 43088 | 67 | 0 |
| ADF-536 | 4.52 | 55713 | 67 | 0 |
| ADF-521 | 5.24 | 47302 | 67 | 0 |
| ADF-493 | 5.08 | 44646 | 67 | 0 |
| ADF-421 | 7.61 | 15176 | 67 | 0 |
| ADF-333 | 7.8 | 20396 | 67 | 0 |
| ADF-506 | 6.68 | 52348 | 67 | 0 |
| ADF-292 | 9.67 | 36144 | 67 | 0 |

Partial amino acid sequences were determined for the ADPIs present in these ADFs. Details of these ADPIs are provided in Tables IV.

6.2.5 Amygdala

These initial experiments identified 188 features that were altered in the amygdala Alzheimer's disease samples as compared with control amygdala samples.

Details of these ADFs are provided in Table I. Each ADF was differentially present in Alzheimer's disease tissue as compared with control tissue. For some preferred ADFs, listed in Table XVI (a), the fold change was greater than 1.5, and for certain preferred ADFs, listed in Table XVI (b), there was a qualitative difference in ADF expression.

Table XVI (a). ADFs altered in Alzheimer's disease amygdala,

TABLE XVI (a)

| ADF # | pI | MW (Da) | Fold Change |
|---|---|---|---|
| ADF-499 | 5.34 | 23685 | −10.320 |
| ADF-279 | 8.09 | 39011 | −8.556 |
| ADF-272 | 8.13 | 40619 | −5.076 |
| ADF-349 | 4.73 | 13198 | −5.020 |
| ADF-237 | 4.67 | 25143 | −4.637 |
| ADF-16 | 5.57 | 48424 | −4.349 |
| ADF-127 | 5.21 | 40033 | −4.029 |
| ADF-521 | 5.24 | 47302 | −3.840 |
| ADF-146 | 5.25 | 11857 | −3.740 |
| ADF-380 | 8.69 | 32065 | −3.661 |
| ADF-243 | 5.35 | 39493 | −3.616 |
| ADF-67 | 7.57 | 49062 | −3.540 |
| ADF-251 | 5.52 | 171859 | −3.486 |
| ADF-284 | 6.28 | 36705 | −3.473 |
| ADF-261 | 5.26 | 41426 | −3.251 |
| ADF-31 | 5.36 | 46573 | −3.222 |
| ADF-92 | 8.98 | 34521 | −2.847 |
| ADF-260 | 9.64 | 42986 | −2.845 |
| ADF-394 | 5.62 | 66442 | −2.671 |
| ADF-531 | 7.73 | 88666 | −2.583 |
| ADF-486 | 4.77 | 47947 | −2.563 |
| ADF-410 | 5.11 | 49646 | −2.559 |
| ADF-432 | 9.42 | 46974 | −2.536 |
| ADF-277 | 4.98 | 39146 | −2.480 |
| ADF-23 | 5.22 | 38803 | −2.361 |
| ADF-400 | 9.14 | 52590 | −2.355 |
| ADF-488 | 7.09 | 46981 | −2.314 |
| ADF-128 | 5.8 | 28231 | −2.222 |
| ADF-528 | 4.93 | 94807 | −2.210 |
| ADF-468 | 6.45 | 58723 | −2.207 |
| ADF-480 | 4.94 | 53281 | −2.192 |
| ADF-473 | 6.33 | 54465 | −2.174 |
| ADF-91 | 6.95 | 39784 | −2.156 |
| ADF-422 | 5.33 | 126111 | −2.065 |
| ADF-470 | 6.5 | 56254 | −2.023 |
| ADF-341 | 4.68 | 15288 | −2.012 |
| ADF-434 | 5.46 | 45846 | −1.966 |
| ADF-543 | 8.85 | 32258 | −1.965 |
| ADF-534 | 8.6 | 44086 | −1.918 |
| ADF-72 | 5.8 | 91999 | −1.893 |
| ADF-305 | 4.6 | 32146 | −1.870 |
| ADF-318 | 7.15 | 24544 | −1.863 |
| ADF-339 | 4.68 | 16126 | −1.848 |
| ADF-459 | 7.67 | 31505 | −1.844 |
| ADF-85 | 5.71 | 41191 | −1.840 |
| ADF-66 | 6.6 | 115748 | −1.817 |
| ADF-151 | 4.84 | 38334 | −1.808 |
| ADF-411 | 5.33 | 49692 | −1.792 |
| ADF-82 | 8.93 | 40442 | −1.789 |
| ADF-312 | 7.39 | 28434 | −1.770 |
| ADF-544 | 5.53 | 38484 | −1.763 |
| ADF-3 | 5.53 | 51762 | −1.760 |
| ADF-465 | 6.94 | 59947 | −1.754 |
| ADF-401 | 6.27 | 51636 | −1.750 |
| ADF-467 | 7.16 | 59914 | −1.749 |
| ADF-456 | 5.05 | 35647 | −1.727 |
| ADF-395 | 7.82 | 53530 | −1.722 |
| NDF-420 | 4.85 | 16376 | −1.720 |
| ADF-479 | 6.09 | 53874 | −1.697 |
| ADF-397 | 5.29 | 51553 | −1.690 |
| ADF-462 | 6.88 | 66241 | −1.679 |
| ADF-375 | 6.12 | 63693 | −1.679 |
| ADF-436 | 6.1 | 44528 | −1.674 |
| ADF-223 | 7.08 | 42854 | −1.660 |
| ADF-283 | 5.58 | 36661 | −1.659 |
| ADF-337 | 6.61 | 16325 | −1.657 |
| ADF-361 | 6.32 | 81004 | −1.655 |
| ADF-125 | 4.84 | 43088 | −1.647 |
| ADF-13 | 5.41 | 67038 | −1.644 |
| ADF-102 | 5.13 | 38984 | −1.641 |
| ADF-365 | 6.47 | 74592 | −1.623 |
| ADF-517 | 4.86 | 40635 | −1.616 |
| ADF-87 | 5.48 | 34110 | −1.616 |
| ADF-131 | 5.13 | 33239 | −1.615 |
| ADF-296 | 4.76 | 34122 | −1.612 |
| ADF-442 | 5.36 | 43816 | −1.609 |
| ADF-294 | 5.16 | 34573 | −1.604 |
| ADF-437 | 4.88 | 45571 | −1.596 |
| ADF-15 | 4.64 | 50029 | −1.585 |
| ADF-105 | 4.81 | 38327 | −1.579 |
| ADF-416 | 6.8 | 49576 | −1.560 |
| ADF-366 | 6.34 | 73530 | −1.535 |
| ADF-445 | 5.55 | 42883 | −1.533 |
| ADF-525 | 4.88 | 43587 | −1.518 |
| ADF-22 | 5.19 | 102608 | −1.518 |
| ADF-320 | 6.44 | 23908 | −1.507 |
| ADF-150 | 5.12 | 46104 | 1.510 |
| ADF-348 | 7.36 | 12068 | 1.511 |
| ADF-209 | 5.03 | 11738 | 1.511 |
| ADF-475 | 4.99 | 54607 | 1.519 |
| ADF-62 | 5.57 | 92331 | 1.523 |
| ADF-438 | 6.15 | 44253 | 1.537 |
| ADF-193 | 5.91 | 64954 | 1.538 |
| ADF-419 | 6.52 | 47975 | 1.546 |
| ADF-355 | 9.64 | 10941 | 1.550 |
| ADF-108 | 5.74 | 34823 | 1.551 |
| ADF-258 | 8.85 | 42977 | 1.554 |
| ADF-119 | 4.95 | 41245 | 1.556 |
| ADF-425 | 7.89 | 50602 | 1.557 |
| ADF-413 | 7.59 | 50213 | 1.561 |
| ADF-27 | 5.02 | 44949 | 1.567 |
| ADF-303 | 4.91 | 32291 | 1.567 |
| ADF-94 | 5.84 | 43662 | 1.581 |
| ADF-24 | 5.47 | 77449 | 1.602 |
| ADF-208 | 5.26 | 19241 | 1.602 |
| ADF-126 | 5.15 | 42785 | 1.632 |
| ADF-444 | 6.48 | 43383 | 1.634 |
| ADF-275 | 4.94 | 39423 | 1.659 |
| ADF-314 | 5.63 | 27914 | 1.660 |
| ADF-6 | 5.39 | 48281 | 1.660 |
| ADF-98 | 6.35 | 14707 | 1.677 |
| ADF-188 | 4.96 | 93776 | 1.681 |
| ADF-313 | 5.04 | 28096 | 1.685 |
| ADF-79 | 6.04 | 51058 | 1.693 |
| ADF-130 | 4.9 | 41328 | 1.696 |
| ADF-149 | 5.13 | 43744 | 1.699 |
| ADF-452 | 8.65 | 42868 | 1.704 |
| ADF-159 | 5.19 | 48581 | 1.704 |
| ADF-334 | 7.08 | 18540 | 1.714 |
| ADF-379 | 7.09 | 37275 | 1.715 |
| ADF-429 | 7.56 | 47861 | 1.755 |
| ADF-386 | 5.94 | 12563 | 1.767 |
| ADF-454 | 6.17 | 40304 | 1.786 |
| ADF-35 | 7.84 | 42531 | 1.790 |
| ADF-265 | 6.77 | 41249 | 1.794 |
| ADF-447 | 5 | 43269 | 1.809 |
| ADF-138 | 9.72 | 26137 | 1.870 |
| ADF-156 | 5.16 | 47248 | 1.879 |
| ADF-97 | 5.12 | 19201 | 1.910 |
| ADF-10 | 5.16 | 46069 | 1.918 |
| ADF-311 | 5.33 | 28896 | 1.926 |
| ADF-288 | 6.17 | 35958 | 1.959 |
| ADF-526 | 4.61 | 38042 | 2.007 |
| ADF-376 | 6.52 | 65065 | 2.018 |
| ADF-111 | 4.87 | 43065 | 2.040 |
| ADF-196 | 7.07 | 51831 | 2.078 |
| ADF-26 | 5.13 | 48692 | 2.146 |
| ADF-144 | 4.96 | 43397 | 2.156 |
| ADF-95 | 6.65 | 40709 | 2.162 |
| ADF-509 | 9.08 | 44787 | 2.199 |
| ADF-466 | 7.05 | 60297 | 2.221 |
| ADF-117 | 4.95 | 47483 | 2.229 |
| ADF-370 | 9.3 | 69099 | 2.230 |
| ADF-163 | 5.03 | 41674 | 2.452 |
| ADF-291 | 7.79 | 36208 | 2.669 |
| ADF-162 | 5.26 | 48579 | 2.690 |
| ADF-308 | 9.64 | 32303 | 2.707 |
| ADF-377 | 9.59 | 44257 | 2.832 |
| ADF-338 | 5.71 | 15618 | 3.041 |
| ADF-382 | 6.36 | 22175 | 4.063 |
| ADF-9 | 4.94 | 55118 | 5.228 |
| ADF-8 | 4.96 | 40177 | 5.460 |
| ADF-496 | 7.63 | 43211 | 6.277 |
| ADF-506 | 6.68 | 52348 | 6.543 |
| ADF-383 | 4.52 | 17290 | 7.935 |
| ADF-508 | 4.94 | 46443 | 8.478 |
| ADF-139 | 9.81 | 44026 | 9.345 |

Table XVI (b). ADFs with a qualitative difference in expression between Alzheimer's disease samples and control samples.

TABLE XVI (b)

| | | | % Feature | |
|---|---|---|---|---|
| | | | presence in | |
| | | | Alzheimer's | % Feature presence in |
| ADF# | pI | MW (Da) | disease (n = 3) | control samples (n = 3) |
| ADF-230 | 4.87 | 53678 | 0 | 67 |
| ADF-289 | 9.18 | 36921 | 0 | 67 |
| ADF-477 | 6.51 | 54406 | 0 | 67 |
| ADF-175 | 9.55 | 36150 | 0 | 67 |
| ADF-390 | 5.01 | 93773 | 0 | 67 |
| ADF-530 | 4.9 | 53903 | 0 | 67 |
| ADF-326 | 9.69 | 23002 | 0 | 67 |
| ADF-547 | 4.89 | 59987 | 0 | 67 |
| ADF-141 | 4.66 | 41227 | 0 | 67 |
| ADF-332 | 10.02 | 21013 | 0 | 67 |
| ADF-433 | 9.55 | 46884 | 0 | 100 |
| ADF-455 | 4.89 | 37155 | 0 | 67 |
| ADF-391 | 7.62 | 88209 | 0 | 67 |
| ADF-523 | 9.71 | 47395 | 0 | 67 |
| ADF-381 | 5.48 | 29061 | 0 | 67 |
| ADF-245 | 5.82 | 26143 | 0 | 100 |
| ADF-335 | 5.13 | 17557 | 0 | 67 |
| ADF-464 | 6.01 | 58817 | 0 | 100 |
| ADF-298 | 5.37 | 33180 | 0 | 67 |
| ADF-472 | 5.24 | 54138 | 0 | 67 |
| ADF-460 | 6.61 | 15951 | 0 | 67 |
| ADF-522 | 5.7 | 46027 | 67 | 0 |
| ADF-489 | 4.96 | 46544 | 67 | 0 |
| ADF-510 | 5.06 | 39851 | 67 | 0 |
| ADF-278 | 7.25 | 39463 | 100 | 0 |
| ADF-78 | 4.88 | 55952 | 67 | 0 |
| ADF-414 | 4.94 | 49167 | 67 | 0 |
| ADF-520 | 4.77 | 51522 | 67 | 0 |
| ADF-424 | 4.83 | 52105 | 67 | 0 |

Partial amino acid sequences were determined for the ADPIs present in these ADFs. Details of these ADPIs are provided in Tables IV.

6.2.6 All Alzheimer's Disease Samples vs. All Control Samples

These initial experiments identified 52 features that were altered in the whole set of Alzheimer's disease samples as compared with control samples.

Details of these ADFs are provided in Table I. Each ADF was differentially present in Alzheimer's disease tissue as compared with control tissue. For some preferred ADFs, listed in Table XVII (a), the fold change was greater than 1.5, and for certain highly preferred ADFs, listed in Table XVII (b), the difference was significant, p<0.05.

Table XVII (a). ADFs altered in Alzheimer's disease hippocampus,

TABLE XVII (a)

| ADF# | pI | MW (Da) | Fold Change |
|---|---|---|---|
| ADF-16 | 5.57 | 48424 | −1.681 |
| ADF-452 | 8.65 | 42868 | −1.672 |
| ADF-82 | 8.93 | 40442 | −1.657 |
| ADF-67 | 7.57 | 49062 | −1.547 |
| ADF-130 | 4.9 | 41328 | −1.519 |
| ADF-427 | 8.66 | 50180 | −1.512 |
| ADF-413 | 7.59 | 50213 | −1.510 |
| ADF-271 | 5.01 | 39858 | 1.558 |
| ADF-77 | 5.28 | 46016 | 1.613 |
| ADF-132 | 5.06 | 40834 | 1.694 |
| ADF-9 | 4.94 | 55118 | 1.744 |
| ADF-27 | 5.02 | 44949 | 1.772 |
| ADF-1 | 5.07 | 41958 | 2.083 |
| ADF-31 | 5.36 | 46573 | 2.154 |
| ADF-150 | 5.12 | 46104 | 2.181 |
| ADF-443 | 4.91 | 43591 | 2.303 |
| ADF-411 | 5.33 | 49692 | 2.422 |
| ADF-507 | 5.47 | 49332 | 4.102 |

Table XVII (b). ADFs altered in Alzheimer's disease hippocampus, p<0.05

TABLE XVII (b)

| ADF# | pI | MW (Da) | Fold Change | p-value |
|---|---|---|---|---|
| ADF-456 | 5.05 | 35647 | −1.938 | 0.004 |
| ADF-297 | 5.03 | 33778 | −1.757 | 0.005 |
| ADF-384 | 9.43 | 16119 | −1.713 | 0.043 |
| ADF-284 | 6.28 | 36705 | −1.677 | 0.006 |
| ADF-237 | 4.67 | 25143 | −1.631 | 0.008 |
| ADF-346 | 4.56 | 14197 | −1.625 | <0.001 |
| ADF-294 | 5.16 | 34573 | −1.592 | 0.032 |
| ADF-328 | 4.94 | 21882 | −1.580 | <0.001 |
| ADF-458 | 5.13 | 32364 | −1.577 | 0.028 |
| ADF-124 | 6.8 | 70243 | −1.523 | 0.010 |
| ADF-318 | 7.15 | 24544 | −1.511 | 0.007 |
| ADF-268 | 5.32 | 39751 | −1.510 | 0.001 |
| ADF-8 | 4.96 | 40177 | 1.503 | 0.045 |
| ADF-419 | 6.52 | 47975 | 1.511 | 0.029 |
| ADF-98 | 6.35 | 14707 | 1.550 | 0.028 |
| ADF-90 | 5.12 | 40883 | 1.558 | 0.010 |
| ADF-204 | 6.29 | 11406 | 1.625 | 0.008 |
| ADF-275 | 4.94 | 39423 | 1.795 | 0.048 |
| ADF-270 | 4.91 | 40409 | 1.932 | 0.009 |
| ADF-119 | 4.95 | 41245 | 2.000 | 0.044 |
| ADF-491 | 5.23 | 45828 | 2.065 | 0.007 |
| ADF-510 | 5.06 | 39851 | 2.282 | <0.001 |
| ADF-163 | 5.03 | 41674 | 2.360 | 0.003 |
| ADF-26 | 5.13 | 48692 | 2.366 | 0.001 |
| ADF-382 | 6.36 | 22175 | 2.447 | 0.001 |
| ADF-120 | 5.06 | 46276 | 2.575 | 0.004 |
| ADF-450 | 5.1 | 42818 | 2.680 | <0.001 |
| ADF-162 | 5.26 | 48579 | 2.967 | <0.001 |
| ADF-159 | 5.19 | 48581 | 3.040 | <0.001 |
| ADF-148 | 5.47 | 48199 | 3.153 | <0.001 |
| ADF-338 | 5.71 | 15618 | 3.201 | <0.001 |
| ADF-10 | 5.16 | 46069 | 3.526 | 0.003 |
| ADF-152 | 5.32 | 48338 | 3.563 | <0.001 |
| ADF-6 | 5.39 | 48281 | 3.895 | <0.001 |

Partial amino acid sequences were determined for the ADPIs present in these ADFs. Details of these ADPIs are provided in Tables IV.

6.2.7 Early Markers of Alzheimer's Disease

These initial experiments identified 118 features that were altered in the hippocampus and entorhinal cortex Alzheimer's disease samples as compared with the neocortex, frontal cortex and amygdala Alzheimer's disease samples. They are useful as markers of early Alzheimer's disease.

Details of these ADFs are provided in Table I. Each ADF was differentially present in Alzheimer's disease tissue as compared with control tissue. For some preferred ADFs, listed in Table XVIII(a), the fold change was greater than 1.5, and for certain highly preferred ADFs, listed in Table XVIII(b), the difference was significant, p<0.05.

Table XVIII (a). ADFs altered in Early Alzheimer's disease

TABLE XVIII (a)

| ADF# | pI | MW (Da) | Fold Change |
|---|---|---|---|
| ADF-452 | 8.65 | 42868 | −2.118 |
| ADF-9 | 4.94 | 55118 | −2.014 |
| ADF-336 | 4.52 | 16749 | −1.802 |
| ADF-282 | 5.69 | 37186 | −1.750 |
| ADF-471 | 6.1 | 55319 | −1.662 |
| ADF-300 | 9.93 | 33122 | −1.653 |
| ADF-260 | 9.64 | 42986 | −1.646 |
| ADF-375 | 6.12 | 63693 | −1.625 |
| ADF-444 | 6.48 | 43383 | −1.590 |
| ADF-25 | 5.06 | 52537 | −1.577 |
| ADF-376 | 6.52 | 65065 | −1.574 |
| ADF-403 | 7.48 | 51603 | −1.555 |
| ADF-400 | 9.14 | 52590 | −1.546 |
| ADF-456 | 5.05 | 35647 | −1.502 |
| ADF-412 | 5.06 | 49556 | 1.528 |
| ADF-442 | 5.36 | 43816 | 1.535 |
| ADF-142 | 6.53 | 39774 | 1.548 |
| ADF-272 | 8.13 | 40619 | 1.550 |
| ADF-381 | 5.48 | 29061 | 1.558 |
| ADF-511 | 7.71 | 35365 | 1.562 |
| ADF-149 | 5.13 | 43744 | 1.569 |
| ADF-355 | 9.64 | 10941 | 1.626 |
| ADF-302 | 7.09 | 32791 | 1.630 |
| ADF-510 | 5.06 | 39851 | 1.665 |
| ADF-485 | 6.27 | 48105 | 1.668 |
| ADF-527 | 8.1 | 13928 | 1.687 |
| ADF-251 | 5.52 | 171859 | 1.759 |
| ADF-271 | 5.01 | 39858 | 1.812 |
| ADF-148 | 5.47 | 48199 | 1.911 |
| ADF-390 | 5.01 | 93773 | 1.964 |
| ADF-535 | 8.78 | 55742 | 2.037 |
| ADF-270 | 4.91 | 40409 | 2.098 |
| ADF-414 | 4.94 | 49167 | 2.146 |
| ADF-124 | 6.8 | 70243 | 2.214 |
| ADF-410 | 5.11 | 49646 | 2.225 |
| ADF-151 | 4.84 | 38334 | 2.269 |
| ADF-443 | 4.91 | 43591 | 2.426 |
| ADF-175 | 9.55 | 36150 | 2.804 |

Table XII(b). ADFs altered in Early Alzheimer's disease, p<0.05

TABLE XVIII (b)

| ADF# | pI | MW (Da) | Fold Change | p-value |
|---|---|---|---|---|
| ADF-495 | 7.46 | 43838 | −2.440 | 0.003 |
| ADF-373 | 6.61 | 65116 | −2.129 | <0.001 |
| ADF-173 | 6.76 | 64255 | −2.062 | 0.006 |
| ADF-518 | 6.1 | 61221 | −1.901 | <0.001 |
| ADF-304 | 5.36 | 31776 | −1.887 | <0.001 |
| ADF-121 | 6.34 | 60952 | −1.886 | <0.001 |
| ADF-79 | 6.04 | 51058 | −1.855 | 0.007 |
| ADF-319 | 9.97 | 24740 | −1.764 | 0.005 |
| ADF-295 | 5.39 | 34366 | −1.763 | 0.033 |
| ADF-423 | 6.21 | 61089 | −1.760 | <0.001 |
| ADF-366 | 6.34 | 73530 | −1.733 | 0.010 |
| ADF-446 | 6.64 | 43189 | −1.713 | 0.001 |
| ADF-202 | 7.14 | 63134 | −1.679 | 0.006 |
| ADF-288 | 6.17 | 35958 | −1.678 | 0.040 |
| ADF-505 | 5.32 | 55892 | −1.669 | <0.001 |
| ADF-155 | 5.56 | 66533 | −1.656 | 0.009 |
| ADF-364 | 6.09 | 73832 | −1.652 | <0.001 |
| ADF-504 | 7.47 | 59218 | −1.648 | 0.001 |
| ADF-176 | 6.94 | 63236 | −1.598 | 0.009 |
| ADF-434 | 5.46 | 45846 | −1.578 | 0.003 |
| ADF-405 | 4.83 | 50675 | −1.562 | 0.016 |
| ADF-23 | 5.22 | 38803 | −1.560 | 0.021 |
| ADF-368 | 5.49 | 65816 | −1.557 | 0.005 |
| ADF-102 | 5.13 | 38984 | −1.542 | 0.001 |
| ADF-193 | 5.91 | 64954 | −1.537 | 0.011 |
| ADF-160 | 4.86 | 47558 | −1.525 | 0.004 |
| ADF-280 | 5.83 | 37149 | −1.518 | 0.029 |
| ADF-157 | 5.45 | 42821 | −1.515 | 0.001 |
| ADF-469 | 5.48 | 55802 | −1.513 | 0.002 |
| ADF-367 | 5.35 | 66839 | −1.505 | 0.001 |
| ADF-399 | 6.63 | 52447 | 1.500 | 0.037 |
| ADF-514 | 6.44 | 52514 | 1.530 | 0.009 |
| ADF-404 | 6.73 | 51384 | 1.545 | 0.003 |
| ADF-331 | 9.32 | 21202 | 1.548 | 0.021 |
| ADF-250 | 5.07 | 167558 | 1.575 | 0.046 |
| ADF-91 | 6.95 | 39784 | 1.598 | 0.032 |
| ADF-321 | 6.28 | 23240 | 1.601 | <0.001 |
| ADF-415 | 5.61 | 48968 | 1.636 | 0.011 |
| ADF-283 | 5.58 | 36661 | 1.670 | 0.025 |
| ADF-387 | 5.33 | 11323 | 1.724 | <0.001 |
| ADF-266 | 7.41 | 40715 | 1.759 | 0.001 |
| ADF-286 | 7.79 | 36912 | 1.774 | 0.005 |
| ADF-335 | 5.13 | 17557 | 1.801 | <0.001 |
| ADF-478 | 5.48 | 53235 | 1.804 | 0.003 |
| ADF-262 | 5.02 | 40870 | 1.840 | 0.043 |
| ADF-90 | 5.12 | 40883 | 1.850 | 0.013 |
| ADF-165 | 6.42 | 24608 | 1.867 | <0.001 |
| ADF-337 | 6.61 | 16325 | 1.880 | 0.003 |
| ADF-119 | 4.95 | 41245 | 1.967 | 0.007 |
| ADF-491 | 5.23 | 45828 | 1.998 | 0.015 |
| ADF-472 | 5.24 | 54138 | 2.091 | <0.001 |
| ADF-419 | 6.52 | 47975 | 2.121 | 0.001 |
| ADF-85 | 5.71 | 41191 | 2.274 | 0.001 |
| ADF-103 | 7.14 | 20196 | 2.287 | 0.015 |
| ADF-8 | 4.96 | 40177 | 2.325 | 0.004 |
| ADF-144 | 4.96 | 43397 | 2.382 | 0.003 |
| ADF-204 | 6.29 | 11406 | 2.416 | <0.001 |
| ADF-26 | 5.13 | 48692 | 2.493 | 0.016 |
| ADF-275 | 4.94 | 39423 | 2.558 | 0.003 |
| ADF-154 | 4.85 | 39486 | 2.597 | 0.004 |
| ADF-67 | 7.57 | 49062 | 2.685 | 0.003 |
| ADF-429 | 7.56 | 47861 | 2.691 | 0.039 |
| ADF-132 | 5.06 | 40834 | 2.857 | 0.019 |
| ADF-77 | 5.28 | 46016 | 2.907 | 0.005 |
| ADF-152 | 5.32 | 48338 | 3.000 | 0.001 |
| ADF-437 | 4.88 | 45571 | 3.000 | 0.033 |
| ADF-150 | 5.12 | 46104 | 3.008 | 0.006 |
| ADF-159 | 5.19 | 48581 | 3.026 | 0.003 |
| ADF-12 | 4.88 | 38224 | 3.107 | 0.003 |
| ADF-6 | 5.39 | 48281 | 3.164 | 0.001 |
| ADF-29 | 4.9 | 39388 | 3.199 | 0.001 |
| ADF-163 | 5.03 | 41674 | 3.360 | 0.001 |
| ADF-120 | 5.06 | 46276 | 3.465 | 0.001 |
| ADF-1 | 5.07 | 41958 | 3.479 | 0.017 |
| ADF-162 | 5.26 | 48579 | 3.559 | <0.001 |
| ADF-27 | 5.02 | 44949 | 3.690 | <0.001 |
| ADF-493 | 5.08 | 44646 | 4.346 | 0.003 |
| ADF-10 | 5.16 | 46069 | 5.130 | <0.001 |
| ADF-524 | 4.96 | 44893 | 5.588 | 0.002 |
| ADF-31 | 5.36 | 46573 | 5.977 | <0.001 |

Partial amino acid sequences were determined for the ADPIs present in these ADFs. Details of these ADPIs are provided in Tables IV.

### EXAMPLE 2

### Cloning and Characterization of ADPI-41

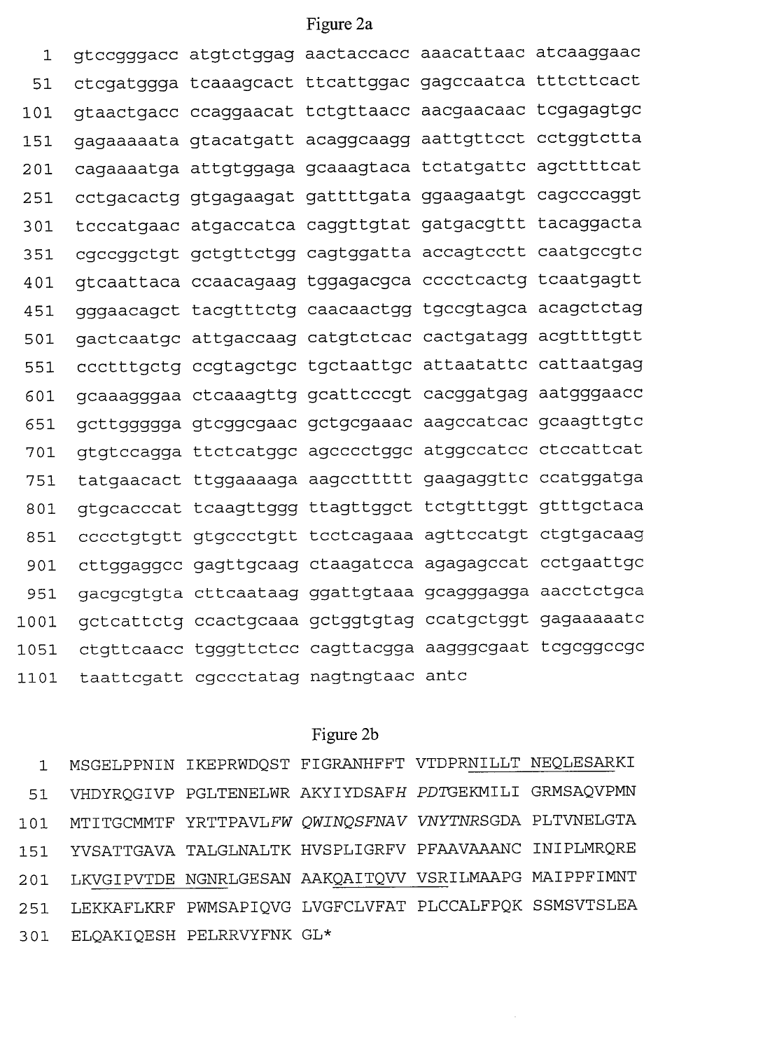

ADPI-41 was isolated, subjected to proteolysis, and analysed by mass spectrometry using the methods and apparatus of the Preferred Technology. Using the SEQUEST search program as described infra, uninterpreted tandem mass spectra of tryptic digest peptides were searched against a database of public domain proteins constructed of protein entries in the non-redundant database held by the National Centre for Biotechnology Information (NCBI), which is accessible at http://www.ncbi.nlm.nih.gov/ and also constructed of Expressed Sequence Tags entries (http://www.ncbi.nlm.nih.gov/dbEST/index.html). As a result of database searching, the following amino acid sequences of tryptic digest peptides of ADPI-41 (shown in FIG. 2a) were determined from matches to tryptic digest peptides in conceptual translation of ESTs:

BE298534, AI014241, and AV655958: NILLTNEQLESAR.

AA568689, AW796078, AA782417: QAITQVVVSR.

BF126487, BG388906, BG577432: VGIPVTDENGNR

These sequences have a very high similarity to tryptic digest peptides in the mouse sideroflexin 1 protein (from mitochondria, accession number 15147224, accessible at http://www.ncbi.nlm.nih.gov/entrez/), identified with a subsequent Blast search (http://www.ncbi.nlm.nih.gov/blast). Cloning of ADPI-41 and identification of splice variants

ADPI 41 was cloned (SEQ ID No. 753, shown in FIG. 2a and SEQ ID No.748, shown in FIG. 2b) using the following primers:

| | |
|---|---|
| Sense (F1)- | |
| 5′ actgagcgggacctgcgagc 3′ | (SEQ ID NO:755) |
| Antisense (R1)- | |
| 5′ tccgtaactgggagaacccagg 3′ | (SEQ ID NO:750) |

A Blast search against High Throughput Genomic Sequencing data (http://www.ncbi.nlm.nih.gov/blast) localised the ADPI 41 sequence to chromosome 5 clone RP11-606P24 (AC025713).

The DNA sequences encoding two of the identified peptides are as follows:

| | |
|---|---|
| aac att ctg tta acc aac gaa caa | (SEQ ID NO:751) |
| ctc gag agt gcg aga | |
| Asn Ile Leu Leu Thr Asn Glu Gln | (SEQ ID NO:568) |
| Leu Glu Ser Ala Arg and | |
| caa gcc atc acg caa gtt gtc gtg | (SEQ ID NO:752) |
| tcc agg | |
| Gln Ala Ile Thr Gln Val Val Val | (SEQ ID NO:594) |
| Ser Arg | |

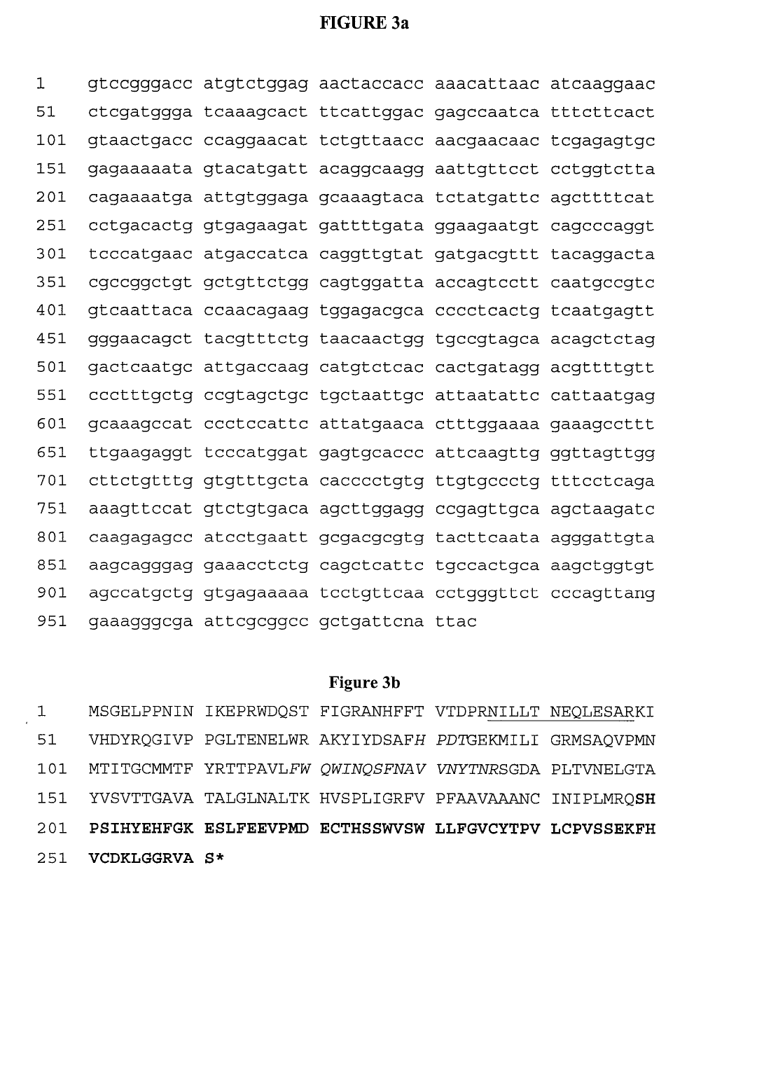

(ESTs AV655958 and AV655932 respectively) suggest there is a splice variant that lacks amino acids 65-82 when translated. These ESTs are both from a liver library. Using the primers described above, a splice variant was amplified from both brain and liver (SEQ ID No. 754 shown in FIG. 3a and SEQ ID No. 749 shown in FIG. 3b), in addition to the full-length clone. When compared to the genomic sequence, the splice variant is lacking a complete exon. The reading frame, however, is not maintained in this shorter version, and so the translated protein is different after the unspliced exon.

Protein Characterisation

Several arguments suggest an important role for this protein as a marker and/or therapeutic target in Alzheimer's disease. Several groups have confirmed an iron dysregulation in Alzheimer's disease associated with an accumulation of iron (Smith, M. A., A. Nunomura, et al. (2000). Antioxid Redox Signal 2(3): 413-20; Kala, S. V., B. B. Hasinoff, et al. (1996). Int J Neurosci 86(3-4): 263-9.). This is in agreement with the proposed role of Sxfn in sideroblastic anaemia where a loss of Sxfn protein is associated with an accumulation of iron in mitochondria of normoblasts (Fleming, M. D., D. R. Campagna, et al. (2001). “A mutation in a mitochondrial transmembrane protein is responsible for the pleiotropic hematological and skeletal phenotype of flexed-tail (f/f) mice.” Genes Dev 15(6): 652-7). Iron is able to mediate free-radical production via the Fenton Reaction and this may well contribute to the oxidative damage seen throughout the brain in Alzheimer's disease patients (Smith, M. A., P. L. Harris, et al. (1997). Proc Natl Acad Sci U S A 94(18): 9866-8.).

In addition, we provide the first evidence that the protein undergoes alternative splicing to result in an isoform of 261 amino acids.

Various publications in addition to the immediately foregoing are cited herein, the disclosures of which are incorporated by reference in their entireties. The citation of any reference herein should not be deemed as an admission that such reference is available as prior art to the instant invention.

### EXAMPLE 3

### Diagnosis and Treatment of Alzheimer's Disease

The following example illustrate the use of an ADPI of the invention for screening or diagnosis of Alzheimer's disease, determining the prognosis of a subject having Alzheimer's disease, or monitoring the effectiveness of Alzheimer's disease therapy. The following example also illustrates the use of modulators (e.g., agonist or antagonists) of an ADPI of the invention to treat or prevent Alzheimer's disease. Mitogen-Activated Protein Kinases (MAPKs) play a central role in response to a broad spectrum of extracellular stimuli including growth factors, neurotrophic factors, cytokines, hormones, neurotransmitters and cellular stress. Receptors activating the MAPK cascade include receptor tyrosine kinases, G-protein coupled receptors, integrins and ion channels. Extracellular signal-regulated kinases (ERKs) are emerging as important regulators of neuronal function. Numerous second messengers, such as cyclic adenosine monophosphate, protein kinase A, calcium, and diacylglycerol, can control ERK signalling via the small G proteins including Ras and Rap1 and may be responsible for the role of ERKs in the regulation of activity-dependent neuronal events, such as synaptic plasticity, long-term potentiation and cell survival. An activated ERK dimer regulates targets in the cytosol and also translocates to the nucleus, where it phosphorylates a variety of transcription factors regulating gene expression.

The expression of ERK-2 with a molecular weight of 40,709 kDa and pI of 6.65 respectively has been shown herein to be significantly increased in the Amygdala and Neocortex of subjects having Alzheimer's disease as compared with the Amygdala and Neocortex of subjects free from Alzheimer's disease. Thus, quantitative detection of ERK-2 in Amygdala and Neocortex can be used to diagnose Alzheimer's disease, determine the progression of Alzheimer's disease or monitor the effectiveness of a therapy for Alzheimer's disease.

In one embodiment of the invention, compounds that modulate (i.e., upregulate or downregulate) the expression, activity or both the expression and activity of ERK-2 are administered to a subject in need of treatment or for prophylaxis of Alzheimer's disease. Antibodies that modulate the expression, activity or both the expression and activity of ERK-2 are suitable for this purpose. In addition, nucleic acids coding for all or a portion of ERK-2, or nucleic acids complementary to all or a portion of ERK-2, may be administered. ERK-2, or fragments of the ERK-2 polypeptide may also be administered.

The invention also provides screening assays to identify additional compounds that modulate the expression of ERK-2 or activity of ERK-2. Compounds that modulate the expression of ERK-2 in vitro can be identified by comparing the expression of ERK-2 in cells treated with a test compound to the expression of ERK-2 in cells treated with a control compound (e.g., saline). Methods for detecting expression of ERK-2 are known in the art and include measuring the level of ERK-2 RNA (e.g., by northern blot analysis or RT-PCR) and measuring ERK-2 protein (e.g., by immunoassay or western blot analysis). Compounds that modulate the activity of ERK-2 can be identified by comparing the ability of a test compound to agonize or antagonize a function of ERK-2, such as its neurotrophic activity, to the ability of a control compound (e.g., saline) to inhibit the same function of ERK-2. Compounds capable of modulating ERK-2 activity are identified as compounds suitable for further development as a compound useful for the treatment of Alzheimer's disease.

Compounds identified in vitro that affect the expression or activity of ERK-2 can be tested in vivo in animal models of Alzheimer's disease or Downs syndrome, or in subjects having Alzheimer's disease, to determine their therapeutic efficacy.

The present invention is not to be limited in terms of the particular embodiments described in this application, which are intended as single illustrations of individual aspects of the invention. Functionally equivalent methods and apparatus within the scope of the invention, in addition to those enumerated herein, will be apparent to those skilled in the art from the foregoing description and accompanying drawings. Such modifications and variations are intended to fall within the scope of the appended claims. The contents of each reference, patent and patent application cited in this application is hereby incorporated by reference in its entirety.

When a reference is made herein to a method of treating of preventing a disease or condition using a particular agent or combination of agents, it is to be understood that such a reference is intended to include the use of that agent or combination of agents in the preparation of a medicament for the treatment of prevention of the disease or condition.

Preferred features of each aspect of the invention are as for each of the other aspects mutatis mutandis.

1. A method for screening, diagnosis or prognosis of Alzheimer's disease in a subject, for determining the stage or severity of Alzheimer's disease in a subject, for identifying a subject at risk of developing Alzheimer's disease, or for monitoring the effect of therapy administered to a subject having Alzheimer's disease, said method comprising:
(a) analysing a test sample of brain tissue from the subject by two dimensional electrophoresis to generate a two-dimensional array of features, said array comprising at least one chosen feature whose relative abundance correlates with the presence, absence, stage or severity of Alzheimer's disease or predicts the onset or course of Alzheimer's disease; and
(b) comparing the abundance of each chosen feature in the test sample with the abundance of that chosen feature in brain tissue from one or more persons free from Alzheimer's disease, or with a previously determined reference range for that feature in subjects free from Alzheimer's disease, or with the abundance at least one Expression Reference Feature (ERF) in the test sample.

2. The method of claim 1, wherein said method is for screening or diagnosis of Alzheimer's disease and the relative abundance of at least one chosen feature correlates with the presence or absence of Alzheimer's disease.

3. The method of claim 1, wherein said method is for monitoring the effect of therapy administered to a subject having Alzheimer's disease and the relative abundance of at least one chosen feature correlates with the severity of Alzheimer's disease.

4. The method of claim 1, 2 or 3, wherein step (b) comprises comparing the abundance of each chosen feature in the sample with the abundance of that chosen feature in brain tissue from one or more persons free from Alzheimer's disease or with a previously determined reference range for that chosen feature in subjects free from Alzheimer's disease.

5. The method of any claim 1, 2, 3 or 4, wherein step (b) comprises quantitatively detecting one or more of the following Alzheimer's Disease-Associated Features (ADFs): ADF-1, ADF-3, ADF-5, ADF-6, ADF-8, ADF-9, ADF-10, ADF-11, ADF-12, ADF-13, ADF-15, ADF-16, ADF-22, ADF-23, ADF-24, ADF-25, ADF-26, ADF-27, ADF-29, ADF-31, ADF-33, ADF-35, ADF-37, ADF-39, ADF-42, ADF-54, ADF-56, ADF-61, ADF-62, ADF-66, ADF-67, ADF-68, ADF-70, ADF-72, ADF-77, ADF-78, ADF-79, ADF-81, ADF-82, ADF-85, ADF-87, ADF-88, ADF-90, ADF-91, ADF-92, ADF-94, ADF-95, ADF-97, ADF-98, ADF-101, ADF-102, ADF-103, ADF-105, ADF-107, ADF-108, ADF-109, ADF-111, ADF-112, ADF-113, ADF-115, ADF-117, ADF-119, ADF-120, ADF-121, ADF-124, ADF-125, ADF-126, ADF-127, ADF-128, ADF-129, ADF-130, ADF-131, ADF-132, ADF-133, ADF-138, ADF-139, ADF-140, ADF-141, ADF-142, ADF-143, ADF-144, ADF-146, ADF-148, ADF-149, ADF-150, ADF-151, ADF-152, ADF-153, ADF-154, ADF-155, ADF-156, ADF-157, ADF-159, ADF-160, ADF-162, ADF-163, ADF-165, ADF-172, ADF-173, ADF-175, ADF-176, ADF-182, ADF-183, ADF-188, ADF-189, ADF-191, ADF-193, ADF-194, ADF-196, ADF-202, ADF-204, ADF-208, ADF-209, ADF-216, ADF-217, ADF-220, ADF-223, ADF-228, ADF-229, ADF-230, ADF-232, ADF-236, ADF-237, ADF-240, ADF-243, ADF-245, ADF-250, ADF-251, ADF-252, ADF-253, ADF-254, ADF-255, ADF-256, ADF-257, ADF-258, ADF-259, ADF-260, ADF-261, ADF-262, ADF-263, ADF-264, ADF-265, ADF-266, ADF-267, ADF-268, ADF-269, ADF-270, ADF-271, ADF-272, ADF-273, ADF-274, ADF-275, ADF-276, ADF-277, ADF-278, ADF-279, ADF-280, ADF-281, ADF-282, ADF-283, ADF-284, ADF-285, ADF-286, ADF-287, ADF-288, ADF-289, ADF-290, ADF-291, ADF-292, ADF-293, ADF-294, ADF-295, ADF-296, ADF-297, ADF-298, ADF-299, ADF-300, ADF-301, ADF-302, ADF-303, ADF-304, ADF-305, ADF-306, ADF-307, ADF-308, ADF-309, ADF-311, ADF-312, ADF-313, ADF-314, ADF-315, ADF-316, ADF-317, ADF-318, ADF-319, ADF-320, ADF-321, ADF-322, ADF-323, ADF-324, ADF-325, ADF-326, ADF-327, ADF-328, ADF-329, ADF-330, ADF-331, ADF-332, ADF-333, ADF-334, ADF-335, ADF-336, ADF-337, ADF-338, ADF-339, ADF-340, ADF-341, ADF-342, ADF-343, ADF-344, ADF-345, ADF-346, ADF-347, ADF-348, ADF-349, ADF-350, ADF-351, ADF-352, ADF-353, ADF-354, ADF-355, ADF-356, ADF-357, ADF-358, ADF-359, ADF-360, ADF-361, ADF-362, ADF-363, ADF-364, ADF-365, ADF-366, ADF-367, ADF-368, ADF-369, ADF-370, ADF-371, ADF-372, ADF-373, ADF-374, ADF-375, ADF-376, ADF-377, ADF-378, ADF-379, ADF-380, ADF-381, ADF-382, ADF-383, ADF-384, ADF-386, ADF-387, ADF-388, ADF-389, ADF-390, ADF-391, ADF-392, ADF-393, ADF-394, ADF-395, ADF-396, ADF-397, ADF-398, ADF-399, ADF-400, ADF-401, ADF-402, ADF-403, ADF-404, ADF-405, ADF-406, ADF-407, ADF-408, ADF-409, ADF-410, ADF-411, ADF-412, ADF-413, ADF-414, ADF-415, ADF-416, ADF-417, ADF-418, ADF-419, ADF-420, ADF-421, ADF-422, ADF-423, ADF-424, ADF-425, ADF-426, ADF-427, ADF-428, ADF-429, ADF-430, ADF-431, ADF-432, ADF-433, ADF-434, ADF-435, ADF-436, ADF-437, ADF-438, ADF-439, ADF-440, ADF-441, ADF-442, ADF-443, ADF-444, ADF-445, ADF-446, ADF-447, ADF-448, ADF-449, ADF-450, ADF-451, ADF-452, ADF-453, ADF-454, ADF-455, ADF-456, ADF-457, ADF-458, ADF-459, ADF-460, ADF-461, ADF-462, ADF-463, ADF-464, ADF-465, ADF-466, ADF-467, ADF-468, ADF-469, ADF-470, ADF-471, ADF-472, ADF-473, ADF-474, ADF-475, ADF-476, ADF-477, ADF-478, ADF-479, ADF-480, ADF-481, ADF-482, ADF-483, ADF-484, ADF-485, ADF-486, ADF-487, ADF-488, ADF-489, ADF-490, ADF-491, ADF-492, ADF-493, ADF-494, ADF-495, ADF-496, ADF-497, ADF-498, ADF-499, ADF-500, ADF-501, ADF-502, ADF-503, ADF-504, ADF-505, ADF-506, ADF-507, ADF-508, ADF-509, ADF-510, ADF-511, ADF-512, ADF-513, ADF-514, ADF-515, ADF-516, ADF-517, ADF-518, ADF-519, ADF-520, ADF-521, ADF-522, ADF-523, ADF-524, ADF-525, ADF-526, ADF-527, ADF-528, ADF-529, ADF-530, ADF-531, ADF-532, ADF-533, ADF-534, ADF-535, ADF-536, ADF-537, ADF-538, ADF-539, ADF-540, ADF-541, ADF-542, ADF-543, ADF-544, ADF-545, ADF-546, ADF-547, ADF-548, ADF-549, optionally in combination with ADF-41.

6. The method of claim 1-4 or 5, wherein step (a) comprises isoelectric focussing followed by sodium dodecyl sulfate polyacrylamide gel electrophoresis (SDS-PAGE).

7. A method for screening, diagnosis or prognosis of Alzheimer's disease in a subject, for determining the stage or severity of Alzheimer's disease in a subject, for identifying a subject at risk of developing Alzheimer's disease, or for monitoring the effect of therapy administered to a subject having Alzheimer's disease, said method comprising:
quantitatively detecting, in a sample of brain tissue from the subject, at least one of the following Alzheimer's Disease-Associated Protein Isoforms (ADPIs): ADPI-1, ADPI-3.1, ADPI-3.2, ADPI-3.3, ADPI-5.3, ADPI-6, ADPI-8, ADPI-9, ADPI-10, ADPI-11, ADPI-12.1, ADPI-12.2, ADPI-13, ADPI-15, ADPI-16, ADPI-22, ADPI-23, ADPI-24, ADPI-25.3, ADPI-25.2, ADPI-26, ADPI-27, ADPI-29, ADPI-31, ADPI-33.1, ADPI-35, ADPI-37, ADPI-39, ADPI-42, ADPI-54, ADPI-56, ADPI-61, ADPI-62, ADPI-66, ADPI-67, ADPI-68, ADPI-70, ADPI-72, ADPI-77, ADPI-78.2, ADPI-78.3, ADPI-79, ADPI-81.2, ADPI-82, ADPI-85, ADPI-87.1, ADPI-88, ADPI-90, ADPI-91.1, ADPI-91.2, ADPI-92, ADPI-94, ADPI-95, ADPI-97, ADPI-98, ADPI-101, ADPI-102.1, ADPI-103, ADPI-105.2, ADPI-105.1, ADPI-107, ADPI-108, ADPI-109, ADPI-111.1, ADPI-112, ADPI-113, ADPI-115, ADPI-117.1, ADPI-19, ADPI-120, ADPI-121, ADPI-124, ADPI-125, ADPI-126, ADPI-126, ADPI-126, ADPI-127, ADPI-128, ADPI-129, ADPI-130, ADPI-131, ADPI-132, ADPI-133, ADPI-138, ADPI-139, ADPI-140, ADPI-141, ADPI-142.2, ADPI-142.1, ADPI-143.1, ADPI-143.2, ADPI-144, ADPI-146.2, ADPI-146.1, ADPI-148, ADPI-149.1, ADPI-150, ADPI-151.2, ADPI-151.1, ADPI-152, ADPI-153.1, ADPI-153.2, ADPI-153.3, ADPI-154, ADPI-155, ADPI-156.2, ADPI-156.1, ADPI-157, ADPI-159.1, ADPI-159.2, ADPI-160, ADPI-162.2, ADPI-162.3, ADPI-162.1, ADPI-163, ADPI-165, ADPI-172, ADPI-173.1, ADPI-173.2, ADPI-175.2, ADPI-175.1, ADPI-176, ADPI-182, ADPI-183, ADPI-188.1, ADPI-189.2, ADPI-191, ADPI-193, ADPI-194.2, ADPI-196.2, ADPI-196.3, ADPI-196.1, ADPI-196.4, ADPI-202, ADPI-204, ADPI-208, ADPI-209, ADPI-216, ADPI-217, ADPI-220, ADPI-223, ADPI-228, ADPI-229, ADPI-230.1, ADPI-230.2, ADPI-232.1, ADPI-232.2, ADPI-236.2, ADPI-236.3, ADPI-237.2, ADPI-237.1, ADPI-240, ADPI-243, ADPI-245, ADPI-250, ADPI-251, ADPI-252.1, ADPI-252.2, ADPI-253, ADPI-254, ADPI-255, ADPI-256, ADPI-257.1, ADPI-257.2, ADPI-258, ADPI-259.1, ADPI-259.2, ADPI-260, ADPI-261, ADPI-262, ADPI-263, ADPI-264, ADPI-265, ADPI-266, ADPI-267, ADPI-268.1, ADPI-268.2, ADPI-269, ADPI-270, ADPI-271, ADPI-272, ADPI-273, ADPI-274, ADPI-275, ADPI-276, ADPI-277, ADPI-278.1, ADPI-278.2, ADPI-279.1, ADPI-279.2, ADPI-280, ADPI-281, ADPI-282, ADPI-283, ADPI-284, ADPI-285, ADPI-286, ADPI-287, ADPI-288, ADPI-289, ADPI-290, ADPI-291, ADPI-292, ADPI-293, ADPI-294, ADPI-295, ADPI-296, ADPI-297, ADPI-298, ADPI-299, ADPI-300, ADPI-301, ADPI-302, ADPI-303, ADPI-304, ADPI-305, ADPI-306, ADPI-307.1, ADPI-307.2, ADPI-308, ADPI-309, ADPI-311, ADPI-312, ADPI-313, ADPI-314, ADPI-315, ADPI-316.1, ADPI-316.2, ADPI-317, ADPI-318, ADPI-319, ADPI-320.1, ADPI-320.2, ADPI-321, ADPI-322, ADPI-323, ADPI-324, ADPI-325, ADPI-326, ADPI-327, ADPI-328, ADPI-329, ADPI-330, ADPI-331, ADPI-332, ADPI-333.1, ADPI-333.2, ADPI-334, ADPI-335, ADPI-336, ADPI-337, ADPI-338, ADPI-339, ADPI-340, ADPI-341, ADPI-342, ADPI-343, ADPI-344, ADPI-345, ADPI-346, ADPI-347, ADPI-348, ADPI-349, ADPI-350, ADPI-351, ADPI-352, ADPI-353, ADPI-354, ADPI-355, ADPI-356, ADPI-357, ADPI-358, ADPI-359, ADPI-360, ADPI-361, ADPI-362, ADPI-363, ADPI-364, ADPI-365.1, ADPI-365.2, ADPI-366, ADPI-367.1, ADPI-367.2, ADPI-368.1, ADPI-368.2, ADPI-369.1, ADPI-369.2, ADPI-370, ADPI-371, ADPI-372, ADPI-373, ADPI-374, ADPI-375, ADPI-376, ADPI-377, ADPI-378, ADPI-379, ADPI-380, ADPI-381, ADPI-382, ADPI-383, ADPI-384, ADPI-386, ADPI-387, ADPI-388, ADPI-389, ADPI-390, ADPI-391, ADPI-392, ADPI-393, ADPI-394, ADPI-395, ADPI-396, ADPI-397, ADPI-398, ADPI-399.1, ADPI-399.2, ADPI-400, ADPI-401, ADPI-402, ADPI-403, ADPI-404.1, ADPI-404.2, ADPI-405, ADPI-406, ADPI-407, ADPI-408, ADPI-409, ADPI-410, ADPI-411, ADPI-412.1, ADPI-412.2, ADPI-413, ADPI-414, ADPI-415, ADPI-416, ADPI-417, ADPI-418, ADPI-419, ADPI-420, ADPI-421, ADPI-422, ADPI-423, ADPI-424, ADPI-425, ADPI-426.1, ADPI-426.2, ADPI-427, ADPI-428, ADPI-429, ADPI-430, ADPI-431, ADPI-432, ADPI-433, ADPI-434, ADPI-435, ADPI-435, ADPI-436, ADPI-437, ADPI-438, ADPI-439, ADPI-440.1, ADPI-440.2, ADPI-441, ADPI-442, ADPI-443, ADPI-444, ADPI-445, ADPI-446, ADPI-447.1, ADPI-447.2, ADPI-448, ADPI-449, ADPI-450.1, ADPI-450.2, ADPI-451, ADPI-452, ADPI-453, ADPI-454, ADPI-455, ADPI-456, ADPI-457, ADPI-458, ADPI-459, ADPI-460, ADPI-461, ADPI-462, ADPI-463, ADPI-464, ADPI-465, ADPI-466.1, ADPI-466.2, ADPI-467, ADPI-468, ADPI-469.1, ADPI-469.2, ADPI-470, ADPI-471, ADPI-472, ADPI-473, ADPI-474, ADPI-475, ADPI-476, ADPI-477, ADPI-478.1, ADPI-478.2, ADPI-479, ADPI-480, ADPI-481, ADPI-482, ADPI-483, ADPI-484, ADPI-485, ADPI-486, ADPI-487, ADPI-488, ADPI-489, ADPI-490, ADPI-491, ADPI-492, ADPI-493.1, ADPI-493.2, ADPI-494, ADPI-495, ADPI-496, ADPI-497, ADPI-498, ADPI-499, ADPI-500, ADPI-501, ADPI-502, ADPI-503, ADPI-504, ADPI-505, ADPI-506, ADPI-507, ADPI-508, ADPI-509, ADPI-510, ADPI-511, ADPI-512, ADPI-513, ADPI-514, ADPI-515, ADPI-516, ADPI-517, ADPI-518, ADPI-519, ADPI-520, ADPI-521, ADPI-522, ADPI-523, ADPI-524, ADPI-525, ADPI-526.1, ADPI-526.2, ADPI-527, ADPI-528, ADPI-529, ADPI-530.1, ADPI-530.2, ADPI-531, ADPI-532, ADPI-533, ADPI-534, ADPI-535, ADPI-536, ADPI-537, ADPI-538, ADPI-539, ADPI-540, ADPI-541, ADPI-542, ADPI-543, ADPI-544, ADPI-545, ADPI-546, ADPI-547.1, ADPI-547.2, ADPI-547.3, ADPI-548.1, ADPI-548.2, ADPI-549, optionally in combination with ADPI-41; and
comparing the level or amount of said isoform or isoforms detected in step (a) with a control.

8. The method of claim 7, wherein the step of quantitatively detecting comprises testing at least one aliquot of the sample, said step of testing comprising:
(a) contacting the aliquot with an antibody that is immunospecific for a preselected ADPI;
(b) quantitatively measuring any binding that has occurred between the antibody and at least one species in the aliquot; and
(c) comparing the results of step (b) to a control.

9. The method of claim 8, wherein the step of quantitatively detecting comprises testing a plurality of aliquots with a plurality of antibodies for quantitative detection of a plurality of preselected ADPIs.

10. The method of claim 8 or claim 9, wherein the or each antibody is/are monoclonal.

11. The method of claim 10, wherein the or each antiboyd is/are chimeric.

12. A preparation comprising at least one of the following isolated Alzheimer's Disease-Associated Protein Isoforms (ADPIs) said ADPI selected from ADPI-1, ADPI-3.1, ADPI-3.2, ADPI-3.3, ADPI-5.3, ADPI-6, ADPI-8, ADPI-9, ADPI-10, ADPI-11, ADPI-12.1, ADPI-12.2, ADPI-13, ADPI-15, ADPI-16, ADPI-22, ADPI-23, ADPI-24, ADPI-25.3, ADPI-25.2, ADPI-26, ADPI-27, ADPI-29, ADPI-31, ADPI-33.1, ADPI-35, ADPI-37, ADPI-39, ADPI-42, ADPI-54, ADPI-56, ADPI-61, ADPI-62, ADPI-66, ADPI-67, ADPI-68, ADPI-70, ADPI-72, ADPI-77, ADPI-78.2, ADPI-78.3, ADPI-79, ADPI-81.2, ADPI-82, ADPI-85, ADPI-87.1, ADPI-88, ADPI-90, ADPI-91.1, ADPI-91.2, ADPI-92, ADPI-94, ADPI-95, ADPI-97, ADPI-98, ADPI-101, ADPI-102.1, ADPI-103, ADPI-105.2, ADPI-105.1, ADPI-107, ADPI-108, ADPI-109, ADPI-l1.l, ADPI-112, ADPI-113, ADPI-115, ADPI-117.1, ADPI-119, ADPI-120, ADPI-121, ADPI-124, ADPI-125, ADPI-126, ADPI-126, ADPI-126, ADPI-127, ADPI-128, ADPI-129, ADPI-130, ADPI-131, ADPI-132, ADPI-133, ADPI-138, ADPI-139, ADPI-140, ADPI-141, ADPI-142.2, ADPI-142.1, ADPI-143.1, ADPI-143.2, ADPI-144, ADPI-146.2, ADPI-146.1, ADPI-148, ADPI-149.1, ADPI-150, ADPI-151.2, ADPI-151.1, ADPI-152, ADPI-153.1, ADPI-153.2, ADPI-153.3, ADPI-154, ADPI-155, ADPI-156.2, ADPI-156.1, ADPI-157, ADPI-159.1, ADPI-159.2, ADPI-160, ADPI-162.2, ADPI-162.3, ADPI-162.1, ADPI-163, ADPI-165, ADPI-172, ADPI-173.1, ADPI-173.2, ADPI-175.2, ADPI-175.1, ADPI-176, ADPI-182, ADPI-183, ADPI-188.1, ADPI-189.2, ADPI-191, ADPI-193, ADPI-194.2, ADPI-196.2, ADPI-196.3, ADPI-196.1, ADPI-196.4, ADPI-202, ADPI-204, ADPI-208, ADPI-209, ADPI-216, ADPI-217, ADPI-220, ADPI-223, ADPI-228, ADPI-229, ADPI-230.1, ADPI-230.2, ADPI-232.1, ADPI-232.2, ADPI-236.2, ADPI-236.3, ADPI-237.2, ADPI-237.1, ADPI-240, ADPI-243, ADPI-245, ADPI-250, ADPI-251, ADPI-252.1, ADPI-252.2, ADPI-253, ADPI-254, ADPI-255, ADPI-256, ADPI-257.1, ADPI-257.2, ADPI-258, ADPI-259.1, ADPI-259.2, ADPI-260, ADPI-261, ADPI-262, ADPI-263, ADPI-264, ADPI-265, ADPI-266, ADPI-267, ADPI-268.1, ADPI-268.2, ADPI-269, ADPI-270, ADPI-271, ADPI-272, ADPI-273, ADPI-274, ADPI-275, ADPI-276, ADPI-277, ADPI-278.1, ADPI-278.2, ADPI-279.1, ADPI-279.2, ADPI-280, ADPI-281, ADPI-282, ADPI-283, ADPI-284, ADPI-285, ADPI-286, ADPI-287, ADPI-288, ADPI-289, ADPI-290, ADPI-291, ADPI-292, ADPI-293, ADPI-294, ADPI-295, ADPI-296, ADPI-297, ADPI-298, ADPI-299, ADPI-300, ADPI-301, ADPI-302, ADPI-303, ADPI-304, ADPI-305, ADPI-306, ADPI-307.1, ADPI-307.2, ADPI-308, ADPI-309, ADPI-311, ADPI-312, ADPI-313, ADPI-314, ADPI-315, ADPI-316.1, ADPI-316.2, ADPI-317, ADPI-318, ADPI-319, ADPI-320.1, ADPI-320.2, ADPI-321, ADPI-322, ADPI-323, ADPI-324, ADPI-325, ADPI-326, ADPI-327, ADPI-328, ADPI-329, ADPI-330, ADPI-331, ADPI-332, ADPI-333.1, ADPI-333.2, ADPI-334, ADPI-335, ADPI-336, ADPI-337, ADPI-338, ADPI-339, ADPI-340, ADPI-341, ADPI-342, ADPI-343, ADPI-344, ADPI-345, ADPI-346, ADPI-347, ADPI-348, ADPI-349, ADPI-350, ADPI-351, ADPI-352, ADPI-353, ADPI-354, ADPI-355, ADPI-356, ADPI-357, ADPI-358, ADPI-359, ADPI-360, ADPI-361, ADPI-362, ADPI-363, ADPI-364, ADPI-365.1, ADPI-365.2, ADPI-366, ADPI-367.1, ADPI-367.2, ADPI-368.1, ADPI-368.2, ADPI-369.1, ADPI-369.2, ADPI-370, ADPI-371, ADPI-372, ADPI-373, ADPI-374, ADPI-375, ADPI-376, ADPI-377, ADPI-378, ADPI-379, ADPI-380, ADPI-381, ADPI-382, ADPI-383, ADPI-384, ADPI-386, ADPI-387, ADPI-388, ADPI-389, ADPI-390, ADPI-391, ADPI-392, ADPI-393, ADPI-394, ADPI-395, ADPI-396, ADPI-397, ADPI-398, ADPI-399.1, ADPI-399.2, ADPI-400, ADPI-401, ADPI-402, ADPI-403, ADPI-404.1, ADPI-404.2, ADPI-405, ADPI-406, ADPI-407, ADPI-408, ADPI-409, ADPI-410, ADPI-411, ADPI-412.1, ADPI-412.2, ADPI-413, ADPI-414, ADPI-415, ADPI-416, ADPI-417, ADPI-418, ADPI-419, ADPI-420, ADPI-421, ADPI-422, ADPI-423, ADPI-424, ADPI-425, ADPI-426.1, ADPI-426.2, ADPI-427, ADPI-428, ADPI-429, ADPI-430, ADPI-431, ADPI-432, ADPI-433, ADPI-434, ADPI-435, ADPI-435, ADPI-436, ADPI-437, ADPI-438, ADPI-439, ADPI-440.1, ADPI-440.2, ADPI-441, ADPI-442, ADPI-443, ADPI-444, ADPI-445, ADPI-446, ADPI-447.1, ADPI-447.2, ADPI-448, ADPI-449, ADPI-450.1, ADPI-450.2, ADPI-451, ADPI-452, ADPI-453, ADPI-454, ADPI-455, ADPI-456, ADPI-457, ADPI-458, ADPI-459, ADPI-460, ADPI-461, ADPI-462, ADPI-463, ADPI-464, ADPI-465, ADPI-466.1, ADPI-466.2, ADPI-467, ADPI-468, ADPI-469.1, ADPI-469.2, ADPI-470, ADPI-471, ADPI-472, ADPI-473, ADPI-474, ADPI-475, ADPI-476, ADPI-477, ADPI-478.1, ADPI-478.2, ADPI-479, ADPI-480, ADPI-481, ADPI-482, ADPI-483, ADPI-484, ADPI-485, ADPI-486, ADPI-487, ADPI-488, ADPI-489, ADPI-490, ADPI-491, ADPI-492, ADPI-493.1, ADPI-493.2, ADPI-494, ADPI-495, ADPI-496, ADPI-497, ADPI-498, ADPI-499, ADPI-500, ADPI-501, ADPI-502, ADPI-503, ADPI-504, ADPI-505, ADPI-506, ADPI-507, ADPI-508, ADPI-509, ADPI-510, ADPI-511, ADPI-512, ADPI-513, ADPI-514, ADPI-515, ADPI-516, ADPI-517, ADPI-518, ADPI-519, ADPI-520, ADPI-521, ADPI-522, ADPI-523, ADPI-524, ADPI-525, ADPI-526.1, ADPI-526.2, ADPI-527, ADPI-528, ADPI-529, ADPI-530.1, ADPI-530.2, ADPI-531, ADPI-532, ADPI-533, ADPI-534, ADPI-535, ADPI-536, ADPI-537, ADPI-538, ADPI-539, ADPI-540, ADPI-541, ADPI-542, ADPI-543, ADPI-544, ADPI-545, ADPI-546, ADPI-547.1, ADPI-547.2, ADPI-547.3, ADPI-548.1, ADPI-548.2, ADPI-549, optionally in combination with ADF-41.

13. The preparation of claim 15, wherein the or each Alzheimer's Disease-Associated Protein Isoform (ADPI) is/are in recombinant or isolated form.

14. A kit comprising the preparation of claim 12 or claim 13, other reagents, and directions for use.

15. A kit comprising a plurality of distinct preparations as claimed in claim 12 or claim 13.

16. An antibody capable of immunospecific binding to one of the following Alzheimer's Disease-Associated Protein Isoforms (ADPIs): ADPI-1, ADPI-3.1, ADPI-3.2, ADPI-3.3, ADPI-5.3, ADPI-6, ADPI-8, ADPI-9, ADPI-10, ADPI-11, ADPI-12.1, ADPI-12.2, ADPI-13, ADPI-15, ADPI-16, ADPI-22, ADPI-23, ADPI-24, ADPI-25.3, ADPI-25.2, ADPI-26, ADPI-27, ADPI-29, ADPI-31, ADPI-33.1, ADPI-35, ADPI-37, ADPI-39, ADPI-42, ADPI-54, ADPI-56, ADPI-61, ADPI-62, ADPI-66, ADPI-67, ADPI-68, ADPI-70, ADPI-72, ADPI-77, ADPI-78.2, ADPI-78.3, ADPI-79, ADPI-81.2, ADPI-82, ADPI-85, ADPI-87.1, ADPI-88, ADPI-90, ADPI-91.1, ADPI-91.2, ADPI-92, ADPI-94, ADPI-95, ADPI-97, ADPI-98, ADPI-101, ADPI-102.1, ADPI-103, ADPI-105.2, ADPI-105.1, ADPI-107, ADPI-108, ADPI-109, ADPI-111.1, ADPI-112, ADPI-113, ADPI-115, ADPI-117.1, ADPI-119, ADPI-120, ADPI-121, ADPI-124, ADPI-125, ADPI-126, ADPI-126, ADPI-126, ADPI-127, ADPI-128, ADPI-129, ADPI-130, ADPI-131, ADPI-132, ADPI-133, ADPI-138, ADPI-139, ADPI-140, ADPI-141, ADPI-142.2, ADPI-142.1, ADPI-143.1, ADPI-143.2, ADPI-144, ADPI-146.2, ADPI-146.1, ADPI-148, ADPI-149.1, ADPI-150, ADPI-151.2, ADPI-151.1, ADPI-152, ADPI-153.1, ADPI-153.2, ADPI-153.3, ADPI-154, ADPI-155, ADPI-156.2, ADPI-156.1, ADPI-157, ADPI-159.1, ADPI-159.2, ADPI-160, ADPI-162.2, ADPI-162.3, ADPI-162.1, ADPI-163, ADPI-165, ADPI-172, ADPI-173.1, ADPI-173.2, ADPI-175.2, ADPI-175.1, ADPI-176, ADPI-182, ADPI-183, ADPI-188.1, ADPI-189.2, ADPI-191, ADPI-193, ADPI-194.2, ADPI-196.2, ADPI-196.3, ADPI-196.1, ADPI-196.4, ADPI-202, ADPI-204, ADPI-208, ADPI-209, ADPI-216, ADPI-217, ADPI-220, ADPI-223, ADPI-228, ADPI-229, ADPI-230.1, ADPI-230.2, ADPI-232.1, ADPI-232.2, ADPI-236.2, ADPI-236.3, ADPI-237.2, ADPI-237.1, ADPI-240, ADPI-243, ADPI-245, ADPI-250, ADPI-251, ADPI-252.1, ADPI-252.2, ADPI-253, ADPI-254, ADPI-255, ADPI-256, ADPI-257.1, ADPI-257.2, ADPI-258, ADPI-259.1, ADPI-259.2, ADPI-260, ADPI-261, ADPI-262, ADPI-263, ADPI-264, ADPI-265, ADPI-266, ADPI-267, ADPI-268.1, ADPI-268.2, ADPI-269, ADPI-270, ADPI-271, ADPI-272, ADPI-273, ADPI-274, ADPI-275, ADPI-276, ADPI-277, ADPI-278.1, ADPI-278.2, ADPI-279.1, ADPI-279.2, ADPI-280, ADPI-281, ADPI-282, ADPI-283, ADPI-284, ADPI-285, ADPI-286, ADPI-287, ADPI-288, ADPI-289, ADPI-290, ADPI-291, ADPI-292, ADPI-293, ADPI-294, ADPI-295, ADPI-296, ADPI-297, ADPI-298, ADPI-299, ADPI-300, ADPI-301, ADPI-302, ADPI-303, ADPI-304, ADPI-305, ADPI-306, ADPI-307.1, ADPI-307.2, ADPI-308, ADPI-309, ADPI-311, ADPI-312, ADPI-313, ADPI-314, ADPI-315, ADPI-316.1, ADPI-316.2, ADPI-317, ADPI-318, ADPI-319, ADPI-320.1, ADPI-320.2, ADPI-321, ADPI-322, ADPI-323, ADPI-324, ADPI-325, ADPI-326, ADPI-327, ADPI-328, ADPI-329, ADPI-330, ADPI-331, ADPI-332, ADPI-333.1, ADPI-333.2, ADPI-334, ADPI-335, ADPI-336, ADPI-337, ADPI-338, ADPI-339, ADPI-340, ADPI-341, ADPI-342, ADPI-343, ADPI-344, ADPI-345, ADPI-346, ADPI-347, ADPI-348, ADPI-349, ADPI-350, ADPI-351, ADPI-352, ADPI-353, ADPI-354, ADPI-355, ADPI-356, ADPI-357, ADPI-358, ADPI-359, ADPI-360, ADPI-361, ADPI-362, ADPI-363, ADPI-364, ADPI-365.1, ADPI-365.2, ADPI-366, ADPI-367.1, ADPI-367.2, ADPI-368.1, ADPI-368.2, ADPI-369.1, ADPI-369.2, ADPI-370, ADPI-371, ADPI-372, ADPI-373, ADPI-374, ADPI-375, ADPI-376, ADPI-377, ADPI-378, ADPI-379, ADPI-380, ADPI-381, ADPI-382, ADPI-383, ADPI-384, ADPI-386, ADPI-387, ADPI-388, ADPI-389, ADPI-390, ADPI-391, ADPI-392, ADPI-393, ADPI-394, ADPI-395, ADPI-396, ADPI-397, ADPI-398, ADPI-399.1, ADPI-399.2, ADPI-400, ADPI-401, ADPI-402, ADPI-403, ADPI-404.1, ADPI-404.2, ADPI-405, ADPI-406, ADPI-407, ADPI-408, ADPI-409, ADPI-410, ADPI-411, ADPI-412.1, ADPI-412.2, ADPI-413, ADPI-414, ADPI-415, ADPI-416, ADPI-417, ADPI-418, ADPI-419, ADPI-420, ADPI-421, ADPI-422, ADPI-423, ADPI-424, ADPI-425, ADPI-426.1, ADPI-426.2, ADPI-427, ADPI-428, ADPI-429, ADPI-430, ADPI-431, ADPI-432, ADPI-433, ADPI-434, ADPI-435, ADPI-435, ADPI-436, ADPI-437, ADPI-438, ADPI-439, ADPI-440.1, ADPI-440.2, ADPI-441, ADPI-442, ADPI-443, ADPI-444, ADPI-445, ADPI-446, ADPI-447.1, ADPI-447.2, ADPI-448, ADPI-449, ADPI-450.1, ADPI-450.2, ADPI-451, ADPI-452, ADPI-453, ADPI-454, ADPI-455, ADPI-456, ADPI-457, ADPI-458, ADPI-459, ADPI-460, ADPI-461, ADPI-462, ADPI-463, ADPI-464, ADPI-465, ADPI-466.1, ADPI-466.2, ADPI-467, ADPI-468, ADPI-469.1, ADPI-469.2, ADPI-470, ADPI-471, ADPI-472, ADPI-473, ADPI-474, ADPI-475, ADPI-476, ADPI-477, ADPI-478.1, ADPI-478.2, ADPI-479, ADPI-480, ADPI-481, ADPI-482, ADPI-483, ADPI-484, ADPI-485, ADPI-486, ADPI-487, ADPI-488, ADPI-489, ADPI-490, ADPI-491, ADPI-492, ADPI-493.1, ADPI-493.2, ADPI-494, ADPI-495, ADPI-496, ADPI-497, ADPI-498, ADPI-499, ADPI-500, ADPI-501, ADPI-502, ADPI-503, ADPI-504, ADPI-505, ADPI-506, ADPI-507, ADPI-508, ADPI-509, ADPI-510, ADPI-511, ADPI-512, ADPI-513, ADPI-514, ADPI-515, ADPI-516, ADPI-517, ADPI-518, ADPI-519, ADPI-520, ADPI-521, ADPI-522, ADPI-523, ADPI-524, ADPI-525, ADPI-526.1, ADPI-526.2, ADPI-527, ADPI-528, ADPI-529, ADPI-530.1, ADPI-530.2, ADPI-531, ADPI-532, ADPI-533, ADPI-534, ADPI-535, ADPI-536, ADPI-537, ADPI-538, ADPI-539, ADPI-540, ADPI-541, ADPI-542, ADPI-543, ADPI-544, ADPI-545, ADPI-546, ADPI-547.1, ADPI-547.2, ADPI-547.3, ADPI-548.1, ADPI-548.2, ADPI-549, optionally in combination with ADPI-41.

17. The antibody of claim 16, which is a monoclonal antibody.

18. The antibody of claim 16 or claim 17, which is a chimeric antibody.

19. The antibody of claim 16, 17 or 18, which binds to the ADPI with greater affinity than to another isoform or variant of the ADPI.

20. A kit comprising an antibody as claimed in any one of claims 16 to 19, other reagents, and directions for use.

21. A kit comprising a plurality of distinct antibodies as claimed in any one of claims 16 to 19.

22. A pharmaceutical composition comprising a therapeutically effective amount of an antibody as claimed in any one of claims 16 to 19 or a fragment or derivative of such an antibody containing the binding domain of the antibody, and a pharmaceutically acceptable carrier.

23. A method of treating or preventing Alzheimer's disease comprising administering to a subject in need of such treatment or prevention a therapeutically effective amount of an antibody as claimed in any one of claims 16 to 19.

24. A method of treating or preventing Alzheimer's disease comprising administering to a subject in need of such treatment or prevention a therapeutically effective amount of one or more of the following Alzheimer's Disease-Associated Protein Isoforms (ADPIs) and/or a nucleic acid encoding such ADPIs: ADPI-1, ADPI-3.1, ADPI-3.2, ADPI-3.3, ADPI-5.3, ADPI-6, ADPI-8, ADPI-9, ADPI-10, ADPI-11, ADPI-12.1, ADPI-12.2, ADPI-13, ADPI-15, ADPI-16, ADPI-22, ADPI-23, ADPI-24, ADPI-25.3, ADPI-25.2, ADPI-26, ADPI-27, ADPI-29, ADPI-31, ADPI-33.1, ADPI-35, ADPI-37, ADPI-39, ADPI-42, ADPI-54, ADPI-56, ADPI-61, ADPI-62, ADPI-66, ADPI-67, ADPI-68, ADPI-70, ADPI-72, ADPI-77, ADPI-78.2, ADPI-78.3, ADPI-79, ADPI-81.2, ADPI-82, ADPI-85, ADPI-87.1, ADPI-88, ADPI-90, ADPI-91.1, ADPI-91.2, ADPI-92, ADPI-94, ADPI-95, ADPI-97, ADPI-98, ADPI-101, ADPI-102.1, ADPI-103, ADPI-105.2, ADPI-105.1, ADPI-107, ADPI-108, ADPI-109, ADPI-111.1, ADPI-112, ADPI-113, ADPI-115, ADPI-117.1, ADPI-119, ADPI-120, ADPI-121, ADPI-124, ADPI-125, ADPI-126, ADPI-126, ADPI-126, ADPI-127, ADPI-128, ADPI-129, ADPI-130, ADPI-131, ADPI-132, ADPI-133, ADPI-138, ADPI-139, ADPI-140, ADPI-141, ADPI-142.2, ADPI-142.1, ADPI-143.1, ADPI-143.2, ADPI-144, ADPI-146.2, ADPI-146.1, ADPI-148, ADPI-149.1, ADPI-150, ADPI-151.2, ADPI-151.1, ADPI-152, ADPI-153.1, ADPI-153.2, ADPI-153.3, ADPI-154, ADPI-155, ADPI-156.2, ADPI-156.1, ADPI-157, ADPI-159.1, ADPI-159.2, ADPI-160, ADPI-162.2, ADPI-162.3, ADPI-162.1, ADPI-163, ADPI-165, ADPI-172, ADPI-173.1, ADPI-173.2, ADPI-175.2, ADPI-175.1, ADPI-176, ADPI-182, ADPI-183, ADPI-188.1, ADPI-189.2, ADPI-191, ADPI-193,ADPI-194.2, ADPI-196.2,ADPI-196.3, ADPI-196.1, ADPI-196.4, ADPI-202, ADPI-204, ADPI-208, ADPI-209, ADPI-216, ADPI-217, ADPI-220, ADPI-223, ADPI-228, ADPI-229, ADPI-230.1, ADPI-230.2, ADPI-232.1, ADPI-232.2, ADPI-236.2, ADPI-236.3, ADPI-237.2, ADPI-237.1, ADPI-240, ADPI-243, ADPI-245, ADPI-250, ADPI-251, ADPI-252.1, ADPI-252.2, ADPI-253, ADPI-254, ADPI-255, ADPI-256, ADPI-257.1, ADPI-257.2, ADPI-258, ADPI-259.1, ADPI-259.2, ADPI-260, ADPI-261, ADPI-262, ADPI-263, ADPI-264, ADPI-265, ADPI-266, ADPI-267, ADPI-268.1, ADPI-268.2, ADPI-269, ADPI-270, ADPI-271, ADPI-272, ADPI-273, ADPI-274, ADPI-275, ADPI-276, ADPI-277, ADPI-278.1, ADPI-278.2, ADPI-279.1, ADPI-279.2, ADPI-280, ADPI-281, ADPI-282, ADPI-283, ADPI-284, ADPI-285, ADPI-286, ADPI-287, ADPI-288, ADPI-289, ADPI-290, ADPI-291, ADPI-292, ADPI-293, ADPI-294, ADPI-295, ADPI-296, ADPI-297, ADPI-298, ADPI-299, ADPI-300, ADPI-301, ADPI-302, ADPI-303, ADPI-304, ADPI-305, ADPI-306, ADPI-307.1, ADPI-307.2, ADPI-308, ADPI-309, ADPI-311, ADPI-312, ADPI-313, ADPI-314, ADPI-315, ADPI-316.1, ADPI-316.2, ADPI-317, ADPI-318, ADPI-319, ADPI-320.1, ADPI-320.2, ADPI-321, ADPI-322, ADPI-323, ADPI-324, ADPI-325, ADPI-326, ADPI-327, ADPI-328, ADPI-329, ADPI-330, ADPI-331, ADPI-332, ADPI-333.1, ADPI-333.2, ADPI-334, ADPI-335, ADPI-336, ADPI-337, ADPI-338, ADPI-339, ADPI-340, ADPI-341, ADPI-342, ADPI-343, ADPI-344, ADPI-345, ADPI-346, ADPI-347, ADPI-348, ADPI-349, ADPI-350, ADPI-351, ADPI-352, ADPI-353, ADPI-354, ADPI-355, ADPI-356, ADPI-357, ADPI-358, ADPI-359, ADPI-360, ADPI-361, ADPI-362, ADPI-363, ADPI-364, ADPI-365.1, ADPI-365.2, ADPI-366, ADPI-367.1, ADPI-367.2, ADPI-368.1, ADPI-368.2, ADPI-369.1, ADPI-369.2, ADPI-370, ADPI-371, ADPI-372, ADPI-373, ADPI-374, ADPI-375, ADPI-376, ADPI-377, ADPI-378, ADPI-379, ADPI-380, ADPI-381, ADPI-382, ADPI-383, ADPI-384, ADPI-386, ADPI-387, ADPI-388, ADPI-389, ADPI-390, ADPI-391, ADPI-392, ADPI-393, ADPI-394, ADPI-395, ADPI-396, ADPI-397, ADPI-398, ADPI-399.1, ADPI-399.2, ADPI-400, ADPI-401, ADPI-402, ADPI-403, ADPI-404.1, ADPI-404.2, ADPI-405, ADPI-406, ADPI-407, ADPI-408, ADPI-409, ADPI-410, ADPI-411, ADPI-412.1, ADPI-412.2, ADPI-413, ADPI-414, ADPI-415, ADPI-416, ADPI-417, ADPI-418, ADPI-419, ADPI-420, ADPI-421, ADPI-422, ADPI-423, ADPI-424, ADPI-425, ADPI-426.1, ADPI-426.2, ADPI-427, ADPI-428, ADPI-429, ADPI-430, ADPI-431, ADPI-432, ADPI-433, ADPI-434, ADPI-435, ADPI-435, ADPI-436, ADPI-437, ADPI-438, ADPI-439, ADPI-440.1, ADPI-440.2, ADPI-441, ADPI-442, ADPI-443, ADPI-444, ADPI-445, ADPI-446, ADPI-447.1, ADPI-447.2, ADPI-448, ADPI-449, ADPI-450.1, ADPI-450.2, ADPI-451, ADPI-452, ADPI-453, ADPI-454, ADPI-455, ADPI-456, ADPI-457, ADPI-458, ADPI-459, ADPI-460, ADPI-461, ADPI-462, ADPI-463, ADPI-464, ADPI-465, ADPI-466.1, ADPI-466.2, ADPI-467, ADPI-468, ADPI-469.1, ADPI-469.2, ADPI-470, ADPI-471, ADPI-472, ADPI-473, ADPI-474, ADPI-475, ADPI-476, ADPI-477, ADPI-478.1, ADPI-478.2, ADPI-479, ADPI-480, ADPI-481, ADPI-482, ADPI-483, ADPI-484, ADPI-485, ADPI-486, ADPI-487, ADPI-488, ADPI-489, ADPI-490, ADPI-491, ADPI-492, ADPI-493.1, ADPI-493.2, ADPI-494, ADPI-495, ADPI-496, ADPI-497, ADPI-498, ADPI-499, ADPI-500, ADPI-501, ADPI-502, ADPI-503, ADPI-504, ADPI-505, ADPI-506, ADPI-507, ADPI-508, ADPI-509, ADPI-510, ADPI-511, ADPI-512, ADPI-513, ADPI-514, ADPI-515, ADPI-516, ADPI-517, ADPI-518, ADPI-519, ADPI-520, ADPI-521, ADPI-522, ADPI-523, ADPI-524, ADPI-525, ADPI-526.1, ADPI-526.2, ADPI-527, ADPI-528, ADPI-529, ADPI-530.1, ADPI-530.2, ADPI-531, ADPI-532, ADPI-533, ADPI-534, ADPI-535, ADPI-536, ADPI-537, ADPI-538, ADPI-539, ADPI-540, ADPI-541, ADPI-542, ADPI-543, ADPI-544, ADPI-545, ADPI-546, ADPI-547.1, ADPI-547.2, ADPI-547.3, ADPI-548.1, ADPI-548.2, ADPI-549, optionally in combination with ADPI-41.

25. A method of treating or preventing Alzheimer's disease comprising administering to a subject in need of such treatment or prevention a therapeutically effective amount of a nucleic acid that inhibits the function of one or more of the following Alzheimer's Disease-Associated Protein Isoforms (ADPIs): ADPI-1, ADPI-3.1, ADPI-3.2, ADPI-3.3, ADPI-5.3, ADPI-6, ADPI-8, ADPI-9, ADPI-10, ADPI-11, ADPI-12.1, ADPI-12.2, ADPI-13, ADPI-15, ADPI-16, ADPI-22, ADPI-23, ADPI-24, ADPI-25.3, ADPI-25.2, ADPI-26, ADPI-27, ADPI-29, ADPI-31, ADPI-33.1, ADPI-35, ADPI-37, ADPI-39, ADPI-42, ADPI-54, ADPI-56, ADPI-61, ADPI-62, ADPI-66, ADPI-67, ADPI-68, ADPI-70, ADPI-72, ADPI-77, ADPI-78.2, ADPI-78.3, ADPI-79, ADPI-81.2, ADPI-82, ADPI-85, ADPI-87.1, ADPI-88, ADPI-90, ADPI-91.1, ADPI-91.2, ADPI-92, ADPI-94, ADPI-95, ADPI-97, ADPI-98, ADPI-101, ADPI-102.1, ADPI-103, ADPI-105.2, ADPI-105.1, ADPI-107, ADPI-108, ADPI-109, ADPI-111.1, ADPI-112, ADPI-113, ADPI-115, ADPI-117.1, ADPI-119, ADPI-120, ADPI-121, ADPI-124, ADPI-125, ADPI-126, ADPI-126, ADPI-126, ADPI-127, ADPI-128, ADPI-129, ADPI-130, ADPI-131, ADPI-132, ADPI-133, ADPI-138, ADPI-139, ADPI-140, ADPI-141, ADPI-142.2, ADPI-142.1, ADPI-143.1, ADPI-143.2, ADPI-144, ADPI-146.2, ADPI-146.1, ADPI-148, ADPI-149.1, ADPI-150, ADPI-151.2, ADPI-151.1, ADPI-152, ADPI-153.1, ADPI-153.2, ADPI-153.3, ADPI-154, ADPI-155, ADPI-156.2, ADPI-156.1, ADPI-157, ADPI-159.1, ADPI-159.2, ADPI-160, ADPI-162.2, ADPI-162.3, ADPI-162.1, ADPI-163, ADPI-165, ADPI-172, ADPI-173.1, ADPI-173.2, ADPI-175.2, ADPI-175.1, ADPI-176, ADPI-182, ADPI-183, ADPI-188.1, ADPI-189.2, ADPI-191, ADPI-193, ADPI-194.2, ADPI-196.2, ADPI-196.3, ADPI-196.1, ADPI-196.4, ADPI-202, ADPI-204, ADPI-208, ADPI-209, ADPI-216, ADPI-217, ADPI-220, ADPI-223, ADPI-228, ADPI-229, ADPI-230.1, ADPI-230.2, ADPI-232.1, ADPI-232.2, ADPI-236.2, ADPI-236.3, ADPI-237.2, ADPI-237.1, ADPI-240, ADPI-243, ADPI-245, ADPI-250, ADPI-251, ADPI-252.1, ADPI-252.2, ADPI-253, ADPI-254, ADPI-255, ADPI-256, ADPI-257.1, ADPI-257.2, ADPI-258, ADPI-259.1, ADPI-259.2, ADPI-260, ADPI-261, ADPI-262, ADPI-263, ADPI-264, ADPI-265, ADPI-266, ADPI-267, ADPI-268.1, ADPI-268.2, ADPI-269, ADPI-270, ADPI-271, ADPI-272, ADPI-273, ADPI-274, ADPI-275, ADPI-276, ADPI-277, ADPI-278.1, ADPI-278.2, ADPI-279.1, ADPI-279.2, ADPI-280, ADPI-281, ADPI-282, ADPI-283, ADPI-284, ADPI-285, ADPI-286, ADPI-287, ADPI-288, ADPI-289, ADPI-290, ADPI-291, ADPI-292, ADPI-293, ADPI-294, ADPI-295, ADPI-296, ADPI-297, ADPI-298, ADPI-299, ADPI-300, ADPI-301, ADPI-302, ADPI-303, ADPI-304, ADPI-305, ADPI-306, ADPI-307.1, ADPI-307.2, ADPI-308, ADPI-309, ADPI-311, ADPI-312, ADPI-313, ADPI-314, ADPI-315, ADPI-316.1, ADPI-316.2, ADPI-317, ADPI-318, ADPI-319, ADPI-320.1, ADPI-320.2, ADPI-321, ADPI-322, ADPI-323, ADPI-324, ADPI-325, ADPI-326, ADPI-327, ADPI-328, ADPI-329, ADPI-330, ADPI-331, ADPI-332, ADPI-333.1, ADPI-333.2, ADPI-334, ADPI-335, ADPI-336, ADPI-337, ADPI-338, ADPI-339, ADPI-340, ADPI-341, ADPI-342, ADPI-343, ADPI-344, ADPI-345, ADPI-346, ADPI-347, ADPI-348, ADPI-349, ADPI-350, ADPI-351, ADPI-352, ADPI-353, ADPI-354, ADPI-355, ADPI-356, ADPI-357, ADPI-358, ADPI-359, ADPI-360, ADPI-361, ADPI-362, ADPI-363, ADPI-364, ADPI-365.1, ADPI-365.2, ADPI-366, ADPI-367.1, ADPI-367.2, ADPI-368.1, ADPI-368.2, ADPI-369.1, ADPI-369.2, ADPI-370, ADPI-371, ADPI-372, ADPI-373, ADPI-374, ADPI-375, ADPI-376, ADPI-377, ADPI-378, ADPI-379, ADPI-380, ADPI-381, ADPI-382, ADPI-383, ADPI-384, ADPI-386, ADPI-387, ADPI-388, ADPI-389, ADPI-390, ADPI-391, ADPI-392, ADPI-393, ADPI-394, ADPI-395, ADPI-396, ADPI-397, ADPI-398, ADPI-399.1, ADPI-399.2, ADPI-400, ADPI-401, ADPI-402, ADPI-403, ADPI-404.1, ADPI-404.2, ADPI-405, ADPI-406, ADPI-407, ADPI-408, ADPI-409, ADPI-410, ADPI-411, ADPI-412.1, ADPI-412.2, ADPI-413, ADPI-414, ADPI-415, ADPI-416, ADPI-417, ADPI-418, ADPI-419, ADPI-420, ADPI-421, ADPI-422, ADPI-423, ADPI-424, ADPI-425, ADPI-426.1, ADPI-426.2, ADPI-427, ADPI-428, ADPI-429, ADPI-430, ADPI-431, ADPI-432, ADPI-433, ADPI-434, ADPI-435, ADPI-435, ADPI-436, ADPI-437, ADPI-438, ADPI-439, ADPI-440.1, ADPI-440.2, ADPI-441, ADPI-442, ADPI-443, ADPI-444, ADPI-445, ADPI-446, ADPI-447.1, ADPI-447.2, ADPI-448, ADPI-449, ADPI-450.1, ADPI-450.2, ADPI-451, ADPI-452, ADPI-453, ADPI-454, ADPI-455, ADPI-456, ADPI-457, ADPI-458, ADPI-459, ADPI-460, ADPI-461, ADPI-462, ADPI-463, ADPI-464, ADPI-465, ADPI-466.1, ADPI-466.2, ADPI-467, ADPI-468, ADPI-469.1, ADPI-469.2, ADPI-470, ADPI-471, ADPI-472, ADPI-473, ADPI-474, ADPI-475, ADPI-476, ADPI-477, ADPI-478.1, ADPI-478.2, ADPI-479, ADPI-480, ADPI-481, ADPI-482, ADPI-483, ADPI-484, ADPI-485, ADPI-486, ADPI-487, ADPI-488, ADPI-489, ADPI-490, ADPI-491, ADPI-492, ADPI-493.1, ADPI-493.2, ADPI-494, ADPI-495, ADPI-496, ADPI-497, ADPI-498, ADPI-499, ADPI-500, ADPI-501, ADPI-502, ADPI-503, ADPI-504, ADPI-505, ADPI-506, ADPI-507, ADPI-508, ADPI-509, ADPI-510, ADPI-511, ADPI-512, ADPI-513, ADPI-514, ADPI-515, ADPI-516, ADPI-517, ADPI-518, ADPI-519, ADPI-520, ADPI-521, ADPI-522, ADPI-523, ADPI-524, ADPI-525, ADPI-526.1, ADPI-526.2, ADPI-527, ADPI-528, ADPI-529, ADPI-530.1, ADPI-530.2, ADPI-531, ADPI-532, ADPI-533, ADPI-534, ADPI-535, ADPI-536, ADPI-537, ADPI-538, ADPI-539, ADPI-540, ADPI-541, ADPI-542, ADPI-543, ADPI-544, ADPI-545, ADPI-546, ADPI-547.1, ADPI-547.2, ADPI-547.3, ADPI-548.1, ADPI-548.2, ADPI-549, optionally in combination with a therapeutically effective amount of a nucleic acid that inhibits the function of ADPI-41.

26. The method of claim 25, wherein the nucleic acid is a ADPI antisense nucleic acid or ribozyme.

27. A method of screening for agents that interact with one or more of the Alzheimer's Disease-Associated Protein Isoforms (ADPIs) as defined in claim 25, a fragment of the or each ADPI (ADPI fragment), a polypeptide related to the or each ADPI (ADPI-related polypeptide), or a ADPI fusion protein, said method comprising:
(a) contacting a ADPI, a biologically active portion of a ADPI, a ADPI-related polypeptide, or a ADPI fusion protein with a candidate agent; and
(b) determining whether or not the candidate agent interacts with the ADPI, the ADPI fragment, the ADPI-related polypeptide, or the ADPI fusion protein.

28. The method of claim 27, wherein the ADPI, the ADPI fragment, the ADPI-related polypeptide, or the ADPI fusion protein is expressed by cells.

29. The method of claim 28, wherein the cells express a recombinant ADPI, a recombinant ADPI fragment, a recombinant ADPI-related polypeptide, or a ADPI fusion protein.

30. A method of screening for agents that modulate the expression or activity of one or more of the Alzheimer's Disease-Associated Protein Isoforms (ADPIs) as defined in claim 25, a fragment of the or each ADPI (ADPI fragment), a polypeptide related to the or each ADPI (ADPI-related polypeptide), or a ADPI fusion protein, said method comprising:
(a) contacting a first population of cells expressing a ADPI, a ADPI fragment, a ADPI-related polypeptide, or a ADPI fusion protein with a candidate agent;
(b) contacting a second population of cells expressing said ADPI, said ADPI fragment, said ADPI-related polypeptide, or said ADPI fusion protein, with a control agent; and
(c) comparing the level of said ADPI, said ADPI fragment, said ADPI-related polypeptide, said ADPI fusion protein, or mRNA encoding said ADPI, said ADPI fragment, said ADPI-related polypeptide, or said ADPI fusion protein, in the first and second populations of cells, or comparing the level of induction of a cellular second messenger in the first and second populations of cells.

31. The method as claimed in claim 30, wherein the level of said ADPI, said ADPI fragment, said ADPI-related polypeptide, said ADPI fusion protein, or mRNA encoding said ADPI, said ADPI fragment, said ADPI-related polypeptide, said ADPI fusion protein, or said cellular second messenger is greater or less in the first population of cells than in the second population of cells.

32. A method of screening for or identifying agents that modulate the expression or activity of one or more of the Alzheimer's Disease-Associated Protein Isoforms (ADPIs) as defined in claim 25, a fragment of the or each ADPI (ADPI fragment), a polypeptide related to the or each ADPI (ADPI-related polypeptide), or a ADPI fusion protein, said method comprising:
(a) administering a candidate agent to a first mammal or group of mammals;
(b) administering a control agent to a second mammal or group of mammals; and
(c) comparing the level of expression of the ADPI, the ADPI fragment, the ADPI-related polypeptide or the ADPI fusion protein or of mRNA encoding the ADPI, the ADPI fragment, the ADPI-related polypeptide or the ADPI fusion protein in the first and second groups, or comparing the level of induction of a cellular second messenger in the first and second groups.

33. The method as claimed in claim 32, wherein the mammals are animal models for Alzheimer's disease.

34. The method as claimed in claim 32 or claim 33, wherein the level of expression of said ADPI, said ADPI fragment, said ADPI-related polypeptide or said ADPI fusion protein, mRNA encoding said ADPI, said ADPI fragment, said ADPI-related polypeptide or said ADPI fusion protein or of said cellular second messenger is greater or less in the first group than in the second group.

35. The method as claimed in claim 33 or claim 34, wherein the levels of said ADPI, said ADPI fragment, said ADPI-related polypeptide or said ADPI fusion protein, mRNA encoding said ADPI, said ADPI fragment, said ADPI-related polypeptide or said ADPI fusion protein, or of said cellular second messenger in the first and second groups are further compared to the level of said ADPI, said ADPI fragment, said ADPI-related polypeptide or said ADPI fusion protein, or said mRNA encoding said ADPI, said ADPI fragment, said ADPI-related polypeptide or said ADPI fusion protein in normal control mammals.

36. The method as claimed in claim 33, 34 or 35, wherein administration of said candidate agent modulates the level of said ADPI, said ADPI fragment, said ADPI-related polypeptide or said ADPI fusion protein, or said mRNA encoding said ADPI, said ADPI fragment, said ADPI-related polypeptide or said ADPI fusion protein, or said cellular second messenger in the first group towards the levels of said ADPI, said ADPI fragment, said ADPI-related polypeptide or said ADPI fusion protein, or said mRNA or said cellular second messenger in the second group.

37. The method as claimed in any of one of claims 32 to 36, wherein said mammals are human subjects having Alzheimer's disease.

38. A method of screening for or identifying agents that interact with one or more of the Alzheimer's Disease-Associated Protein Isoforms (ADPIs) as defined in claim 25, a fragment of the or each ADPI (ADPI fragment), a polypeptide related to the or each ADPI (ADPI-related polypeptide), or a ADPI fusion protein, said method comprising:
(a) contacting a candidate agent with the ADPI, the ADPI fragment, the ADPI-related polypeptide, or the ADPI fusion protein, and
(b) quantitatively detecting binding, if any, between the agent and the ADPI, the ADPI fragment, the ADPI-related polypeptide, or the ADPI fusion protein.

39. A method of screening for or identifying agents that modulate the activity of one or more of the Alzheimer's Disease-Associated Protein Isoforms (ADPIs) as defined in claim 25, a fragment of the or each ADPI (ADPI fragment), a polypeptide related to the or each ADPI (ADPI-related polypeptide), or a ADPI fusion protein, said method comprising:
(a) in a first aliquot, contacting a candidate agent with the ADPI, the ADPI fragment, the ADPI-related polypeptide, or the ADPI fusion protein, and
(b) comparing the activity of the ADPI, the ADPI fragment, the ADPI-related polypeptide, or the ADPI fusion protein in the first aliquot after addition of the candidate agent with the activity of the ADPI, the ADPI fragment, the ADPI-related polypeptide, or the ADPI fusion protein in a control aliquot, or with a previously determined reference range.

40. The method as claimed in claim 38 or claim 39, wherein the ADPI, the ADPI fragment, the ADPI-related polypeptide, or the ADPI fusion protein is a recombinant protein.

41. The method as claimed in claim 38, 39 or 40, wherein the ADPI, the ADPI fragment, the ADPI-related polypeptide, or the ADPI fusion protein is immobilized on a solid phase.

42. A method for screening, diagnosis or prognosis of Alzheimer's disease in a subject or for monitoring the effect of an anti-Alzheimer's disease drug or therapy administered to a subject, comprising:
(a) contacting at least one oligonucleotide probe comprising 10 or more consecutive nucleotides complementary to a nucleotide sequence encoding a ADPI chosen from ADPI-1, ADPI-3.1, ADPI-3.2, ADPI-3.3, ADPI-5.3, ADPI-6, ADPI-8, ADPI-9, ADPI-10, ADPI-11, ADPI-12.1, ADPI-12.2, ADPI-13, ADPI-15, ADPI-16, ADPI-22, ADPI-23, ADPI-24, ADPI-25.3, ADPI-25.2, ADPI-26, ADPI-27, ADPI-29, ADPI-31, ADPI-33.1, ADPI-35, ADPI-37, ADPI-39, ADPI-42, ADPI-54, ADPI-56, ADPI-61, ADPI-62, ADPI-66, ADPI-67, ADPI-68, ADPI-70, ADPI-72, ADPI-77, ADPI-78.2, ADPI-78.3, ADPI-79, ADPI-81.2, ADPI-82, ADPI-85, ADPI-87.1, ADPI-88, ADPI-90, ADPI-91.1, ADPI-91.2, ADPI-92, ADPI-94, ADPI-95, ADPI-97, ADPI-98, ADPI-101, ADPI-102.1, ADPI-103, ADPI-105.2, ADPI-105.1, ADPI-107, ADPI-108, ADPI-109, ADPI-111.1, ADPI-112, ADPI-113, ADPI-115, ADPI-117.1, ADPI-119, ADPI-120, ADPI-121, ADPI-124, ADPI-125, ADPI-126, ADPI-126, ADPI-126, ADPI-127, ADPI-128, ADPI-129, ADPI-130, ADPI-131, ADPI-132, ADPI-133, ADPI-138, ADPI-139, ADPI-140, ADPI-141, ADPI-142.2, ADPI-142.1, ADPI-143.1, ADPI-143.2, ADPI-144, ADPI-146.2, ADPI-146.1, ADPI-148, ADPI-149.1, ADPI-150, ADPI-151.2, ADPI-151.1, ADPI-152, ADPI-153.1, ADPI-153.2, ADPI-153.3, ADPI-154, ADPI-155, ADPI-156.2, ADPI-156.1, ADPI-157, ADPI-159.1, ADPI-159.2, ADPI-160, ADPI-162.2, ADPI-162.3, ADPI-162.1, ADPI-163, ADPI-165, ADPI-172, ADPI-173.1, ADPI-173.2, ADPI-175.2, ADPI-175.1, ADPI-176, ADPI-182, ADPI-183, ADPI-188.1, ADPI-189.2, ADPI-191, ADPI-193, ADPI-194.2, ADPI-196.2, ADPI-196.3, ADPI-196.1, ADPI-196.4, ADPI-202, ADPI-204, ADPI-208, ADPI-209, ADPI-216, ADPI-217, ADPI-220, ADPI-223, ADPI-228, ADPI-229, ADPI-230.1, ADPI-230.2, ADPI-232.1, ADPI-232.2, ADPI-236.2, ADPI-236.3, ADPI-237.2, ADPI-237.1, ADPI-240, ADPI-243, ADPI-245, ADPI-250, ADPI-251, ADPI-252.1, ADPI-252.2, ADPI-253, ADPI-254, ADPI-255, ADPI-256, ADPI-257.1, ADPI-257.2, ADPI-258, ADPI-259.1, ADPI-259.2, ADPI-260, ADPI-261, ADPI-262, ADPI-263, ADPI-264, ADPI-265, ADPI-266, ADPI-267, ADPI-268.1, ADPI-268.2, ADPI-269, ADPI-270, ADPI-271, ADPI-272, ADPI-273, ADPI-274, ADPI-275, ADPI-276, ADPI-277, ADPI-278.1, ADPI-278.2, ADPI-279.1, ADPI-279.2, ADPI-280, ADPI-281, ADPI-282, ADPI-283, ADPI-284, ADPI-285, ADPI-286, ADPI-287, ADPI-288, ADPI-289, ADPI-290, ADPI-291, ADPI-292, ADPI-293, ADPI-294, ADPI-295, ADPI-296, ADPI-297, ADPI-298, ADPI-299, ADPI-300, ADPI-301, ADPI-302, ADPI-303, ADPI-304, ADPI-305, ADPI-306, ADPI-307.1, ADPI-307.2, ADPI-308, ADPI-309, ADPI-311, ADPI-312, ADPI-313, ADPI-314, ADPI-315, ADPI-316.1, ADPI-316.2, ADPI-317, ADPI-318, ADPI-319, ADPI-320.1, ADPI-320.2, ADPI-321, ADPI-322, ADPI-323, ADPI-324, ADPI-325, ADPI-326, ADPI-327, ADPI-328, ADPI-329, ADPI-330, ADPI-331, ADPI-332, ADPI-333.1, ADPI-333.2, ADPI-334, ADPI-335, ADPI-336, ADPI-337, ADPI-338, ADPI-339, ADPI-340, ADPI-341, ADPI-342, ADPI-343, ADPI-344, ADPI-345, ADPI-346, ADPI-347, ADPI-348, ADPI-349, ADPI-350, ADPI-351, ADPI-352, ADPI-353, ADPI-354, ADPI-355, ADPI-356, ADPI-357, ADPI-358, ADPI-359, ADPI-360, ADPI-361, ADPI-362, ADPI-363, ADPI-364, ADPI-365.1, ADPI-365.2, ADPI-366, ADPI-367.1, ADPI-367.2, ADPI-368.1, ADPI-368.2, ADPI-369.1, ADPI-369.2, ADPI-370, ADPI-371, ADPI-372, ADPI-373, ADPI-374, ADPI-375, ADPI-376, ADPI-377, ADPI-378, ADPI-379, ADPI-380, ADPI-381, ADPI-382, ADPI-383, ADPI-384, ADPI-386, ADPI-387, ADPI-388, ADPI-389, ADPI-390, ADPI-391, ADPI-392, ADPI-393, ADPI-394, ADPI-395, ADPI-396, ADPI-397, ADPI-398, ADPI-399.1, ADPI-399.2, ADPI-400, ADPI-401, ADPI-402, ADPI-403, ADPI-404.1, ADPI-404.2, ADPI-405, ADPI-406, ADPI-407, ADPI-408, ADPI-409, ADPI-410, ADPI-411, ADPI-412.1, ADPI-412.2, ADPI-413, ADPI-414, ADPI-415, ADPI-416, ADPI-417, ADPI-418, ADPI-419, ADPI-420, ADPI-421, ADPI-422, ADPI-423, ADPI-424, ADPI-425, ADPI-426.1, ADPI-426.2, ADPI-427, ADPI-428, ADPI-429, ADPI-430, ADPI-431, ADPI-432, ADPI-433, ADPI-434, ADPI-435, ADPI-435, ADPI-436, ADPI-437, ADPI-438, ADPI-439, ADPI-440.1, ADPI-440.2, ADPI-441, ADPI-442, ADPI-443, ADPI-444, ADPI-445, ADPI-446, ADPI-447.1, ADPI-447.2, ADPI-448, ADPI-449, ADPI-450.1, ADPI-450.2, ADPI-451, ADPI-452, ADPI-453, ADPI-454, ADPI-455, ADPI-456, ADPI-457, ADPI-458, ADPI-459, ADPI-460, ADPI-461, ADPI-462, ADPI-463, ADPI-464, ADPI-465, ADPI-466.1, ADPI-466.2, ADPI-467, ADPI-468, ADPI-469.1, ADPI-469.2, ADPI-470, ADPI-471, ADPI-472, ADPI-473, ADPI-474, ADPI-475, ADPI-476, ADPI-477, ADPI-478.1, ADPI-478.2, ADPI-479, ADPI-480, ADPI-481, ADPI-482, ADPI-483, ADPI-484, ADPI-485, ADPI-486, ADPI-487, ADPI-488, ADPI-489, ADPI-490, ADPI-491, ADPI-492, ADPI-493.1, ADPI-493.2, ADPI-494, ADPI-495, ADPI-496, ADPI-497, ADPI-498, ADPI-499, ADPI-500, ADPI-501, ADPI-502, ADPI-503, ADPI-504, ADPI-505, ADPI-506, ADPI-507, ADPI-508, ADPI-509, ADPI-510, ADPI-511, ADPI-512, ADPI-513, ADPI-514, ADPI-515, ADPI-516, ADPI-517, ADPI-518, ADPI-519, ADPI-520, ADPI-521, ADPI-522, ADPI-523, ADPI-524, ADPI-525, ADPI-526.1, ADPI-526.2, ADPI-527, ADPI-528, ADPI-529, ADPI-530.1, ADPI-530.2, ADPI-531, ADPI-532, ADPI-533, ADPI-534, ADPI-535, ADPI-536, ADPI-537, ADPI-538, ADPI-539, ADPI-540, ADPI-541, ADPI-542, ADPI-543, ADPI-544, ADPI-545, ADPI-546, ADPI-547.1, ADPI-547.2, ADPI-547.3, ADPI-548.1, ADPI-548.2, ADPI-549 with an RNA obtained from a biological sample from the subject or with cDNA copied from the RNA wherein said contacting occurs under conditions that permit hybridization of the probe to the nucleotide sequence if present;
(b) detecting hybridization, if any, between the probe and the nucleotide sequence; and
(c) comparing the hybridization, if any, detected in step (b) with the hybridization detected in a control sample, or with a previously determined reference range.

43. The method as claimed in claim 42, wherein step (a) comprises contacting a plurality of oligonucleotide probes comprising 10 or more consecutive nucleotides complementary to a nucleotide sequence encoding a ADPI chosen from ADPI-1, ADPI-3.1, ADPI-3.2, ADPI-3.3, ADPI-5.3, ADPI-6, ADPI-8, ADPI-9, ADPI-10, ADPI-11, ADPI-12.1, ADPI-12.2, ADPI-13, ADPI-15, ADPI-16, ADPI-22, ADPI-23, ADPI-24, ADPI-25.3, ADPI-25.2, ADPI-26, ADPI-27, ADPI-29, ADPI-31, ADPI-33.1, ADPI-35, ADPI-37, ADPI-39, ADPI-42, ADPI-54, ADPI-56, ADPI-61, ADPI-62, ADPI-66, ADPI-67, ADPI-68, ADPI-70, ADPI-72, ADPI-77, ADPI-78.2, ADPI-78.3, ADPI-79, ADPI-81.2, ADPI-82, ADPI-85, ADPI-87.1, ADPI-88, ADPI-90, ADPI-91.1, ADPI-91.2, ADPI-92, ADPI-94, ADPI-95, ADPI-97, ADPI-98, ADPI-101, ADPI-102.1, ADPI-103, ADPI-105.2, ADPI-105.1, ADPI-107, ADPI-108, ADPI-109, ADPI-111.1, ADPI-112, ADPI-113, ADPI-115, ADPI-117.1, ADPI-119, ADPI-120, ADPI-121, ADPI-124, ADPI-125, ADPI-126, ADPI-126, ADPI-126, ADPI-127, ADPI-128, ADPI-129, ADPI-130, ADPI-131, ADPI-132, ADPI-133, ADPI-138, ADPI-139, ADPI-140, ADPI-141, ADPI-142.2, ADPI-142.1, ADPI-143.1, ADPI-143.2, ADPI-144, ADPI-146.2, ADPI-146.1, ADPI-148, ADPI-149.1, ADPI-150, ADPI-151.2, ADPI-151.1, ADPI-152, ADPI-153.1, ADPI-153.2, ADPI-153.3, ADPI-154, ADPI-155, ADPI-156.2, ADPI-156.1, ADPI-157, ADPI-159.1, ADPI-159.2, ADPI-160, ADPI-162.2, ADPI-162.3, ADPI-162.1, ADPI-163, ADPI-165, ADPI-172, ADPI-173.1, ADPI-173.2, ADPI-175.2, ADPI-175.1, ADPI-176, ADPI-182, ADPI-183, ADPI-188.1, ADPI-189.2, ADPI-191, ADPI-193, ADPI-194.2, ADPI-196.2, ADPI-196.3, ADPI-196.1, ADPI-196.4, ADPI-202, ADPI-204, ADPI-208, ADPI-209, ADPI-216, ADPI-217, ADPI-220, ADPI-223, ADPI-228, ADPI-229, ADPI-230.1, ADPI-230.2, ADPI-232.1, ADPI-232.2, ADPI-236.2, ADPI-236.3, ADPI-237.2, ADPI-237.1, ADPI-240, ADPI-243, ADPI-245, ADPI-250, ADPI-251, ADPI-252.1, ADPI-252.2, ADPI-253, ADPI-254, ADPI-255, ADPI-256, ADPI-257.1, ADPI-257.2, ADPI-258, ADPI-259.1, ADPI-259.2, ADPI-260, ADPI-261, ADPI-262, ADPI-263, ADPI-264, ADPI-265, ADPI-266, ADPI-267, ADPI-268.1, ADPI-268.2, ADPI-269, ADPI-270, ADPI-271, ADPI-272, ADPI-273, ADPI-274, ADPI-275, ADPI-276, ADPI-277, ADPI-278.1, ADPI-278.2, ADPI-279.1, ADPI-279.2, ADPI-280, ADPI-281, ADPI-282, ADPI-283, ADPI-284, ADPI-285, ADPI-286, ADPI-287, ADPI-288, ADPI-289, ADPI-290, ADPI-291, ADPI-292, ADPI-293, ADPI-294, ADPI-295, ADPI-296, ADPI-297, ADPI-298, ADPI-299, ADPI-300, ADPI-301, ADPI-302, ADPI-303, ADPI-304, ADPI-305, ADPI-306, ADPI-307.1, ADPI-307.2, ADPI-308, ADPI-309, ADPI-311, ADPI-312, ADPI-313, ADPI-314, ADPI-315, ADPI-316.1, ADPI-316.2, ADPI-317, ADPI-318, ADPI-319, ADPI-320.1, ADPI-320.2, ADPI-321, ADPI-322, ADPI-323, ADPI-324, ADPI-325, ADPI-326, ADPI-327, ADPI-328, ADPI-329, ADPI-330, ADPI-331, ADPI-332, ADPI-333.1, ADPI-333.2, ADPI-334, ADPI-335, ADPI-336, ADPI-337, ADPI-338, ADPI-339, ADPI-340, ADPI-341, ADPI-342, ADPI-343, ADPI-344, ADPI-345, ADPI-346, ADPI-347, ADPI-348, ADPI-349, ADPI-350, ADPI-351, ADPI-352, ADPI-353, ADPI-354, ADPI-355, ADPI-356, ADPI-357, ADPI-358, ADPI-359, ADPI-360, ADPI-361, ADPI-362, ADPI-363, ADPI-364, ADPI-365.1, ADPI-365.2, ADPI-366, ADPI-367.1, ADPI-367.2, ADPI-368.1, ADPI-368.2, ADPI-369.1, ADPI-369.2, ADPI-370, ADPI-371, ADPI-372, ADPI-373, ADPI-374, ADPI-375, ADPI-376, ADPI-377, ADPI-378, ADPI-379, ADPI-380, ADPI-381, ADPI-382, ADPI-383, ADPI-384, ADPI-386, ADPI-387, ADPI-388, ADPI-389, ADPI-390, ADPI-391, ADPI-392, ADPI-393, ADPI-394, ADPI-395, ADPI-396, ADPI-397, ADPI-398, ADPI-399.1, ADPI-399.2, ADPI-400, ADPI-401, ADPI-402, ADPI-403, ADPI-404.1, ADPI-404.2, ADPI-405, ADPI-406, ADPI-407, ADPI-408, ADPI-409, ADPI-410, ADPI-411, ADPI-412.1, ADPI-412.2, ADPI-413, ADPI-414, ADPI-415, ADPI-416, ADPI-417, ADPI-418, ADPI-419, ADPI-420, ADPI-421, ADPI-422, ADPI-423, ADPI-424, ADPI-425, ADPI-426.1, ADPI-426.2, ADPI-427, ADPI-428, ADPI-429, ADPI-430, ADPI-431, ADPI-432, ADPI-433, ADPI-434, ADPI-435, ADPI-435, ADPI-436, ADPI-437, ADPI-438, ADPI-439, ADPI-440.1, ADPI-440.2, ADPI-441, ADPI-442, ADPI-443, ADPI-444, ADPI-445, ADPI-446, ADPI-447.1, ADPI-447.2, ADPI-448, ADPI-449, ADPI-450.1, ADPI-450.2, ADPI-451, ADPI-452, ADPI-453, ADPI-454, ADPI-455, ADPI-456, ADPI-457, ADPI-458, ADPI-459, ADPI-460, ADPI-461, ADPI-462, ADPI-463, ADPI-464, ADPI-465, ADPI-466.1, ADPI-466.2, ADPI-467, ADPI-468, ADPI-469.1, ADPI-469.2, ADPI-470, ADPI-471, ADPI-472, ADPI-473, ADPI-474, ADPI-475, ADPI-476, ADPI-477, ADPI-478.1, ADPI-478.2, ADPI-479, ADPI-480, ADPI-481, ADPI-482, ADPI-483, ADPI-484, ADPI-485, ADPI-486, ADPI-487, ADPI-488, ADPI-489, ADPI-490, ADPI-491, ADPI-492, ADPI-493.1, ADPI-493.2, ADPI-494, ADPI-495, ADPI-496, ADPI-497, ADPI-498, ADPI-499, ADPI-500, ADPI-501, ADPI-502, ADPI-503, ADPI-504, ADPI-505, ADPI-506, ADPI-507, ADPI-508, ADPI-509, ADPI-510, ADPI-511, ADPI-512, ADPI-513, ADPI-514, ADPI-515, ADPI-516, ADPI-517, ADPI-518, ADPI-519, ADPI-520, ADPI-521, ADPI-522, ADPI-523, ADPI-524, ADPI-525, ADPI-526.1, ADPI-526.2, ADPI-527, ADPI-528, ADPI-529, ADPI-530.1, ADPI-530.2, ADPI-531, ADPI-532, ADPI-533, ADPI-534, ADPI-535, ADPI-536, ADPI-537, ADPI-538, ADPI-539, ADPI-540, ADPI-541, ADPI-542, ADPI-543, ADPI-544, ADPI-545, ADPI-546, ADPI-547.1, ADPI-547.2, ADPI-547.3, ADPI-548.1, ADPI-548.2, ADPI-549 with an RNA obtained from a biological sample from the subject or with cDNA copied from the RNA wherein said contacting occurs under conditions that permit hybridization of the probe to the nucleotide sequence if present.

44. The method as claimed in claim 43, wherein step (a) includes the step of hybridizing the nucleotide sequence to a DNA array, wherein one or more members of the array are the probes complementary to a plurality of nucleotide sequences encoding distinct ADPIs.

45. A method of modulating the activity of one or more of the following Alzheimer's Disease-Associated Protein Isoforms (ADPIs): ADPI-1, ADPI-3.1, ADPI-3.2, ADPI-3.3, ADPI-5.3, ADPI-6, ADPI-8, ADPI-9, ADPI-10, ADPI-11, ADPI-12.1, ADPI-12.2, ADPI-13, ADPI-15, ADPI-16, ADPI-22, ADPI-23, ADPI-24, ADPI-25.3, ADPI-25.2, ADPI-26, ADPI-27, ADPI-29, ADPI-31, ADPI-33.1, ADPI-35, ADPI-37, ADPI-39, ADPI-42, ADPI-54, ADPI-56, ADPI-61, ADPI-62, ADPI-66, ADPI-67, ADPI-68, ADPI-70, ADPI-72, ADPI-77, ADPI-78.2, ADPI-78.3, ADPI-79, ADPI-81.2, ADPI-82, ADPI-85, ADPI-87.1, ADPI-88, ADPI-90, ADPI-91.1, ADPI-91.2, ADPI-92, ADPI-94, ADPI-95, ADPI-97, ADPI-98, ADPI-101, ADPI-102.1, ADPI-103, ADPI-105.2, ADPI-105.1, ADPI-107, ADPI-108, ADPI-109, ADPI-111.1, ADPI-112, ADPI-113, ADPI-115, ADPI-117.1, ADPI-119, ADPI-120, ADPI-121, ADPI-124, ADPI-125, ADPI-126, ADPI-126, ADPI-126, ADPI-127, ADPI-128, ADPI-129, ADPI-130, ADPI-131, ADPI-132, ADPI-133, ADPI-138, ADPI-139, ADPI-140, ADPI-141, ADPI-142.2, ADPI-142.1, ADPI-143.1, ADPI-143.2, ADPI-144, ADPI-146.2, ADPI-146.1, ADPI-148, ADPI-149.1, ADPI-150, ADPI-151.2, ADPI-151.1, ADPI-152, ADPI-153.1, ADPI-153.2, ADPI-153.3, ADPI-154, ADPI-155, ADPI-156.2, ADPI-156.1, ADPI-157, ADPI-159.1, ADPI-159.2, ADPI-160, ADPI-162.2, ADPI-162.3, ADPI-162.1, ADPI-163, ADPI-165, ADPI-172, ADPI-173.1, ADPI-173.2, ADPI-175.2, ADPI-175.1, ADPI-176, ADPI-182, ADPI-183, ADPI-188.1, ADPI-189.2, ADPI-191, ADPI-193, ADPI-194.2, ADPI-196.2, ADPI-196.3, ADPI-196.1, ADPI-196.4, ADPI-202, ADPI-204, ADPI-208, ADPI-209, ADPI-216, ADPI-217, ADPI-220, ADPI-223, ADPI-228, ADPI-229, ADPI-230.1, ADPI-230.2, ADPI-232.1, ADPI-232.2, ADPI-236.2, ADPI-236.3, ADPI-237.2, ADPI-237.1, ADPI-240, ADPI-243, ADPI-245, ADPI-250, ADPI-251, ADPI-252.1, ADPI-252.2, ADPI-253, ADPI-254, ADPI-255, ADPI-256, ADPI-257.1, ADPI-257.2, ADPI-258, ADPI-259.1, ADPI-259.2, ADPI-260, ADPI-261, ADPI-262, ADPI-263, ADPI-264, ADPI-265, ADPI-266, ADPI-267, ADPI-268.1, ADPI-268.2, ADPI-269, ADPI-270, ADPI-271, ADPI-272, ADPI-273, ADPI-274, ADPI-275, ADPI-276, ADPI-277, ADPI-278.1, ADPI-278.2, ADPI-279.1, ADPI-279.2, ADPI-280, ADPI-281, ADPI-282, ADPI-283, ADPI-284, ADPI-285, ADPI-286, ADPI-287, ADPI-288, ADPI-289, ADPI-290, ADPI-291, ADPI-292, ADPI-293, ADPI-294, ADPI-295, ADPI-296, ADPI-297, ADPI-298, ADPI-299, ADPI-300, ADPI-301, ADPI-302, ADPI-303, ADPI-304, ADPI-305, ADPI-306, ADPI-307.1, ADPI-307.2, ADPI-308, ADPI-309, ADPI-311, ADPI-312, ADPI-313, ADPI-314, ADPI-315, ADPI-316.1, ADPI-316.2, ADPI-317, ADPI-318, ADPI-319, ADPI-320.1, ADPI-320.2, ADPI-321, ADPI-322, ADPI-323, ADPI-324, ADPI-325, ADPI-326, ADPI-327, ADPI-328, ADPI-329, ADPI-330, ADPI-331, ADPI-332, ADPI-333.1, ADPI-333.2, ADPI-334, ADPI-335, ADPI-336, ADPI-337, ADPI-338, ADPI-339, ADPI-340, ADPI-341, ADPI-342, ADPI-343, ADPI-344, ADPI-345, ADPI-346, ADPI-347, ADPI-348, ADPI-349, ADPI-350, ADPI-351, ADPI-352, ADPI-353, ADPI-354, ADPI-355, ADPI-356, ADPI-357, ADPI-358, ADPI-359, ADPI-360, ADPI-361, ADPI-362, ADPI-363, ADPI-364, ADPI-365.1, ADPI-365.2, ADPI-366, ADPI-367.1, ADPI-367.2, ADPI-368.1, ADPI-368.2, ADPI-369.1, ADPI-369.2, ADPI-370, ADPI-371, ADPI-372, ADPI-373, ADPI-374, ADPI-375, ADPI-376, ADPI-377, ADPI-378, ADPI-379, ADPI-380, ADPI-381, ADPI-382, ADPI-383, ADPI-384, ADPI-386, ADPI-387, ADPI-388, ADPI-389, ADPI-390, ADPI-391, ADPI-392, ADPI-393, ADPI-394, ADPI-395, ADPI-396, ADPI-397, ADPI-398, ADPI-399.1, ADPI-399.2, ADPI-400, ADPI-401, ADPI-402, ADPI-403, ADPI-404.1, ADPI-404.2, ADPI-405, ADPI-406, ADPI-407, ADPI-408, ADPI-409, ADPI-410, ADPI-411, ADPI-412.1, ADPI-412.2, ADPI-413, ADPI-414, ADPI-415, ADPI-416, ADPI-417, ADPI-418, ADPI-419, ADPI-420, ADPI-421, ADPI-422, ADPI-423, ADPI-424, ADPI-425, ADPI-426.1, ADPI-426.2, ADPI-427, ADPI-428, ADPI-429, ADPI-430, ADPI-431, ADPI-432, ADPI-433, ADPI-434, ADPI-435, ADPI-435, ADPI-436, ADPI-437, ADPI-438, ADPI-439, ADPI-440.1, ADPI-440.2, ADPI-441, ADPI-442, ADPI-443, ADPI-444, ADPI-445, ADPI-446, ADPI-447.1, ADPI-447.2, ADPI-448, ADPI-449, ADPI-450.1, ADPI-450.2, ADPI-451, ADPI-452, ADPI-453, ADPI-454, ADPI-455, ADPI-456, ADPI-457, ADPI-458, ADPI-459, ADPI-460, ADPI-461, ADPI-462, ADPI-463, ADPI-464, ADPI-465, ADPI-466.1, ADPI-466.2, ADPI-467, ADPI-468, ADPI-469.1, ADPI-469.2, ADPI-470, ADPI-471, ADPI-472, ADPI-473, ADPI-474, ADPI-475, ADPI-476, ADPI-477, ADPI-478.1, ADPI-478.2, ADPI-479, ADPI-480, ADPI-481, ADPI-482, ADPI-483, ADPI-484, ADPI-485, ADPI-486, ADPI-487, ADPI-488, ADPI-489, ADPI-490, ADPI-491, ADPI-492, ADPI-493.1, ADPI-493.2, ADPI-494, ADPI-495, ADPI-496, ADPI-497, ADPI-498, ADPI-499, ADPI-500, ADPI-501, ADPI-502, ADPI-503, ADPI-504, ADPI-505, ADPI-506, ADPI-507, ADPI-508, ADPI-509, ADPI-510, ADPI-511, ADPI-512, ADPI-513, ADPI-514, ADPI-515, ADPI-516, ADPI-517, ADPI-518, ADPI-519, ADPI-520, ADPI-521, ADPI-522, ADPI-523, ADPI-524, ADPI-525, ADPI-526.1, ADPI-526.2, ADPI-527, ADPI-528, ADPI-529, ADPI-530.1, ADPI-530.2, ADPI-531, ADPI-532, ADPI-533, ADPI-534, ADPI-535, ADPI-536, ADPI-537, ADPI-538, ADPI-539, ADPI-540, ADPI-541, ADPI-542, ADPI-543, ADPI-544, ADPI-545, ADPI-546, ADPI-547.1, ADPI-547.2, ADPI-547.3, ADPI-548.1, ADPI-548.2, ADPI-549, comprising the step of contacting a cell with an agent which specifically binds to one or more of the following Alzheimer's Disease-Associated Protein Isoforms (ADPIs): ADPI-1, ADPI-3.1, ADPI-3.2, ADPI-3.3, ADPI-5.3, ADPI-6, ADPI-8, ADPI-9, ADPI-10, ADPI-11, ADPI-12.1, ADPI-12.2, ADPI-13, ADPI-15, ADPI-16, ADPI-22, ADPI-23, ADPI-24, ADPI-25.3, ADPI-25.2, ADPI-26, ADPI-27, ADPI-29, ADPI-31, ADPI-33.1, ADPI-35, ADPI-37, ADPI-39, ADPI-42, ADPI-54, ADPI-56, ADPI-61, ADPI-62, ADPI-66, ADPI-67, ADPI-68, ADPI-70, ADPI-72, ADPI-77, ADPI-78.2, ADPI-78.3, ADPI-79, ADPI-81.2, ADPI-82, ADPI-85, ADPI-87.1, ADPI-88, ADPI-90, ADPI-91.1, ADPI-91.2, ADPI-92, ADPI-94, ADPI-95, ADPI-97, ADPI-98, ADPI-101, ADPI-102.1, ADPI-103, ADPI-105.2, ADPI-105.1, ADPI-107, ADPI-108, ADPI-109, ADPI-111.1, ADPI-112, ADPI-113, ADPI-115, ADPI-117.1, ADPI-119, ADPI-120, ADPI-121, ADPI-124, ADPI-125, ADPI-126, ADPI-126, ADPI-126, ADPI-127, ADPI-128, ADPI-129, ADPI-130, ADPI-131, ADPI-132, ADPI-133, ADPI-138, ADPI-139, ADPI-140, ADPI-141, ADPI-142.2, ADPI-142.1, ADPI-143.1, ADPI-143.2, ADPI-144, ADPI-146.2, ADPI-146.1, ADPI-148, ADPI-149.1, ADPI-150, ADPI-151.2, ADPI-151.1, ADPI-152, ADPI-153.1, ADPI-153.2, ADPI-153.3, ADPI-154, ADPI-155, ADPI-156.2, ADPI-156.1, ADPI-157, ADPI-159.1, ADPI-159.2, ADPI-160, ADPI-162.2, ADPI-162.3, ADPI-162.1, ADPI-163, ADPI-165, ADPI-172, ADPI-173.1, ADPI-173.2, ADPI-175.2, ADPI-175.1, ADPI-176, ADPI-182, ADPI-183, ADPI-188.1, ADPI-189.2, ADPI-191, ADPI-193, ADPI-194.2, ADPI-196.2, ADPI-196.3, ADPI-196.1, ADPI-196.4, ADPI-202, ADPI-204, ADPI-208, ADPI-209, ADPI-216, ADPI-217, ADPI-220, ADPI-223, ADPI-228, ADPI-229, ADPI-230.1, ADPI-230.2, ADPI-232.1, ADPI-232.2, ADPI-236.2, ADPI-236.3, ADPI-237.2, ADPI-237.1, ADPI-240, ADPI-243, ADPI-245, ADPI-250, ADPI-251, ADPI-252.1, ADPI-252.2, ADPI-253, ADPI-254, ADPI-255, ADPI-256, ADPI-257.1, ADPI-257.2, ADPI-258, ADPI-259.1, ADPI-259.2, ADPI-260, ADPI-261, ADPI-262, ADPI-263, ADPI-264, ADPI-265, ADPI-266, ADPI-267, ADPI-268.1, ADPI-268.2, ADPI-269, ADPI-270, ADPI-271, ADPI-272, ADPI-273, ADPI-274, ADPI-275, ADPI-276, ADPI-277, ADPI-278.1, ADPI-278.2, ADPI-279.1, ADPI-279.2, ADPI-280, ADPI-281, ADPI-282, ADPI-283, ADPI-284, ADPI-285, ADPI-286, ADPI-287, ADPI-288, ADPI-289, ADPI-290, ADPI-291, ADPI-292, ADPI-293, ADPI-294, ADPI-295, ADPI-296, ADPI-297, ADPI-298, ADPI-299, ADPI-300, ADPI-301, ADPI-302, ADPI-303, ADPI-304, ADPI-305, ADPI-306, ADPI-307.1, ADPI-307.2, ADPI-308, ADPI-309, ADPI-311, ADPI-312, ADPI-313, ADPI-314, ADPI-315, ADPI-316.1, ADPI-316.2, ADPI-317, ADPI-318, ADPI-319, ADPI-320.1, ADPI-320.2, ADPI-321, ADPI-322, ADPI-323, ADPI-324, ADPI-325, ADPI-326, ADPI-327, ADPI-328, ADPI-329, ADPI-330, ADPI-331, ADPI-332, ADPI-333.1, ADPI-333.2, ADPI-334, ADPI-335, ADPI-336, ADPI-337, ADPI-338, ADPI-339, ADPI-340, ADPI-341, ADPI-342, ADPI-343, ADPI-344, ADPI-345, ADPI-346, ADPI-347, ADPI-348, ADPI-349, ADPI-350, ADPI-351, ADPI-352, ADPI-353, ADPI-354, ADPI-355, ADPI-356, ADPI-357, ADPI-358, ADPI-359, ADPI-360, ADPI-361, ADPI-362, ADPI-363, ADPI-364, ADPI-365.1, ADPI-365.2, ADPI-366, ADPI-367.1, ADPI-367.2, ADPI-368.1, ADPI-368.2, ADPI-369.1, ADPI-369.2, ADPI-370, ADPI-371, ADPI-372, ADPI-373, ADPI-374, ADPI-375, ADPI-376, ADPI-377, ADPI-378, ADPI-379, ADPI-380, ADPI-381, ADPI-382, ADPI-383, ADPI-384, ADPI-386, ADPI-387, ADPI-388, ADPI-389, ADPI-390, ADPI-391, ADPI-392, ADPI-393, ADPI-394, ADPI-395, ADPI-396, ADPI-397, ADPI-398, ADPI-399.1, ADPI-399.2, ADPI-400, ADPI-401, ADPI-402, ADPI-403, ADPI-404.1, ADPI-404.2, ADPI-405, ADPI-406, ADPI-407, ADPI-408, ADPI-409, ADPI-410, ADPI-411, ADPI-412.1, ADPI-412.2, ADPI-413, ADPI-414, ADPI-415, ADPI-416, ADPI-417, ADPI-418, ADPI-419, ADPI-420, ADPI-421, ADPI-422, ADPI-423, ADPI-424, ADPI-425, ADPI-426.1, ADPI-426.2, ADPI-427, ADPI-428, ADPI-429, ADPI-430, ADPI-431, ADPI-432, ADPI-433, ADPI-434, ADPI-435, ADPI-435, ADPI-436, ADPI-437, ADPI-438, ADPI-439, ADPI-440.1, ADPI-440.2, ADPI-441, ADPI-442, ADPI-443, ADPI-444, ADPI-445, ADPI-446, ADPI-447.1, ADPI-447.2, ADPI-448, ADPI-449, ADPI-450.1, ADPI-450.2, ADPI-451, ADPI-452, ADPI-453, ADPI-454, ADPI-455, ADPI-456, ADPI-457, ADPI-458, ADPI-459, ADPI-460, ADPI-461, ADPI-462, ADPI-463, ADPI-464, ADPI-465, ADPI-466.1, ADPI-466.2, ADPI-467, ADPI-468, ADPI-469.1, ADPI-469.2, ADPI-470, ADPI-471, ADPI-472, ADPI-473, ADPI-474, ADPI-475, ADPI-476, ADPI-477, ADPI-478.1, ADPI-478.2, ADPI-479, ADPI-480, ADPI-481, ADPI-482, ADPI-483, ADPI-484, ADPI-485, ADPI-486, ADPI-487, ADPI-488, ADPI-489, ADPI-490, ADPI-491, ADPI-492, ADPI-493.1, ADPI-493.2, ADPI-494, ADPI-495, ADPI-496, ADPI-497, ADPI-498, ADPI-499, ADPI-500, ADPI-501, ADPI-502, ADPI-503, ADPI-504, ADPI-505, ADPI-506, ADPI-507, ADPI-508, ADPI-509, ADPI-510, ADPI-511, ADPI-512, ADPI-513, ADPI-514, ADPI-515, ADPI-516, ADPI-517, ADPI-518, ADPI-519, ADPI-520, ADPI-521, ADPI-522, ADPI-523, ADPI-524, ADPI-525, ADPI-526.1, ADPI-526.2, ADPI-527, ADPI-528, ADPI-529, ADPI-530.1, ADPI-530.2, ADPI-531, ADPI-532, ADPI-533, ADPI-534, ADPI-535, ADPI-536, ADPI-537, ADPI-538, ADPI-539, ADPI-540, ADPI-541, ADPI-542, ADPI-543, ADPI-544, ADPI-545, ADPI-546, ADPI-547.1, ADPI-547.2, ADPI-547.3, ADPI-548.1, ADPI-548.2, ADPI-549, whereby the activity of the respective ADPI is modulated.

46. An agent that modulates the activity of one or more of the following Alzheimer's Disease-Associated Protein Isoforms (ADPIs): ADPI-1, ADPI-3.1, ADPI-3.2, ADPI-3.3, ADPI-5.3, ADPI-6, ADPI-8, ADPI-9, ADPI-10, ADPI-11, ADPI-12.1, ADPI-12.2, ADPI-13, ADPI-15, ADPI-16, ADPI-22, ADPI-23, ADPI-24, ADPI-25.3, ADPI-25.2, ADPI-26, ADPI-27, ADPI-29, ADPI-31, ADPI-33.1, ADPI-35, ADPI-37, ADPI-39, ADPI-42, ADPI-54, ADPI-56, ADPI-61, ADPI-62, ADPI-66, ADPI-67, ADPI-68, ADPI-70, ADPI-72, ADPI-77, ADPI-78.2, ADPI-78.3, ADPI-79, ADPI-81.2, ADPI-82, ADPI-85, ADPI-87.1, ADPI-88, ADPI-90, ADPI-91.1, ADPI-91.2, ADPI-92, ADPI-94, ADPI-95, ADPI-97, ADPI-98, ADPI-101, ADPI-102.1, ADPI-103, ADPI-105.2, ADPI-105.1, ADPI-107, ADPI-108, ADPI-109, ADPI-111.1, ADPI-112, ADPI-113, ADPI-115, ADPI-117.1, ADPI-119, ADPI-120, ADPI-121, ADPI-124, ADPI-125, ADPI-126, ADPI-126, ADPI-126, ADPI-127, ADPI-128, ADPI-129, ADPI-130, ADPI-131, ADPI-132, ADPI-133, ADPI-138, ADPI-139, ADPI-140, ADPI-141, ADPI-142.2, ADPI-142.1, ADPI-143.1, ADPI-143.2, ADPI-144, ADPI-146.2, ADPI-146.1, ADPI-148, ADPI-149.1, ADPI-150, ADPI-151.2, ADPI-151.1, ADPI-152, ADPI-153.1, ADPI-153.2, ADPI-153.3, ADPI-154, ADPI-155, ADPI-156.2, ADPI-156.1, ADPI-157, ADPI-159.1, ADPI-159.2, ADPI-160, ADPI-162.2, ADPI-162.3, ADPI-162.1, ADPI-163, ADPI-165, ADPI-172, ADPI-173.1, ADPI-173.2, ADPI-175.2, ADPI-175.1, ADPI-176, ADPI-182, ADPI-183, ADPI-188.1, ADPI-189.2, ADPI-191, ADPI-193, ADPI-194.2, ADPI-196.2, ADPI-196.3, ADPI-196.1, ADPI-196.4, ADPI-202, ADPI-204, ADPI-208, ADPI-209, ADPI-216, ADPI-217, ADPI-220, ADPI-223, ADPI-228, ADPI-229, ADPI-230.1, ADPI-230.2, ADPI-232.1, ADPI-232.2, ADPI-236.2, ADPI-236.3, ADPI-237.2, ADPI-237.1, ADPI-240, ADPI-243, ADPI-245, ADPI-250, ADPI-251, ADPI-252.1, ADPI-252.2, ADPI-253, ADPI-254, ADPI-255, ADPI-256, ADPI-257.1, ADPI-257.2, ADPI-258, ADPI-259.1, ADPI-259.2, ADPI-260, ADPI-261, ADPI-262, ADPI-263, ADPI-264, ADPI-265, ADPI-266, ADPI-267, ADPI-268.1, ADPI-268.2, ADPI-269, ADPI-270, ADPI-271, ADPI-272, ADPI-273, ADPI-274, ADPI-275, ADPI-276, ADPI-277, ADPI-278.1, ADPI-278.2, ADPI-279.1, ADPI-279.2, ADPI-280, ADPI-281, ADPI-282, ADPI-283, ADPI-284, ADPI-285, ADPI-286, ADPI-287, ADPI-288, ADPI-289, ADPI-290, ADPI-291, ADPI-292, ADPI-293, ADPI-294, ADPI-295, ADPI-296, ADPI-297, ADPI-298, ADPI-299, ADPI-300, ADPI-301, ADPI-302, ADPI-303, ADPI-304, ADPI-305, ADPI-306, ADPI-307.1, ADPI-307.2, ADPI-308, ADPI-309, ADPI-311, ADPI-312, ADPI-313, ADPI-314, ADPI-315, ADPI-316.1, ADPI-316.2, ADPI-317, ADPI-318, ADPI-319, ADPI-320.1, ADPI-320.2, ADPI-321, ADPI-322, ADPI-323, ADPI-324, ADPI-325, ADPI-326, ADPI-327, ADPI-328, ADPI-329, ADPI-330, ADPI-331, ADPI-332, ADPI-333.1, ADPI-333.2, ADPI-334, ADPI-335, ADPI-336, ADPI-337, ADPI-338, ADPI-339, ADPI-340, ADPI-341, ADPI-342, ADPI-343, ADPI-344, ADPI-345, ADPI-346, ADPI-347, ADPI-348, ADPI-349, ADPI-350, ADPI-351, ADPI-352, ADPI-353, ADPI-354, ADPI-355, ADPI-356, ADPI-357, ADPI-358, ADPI-359, ADPI-360, ADPI-361, ADPI-362, ADPI-363, ADPI-364, ADPI-365.1, ADPI-365.2, ADPI-366, ADPI-367.1, ADPI-367.2, ADPI-368.1, ADPI-368.2, ADPI-369.1, ADPI-369.2, ADPI-370, ADPI-371, ADPI-372, ADPI-373, ADPI-374, ADPI-375, ADPI-376, ADPI-377, ADPI-378, ADPI-379, ADPI-380, ADPI-381, ADPI-382, ADPI-383, ADPI-384, ADPI-386, ADPI-387, ADPI-388, ADPI-389, ADPI-390, ADPI-391, ADPI-392, ADPI-393, ADPI-394, ADPI-395, ADPI-396, ADPI-397, ADPI-398, ADPI-399.1, ADPI-399.2, ADPI-400, ADPI-401, ADPI-402, ADPI-403, ADPI-404.1, ADPI-404.2, ADPI-405, ADPI-406, ADPI-407, ADPI-408, ADPI-409, ADPI-410, ADPI-411, ADPI-412.1, ADPI-412.2, ADPI-413, ADPI-414, ADPI-415, ADPI-416, ADPI-417, ADPI-418, ADPI-419, ADPI-420, ADPI-421, ADPI-422, ADPI-423, ADPI-424, ADPI-425, ADPI-426.1, ADPI-426.2, ADPI-427, ADPI-428, ADPI-429, ADPI-430, ADPI-431, ADPI-432, ADPI-433, ADPI-434, ADPI-435, ADPI-435, ADPI-436, ADPI-437, ADPI-438, ADPI-439, ADPI-440.1, ADPI-440.2, ADPI-441, ADPI-442, ADPI-443, ADPI-444, ADPI-445, ADPI-446, ADPI-447.1, ADPI-447.2, ADPI-448, ADPI-449, ADPI-450.1, ADPI-450.2, ADPI-451, ADPI-452, ADPI-453, ADPI-454, ADPI-455, ADPI-456, ADPI-457, ADPI-458, ADPI-459, ADPI-460, ADPI-461, ADPI-462, ADPI-463, ADPI-464, ADPI-465, ADPI-466.1, ADPI-466.2, ADPI-467, ADPI-468, ADPI-469.1, ADPI-469.2, ADPI-470, ADPI-471, ADPI-472, ADPI-473, ADPI-474, ADPI-475, ADPI-476, ADPI-477, ADPI-478.1, ADPI-478.2, ADPI-479, ADPI-480, ADPI-48 1, ADPI-482, ADPI-483, ADPI-484, ADPI-485, ADPI-486, ADPI-487, ADPI-488, ADPI-489, ADPI-490, ADPI-491, ADPI-492, ADPI-493.1, ADPI-493.2, ADPI-494, ADPI-495, ADPI-496, ADPI-497, ADPI-498, ADPI-499, ADPI-500, ADPI-501, ADPI-502, ADPI-503, ADPI-504, ADPI-505, ADPI-506, ADPI-507, ADPI-508, ADPI-509, ADPI-510, ADPI-511, ADPI-512, ADPI-513, ADPI-514, ADPI-515, ADPI-516, ADPI-517, ADPI-518, ADPI-519, ADPI-520, ADPI-521, ADPI-522, ADPI-523, ADPI-524, ADPI-525, ADPI-526.1, ADPI-526.2, ADPI-527, ADPI-528, ADPI-529, ADPI-530.1, ADPI-530.2, ADPI-531, ADPI-532, ADPI-533, ADPI-534, ADPI-535, ADPI-536, ADPI-537, ADPI-538, ADPI-539, ADPI-540, ADPI-541, ADPI-542, ADPI-543, ADPI-544, ADPI-545, ADPI-546, ADPI-547.1, ADPI-547.2, ADPI-547.3, ADPI-548.1, ADPI-548.2, ADPI-549, wherein said agent is identified by the method of any of claims 27-41.

47. A pharmaceutical composition, comprising the agent of claim 46 and a pharmaceutically acceptable carrier.

48. The use of an agent of claim 46 in the manufacture of a medicament for the treatment of Alzheimer's disease.

49. A method of treating or preventing Alzheimer's disease comprising administering to a subject in need of such treatment or prevention a therapeutically effective dose of an agent that modulates the activity of one or more of the following Alzheimer's Disease-Associated Protein Isoforms (ADPIs): ADPI-1, ADPI-3.1, ADPI-3.2, ADPI-3.3, ADPI-5.3, ADPI-6, ADPI-8, ADPI-9, ADPI-10, ADPI-11, ADPI-12.1, ADPI-12.2, ADPI-13, ADPI-15, ADPI-16, ADPI-22, ADPI-23, ADPI-24, ADPI-25.3, ADPI-25.2, ADPI-26, ADPI-27, ADPI-29, ADPI-31, ADPI-33.1, ADPI-35, ADPI-37, ADPI-39, ADPI-42, ADPI-54, ADPI-56, ADPI-61, ADPI-62, ADPI-66, ADPI-67, ADPI-68, ADPI-70, ADPI-72, ADPI-77, ADPI-78.2, ADPI-78.3, ADPI-79, ADPI-81.2, ADPI-82, ADPI-85, ADPI-87.1, ADPI-88, ADPI-90, ADPI-91.1, ADPI-91.2, ADPI-92, ADPI-94, ADPI-95, ADPI-97, ADPI-98, ADPI-100, ADPI-102.1, ADPI-103, ADPI-105.2, ADPI-105.1, ADPI-107, ADPI-108, ADPI-109, ADPI-111.1, ADPI-112, ADPI-113, ADPI-115, ADPI-117.1, ADPI-119, ADPI-120, ADPI-121, ADPI-124, ADPI-125, ADPI-126, ADPI-126, ADPI-126, ADPI-127, ADPI-128, ADPI-129, ADPI-130, ADPI-131, ADPI-132, ADPI-133, ADPI-138, ADPI-139, ADPI-140, ADPI-141, ADPI-142.2, ADPI-142.1, ADPI-143.1, ADPI-143.2, ADPI-144, ADPI-146.2, ADPI-146.1, ADPI-148, ADPI-149.1, ADPI-150, ADPI-151.2, ADPI-151.1, ADPI-152, ADPI-153.1, ADPI-153.2, ADPI-153.3, ADPI-154, ADPI-155, ADPI-156.2, ADPI-156.1, ADPI-157, ADPI-159.1, ADPI-159.2, ADPI-160, ADPI-162.2, ADPI-162.3, ADPI-162.1, ADPI-163, ADPI-165, ADPI-172, ADPI-173.1, ADPI-173.2, ADPI-175.2, ADPI-175.1, ADPI-176, ADPI-182, ADPI-183, ADPI-188.1, ADPI-189.2, ADPI-191, ADPI-193, ADPI-194.2, ADPI-196.2, ADPI-196.3, ADPI-196.1, ADPI-196.4, ADPI-202, ADPI-204, ADPI-208, ADPI-209, ADPI-216, ADPI-217, ADPI-220, ADPI-223, ADPI-228, ADPI-229, ADPI-230.1, ADPI-230.2, ADPI-232.1, ADPI-232.2, ADPI-236.2, ADPI-236.3, ADPI-237.2, ADPI-237.1, ADPI-240, ADPI-243, ADPI-245, ADPI-250, ADPI-251, ADPI-252.1, ADPI-252.2, ADPI-253, ADPI-254, ADPI-255, ADPI-256, ADPI-257.1, ADPI-257.2, ADPI-258, ADPI-259.1, ADPI-259.2, ADPI-260, ADPI-261, ADPI-262, ADPI-263, ADPI-264, ADPI-265, ADPI-266, ADPI-267, ADPI-268.1, ADPI-268.2, ADPI-269, ADPI-270, ADPI-271, ADPI-272, ADPI-273, ADPI-274, ADPI-275, ADPI-276, ADPI-277, ADPI-278.1, ADPI-278.2, ADPI-279.1, ADPI-279.2, ADPI-280, ADPI-281, ADPI-282, ADPI-283, ADPI-284, ADPI-285, ADPI-286, ADPI-287, ADPI-288, ADPI-289, ADPI-290, ADPI-291, ADPI-292, ADPI-293, ADPI-294, ADPI-295, ADPI-296, ADPI-297, ADPI-298, ADPI-299, ADPI-300, ADPI-301, ADPI-302, ADPI-303, ADPI-304, ADPI-305, ADPI-306, ADPI-307.1, ADPI-307.2, ADPI-308, ADPI-309, ADPI-311, ADPI-312, ADPI-313, ADPI-314, ADPI-315, ADPI-316.1, ADPI-316.2, ADPI-317, ADPI-318, ADPI-319, ADPI-320.1, ADPI-320.2, ADPI-321, ADPI-322, ADPI-323, ADPI-324, ADPI-325, ADPI-326, ADPI-327, ADPI-328, ADPI-329, ADPI-330, ADPI-331, ADPI-332, ADPI-333.1, ADPI-333.2, ADPI-334, ADPI-335, ADPI-336, ADPI-337, ADPI-338, ADPI-339, ADPI-340, ADPI-341, ADPI-342, ADPI-343, ADPI-344, ADPI-345, ADPI-346, ADPI-347, ADPI-348, ADPI-349, ADPI-350, ADPI-351, ADPI-352, ADPI-353, ADPI-354, ADPI-355, ADPI-356, ADPI-357, ADPI-358, ADPI-359, ADPI-360, ADPI-361, ADPI-362, ADPI-363, ADPI-364, ADPI-365.1, ADPI-365.2, ADPI-366, ADPI-367.1, ADPI-367.2, ADPI-368.1, ADPI-368.2, ADPI-369.1, ADPI-369.2, ADPI-370, ADPI-371, ADPI-372, ADPI-373, ADPI-374, ADPI-375, ADPI-376, ADPI-377, ADPI-378, ADPI-379, ADPI-380, ADPI-381, ADPI-382, ADPI-383, ADPI-384, ADPI-386, ADPI-387, ADPI-388, ADPI-389, ADPI-390, ADPI-391, ADPI-392, ADPI-393, ADPI-394, ADPI-395, ADPI-396, ADPI-397, ADPI-398, ADPI-399.1, ADPI-399.2, ADPI-400, ADPI-401, ADPI-402, ADPI-403, ADPI-404.1, ADPI-404.2, ADPI-405, ADPI-406, ADPI-407, ADPI-408, ADPI-409, ADPI-410, ADPI-411, ADPI-412.1, ADPI-412.2, ADPI-413, ADPI-414, ADPI-415, ADPI-416, ADPI-417, ADPI-418, ADPI-419, ADPI-420, ADPI-421, ADPI-422, ADPI-423, ADPI-424, ADPI-425, ADPI-426.1, ADPI-426.2, ADPI-427, ADPI-428, ADPI-429, ADPI-430, ADPI-431, ADPI-432, ADPI-433, ADPI-434, ADPI-435, ADPI-435, ADPI-436, ADPI-437, ADPI-438, ADPI-439, ADPI-440.1, ADPI-440.2, ADPI-441, ADPI-442, ADPI-443, ADPI-444, ADPI-445, ADPI-446, ADPI-447.1, ADPI-447.2, ADPI-448, ADPI-449, ADPI-450.1, ADPI-450.2, ADPI-451, ADPI-452, ADPI-453, ADPI-454, ADPI-455, ADPI-456, ADPI-457, ADPI-458, ADPI-459, ADPI-460, ADPI-461, ADPI-462, ADPI-463, ADPI-464, ADPI-465, ADPI-466.1, ADPI-466.2, ADPI-467, ADPI-468, ADPI-469.1, ADPI-469.2, ADPI-470, ADPI-471, ADPI-472, ADPI-473, ADPI-474, ADPI-475, ADPI-476, ADPI-477, ADPI-478.1, ADPI-478.2, ADPI-479, ADPI-480, ADPI-481, ADPI-482, ADPI-483, ADPI-484, ADPI-485, ADPI-486, ADPI-487, ADPI-488, ADPI-489, ADPI-490, ADPI-491, ADPI-492, ADPI-493.1, ADPI-493.2, ADPI-494, ADPI-495, ADPI-496, ADPI-497, ADPI-498, ADPI-499, ADPI-500, ADPI-501, ADPI-502, ADPI-503, ADPI-504, ADPI-505, ADPI-506, ADPI-507, ADPI-508, ADPI-509, ADPI-510, ADPI-511, ADPI-512, ADPI-513, ADPI-514, ADPI-515, ADPI-516, ADPI-517, ADPI-518, ADPI-519, ADPI-520, ADPI-521, ADPI-522, ADPI-523, ADPI-524, ADPI-525, ADPI-526.1, ADPI-526.2, ADPI-527, ADPI-528, ADPI-529, ADPI-530.1, ADPI-530.2, ADPI-531, ADPI-532, ADPI-533, ADPI-534, ADPI-535, ADPI-536, ADPI-537, ADPI-538, ADPI-539, ADPI-540, ADPI-541, ADPI-542, ADPI-543, ADPI-544, ADPI-545, ADPI-546, ADPI-547.1, ADPI-547.2, ADPI-547.3, ADPI-548.1, ADPI-548.2, ADPI-549, whereby the symptoms of Alzheimer's disease are ameliorated.

50. A method for identifying targets for therapeutic modulation of Alzheimer's disease wherein the activity of one or more of the following Alzheimer's Disease-Associated Protein Isoforms (ADPIs): ADPI-1, ADPI-3.1, ADPI-3.2, ADPI-3.3, ADPI-5.3, ADPI-6, ADPI-8, ADPI-9, ADPI-10, ADPI-11, ADPI-12.1, ADPI-12.2, ADPI-13, ADPI-15, ADPI-16, ADPI-22, ADPI-23, ADPI-24, ADPI-25.3, ADPI-25.2, ADPI-26, ADPI-27, ADPI-29, ADPI-31, ADPI-33.1, ADPI-35, ADPI-37, ADPI-39, ADPI-42, ADPI-54, ADPI-56, ADPI-61, ADPI-62, ADPI-66, ADPI-67, ADPI-68, ADPI-70, ADPI-72, ADPI-77, ADPI-78.2, ADPI-78.3, ADPI-79, ADPI-81.2, ADPI -82, ADPI-85, ADPI-87.1, ADPI-88, ADPI-90, ADPI-91.1, ADPI-91.2, ADPI-92, ADPI-94, ADPI-95, ADPI-97, ADPI-98, ADPI-101, ADPI-102.1, ADPI-103, ADPI-105.2, ADPI-105.1, ADPI-107, ADPI-108, ADPI-109, ADPI-111.1, ADPI-112, ADPI-113, ADPI-115, ADPI-117.1, ADPI-119, ADPI-120, ADPI-121, ADPI-124, ADPI-125, ADPI-126, ADPI-126, ADPI-126, ADPI-127, ADPI-128, ADPI-129, ADPI-130, ADPI-131, ADPI-132, ADPI-133, ADPI-138, ADPI-139, ADPI-140, ADPI-141, ADPI-142.2, ADPI-142.1, ADPI-143.1, ADPI-143.2, ADPI-144, ADPI-146.2, ADPI-146.1, ADPI-148, ADPI-149.1, ADPI-150, ADPI-151.2, ADPI-151.1, ADPI-152, ADPI-153.1, ADPI-153.2, ADPI-153.3, ADPI-154, ADPI-155, ADPI-156.2, ADPI-156.1, ADPI-157, ADPI-159.1,ADPI-159.2,ADPI-160,ADPI-162.2,ADPI-162.3, ADPI-162.1, ADPI-163, ADPI-165, ADPI-172, ADPI-173.1, ADPI-173.2, ADPI-175.2, ADPI-175.1, ADPI-176, ADPI-182, ADPI-183, ADPI-188.1, ADPI-189.2, ADPI-191, ADPI-193, ADPI-194.2, ADPI-196.2, ADPI-196.3, ADPI-196.1, ADPI-196.4, ADPI-202, ADPI-204, ADPI-208, ADPI-209, ADPI-216, ADPI-217, ADPI-220, ADPI-223, ADPI-228, ADPI-229, ADPI-230.1, ADPI-230.2, ADPI-232.1, ADPI-232.2, ADPI-236.2, ADPI-236.3, ADPI-237.2, ADPI-237.1, ADPI-240, ADPI-243, ADPI-245, ADPI-250, ADPI-251, ADPI-252.1, ADPI-252.2, ADPI-253, ADPI-254, ADPI-255, ADPI-256, ADPI-257.1, ADPI-257.2, ADPI-258, ADPI-259.1, ADPI-259.2, ADPI-260, ADPI-261, ADPI-262, ADPI-263, ADPI-264, ADPI-265, ADPI-266, ADPI-267, ADPI-268.1, ADPI-268.2, ADPI-269, ADPI-270, ADPI-271, ADPI-272, ADPI-273, ADPI-274, ADPI-275, ADPI-276, ADPI-277, ADPI-278.1, ADPI-278.2, ADPI-279.1, ADPI-279.2, ADPI-280, ADPI-281, ADPI-282, ADPI-283, ADPI-284, ADPI-285, ADPI-286, ADPI-287, ADPI-288, ADPI-289, ADPI-290, ADPI-291, ADPI-292, ADPI-293, ADPI-294, ADPI-295, ADPI-296, ADPI-297, ADPI-298, ADPI-299, ADPI-300, ADPI-301, ADPI-302, ADPI-303, ADPI-304, ADPI-305, ADPI-306, ADPI-307.1, ADPI-307.2, ADPI-308, ADPI-309, ADPI-311, ADPI-312, ADPI-313, ADPI-314, ADPI-315, ADPI-316.1, ADPI-316.2, ADPI-317, ADPI-318, ADPI-319, ADPI-320.1, ADPI-320.2, ADPI-321, ADPI-322, ADPI-323, ADPI-324, ADPI-325, ADPI-326, ADPI-327, ADPI-328, ADPI-329, ADPI-330, ADPI-331, ADPI-332, ADPI-333.1, ADPI-333.2, ADPI-334, ADPI-335, ADPI-336, ADPI-337, ADPI-338, ADPI-339, ADPI-340, ADPI-341, ADPI-342, ADPI-343, ADPI-344, ADPI-345, ADPI-346, ADPI-347, ADPI-348, ADPI-349, ADPI-350, ADPI-351, ADPI-352, ADPI-353, ADPI-354, ADPI-355, ADPI-356, ADPI-357, ADPI-358, ADPI-359, ADPI-360, ADPI-361, ADPI-362, ADPI-363, ADPI-364, ADPI-365.1, ADPI-365.2, ADPI-366, ADPI-367.1, ADPI-367.2, ADPI-368.1, ADPI-368.2, ADPI-369.1, ADPI-369.2, ADPI-370, ADPI-371, ADPI-372, ADPI-373, ADPI-374, ADPI-375, ADPI-376, ADPI-377, ADPI-378, ADPI-379, ADPI-380, ADPI-381, ADPI-382, ADPI-383, ADPI-384, ADPI-386, ADPI-387, ADPI-388, ADPI-389, ADPI-390, ADPI-391, ADPI-392, ADPI-393, ADPI-394, ADPI-395, ADPI-396, ADPI-397, ADPI-398, ADPI-399.1, ADPI-399.2, ADPI-400, ADPI-401, ADPI-402, ADPI-403, ADPI-404.1, ADPI-404.2, ADPI-405, ADPI-406, ADPI-407, ADPI-408, ADPI-409, ADPI-410, ADPI-411, ADPI-412.1, ADPI-412.2, ADPI-413, ADPI-414, ADPI-415, ADPI-416, ADPI-417, ADPI-418, ADPI-419, ADPI-420, ADPI-421, ADPI-422, ADPI-423, ADPI-424, ADPI-425, ADPI-426.1, ADPI-426.2, ADPI-427, ADPI-428, ADPI-429, ADPI-430, ADPI-431, ADPI-432, ADPI-433, ADPI-434, ADPI-435, ADPI-435, ADPI-436, ADPI-437, ADPI-438, ADPI-439, ADPI-440.1, ADPI-440.2, ADPI-441, ADPI-442, ADPI-443, ADPI-444, ADPI-445, ADPI-446, ADPI-447.1, ADPI-447.2, ADPI-448, ADPI-449, ADPI-450.1, ADPI-450.2, ADPI-451, ADPI-452, ADPI-453, ADPI-454, ADPI-455, ADPI-456, ADPI-457, ADPI-458, ADPI-459, ADPI-460, ADPI-461, ADPI-462, ADPI-463, ADPI-464, ADPI-465, ADPI-466.1, ADPI-466.2, ADPI-467, ADPI-468, ADPI-469.1, ADPI-469.2, ADPI-470, ADPI-471, ADPI-472, ADPI-473, ADPI-474, ADPI-475, ADPI-476, ADPI-477, ADPI-478.1, ADPI-478.2, ADPI-479, ADPI-480, ADPI-481, ADPI-482, ADPI-483, ADPI-484, ADPI-485, ADPI-486, ADPI-487, ADPI-488, ADPI-489, ADPI-490, ADPI-491, ADPI-492, ADPI-493.1, ADPI-493.2, ADPI-494, ADPI-495, ADPI-496, ADPI-497, ADPI-498, ADPI-499, ADPI-500, ADPI-501, ADPI-502, ADPI-503, ADPI-504, ADPI-505, ADPI-506, ADPI-507, ADPI-508, ADPI-509, ADPI-510, ADPI-511, ADPI-512, ADPI-513, ADPI-514, ADPI-515, ADPI-516, ADPI-517, ADPI-518, ADPI-519, ADPI-520, ADPI-521, ADPI-522, ADPI-523, ADPI-524, ADPI-525, ADPI-526.1, ADPI-526.2, ADPI-527, ADPI-528, ADPI-529, ADPI-530.1, ADPI-530.2, ADPI-531, ADPI-532, ADPI-533, ADPI-534, ADPI-535, ADPI-536, ADPI-537, ADPI-538, ADPI-539, ADPI-540, ADPI-541, ADPI-542, ADPI-543, ADPI-544, ADPI-545, ADPI-546, ADPI-547.1, ADPI-547.2, ADPI-547.3, ADPI-548.1, ADPI-548.2, ADPI-549 is utilized as a measure to determine whether a candidate target is effective for modulation of Alzheimer's disease.

51. A method of screening for agents effective for the treatment of Alzheimer's disease comprising:
(a) contacting ERK-2 with a first population of cells expressing a receptor for ERK-2 in the presence of a candidate agent;
(b) contacting ERK-2 with a second population of cells expressing said receptor in the presence of a control agent;
(c) comparing the binding of said ERK-2 to the first and second populations of cells, or comparing the level of induction of a cellular second messenger in the first and second populations of cells, or comparing the level of an ERK-2 mediated activity in the first and second population of cells; and
(d) testing for the ability of agents able to modulate the activity of ERK-2 to decrease clinical features of Alzheimer's disease in a Alzheimer's disease model system.

52. The method as claimed in claim 51, wherein the ERK-2 and/or the receptor are isolated and recombinantly produced.

53. The method as claimed in claim 51 or claim 52, wherein the cells are cerebellar granule cells and the ERK-2 mediated activity is protection of immature cerebellar cells from apoptotic death.

54. The method as claimed in claim 53, wherein the cells are retinoblastoma or cerebellar granule cells and the binding of ERK-2 to the cells is compared.

55. The method as claimed in any one of claims 51 to 54, wherein the candidate agent comprises at least 10 consecutive amino acids of ERK-2.

56. A method of screening for agents that modulate the binding of ERK-2 to a binding partner comprising:
(a) contacting ERK-2 with the ERK-2 binding partner in the presence of a candidate agent;
(b) contacting ERK-2 with a ERK-2 binding partner in the presence of a control agent; and
(c) comparing the binding of said ERK-2 to the binding partner in step (a) to the binding of said ERK-2 to the binding partner in step (b).

57. The method of claim 56, wherein the binding partner is an isolated 41 kDa ERK-2 receptor.
